(19) United States
(12) Patent Application Publication
(10) Pub. No.: US 2022/0351806 A1
(43) Pub. Date: Nov. 3, 2022

(54) Biomarker Panels for Guiding Dysregulated Host Response Therapy

(71) Applicant: Endpoint Health Inc., Palo Alto (US)

(72) Inventors: Diego Ariel Rey, Palo Alto, CA (US); Leonardo Maestri Teixeira, Palo Alto, CA (US); Hugo Yk Lam, Palo Alto, CA (US); Bayo Yh Lau, Palo Also, CA (US); Lijing Yao, Palo Alto, CA (US)

(21) Appl. No.: 17/766,018

(22) PCT Filed: Oct. 2, 2020
(86) PCT No.: PCT/US2020/054033

§ 371 Date: Apr. 1, 2022

Related U.S. Application Data
(60) Provisional application No. 62/909,530, filed on Oct. 2, 2019.
(60) Provisional application No. 63/009,331, filed on Apr. 13, 2020.

(51) Int. Cl. G16B 40/20; G16B 25/10; G06N 20/10

(52) U.S. Cl. CPC G16B 40/20; G16B 25/10; G06N 20/10

(57) ABSTRACT

A method for identifying a therapy recommendation for a subject exhibiting dysregulated host response is provided. A classification of the subject of subtype A, subtype B, or subtype C is obtained. The therapy recommendation for the subject is identified based at least in part on the classification. Responsive to the classification of the subject comprising subtype A, the therapy recommendation can be no immunosuppressive therapy. Responsive to the classification of the subject comprising subtype B, the therapy recommendation can be no therapy recommendation, immune stimulation therapy, suppression of immune regulation therapy, blocking of immune suppression therapy, blocking of complement activity therapy, and/or anti-inflammatory therapy. Responsive to the classification of the subject comprising subtype C, the therapy recommendation can be no therapy recommendation, immune stimulation therapy, suppression of immune regulation therapy, blocking of immune suppression therapy, modulators of coagulation therapy, and or modulators of vascular permeability therapy.

### CROSS REFERENCE TO RELATED APPLICATIONS

This application claims the benefit of and priority to U.S. Provisional Patent Application No. 62/909,530 filed Oct. 2, 2019 and U.S. Provisional Patent Application No. 63/009,331 filed Apr. 13, 2020, the entire disclosures of which are each hereby incorporated by reference in its entirety for all purposes.

### BACKGROUND

Host response is a complex pathophysiologic process arising from an insult such as infection, trauma, burns, and other injuries. Diverse host responses can manifest clinically, including immune response, inflammatory response, coagulopathic response, and any other type of response to bodily insult. In some cases, host response to bodily insult can go awry, causing acute, life-threatening syndromes. As referred to herein, “dysregulated host response” refers to such cases in which host response to bodily insult goes awry, and thereby causes acute, life-threatening syndromes. For example, dysregulated immune response to infection can manifest clinically as sepsis. As another example, dysregulated immune response to a non-infection insult, such as, for example, burns, can manifest clinically as Systemic Inflammatory Response Syndrome (SIRS)⁵².

Sepsis is an acute, life-threatening syndrome caused by a dysregulated immune response to infection.^{1,2} Approximately 1.7 million patients are diagnosed with sepsis each year.¹⁵ According to a recent study based on electronic medical record data from more than 7 million hospitalizations across 409 US hospitals, sepsis has an estimated 6% hospital admission rate.¹⁵ The average length of stay for septic patients is 75% greater than most other conditions, and its mortality accounts for more than 50% of hospital deaths in hospitals.¹⁶ Sepsis ranks as one of the highest costs among all hospital admissions, representing approximately 13% of total US hospital costs, or more than $24 billion in hospital expenses.¹⁶ Sepsis costs increase based on sepsis severity level and timing of clinical presentation (e.g., at the hospital admission or during the hospital stay). Sepsis cases that were not present at hospital admission spend almost twice the amount of time in the hospital, in the intensive care unit, and on mechanical ventilation, compared to patients in which sepsis was presented at the hospital admission.¹⁷

Beyond early recognition, according to the Surviving Sepsis Campaign guidelines, the cornerstone for initial sepsis management is currently based on five main actions known as the “1-hour bundle”. The “1-hour bundle” includes: (1) lactate level measurement; (2) blood cultures collection; (3) broad-spectrum antibiotics administration; (4) rapid fluid administration of 30 ml/kg crystalloid for hypotension or lactate ≥4 mmol/L and (5) vasopressors for patients that remain hypotensive during or after resuscitation to maintain mean arterial pressure ≥65 mmHg.¹⁸

After applying this initial approach, patients are frequently assessed over the following hours according to their clinical response. For those patients with poor clinical response, further adjustments, in terms of the amount of fluids given and/or in terms of the choice of antibiotic therapy and measurements for source control (e.g. device removal, surgical procedures, or additional investigation), can be made.

Despite the appropriate application of these actions, close to 30% of septic patients remain hypotensive, requiring vasopressors to maintain a mean arterial pressure ≥65 mmHg, and then are characterized as having septic shock,¹⁹ a subtype of sepsis and a condition that has an expected hospital mortality in excess of 40%.¹ Of septic shock patients, close to 40% continue to show no clinical improvement (refractory septic shock), defined as a systolic blood pressure <90 mmHg for more than one hour following both adequate fluid resuscitation and vasopressor therapy. In this set of refractory septic shock patients, glucocorticoid therapy may provide improvement.¹

Corticosteroids remain a controversial therapy for sepsis patients. Specifically, current guidelines provide a weak recommendation for corticosteroids sepsis patients by stating that either steroids and no steroids are reasonable management options.²⁰

Despite often-promising preclinical studies, more than 100 interventional trials have failed to demonstrate significantly improved survival among sepsis patients, leaving clinicians with limited interventions, and patients with mortality rates as high as 40% among those who develop septic shock.⁴⁻¹²

Similar trends have also been observed for other manifestations of dysregulated host response not caused by infection, such as, for examples SIRS, which can be caused by severe burns.

### SUMMARY

Embodiments disclosed herein relate to methods, non-transitory computer-readable mediums, systems, and kits for determining patient subtypes, determining therapy recommendations for patients, and generating therapeutic hypotheses for patient subtypes. In various embodiments described herein, the methods involve analyzing quantitative data of one or more biomarker sets derived from a sample obtained from a patient using a patient subtype classifier. The patient subtype classifier outputs a classification for the patient that guides the determination of a therapy recommendation.

Disclosed herein is a method for determining a patient subtype, the method comprising: obtaining or having obtained quantitative data for at least one biomarker set selected from the group consisting of the biomarker sets of group 1, group 2, group 3, group 4, or group 5, wherein group 1 comprises biomarker 1, biomarker 2, and biomarker 3, wherein biomarker 1 is one or more of EVL, BTN3A2, HLA-DPA1, IDH3A, ACBD3, EXOSC10, SNRK, or MMP8, wherein biomarker 2 is one or more of SERPINB1 or GSPT1, and wherein biomarker 3 is one or more of MPP1, HMBS, TAL1, C9orf78, POLR2L, SLC27A3, BTN3A2, DDX50, FCHSD2, GSTK1, UBE2E1, TNFRSF1A, PRPF3, or TOMM70A, wherein group 2 comprises biomarker 4, biomarker 5, and biomarker 6, wherein biomarker 4 is one or more of ZNF831, MME, CD3G, or STOM, wherein biomarker 5 is one or more of ECSIT, LAT, or NCOA4, and wherein biomarker 6 is one or more of SLC1A5, IGF2BP2, or ANXA3, wherein group 3 comprises biomarker 7, biomarker 8, and biomarker 9, wherein biomarker 7 is one or more of C14orf159 or PUM2, wherein biomarker 8 is one or more of EPB42 or RPS6KA5, and wherein biomarker 9 is one or more of EPB42 or GBP2; and wherein group 4 comprises biomarker 10, biomarker 11, and biomarker 12, wherein biomarker 10 is one or more of MSH2, DCTD, or MMP8, wherein biomarker 11 is one or more of HK3, UCP2, or NUP88, and wherein biomarker 12 is one or more of GABARAPL2 or CASP4; and wherein group 5 comprises biomarker 13, biomarker 14, and biomarker 15, wherein biomarker 13 is one or more of STOM, MME, BNT3A2, HLA-DPA1, ZNF831, or CD3G, wherein biomarker 14 is one or more of EPB42, GSPT1, LAT, HK3, or SERPINB1, and wherein biomarker 15 is one or more of SLC1A5, IGF2BP2, ANXA3, GBP2, TNFRSF1, BTN3A2, OR TNFRSF1A; and determining a classification of a subject based on the quantitative data using a patient subtype classifier.

In various embodiments, the at least one biomarker set is group 5, and wherein biomarker 13 is one or more of STOM, MME, BNT3A2, or HLA-DPA1. In various embodiments, wherein the at least one biomarker set is group 5, and wherein biomarker 14 is one or more of EPB42, GSPT1, LAT, HK3, or SERPINB1. In various embodiments, the at least one biomarker set is group 5, and wherein biomarker 15 is one or more of SLC1A5, IGF2BP2, or ANXA3.

Additionally disclosed herein is a method for determining a therapy recommendation for a patient, the method comprising: obtaining or having obtained quantitative data for two or more biomarkers selected from the group consisting of EVL, BTN3A2, HLA-DPA1, IDH3A, ACBD3, EXOSC10, SNRK, MMP8, SERPINB1, GSPT1, MPP1, HMBS, TAL1, C9orf78, POLR2L, SLC27A3, DDX50, FCHSD2, GSTK1, UBE2E1, TNFRSF1A, PRPF3, TOMM70A, ZNF831, MME, CD3G, STOM, ECSIT, LAT, NCOA4, SLC1A5, IGF2BP2, ANXA3, C14orf159, PUM2, EPB42, RPS6KA5, GBP2, MSH2, DCTD, HK3, UCP2, NUP88, GABARAPL2, and CASP4; and determining a classification of a subject based on the quantitative data using a patient subtype classifier.

Additionally disclosed herein is a method for determining a therapy recommendation for a patient, the method comprising: obtaining or having obtained quantitative data for at least one biomarker set selected from the group consisting of the biomarker sets of group 1, group 2, group 3, group 4, or group 5, wherein group 1 comprises two or more biomarkers selected from a group consisting of EVL, BTN3A2, HLA-DPA1, IDH3A, ACBD3, EXOSC10, SNRK, MMP8, SERPINB1 GSPT1, MPP1, HMBS, TAL1, C9orf78, POLR2L, SLC27A3, BTN3A2, DDX50, FCHSD2, GSTK1, UBE2E1, TNFRSF1A, PRPF3, and TOMM70A, wherein group 2 comprises two or more biomarkers selected from a group consisting of ZNF831, MME, CD3G, STOM, ECSIT, LAT, NCOA4, SLC1A5, IGF2BP2, and ANXA3, wherein group 3 comprises two or more biomarkers selected from a group consisting of C14orf159, PUM2, EPB42, RPS6KA5, EPB42, and GBP2; and wherein group 4 comprises two or more biomarkers selected from a group consisting of MSH2, DCTD, MMP8, HK3, UCP2, NUP88, GABARAPL2, and CASP4; and wherein group 5 comprises two or more biomarkers selected from a group consisting of STOM, MME, BNT3A2, HLA-DPA1, ZNF831, CD3G, EPB42, GSPT1, LAT, HK3, SERPINB1, SLC1A5, IGF2BP2, ANXA3, GBP2, TNFRSF1, BTN3A2, and TNFRSF1A; and determining a classification of a subject based on the quantitative data using a patient subtype classifier. In various embodiments, methods described herein further comprise identifying a therapy recommendation for the subject based at least in part on the classification.

Additionally disclosed herein is a method for determining a therapy recommendation for a patient, the method comprising: obtaining a classification of a subject exhibiting a dysregulated host response, the classification having been determined by: obtaining or having obtained quantitative data for at least one biomarker set selected from the group consisting of the biomarker sets of group 1, group 2, group 3, group 4, or group 5, wherein group 1 comprises biomarker 1, biomarker 2, and biomarker 3, wherein biomarker 1 is one or more of EVL, BTN3A2, HLA-DPA1, IDH3A, ACBD3, EXOSC10, SNRK, or MMP8, wherein biomarker 2 is one or more of SERPINB1 or GSPT1, and wherein biomarker 3 is one or more of MPP1, HMBS, TAL1, C9orf78, POLR2L, SLC27A3, BTN3A2, DDX50, FCHSD2, GSTK1, UBE2E1, TNFRSF1A, PRPF3, or TOMM70A, wherein group 2 comprises biomarker 4, biomarker 5, and biomarker 6, wherein biomarker 4 is one or more of ZNF831, MME, CD3G, or STOM, wherein biomarker 5 is one or more of ECSIT, LAT, or NCOA4, and wherein biomarker 6 is one or more of SLC1A5, IGF2BP2, or ANXA3, wherein group 3 comprises biomarker 7, biomarker 8, and biomarker 9, wherein biomarker 7 is one or more of C14orf159 or PUM2, wherein biomarker 8 is one or more of EPB42 or RPS6KA5, and wherein biomarker 9 is one or more of EPB42 or GBP2; and wherein group 4 comprises biomarker 10, biomarker 11, and biomarker 12, wherein biomarker 10 is one or more of MSH2, DCTD, or MMP8, wherein biomarker 11 is one or more of HK3, UCP2, or NUP88, and wherein biomarker 12 is one or more of GABARAPL2 or CASP4; and wherein group 5 comprises biomarker 13, biomarker 14, and biomarker 15, wherein biomarker 13 is one or more of STOM, MME, BNT3A2, HLA-DPA1, ZNF831, or CD3G, wherein biomarker 14 is one or more of EPB42, GSPT1, LAT, HK3, or SERPINB1, and wherein biomarker 15 is one or more of SLC1A5, IGF2BP2, ANXA3, GBP2, TNFRSF1, BTN3A2, OR TNFRSF1A; and determining the classification based on the quantitative data using a patient subtype classifier; and identifying a therapy recommendation for the subject based at least in part on the classification.

In various embodiments, the dysregulated host response of the subject comprises one of sepsis and dysregulated host response not caused by infection. In various embodiments, the classification of the subject comprises one of subtype A or subtype B. In various embodiments, the classification of the subject comprises one of subtype A, subtype B, or subtype C. In various embodiments, responsive to the classification of the subject comprising subtype A, the therapy recommendation identified for the subject comprises at least no immunosuppressive therapy. In various embodiments, responsive to the classification of the subject comprising subtype A, the therapy recommendation identified for the subject further comprises at least no corticosteroid therapy. In various embodiments, the therapy recommendation identified for the subject further comprises no hydrocortisone.

In various embodiments, responsive to the classification of the subject comprising subtype B, the therapy recommendation identified for the subject comprises at least one of no therapy recommendation, immune stimulation therapy, suppression of immune regulation therapy, blocking of immune suppression therapy, blocking of complement activity therapy, and anti-inflammatory therapy. In various embodiments, responsive to the classification of the subject comprising subtype B, the therapy recommendation identified for the subject further comprises at least one of a checkpoint inhibitor, a blocker of complement components, a blocker of complement component receptors, and a blocker of a pro-inflammatory cytokine. In various embodiments, the therapy recommendation identified for the subject further comprises at least one of GM-CSF, anti-PD-1, anti-PD-L1, anti-CLTA-4, anti-CEACAM-1, anti-TIM-3, anti-BTLA, INF-gamma, IFN-beta 1a regulator, IL-22 agonist, IFN-alpha regulator, IFN-lambda regulator, IFN-alpha 2b stimulant, anti-C5a, anti-C3a, anti-C5aR, anti-C3aR, anti-TNF-alpha, and anti-IL-6, Anti-HMGB1, ST2 antibody, IL-33 antibody.

In various embodiments, responsive to the classification of the subject comprising subtype C, the therapy recommendation identified for the subject comprises at least one of no therapy recommendation, immune stimulation therapy, suppression of immune regulation therapy, blocking of immune suppression therapy, modulators of coagulation therapy, and modulators of vascular permeability therapy. In various embodiments, responsive to the classification of the subject comprising subtype C, the therapy recommendation identified for the subject further comprises at least one of a checkpoint inhibitor and an anticoagulant. In various embodiments, the therapy recommendation identified for the subject further comprises at least one of GM-CSF, anti-PD-1, anti-PD-L1, anti-CLTA-4, anti-CEACAM-1, anti-TIM-3, anti-BTLA, INF-gamma, IFN-beta 1a regulator, IL-22 agonist, IFN-alpha regulator, IFN-lambda regulator, IFN-alpha 2b stimulant, activated protein C, antithrombin, and thrombomodulin. In various embodiments, methods further comprise administering or having administered therapy to the subject based on the therapy recommendation.

In various embodiments, obtaining or having obtained quantitative data comprises: obtaining a sample from a subject exhibiting dysregulated host response, wherein the sample comprises a plurality of biomarkers; and determining the quantitative data from the obtained sample. In various embodiments, the obtained sample comprises a blood sample from the subject. In various embodiments, the subject exhibiting dysregulated host response does not exhibit shock, and wherein the at least one biomarker set is one of group 1, group 3, or group 4. In various embodiments, the subject exhibiting dysregulated host response is further exhibiting shock, and wherein the at least one biomarker set is one of group 1, group 2, group 4, group 5, group 6, group 7, or group 8. In various embodiments, the subject exhibiting dysregulated host response is an adult subject, and wherein the at least one biomarker set is one of group 1, group 2, group 3, group 5, group 6, group 7, or group 8. In various embodiments, the subject exhibiting dysregulated host response is a pediatric subject, and wherein the at least one biomarker set is one of group 1, group 4, group 5, group 6, group 7, or group 8.

In various embodiments, the quantitative data is determined by one of RT-qPCR (quantitative reverse transcription polymerase chain reaction), qPCR (quantitative polymerase chain reaction), PCR (polymerase chain reaction), RT-PCR (reverse transcription polymerase chain reaction), SDA (strand displacement amplification), RPA (recombinase polymerase amplification), MDA (multiple displacement amplification), HDA (helicase dependent amplification), LAMP (loop-mediated isothermal amplification), RCA (rolling circle amplification), NASBA (nucleic acid-sequence-based amplification), and any other isothermal or thermocycled amplification reaction.

In various embodiments, the quantitative data is determined by: contacting a sample with a reagent; generating a plurality of complexes between the reagent and the plurality of biomarkers in the sample; and detecting the plurality of complexes to obtain a dataset associated with the sample, wherein the dataset comprises the quantitative data. In various embodiments, the classification of the subject is determined by: determining, for at least one candidate classification of the subject, a classification-specific score for the subject; determining, by the patient subtype classifier, based on the classification-specific score, the classification of the subject.

In various embodiments, determining the classification-specific score comprises: determining a first subscore of the quantitative data for the subject for one or more biomarkers of the candidate classification, wherein the quantitative data for the subject for the one or more biomarkers of the candidate classification are increased relative to the quantitative data for the one or more biomarkers for one or more control subjects; determining a second subscore of the quantitative expression for the subject for one or more additional biomarkers of the candidate classification, wherein the quantitative data for the subject for the one or more additional biomarkers of the candidate classification are decreased relative to the quantitative data for the one or more additional biomarkers for the one or more control subjects; and determining a difference between the first subscore and the second subscore, the first and second geometric subscore optionally subject to scaling, and the difference comprising the classification-specific score for the subject. In various embodiments, one or both of the first subscore and the second subscore are geometric means.

In various embodiments, the patient subtype classifier is a machine-learned model. In various embodiments, the machine-learned model is a support vector machine (SVM). In various embodiments, the support vector machine receives, as input, one or more classification-specific scores and outputs the classification of the subject. In various embodiments, the patient subtype classifier determines the classification of the subject by: comparing the classification-specific scores to one or more threshold values; and determining the classification of the subject based on the comparisons. In various embodiments, at least one of the one or more threshold values is a fixed value. In various embodiments, at least one of the one or more threshold values is determined using training samples, the at least one threshold value representing a value on a ROC curve nearest to maximum sensitivity or maximum specificity.

In various embodiments, methods disclosed herein further comprise, prior to determining a classification of the subject using a patient subtype classifier, normalizing the quantitative data based on quantitative data for one or more housekeeping genes. In various embodiments, the candidate classifications of the subject comprise subtype A, subtype B, and subtype C. In various embodiments, the at least one biomarker set is group 1, and wherein the patient subtype classifier has an average accuracy of at least 82.93%. In various embodiments, the at least one biomarker set is group 2, and wherein the patient subtype classifier has an average accuracy of at least 89.6%. In various embodiments, the at least one biomarker set is group 3, and wherein the patient subtype classifier has an average accuracy of at least 86.3%. In various embodiments, the at least one biomarker set is group 4, and wherein the patient subtype classifier has an average accuracy of at least 98.3%.

In various embodiments, the therapy recommendation identified for the subject further comprises corticosteroid therapy, no corticosteroid therapy, or no therapy recommendation. In various embodiments, the therapy recommendation comprises a no corticosteroid therapy, wherein the no corticosteroid therapy is identified by determining that a statistical significance of a reduction in mortality of subjects exhibiting dysregulated host response not provided corticosteroid therapy is greater than or equal to a threshold statistical significance. In various embodiments, the therapy recommendation comprises a no corticosteroid therapy, wherein the no corticosteroid therapy is identified by determining that the classification of the subject comprises a subtype likely to be adversely responsive to corticosteroid therapy. In various embodiments, the subtype is subtype A or subtype C. In various embodiments, the therapy recommendation comprises a corticosteroid therapy, wherein the corticosteroid therapy is identified by determining that a statistical significance of a reduction in mortality of subjects exhibiting dysregulated host response and provided corticosteroid therapy is greater than or equal to a threshold statistical significance.

In various embodiments, the therapy recommendation comprises a corticosteroid therapy, wherein the corticosteroid therapy is identified by determining that the classification of the subject comprises a subtype likely to be favorably responsive to corticosteroid therapy. In various embodiments, the subtype is subtype B.

In various embodiments, the therapy recommendation identified for the subject comprises a no therapy recommendation, wherein the no therapy recommendation is identified at least by: determining that a statistical significance of a reduction in mortality of subjects exhibiting dysregulated host response and not provided corticosteroid therapy is less than a threshold statistical significance; and determining that a statistical significance of a reduction in mortality of subjects exhibiting dysregulated host response and provided corticosteroid therapy is less than a threshold statistical significance. In various embodiments, a statistical significance comprises a p-value, and wherein the threshold statistical significance comprises at least 0.1.

In various embodiments, the therapy recommendation identified for the subject comprises a no corticosteroid therapy, wherein the dysregulated host response comprises dysregulated host response not caused by infection, and wherein the at least one biomarker set is group 1 or group 4. In various embodiments, the no corticosteroid therapy is identified by determining that the classification of the subject comprises a subtype likely to be adversely responsive to corticosteroid therapy. In various embodiments, the subtype is subtype A or subtype C.

In various embodiments, the therapy recommendation identified for the subject further comprises no therapy recommendation, wherein the no therapy recommendation is identified by determining that the classification of the subject comprises subtype B. In various embodiments, the therapy recommendation identified for the subject comprises a no corticosteroid therapy, wherein the dysregulated host response comprises sepsis, wherein the at least one biomarker set is one of group 2, group 3, or group 4.

In various embodiments, the no corticosteroid therapy is identified by determining that the classification of the subject comprises a subtype likely to be adversely responsive to corticosteroid therapy. In various embodiments, the subtype is subtype A. In various embodiments, the therapy recommendation identified for the subject further comprises a no therapy recommendation, wherein the no therapy recommendation is identified by determining that the classification of the subject comprises a subtype likely to be non-responsive to corticosteroid therapy. In various embodiments, the subtype is subtype B or subtype C.

In various embodiments, the therapy recommendation identified for the subject further comprises a no corticosteroid therapy, wherein the dysregulated host response comprises dysregulated host response not caused by infection, and wherein the at least one biomarker set is group 2. In various embodiments, the no corticosteroid therapy is identified by determining that the classification of the subject comprises a subtype like to be adversely responsive to corticosteroid therapy. In various embodiments, the subtype is subtype C.

In various embodiments, the therapy recommendation identified for the subject further comprises a no therapy recommendation, wherein the no therapy recommendation is identified by determining that the classification of the subject comprises a subtype like to be non-responsive to corticosteroid therapy. In various embodiments, the subtype is subtype A or subtype B. In various embodiments, the therapy recommendation identified for the subject comprises a no corticosteroid therapy, wherein the dysregulated host response comprises dysregulated host response not caused by infection, and wherein the at least one biomarker set is group 3. In various embodiments, the no corticosteroid therapy is identified by determining that the classification of the subject comprises a subtype like to be non-responsive to corticosteroid therapy. In various embodiments, the subtype is subtype A or subtype C. In various embodiments, the therapy recommendation identified for the subject further comprises corticosteroid therapy, wherein the corticosteroid therapy is identified by determining that the classification of the subject comprises a subtype likely to be responsive to corticosteroid therapy. In various embodiments, the subtype is subtype B.

Additionally disclosed herein is a method for identifying a candidate therapeutic, the method comprising: accessing a differentially expressed gene database comprising gene level fold changes between patients of different subtypes; determining at least a threshold number of genes are differentially expressed in patients of a first subtype in comparison to patients of a second subtype, wherein each of the differentially expressed genes is involved in a common biological pathway; and determining a candidate therapeutic likely to be effective for patients of the first subtype, wherein the candidate therapeutic is effective in modulating expression of at least one of the genes that are differentially expressed in patients of the first subtype. In various embodiments, the differentially expressed gene database is generated by: obtaining labeled patient data, wherein labels of the labeled patient data identify patients that are classified into one of two or more subtypes; generating the differentially expressed gene database for at least one or more genes by at least determining gene-level fold changes between patient data with a label indicating a first subtype and patient data with a label indicating a second subtype. In various embodiments, the labels of the labeled patient data are generated by applying a clustering analysis or by applying a patient subtype classifier. In various embodiments, at least the threshold number of genes is at least three genes, at least four genes, at least five genes, at least six genes, at least seven genes, at least eight genes, at least nine genes, or at least ten genes. In various embodiments, determining a candidate therapeutic for patients of the first subtype further comprises: analyzing one or both of: therapeutic pharmacology data comprising data for the candidate therapeutic; and host response pathobiology comprising data for patients of the first subtype.

Additionally disclosed herein a non-transitory computer readable medium for determining a patient subtype, the non-transitory computer readable medium comprising instructions that, when executed by a processor, cause the processor to: obtain quantitative data for at least one biomarker set selected from the group consisting of the biomarker sets of group 1, group 2, group 3, group 4, or group 5, wherein group 1 comprises biomarker 1, biomarker 2, and biomarker 3, wherein biomarker 1 is one or more of EVL, BTN3A2, HLA-DPA1, IDH3A, ACBD3, EXOSC10, SNRK, or MMP8, wherein biomarker 2 is one or more of SERPINB1 or GSPT1, and wherein biomarker 3 is one or more of MPP1, HMBS, TAL1, C9orf78, POLR2L, SLC27A3, BTN3A2, DDX50, FCHSD2, GSTK1, UBE2E1, TNFRSF1A, PRPF3, or TOMM70A, wherein group 2 comprises biomarker 4, biomarker 5, and biomarker 6, wherein biomarker 4 is one or more of ZNF831, MME, CD3G, or STOM, wherein biomarker 5 is one or more of ECSIT, LAT, or NCOA4, and wherein biomarker 6 is one or more of SLC1A5, IGF2BP2, or ANXA3, wherein group 3 comprises biomarker 7, biomarker 8, and biomarker 9, wherein biomarker 7 is one or more of C14orf159 or PUM2, wherein biomarker 8 is one or more of EPB42 or RPS6KA5, and wherein biomarker 9 is one or more of EPB42 or GBP2; and wherein group 4 comprises biomarker 10, biomarker 11, and biomarker 12, wherein biomarker 10 is one or more of MSH2, DCTD, or MMP8, wherein biomarker 11 is one or more of HK3, UCP2, or NUP88, and wherein biomarker 12 is one or more of GABARAPL2 or CASP4; and wherein group 5 comprises biomarker 13, biomarker 14, and biomarker 15, wherein biomarker 13 is one or more of STOM, MME, BNT3A2, HLA-DPA1, ZNF831, or CD3G, wherein biomarker 14 is one or more of EPB42, GSPT1, LAT, HK3, or SERPINB1, and wherein biomarker 15 is one or more of SLC1A5, IGF2BP2, ANXA3, GBP2, TNFRSF1, BTN3A2, OR TNFRSF1A; and determine a classification of a subject based on the quantitative data using a patient subtype classifier.

In various embodiments, the at least one biomarker set is group 5, and wherein biomarker 13 is one or more of STOM, MME, BNT3A2, or HLA-DPA1. In various embodiments, the at least one biomarker set is group 5, and wherein biomarker 14 is one or more of EPB42, GSPT1, LAT, HK3, or SERPINB1. In various embodiments, the at least one biomarker set is group 5, and wherein biomarker 15 is one or more of SLC1A5, IGF2BP2, or ANXA3.

Additionally disclosed herein is a non-transitory computer readable medium for determining a therapy recommendation for a patient, the non-transitory computer readable medium comprising instructions that, when executed by a processor, cause the processor to: obtain quantitative data for two or more biomarkers selected from the group consisting of EVL, BTN3A2, HLA-DPA1, IDH3A, ACBD3, EXOSC10, SNRK, MMP8, SERPINB1, GSPT1, MPP1, HMBS, TAL1, C9orf78, POLR2L, SLC27A3, DDX50, FCHSD2, GSTK1, UBE2E1, TNFRSF1A, PRPF3, TOMM70A, ZNF831, MME, CD3G, STOM, ECSIT, LAT, NCOA4, SLC1A5, IGF2BP2, ANXA3, C14orf159, PUM2, EPB42, RPS6KA5, GBP2, MSH2, DCTD, HK3, UCP2, NUP88, GABARAPL2, and CASP4; and determine a classification of a subject based on the quantitative data using a patient subtype classifier.

Additionally disclosed herein is a non-transitory computer readable medium for determining a therapy recommendation for a patient, the non-transitory computer readable medium comprising instructions that, when executed by a processor, cause the processor to: obtain quantitative data for at least one biomarker set selected from the group consisting of the biomarker sets of group 1, group 2, group 3, group 4, or group 5, wherein group 1 comprises two or more biomarkers selected from a group consisting of EVL, BTN3A2, HLA-DPA1, IDH3A, ACBD3, EXOSC10, SNRK, MMP8, SERPINB1 GSPT1, MPP1, HMBS, TAL1, C9orf78, POLR2L, SLC27A3, BTN3A2, DDX50, FCHSD2, GSTK1, UBE2E1, TNFRSF1A, PRPF3, and TOMM70A, wherein group 2 comprises two or more biomarkers selected from a group consisting of ZNF831, MME, CD3G, STOM, ECSIT, LAT, NCOA4, SLC1A5, IGF2BP2, and ANXA3, wherein group 3 comprises two or more biomarkers selected from a group consisting of C14orf159, PUM2, EPB42, RPS6KA5, EPB42, and GBP2; and wherein group 4 comprises two or more biomarkers selected from a group consisting of MSH2, DCTD, MMP8, HK3, UCP2, NUP88, GABARAPL2, and CASP4; and wherein group 5 comprises two or more biomarkers selected from a group consisting of STOM, MME, BNT3A2, HLA-DPA1, ZNF831, CD3G, EPB42, GSPT1, LAT, HK3, SERPINB1, SLC1A5, IGF2BP2, ANXA3, GBP2, TNFRSF1, BTN3A2, and TNFRSF1A; and determine a classification of a subject based on the quantitative data using a patient subtype classifier. In various embodiments, the instructions further comprise instructions that, when executed by the processor, cause the processor to identify a therapy recommendation for the subject based at least in part on the classification.

Additionally disclosed herein is a non-transitory computer readable medium for determining a therapy recommendation for a subject, the non-transitory computer readable medium comprising instructions that, when executed by a processor, cause the processor to: obtain a classification of the subject exhibiting a dysregulated host response, the classification having been determined by: obtaining or having obtained quantitative data for at least one biomarker set selected from the group consisting of the biomarker sets of group 1, group 2, group 3, group 4, or group 5, wherein group 1 comprises biomarker 1, biomarker 2, and biomarker 3, wherein biomarker 1 is one or more of EVL, BTN3A2, HLA-DPA1, IDH3A, ACBD3, EXOSC10, SNRK, or MMP8, wherein biomarker 2 is one or more of SERPINB1 or GSPT1, and wherein biomarker 3 is one or more of MPP1, HMBS, TAL1, C9orf78, POLR2L, SLC27A3, BTN3A2, DDX50, FCHSD2, GSTK1, UBE2E1, TNFRSF1A, PRPF3, or TOMM70A, wherein group 2 comprises biomarker 4, biomarker 5, and biomarker 6, wherein biomarker 4 is one or more of ZNF831, MME, CD3G, or STOM, wherein biomarker 5 is one or more of ECSIT, LAT, or NCOA4, and wherein biomarker 6 is one or more of SLC1A5, IGF2BP2, or ANXA3, wherein group 3 comprises biomarker 7, biomarker 8, and biomarker 9, wherein biomarker 7 is one or more of C14orf159 or PUM2, wherein biomarker 8 is one or more of EPB42 or RPS6KA5, and wherein biomarker 9 is one or more of EPB42 or GBP2; and wherein group 4 comprises biomarker 10, biomarker 11, and biomarker 12, wherein biomarker 10 is one or more of MSH2, DCTD, or MMP8, wherein biomarker 11 is one or more of HK3, UCP2, or NUP88, and wherein biomarker 12 is one or more of GABARAPL2 or CASP4; and wherein group 5 comprises biomarker 13, biomarker 14, and biomarker 15, wherein biomarker 13 is one or more of STOM, MME, BNT3A2, HLA-DPA1, ZNF831, or CD3G, wherein biomarker 14 is one or more of EPB42, GSPT1, LAT, HK3, or SERPINB1, and wherein biomarker 15 is one or more of SLC1A5, IGF2BP2, ANXA3, GBP2, TNFRSF1, BTN3A2, OR TNFRSF1A; and determining the classification based on the quantitative data using a patient subtype classifier; and identify a therapy recommendation for the subject based at least in part on the classification.

In various embodiments, the dysregulated host response of the subject comprises one of sepsis and dysregulated host response not caused by infection. In various embodiments, the classification of the subject comprises one of subtype A or subtype B. In various embodiments, the classification of the subject comprises one of subtype A, subtype B, or subtype C. In various embodiments, responsive to the classification of the subject comprising subtype A, the therapy recommendation identified for the subject comprises at least no immunosuppressive therapy. In various embodiments, responsive to the classification of the subject comprising subtype A, the therapy recommendation identified for the subject further comprises at least no corticosteroid therapy. In various embodiments, the therapy recommendation identified for the subject further comprises no hydrocortisone.

In various embodiments, responsive to the classification of the subject comprising subtype B, the therapy recommendation identified for the subject comprises at least one of no therapy recommendation, immune stimulation therapy, suppression of immune regulation therapy, blocking of immune suppression therapy, blocking of complement activity therapy, and anti-inflammatory therapy. In various embodiments, responsive to the classification of the subject comprising subtype B, the therapy recommendation identified for the subject further comprises at least one of a checkpoint inhibitor, a blocker of complement components, a blocker of complement component receptors, and a blocker of a pro-inflammatory cytokine. In various embodiments, the therapy recommendation identified for the subject further comprises at least one of GM-CSF, anti-PD-1, anti-PD-L1, anti-CLTA-4, anti-CEACAM-1, anti-TIM-3, anti-BTLA, IL-7, INF-gamma, IFN-beta 1a regulator, IL-22 agonist, IFN-alpha regulator, IFN-lambda regulator, IFN-alpha 2b stimulant, anti-C5a, anti-C3a, anti-C5aR, anti-C3aR, anti-TNF-alpha, and anti-IL-6, Anti-HMGB1, ST2 antibody, IL-33 antibody. In various embodiments, responsive to the classification of the subject comprising subtype C, the therapy recommendation identified for the subject comprises at least one of no therapy recommendation, immune stimulation therapy, suppression of immune regulation therapy, blocking of immune suppression therapy, modulators of coagulation therapy, and modulators of vascular permeability therapy.

In various embodiments, responsive to the classification of the subject comprising subtype C, the therapy recommendation identified for the subject further comprises at least one of a checkpoint inhibitor and an anticoagulant. In various embodiments, the therapy recommendation identified for the subject further comprises at least one of GM-CSF, anti-PD-1, anti-PD-L1, anti-CLTA-4, anti-CEACAM-1, anti-TIM-3, anti-BTLA, IL-7, INF-gamma, IFN-beta 1a regulator, IL-22 agonist, IFN-alpha regulator, IFN-lambda regulator, IFN-alpha 2b stimulant, activated protein C, antithrombin, and thrombomodulin.

In various embodiments, the instructions that cause the processor to obtain quantitative data further comprises instructions that, when executed by the processor, cause the processor to: obtain a sample from a subject exhibiting dysregulated host response, wherein the sample comprises a plurality of biomarkers; and determine the quantitative data from the obtained sample. In various embodiments, the obtained sample comprises a blood sample from the subject.

In various embodiments, the subject exhibiting dysregulated host response does not exhibit shock, and wherein the at least one biomarker set is one of group 1, group 3, or group 4. In various embodiments, the subject exhibiting dysregulated host response is further exhibiting shock, and wherein the at least one biomarker set is one of group 1, group 2, group 4, group 5, group 6, group 7, or group 8. In various embodiments, the subject exhibiting dysregulated host response is an adult subject, and wherein the at least one biomarker set is one of group 1, group 2, group 3, group 5, group 6, group 7, or group 8. In various embodiments, the subject exhibiting dysregulated host response is a pediatric subject, and wherein the at least one biomarker set is one of group 1, group 4, group 5, group 6, group 7, or group 8. In various embodiments, the quantitative data is determined by one of RT-qPCR (quantitative reverse transcription polymerase chain reaction), qPCR (quantitative polymerase chain reaction), PCR (polymerase chain reaction), RT-PCR (reverse transcription polymerase chain reaction), SDA (strand displacement amplification), RPA (recombinase polymerase amplification), MDA (multiple displacement amplification), HDA (helicase dependent amplification), LAMP (loop-mediated isothermal amplification), RCA (rolling circle amplification), NASBA (nucleic acid-sequence-based amplification), and any other isothermal or thermocycled amplification reaction.

In various embodiments, the quantitative data is determined by: contacting a sample with a reagent; generating a plurality of complexes between the reagent and the plurality of biomarkers in the sample; and detecting the plurality of complexes to obtain a dataset associated with the sample, wherein the dataset comprises the quantitative data.

In various embodiments, the classification of the subject is determined by: determining, for at least one candidate classification of the subject, a classification-specific score for the subject; determining, by the patient subtype classifier, based on the classification-specific score, the classification of the subject. In various embodiments, the instructions that cause the processor to determine the classification-specific score further comprises instructions that, when executed by the processor, cause the processor to: determine a first subscore of the quantitative data for the subject for one or more biomarkers of the candidate classification, wherein the quantitative data for the subject for the one or more biomarkers of the candidate classification are increased relative to the quantitative data for the one or more biomarkers for one or more control subjects; determine a second subscore of the quantitative expression for the subject for one or more additional biomarkers of the candidate classification, wherein the quantitative data for the subject for the one or more additional biomarkers of the candidate classification are decreased relative to the quantitative data for the one or more additional biomarkers for the one or more control subjects; and determine a difference between the first subscore and the second subscore, the first and second geometric subscore optionally subject to scaling, and the difference comprising the classification-specific score for the subject. In various embodiments, one or both of the first subscore and the second subscore are geometric means. In various embodiments, the patient subtype classifier is a machine-learned model. In various embodiments, the machine-learned model is a support vector machine (SVM). In various embodiments, the support vector machine receives, as input, one or more classification-specific scores and outputs the classification of the subject.

In various embodiments, the patient subtype classifier determines the classification of the subject by: comparing the classification-specific scores to one or more threshold values; and determining the classification of the subject based on the comparisons. In various embodiments, at least one of the one or more threshold values is a fixed value. In various embodiments, at least one of the one or more threshold values is determined using training samples, the at least one threshold value representing a value on a ROC curve nearest to maximum sensitivity or maximum specificity. In various embodiments, further comprising, prior to determining a classification of the subject using a patient subtype classifier, normalizing the quantitative data based on quantitative data for one or more housekeeping genes.

In various embodiments, wherein the candidate classifications of the subject comprise subtype A, subtype B, and subtype C. In various embodiments, the at least one biomarker set is group 1, and wherein the patient subtype classifier has an average accuracy of at least 82.93%. In various embodiments, the patient subtype classifier has an average accuracy of at least 89.6%. In various embodiments, the patient subtype classifier has an average accuracy of at least 86.3%. In various embodiments, the at least one biomarker set is group 4, and wherein the patient subtype classifier has an average accuracy of at least 98.3%.

In various embodiments, the therapy recommendation identified for the subject further comprises corticosteroid therapy, no corticosteroid therapy, or no therapy recommendation. In various embodiments, the therapy recommendation comprises a no corticosteroid therapy, wherein the no corticosteroid therapy is identified by determining that a statistical significance of a reduction in mortality of subjects exhibiting dysregulated host response not provided corticosteroid therapy is greater than or equal to a threshold statistical significance. In various embodiments, the therapy recommendation comprises a no corticosteroid therapy, wherein the no corticosteroid therapy is identified by determining that the classification of the subject comprises a subtype likely to be adversely responsive to corticosteroid therapy. In various embodiments, the subtype is subtype A or subtype C.

In various embodiments, the therapy recommendation comprises a corticosteroid therapy, wherein the corticosteroid therapy is identified by determining that a statistical significance of a reduction in mortality of subjects exhibiting dysregulated host response and provided corticosteroid therapy is greater than or equal to a threshold statistical significance. In various embodiments, the therapy recommendation comprises a corticosteroid therapy, wherein the corticosteroid therapy is identified by determining that the classification of the subject comprises a subtype likely to be favorably responsive to corticosteroid therapy. In various embodiments, the subtype is subtype B.

In various embodiments, the therapy recommendation identified for the subject comprises a no therapy recommendation, wherein the no therapy recommendation is identified at least by: determining that a statistical significance of a reduction in mortality of subjects exhibiting dysregulated host response and not provided corticosteroid therapy is less than a threshold statistical significance; and determining that a statistical significance of a reduction in mortality of subjects exhibiting dysregulated host response and provided corticosteroid therapy is less than a threshold statistical significance. In various embodiments, a statistical significance comprises a p-value, and wherein the threshold statistical significance comprises at least 0.1.

In various embodiments, the therapy recommendation identified for the subject comprises a no corticosteroid therapy, wherein the dysregulated host response comprises dysregulated host response not caused by infection, and wherein the at least one biomarker set is group 1 or group 4. In various embodiments, the no corticosteroid therapy is identified by determining that the classification of the subject comprises a subtype likely to be adversely responsive to corticosteroid therapy. In various embodiments, the subtype is subtype A or subtype C.

In various embodiments, the therapy recommendation identified for the subject further comprises no therapy recommendation, wherein the no therapy recommendation is identified by determining that the classification of the subject comprises subtype B. In various embodiments, the therapy recommendation identified for the subject comprises a no corticosteroid therapy, wherein the dysregulated host response comprises sepsis, wherein the at least one biomarker set is one of group 2, group 3, or group 4. In various embodiments, the no corticosteroid therapy is identified by determining that the classification of the subject comprises a subtype likely to be adversely responsive to corticosteroid therapy. In various embodiments, the subtype is subtype A.

In various embodiments, the therapy recommendation identified for the subject further comprises a no therapy recommendation, wherein the no therapy recommendation is identified by determining that the classification of the subject comprises a subtype likely to be non-responsive to corticosteroid therapy. In various embodiments, the subtype is subtype B or subtype C.

In various embodiments, the therapy recommendation identified for the subject further comprises a no corticosteroid therapy, wherein the dysregulated host response comprises dysregulated host response not caused by infection, and wherein the at least one biomarker set is group 2. In various embodiments, the no corticosteroid therapy is identified by determining that the classification of the subject comprises a subtype like to be adversely responsive to corticosteroid therapy. In various embodiments, the subtype is subtype C.

In various embodiments, the therapy recommendation identified for the subject further comprises a no therapy recommendation, wherein the no therapy recommendation is identified by determining that the classification of the subject comprises a subtype like to be non-responsive to corticosteroid therapy. In various embodiments, the subtype is subtype A or subtype B.

In various embodiments, the therapy recommendation identified for the subject comprises a no corticosteroid therapy, wherein the dysregulated host response comprises dysregulated host response not caused by infection, and wherein the at least one biomarker set is group 3. In various embodiments, the no corticosteroid therapy is identified by determining that the classification of the subject comprises a subtype like to be non-responsive to corticosteroid therapy. In various embodiments, the subtype is subtype A or subtype C.

In various embodiments, the therapy recommendation identified for the subject further comprises corticosteroid therapy, wherein the corticosteroid therapy is identified by determining that the classification of the subject comprises a subtype likely to be responsive to corticosteroid therapy. In various embodiments, the subtype is subtype B.

Additionally disclosed herein is a non-transitory computer readable medium for identifying a candidate therapeutic, the non-transitory computer readable medium comprising instructions that, when executed by a processor, cause the processor to: access a differentially expressed gene database comprising gene level fold changes between patients of different subtypes; determine at least a threshold number of genes are differentially expressed in patients of a first subtype in comparison to patients of a second subtype, wherein each of the differentially expressed genes is involved in a common biological pathway; and determine a candidate therapeutic likely to be effective for patients of the first subtype, wherein the candidate therapeutic is effective in modulating expression of at least one of the genes that are differentially expressed in patients of the first subtype. In various embodiments, the differentially expressed gene database is generated by: obtaining labeled patient data, wherein labels of the labeled patient data identify patients that are classified into one of two or more subtypes; generating the differentially expressed gene database for at least one or more genes by at least determining gene-level fold changes between patient data with a label indicating a first subtype and patient data with a label indicating a second subtype. In various embodiments, the labels of the labeled patient data are generated by applying a clustering analysis or by applying a patient subtype classifier. In various embodiments, at least the threshold number of genes is at least three genes, at least four genes, at least five genes, at least six genes, at least seven genes, at least eight genes, at least nine genes, or at least ten genes. In various embodiments, determining a candidate therapeutic for patients of the first subtype further comprises: analyzing one or both of: therapeutic pharmacology data comprising data for the candidate therapeutic; and host response pathobiology comprising data for patients of the first subtype.

Additionally disclosed herein is a system for determining a patient subtype, the system comprising: a set of reagents used for determining quantitative data for at least one biomarker set from a test sample from a subject, the at least one biomarker set selected from the group consisting of the biomarker sets of group 1, group 2, group 3, group 4, or group 5, wherein group 1 comprises biomarker 1, biomarker 2, and biomarker 3, wherein biomarker 1 is one or more of EVL, BTN3A2, HLA-DPA1, IDH3A, ACBD3, EXOSC10, SNRK, or MMP8, wherein biomarker 2 is one or more of SERPINB1 or GSPT1, and wherein biomarker 3 is one or more of MPP1, HMBS, TAL1, C9orf78, POLR2L, SLC27A3, BTN3A2, DDX50, FCHSD2, GSTK1, UBE2E1, TNFRSF1A, PRPF3, or TOMM70A, wherein group 2 comprises biomarker 4, biomarker 5, and biomarker 6, wherein biomarker 4 is one or more of ZNF831, MME, CD3G, or STOM, wherein biomarker 5 is one or more of ECSIT, LAT, or NCOA4, and wherein biomarker 6 is one or more of SLC1A5, IGF2BP2, or ANXA3, wherein group 3 comprises biomarker 7, biomarker 8, and biomarker 9, wherein biomarker 7 is one or more of C14orf159 or PUM2, wherein biomarker 8 is one or more of EPB42 or RPS6KA5, and wherein biomarker 9 is one or more of EPB42 or GBP2; and wherein group 4 comprises biomarker 10, biomarker 11, and biomarker 12, wherein biomarker 10 is one or more of MSH2, DCTD, or MMP8, wherein biomarker 11 is one or more of HK3, UCP2, or NUP88, and wherein biomarker 12 is one or more of GABARAPL2 or CASP4; and wherein group 5 comprises biomarker 13, biomarker 14, and biomarker 15, wherein biomarker 13 is one or more of STOM, MME, BNT3A2, HLA-DPA1, ZNF831, or CD3G, wherein biomarker 14 is one or more of EPB42, GSPT1, LAT, HK3, or SERPINB1, and wherein biomarker 15 is one or more of SLC1A5, IGF2BP2, ANXA3, GBP2, TNFRSF1, BTN3A2, OR TNFRSF1A; and an apparatus configured to receive a mixture of one or more reagents in the set and the test sample and to measure the quantitative data for the at least one biomarker set from the test sample; and a computer system communicatively coupled to the apparatus to obtain the quantitative data for the at least one biomarker set and to determine a classification of the subject based on the quantitative data using a patient subtype classifier.

In various embodiments, the at least one biomarker set is group 5, and wherein biomarker 13 is one or more of STOM, MME, BNT3A2, or HLA-DPA1. In various embodiments, biomarker 14 is one or more of EPB42, GSPT1, LAT, HK3, or SERPINB1. In various embodiments, the at least one biomarker set is group 5, and wherein biomarker 15 is one or more of SLC1A5, IGF2BP2, or ANXA3.

Additionally disclosed herein is a system for determining a patient subtype, the system comprising: a set of reagents used for determining quantitative data for two or more biomarkers selected from the group consisting of EVL, BTN3A2, HLA-DPA1, IDH3A, ACBD3, EXOSC10, SNRK, MMP8, SERPINB1, GSPT1, MPP1, HMBS, TAL1, C9orf78, POLR2L, SLC27A3, DDX50, FCHSD2, GSTK1, UBE2E1, TNFRSF1A, PRPF3, TOMM70A, ZNF831, MME, CD3G, STOM, ECSIT, LAT, NCOA4, SLC1A5, IGF2BP2, ANXA3, C14orf159, PUM2, EPB42, RPS6KA5, GBP2, MSH2, DCTD, HK3, UCP2, NUP88, GABARAPL2, and CASP4; and an apparatus configured to receive a mixture of one or more reagents in the set and the test sample and to measure the quantitative data for the at least one biomarker set from the test sample; and a computer system communicatively coupled to the apparatus to obtain the quantitative data for the at least one biomarker set and to determine a classification of the subject based on the quantitative data using a patient subtype classifier.

Additionally disclosed herein is a system for determining a patient subtype, the system comprising: a set of reagents used for determining quantitative data for at least one biomarker set selected from the group consisting of the biomarker sets of group 1, group 2, group 3, group 4, or group 5, wherein group 1 comprises two or more biomarkers selected from a group consisting of EVL, BTN3A2, HLA-DPA1, IDH3A, ACBD3, EXOSC10, SNRK, MMP8, SERPINB1 GSPT1, MPP1, HMBS, TAL1, C9orf78, POLR2L, SLC27A3, BTN3A2, DDX50, FCHSD2, GSTK1, UBE2E1, TNFRSF1A, PRPF3, and TOMM70A, wherein group 2 comprises two or more biomarkers selected from a group consisting of ZNF831, MME, CD3G, STOM, ECSIT, LAT, NCOA4, SLC1A5, IGF2BP2, and ANXA3, wherein group 3 comprises two or more biomarkers selected from a group consisting of C14orf159, PUM2, EPB42, RPS6KA5, EPB42, and GBP2; and wherein group 4 comprises two or more biomarkers selected from a group consisting of MSH2, DCTD, MMP8, HK3, UCP2, NUP88, GABARAPL2, and CASP4; and wherein group 5 comprises two or more biomarkers selected from a group consisting of STOM, MME, BNT3A2, HLA-DPA1, ZNF831, CD3G, EPB42, GSPT1, LAT, HK3, SERPINB1, SLC1A5, IGF2BP2, ANXA3, GBP2, TNFRSF1, BTN3A2, and TNFRSF1A; and an apparatus configured to receive a mixture of one or more reagents in the set and the test sample and to measure the quantitative data for the at least one biomarker set from the test sample; and a computer system communicatively coupled to the apparatus to obtain the quantitative data for the at least one biomarker set and to determine a classification of the subject based on the quantitative data using a patient subtype classifier. In various embodiments, the computer system is configured to identify a therapy recommendation for the subject based at least in part on the classification.

Additionally disclosed herein is a system for determining a therapy recommendation for a subject, the system comprising: a computer system configured to: obtain a classification of the subject exhibiting a dysregulated host response, the classification having been determined by: obtaining or having obtained quantitative data for at least one biomarker set obtained from the subject, the at least one biomarker set selected from the group consisting of the biomarker sets of group 1, group 2, group 3, group 4, or group 5, wherein group 1 comprises biomarker 1, biomarker 2, and biomarker 3, wherein biomarker 1 is one or more of EVL, BTN3A2, HLA-DPA1, IDH3A, ACBD3, EXOSC10, SNRK, or MMP8, wherein biomarker 2 is one or more of SERPINB1 or GSPT1, and wherein biomarker 3 is one or more of MPP1, HMBS, TAL1, C9orf78, POLR2L, SLC27A3, BTN3A2, DDX50, FCHSD2, GSTK1, UBE2E1, TNFRSF1A, PRPF3, or TOMM70A, wherein group 2 comprises biomarker 4, biomarker 5, and biomarker 6, wherein biomarker 4 is one or more of ZNF831, MME, CD3G, or STOM, wherein biomarker 5 is one or more of ECSIT, LAT, or NCOA4, and wherein biomarker 6 is one or more of SLC1A5, IGF2BP2, or ANXA3, wherein group 3 comprises biomarker 7, biomarker 8, and biomarker 9, wherein biomarker 7 is one or more of C14orf159 or PUM2, wherein biomarker 8 is one or more of EPB42 or RPS6KA5, and wherein biomarker 9 is one or more of EPB42 or GBP2; and wherein group 4 comprises biomarker 10, biomarker 11, and biomarker 12, wherein biomarker 10 is one or more of MSH2, DCTD, or MMP8, wherein biomarker 11 is one or more of HK3, UCP2, or NUP88, and wherein biomarker 12 is one or more of GABARAPL2 or CASP4; and wherein group 5 comprises biomarker 13, biomarker 14, and biomarker 15, wherein biomarker 13 is one or more of STOM, MME, BNT3A2, HLA-DPA1, ZNF831, or CD3G, wherein biomarker 14 is one or more of EPB42, GSPT1, LAT, HK3, or SERPINB1, and wherein biomarker 15 is one or more of SLC1A5, IGF2BP2, ANXA3, GBP2, TNFRSF1, BTN3A2, OR TNFRSF1A; and determine the classification based on the quantitative data using a patient subtype classifier; and identify a therapy recommendation for the subject based at least in part on the classification.

In various embodiments, the dysregulated host response of the subject comprises one of sepsis and dysregulated host response not caused by infection. In various embodiments, the classification of the subject comprises one of subtype A or subtype B. In various embodiments, the classification of the subject comprises one of subtype A, subtype B, or subtype C. In various embodiments, responsive to the classification of the subject comprising subtype A, the therapy recommendation identified for the subject comprises at least no immunosuppressive therapy.

In various embodiments, responsive to the classification of the subject comprising subtype A, the therapy recommendation identified for the subject further comprises at least no corticosteroid therapy. In various embodiments, the therapy recommendation identified for the subject further comprises no hydrocortisone. In various embodiments, responsive to the classification of the subject comprising subtype B, the therapy recommendation identified for the subject comprises at least one of no therapy recommendation, immune stimulation therapy, suppression of immune regulation therapy, blocking of immune suppression therapy, blocking of complement activity therapy, and anti-inflammatory therapy. In various embodiments, responsive to the classification of the subject comprising subtype B, the therapy recommendation identified for the subject further comprises at least one of a checkpoint inhibitor, a blocker of complement components, a blocker of complement component receptors, and a blocker of a pro-inflammatory cytokine. In various embodiments, the therapy recommendation identified for the subject further comprises at least one of GM-CSF, anti-PD-1, anti-PD-L1, anti-CLTA-4, anti-CEACAM-1, anti-TIM-3, anti-BTLA, IL-7, INF-gamma, IFN-beta 1a regulator, IL-22 agonist, IFN-alpha regulator, IFN-lambda regulator, IFN-alpha 2b stimulant, anti-C5a, anti-C3a, anti-C5aR, anti-C3aR, anti-TNF-alpha, and anti-IL-6, Anti-HMGB1, ST2 antibody, IL-33 antibody.

In various embodiments, responsive to the classification of the subject comprising subtype C, the therapy recommendation identified for the subject comprises at least one of no therapy recommendation, immune stimulation therapy, suppression of immune regulation therapy, blocking of immune suppression therapy, modulators of coagulation therapy, and modulators of vascular permeability therapy. In various embodiments, responsive to the classification of the subject comprising subtype C, the therapy recommendation identified for the subject further comprises at least one of a checkpoint inhibitor and an anticoagulant. In various embodiments, the therapy recommendation identified for the subject further comprises at least one of GM-CSF, anti-PD-1, anti-PD-L1, anti-CLTA-4, anti-CEACAM-1, anti-TIM-3, anti-BTLA, INF-gamma, IFN-beta 1a regulator, IL-22 agonist, IFN-alpha regulator, IFN-lambda regulator, IFN-alpha 2b stimulant, activated protein C, antithrombin, and thrombomodulin.

In various embodiments, the sample comprises a blood sample from the subject. In various embodiments, the subject exhibiting dysregulated host response does not exhibit shock, and wherein the at least one biomarker set is one of group 1, group 3, or group 4. In various embodiments, the subject exhibiting dysregulated host response is further exhibiting shock, and wherein the at least one biomarker set is one of group 1, group 2, group 4, group 5, group 6, group 7, or group 8. In various embodiments, the subject exhibiting dysregulated host response is an adult subject, and wherein the at least one biomarker set is one of group 1, group 2, group 3, group 5, group 6, group 7, or group 8. In various embodiments, the subject exhibiting dysregulated host response is a pediatric subject, and wherein the at least one biomarker set is one of group 1, group 4, group 5, group 6, group 7, or group 8.

In various embodiments, the quantitative data is determined by one of RT-qPCR (quantitative reverse transcription polymerase chain reaction), qPCR (quantitative polymerase chain reaction), PCR (polymerase chain reaction), RT-PCR (reverse transcription polymerase chain reaction), SDA (strand displacement amplification), RPA (recombinase polymerase amplification), MDA (multiple displacement amplification), HDA (helicase dependent amplification), LAMP (loop-mediated isothermal amplification), RCA (rolling circle amplification), NASBA (nucleic acid-sequence-based amplification), and any other isothermal or thermocycled amplification reaction.

In various embodiments, the classification of the subject is determined by: determining, for at least one candidate classification of the subject, a classification-specific score for the subject; determining, by the patient subtype classifier, based on the classification-specific score, the classification of the subject. In various embodiments, determine the classification-specific score further comprises: determine a first subscore of the quantitative data for the subject for one or more biomarkers of the candidate classification, wherein the quantitative data for the subject for the one or more biomarkers of the candidate classification are increased relative to the quantitative data for the one or more biomarkers for one or more control subjects; determine a second subscore of the quantitative expression for the subject for one or more additional biomarkers of the candidate classification, wherein the quantitative data for the subject for the one or more additional biomarkers of the candidate classification are decreased relative to the quantitative data for the one or more additional biomarkers for the one or more control subjects; and determine a difference between the first subscore and the second subscore, the first and second geometric subscore optionally subject to scaling, and the difference comprising the classification-specific score for the subject. In various embodiments, one or both of the first subscore and the second subscore are geometric means.

In various embodiments, the patient subtype classifier is a machine-learned model. In various embodiments, the machine-learned model is a support vector machine (SVM). In various embodiments, the support vector machine receives, as input, one or more classification-specific scores and outputs the classification of the subject. In various embodiments, the patient subtype classifier determines the classification of the subject by: comparing the classification-specific scores to one or more threshold values; and determining the classification of the subject based on the comparisons.

In various embodiments, at least one of the one or more threshold values is a fixed value. In various embodiments, at least one of the one or more threshold values is determined using training samples, the at least one threshold value representing a value on a ROC curve nearest to maximum sensitivity or maximum specificity. In various embodiments, prior to determining a classification of the subject using a patient subtype classifier, normalizing the quantitative data based on quantitative data for one or more housekeeping genes.

In various embodiments, the candidate classifications of the subject comprise subtype A, subtype B, and subtype C. In various embodiments, the at least one biomarker set is group 1, and wherein the patient subtype classifier has an average accuracy of at least 82.93%. In various embodiments, the patient subtype classifier has an average accuracy of at least 89.6%. In various embodiments, the patient subtype classifier has an average accuracy of at least 86.3%. In various embodiments, the at least one biomarker set is group 4, and wherein the patient subtype classifier has an average accuracy of at least 98.3%.

In various embodiments, the therapy recommendation identified for the subject further comprises corticosteroid therapy, no corticosteroid therapy, or no therapy recommendation. In various embodiments, the therapy recommendation comprises a no corticosteroid therapy, wherein the no corticosteroid therapy is identified by determining that a statistical significance of a reduction in mortality of subjects exhibiting dysregulated host response not provided corticosteroid therapy is greater than or equal to a threshold statistical significance. In various embodiments, the therapy recommendation comprises a no corticosteroid therapy, wherein the no corticosteroid therapy is identified by determining that the classification of the subject comprises a subtype likely to be adversely responsive to corticosteroid therapy. In various embodiments, the subtype is subtype A or subtype C. In various embodiments, the therapy recommendation comprises a corticosteroid therapy, wherein the corticosteroid therapy is identified by determining that a statistical significance of a reduction in mortality of subjects exhibiting dysregulated host response and provided corticosteroid therapy is greater than or equal to a threshold statistical significance.

In various embodiments, the therapy recommendation comprises a corticosteroid therapy, wherein the corticosteroid therapy is identified by determining that the classification of the subject comprises a subtype likely to be favorably responsive to corticosteroid therapy. In various embodiments, the subtype is subtype B.

In various embodiments, the therapy recommendation identified for the subject comprises a no therapy recommendation, wherein the no therapy recommendation is identified at least by: determining that a statistical significance of a reduction in mortality of subjects exhibiting dysregulated host response and not provided corticosteroid therapy is less than a threshold statistical significance; and determining that a statistical significance of a reduction in mortality of subjects exhibiting dysregulated host response and provided corticosteroid therapy is less than a threshold statistical significance. In various embodiments, a statistical significance comprises a p-value, and wherein the threshold statistical significance comprises at least 0.1.

In various embodiments, the therapy recommendation identified for the subject comprises a no corticosteroid therapy, wherein the dysregulated host response comprises dysregulated host response not caused by infection, and wherein the at least one biomarker set is group 1 or group 4. In various embodiments, the no corticosteroid therapy is identified by determining that the classification of the subject comprises a subtype likely to be adversely responsive to corticosteroid therapy. In various embodiments, the subtype is subtype A or subtype C.

In various embodiments, the therapy recommendation identified for the subject further comprises no therapy recommendation, wherein the no therapy recommendation is identified by determining that the classification of the subject comprises subtype B.

In various embodiments, the therapy recommendation identified for the subject comprises a no corticosteroid therapy, wherein the dysregulated host response comprises sepsis, wherein the at least one biomarker set is one of group 2, group 3, or group 4. In various embodiments, the no corticosteroid therapy is identified by determining that the classification of the subject comprises a subtype likely to be adversely responsive to corticosteroid therapy. In various embodiments, the subtype is subtype A.

In various embodiments, the therapy recommendation identified for the subject further comprises a no therapy recommendation, wherein the no therapy recommendation is identified by determining that the classification of the subject comprises a subtype likely to be non-responsive to corticosteroid therapy. In various embodiments, the subtype is subtype B or subtype C.

In various embodiments, the therapy recommendation identified for the subject further comprises a no corticosteroid therapy, wherein the dysregulated host response comprises dysregulated host response not caused by infection, and wherein the at least one biomarker set is group 2. In various embodiments, the no corticosteroid therapy is identified by determining that the classification of the subject comprises a subtype like to be adversely responsive to corticosteroid therapy. In various embodiments, the subtype is subtype C.

In various embodiments, the therapy recommendation identified for the subject further comprises a no therapy recommendation, wherein the no therapy recommendation is identified by determining that the classification of the subject comprises a subtype like to be non-responsive to corticosteroid therapy. In various embodiments, the subtype is subtype A or subtype B.

In various embodiments, the therapy recommendation identified for the subject comprises a no corticosteroid therapy, wherein the dysregulated host response comprises dysregulated host response not caused by infection, and wherein the at least one biomarker set is group 3. In various embodiments, the no corticosteroid therapy is identified by determining that the classification of the subject comprises a subtype like to be non-responsive to corticosteroid therapy. In various embodiments, the subtype is subtype A or subtype C.

In various embodiments, the therapy recommendation identified for the subject further comprises corticosteroid therapy, wherein the corticosteroid therapy is identified by determining that the classification of the subject comprises a subtype likely to be responsive to corticosteroid therapy. In various embodiments, the subtype is subtype B.

Additionally disclosed herein is a a system for identifying a candidate therapeutic, the system comprising: a storage device storing a differentially expressed gene database comprising gene level fold changes between patients of different subtypes; a computational device configured to: access one or more gene level fold changes corresponding to differentially expressed genes in the differentially expressed gene database; determine at least a threshold number of genes are differentially expressed in patients of a first subtype in comparison to patients of a second subtype, wherein each of the differentially expressed genes is involved in a common biological pathway; and determine a candidate therapeutic likely to be effective for patients of the first subtype, wherein the candidate therapeutic is effective in modulating expression of at least one of the genes that are differentially expressed in patients of the first subtype. In various embodiments, the differentially expressed gene database is generated by: obtaining labeled patient data, wherein labels of the labeled patient data identify patients that are classified into one of two or more subtypes; generating the differentially expressed gene database for at least one or more genes by at least determining gene-level fold changes between patient data with a label indicating a first subtype and patient data with a label indicating a second subtype. In various embodiments, the labels of the labeled patient data are generated by applying a clustering analysis or by applying a patient subtype classifier. In various embodiments, at least the threshold number of genes is at least three genes, at least four genes, at least five genes, at least six genes, at least seven genes, at least eight genes, at least nine genes, or at least ten genes. In various embodiments, determining a candidate therapeutic for patients of the first subtype further comprises: analyzing one or both of: therapeutic pharmacology data comprising data for the candidate therapeutic; and host response pathobiology comprising data for patients of the first subtype.

Additionally disclosed herein is a kit for determining a patient subtype, the kit comprising: a set of reagents for determining quantitative data for at least one biomarker set from a test sample from a subject, the at least one biomarker set selected from the group consisting of the biomarker sets of group 1, group 2, group 3, group 4, or group 5, wherein group 1 comprises biomarker 1, biomarker 2, and biomarker 3, wherein biomarker 1 is one or more of EVL, BTN3A2, HLA-DPA1, IDH3A, ACBD3, EXOSC10, SNRK, or MMP8, wherein biomarker 2 is one or more of SERPINB1 or GSPT1, and wherein biomarker 3 is one or more of MPP1, HMBS, TAL1, C9orf78, POLR2L, SLC27A3, BTN3A2, DDX50, FCHSD2, GSTK1, UBE2E1, TNFRSF1A, PRPF3, or TOMM70A, wherein group 2 comprises biomarker 4, biomarker 5, and biomarker 6, wherein biomarker 4 is one or more of ZNF831, MME, CD3G, or STOM, wherein biomarker 5 is one or more of ECSIT, LAT, or NCOA4, and wherein biomarker 6 is one or more of SLC1A5, IGF2BP2, or ANXA3, wherein group 3 comprises biomarker 7, biomarker 8, and biomarker 9, wherein biomarker 7 is one or more of C14orf159 or PUM2, wherein biomarker 8 is one or more of EPB42 or RPS6KA5, and wherein biomarker 9 is one or more of EPB42 or GBP2; and wherein group 4 comprises biomarker 10, biomarker 11, and biomarker 12, wherein biomarker 10 is one or more of MSH2, DCTD, or MMP8, wherein biomarker 11 is one or more of HK3, UCP2, or NUP88, and wherein biomarker 12 is one or more of GABARAPL2 or CASP4; and wherein group 5 comprises biomarker 13, biomarker 14, and biomarker 15, wherein biomarker 13 is one or more of STOM, MME, BNT3A2, HLA-DPA1, ZNF831, or CD3G, wherein biomarker 14 is one or more of EPB42, GSPT1, LAT, HK3, or SERPINB1, and wherein biomarker 15 is one or more of SLC1A5, IGF2BP2, ANXA3, GBP2, TNFRSF1, BTN3A2, OR TNFRSF1A; and instructions for using the set of reagents to determine the quantitative data for the at least one biomarker set.

In various embodiments, the at least one biomarker set is group 5, and wherein biomarker 13 is one or more of STOM, MME, BNT3A2, or HLA-DPA1. In various embodiments, the at least one biomarker set is group 5, and wherein biomarker 14 is one or more of EPB42, GSPT1, LAT, HK3, or SERPINB1. In various embodiments, the at least one biomarker set is group 5, and wherein biomarker 15 is one or more of SLC1A5, IGF2BP2, or ANXA3.

Additionally disclosed herein is a kit for determining a patient subtype, the kit comprising: a set of reagents for determining quantitative data for two or more biomarkers selected from the group consisting of EVL, BTN3A2, HLA-DPA1, IDH3A, ACBD3, EXOSC10, SNRK, MMP8, SERPINB1, GSPT1, MPP1, HMBS, TAL1, C9orf78, POLR2L, SLC27A3, DDX50, FCHSD2, GSTK1, UBE2E1, TNFRSF1A, PRPF3, TOMM70A, ZNF831, MME, CD3G, STOM, ECSIT, LAT, NCOA4, SLC1A5, IGF2BP2, ANXA3, C14orf159, PUM2, EPB42, RPS6KA5, GBP2, MSH2, DCTD, HK3, UCP2, NUP88, GABARAPL2, and CASP4; and instructions for using the set of reagents to determine the quantitative data for the at least one biomarker set.

Additionally disclosed herein is a kit for determining a patient subtype, the kit comprising: a set of reagents for determining quantitative data for at least one biomarker set selected from the group consisting of the biomarker sets of group 1, group 2, group 3, group 4, or group 5, wherein group 1 comprises two or more biomarkers selected from a group consisting of EVL, BTN3A2, HLA-DPA1, IDH3A, ACBD3, EXOSC10, SNRK, MMP8, SERPINB1 GSPT1, MPP1, HMBS, TAL1, C9orf78, POLR2L, SLC27A3, BTN3A2, DDX50, FCHSD2, GSTK1, UBE2E1, TNFRSF1A, PRPF3, and TOMM70A, wherein group 2 comprises two or more biomarkers selected from a group consisting of ZNF831, MME, CD3G, STOM, ECSIT, LAT, NCOA4, SLC1A5, IGF2BP2, and ANXA3, wherein group 3 comprises two or more biomarkers selected from a group consisting of C14orf159, PUM2, EPB42, RPS6KA5, EPB42, and GBP2; and wherein group 4 comprises two or more biomarkers selected from a group consisting of MSH2, DCTD, MMP8, HK3, UCP2, NUP88, GABARAPL2, and CASP4; and wherein group 5 comprises two or more biomarkers selected from a group consisting of STOM, MME, BNT3A2, HLA-DPA1, ZNF831, CD3G, EPB42, GSPT1, LAT, HK3, SERPINB1, SLC1A5, IGF2BP2, ANXA3, GBP2, TNFRSF1, BTN3A2, and TNFRSF1A; and instructions for using the set of reagents to determine the quantitative data for the at least one biomarker set.

In various embodiments, the instructions comprise instructions for determining the quantitative data by performing one of RT-qPCR (quantitative reverse transcription polymerase chain reaction), qPCR (quantitative polymerase chain reaction), PCR (polymerase chain reaction), RT-PCR (reverse transcription polymerase chain reaction), SDA (strand displacement amplification), RPA (recombinase polymerase amplification), MDA (multiple displacement amplification), HDA (helicase dependent amplification), LAMP (loop-mediated isothermal amplification), RCA (rolling circle amplification), NASBA (nucleic acid-sequence-based amplification), and any other isothermal or thermocycled amplification reaction.

In various embodiments, the set of reagents comprises at least three primer sets for amplifying at least three biomarkers, wherein the at least three primer sets comprise pairs of single-stranded DNA primers for amplifying the at least three biomarkers, and wherein at least one of the at least three biomarkers is selected from the group consisting of the biomarkers EVL, BTN3A2, HLA-DPA1, IDH3A, ACBD3, EXOSC10, SNRK, MMP8, ZNF831, MME, CD3G, STOM, C14orf159, PUM2, MSH2, DCTD, BNT3A2, or HLA-DPA1, at least one biomarker of the at least three biomarkers is selected from the group consisting of the biomarkers SERPINB1, GSPT1, ECSIT, LAT, NCOA4, EPB42, RPS6KA5, HK3, UCP2, or NUP88, and at least one biomarker of the at least three biomarkers is selected from the group consisting of the biomarkers MPP1, HMBS, TAL1, C9orf78, POLR2L, SLC27A3, BTN3A2, DDX50, FCHSD2, GSTK1, UBE2E1, PRPF3, TOMM70A, EPB42, GABARAPL2, CASP4, SLC1A5, IGF2BP2, ANXA3, GBP2, TNFRSF1, OR TNFRSF1A.

In various embodiments, the at least one of the at least three primer sets is selected from the group consisting of: a forward primer comprising at least 15 contiguous nucleotides of SEQ ID NO. 7 and a reverse primer comprising at least 15 contiguous nucleotides of SEQ ID NO. 8, a forward primer comprising at least 15 contiguous nucleotides of SEQ ID NO. 9 and a reverse primer comprising at least 15 contiguous nucleotides of SEQ ID NO. 10, a forward primer comprising at least 15 contiguous nucleotides of SEQ ID NO. 11 and a reverse primer comprising at least 15 contiguous nucleotides of SEQ ID NO. 12, and a forward primer comprising at least 15 contiguous nucleotides of SEQ ID NO. 13 and a reverse primer comprising at least 15 contiguous nucleotides of SEQ ID NO. 14, wherein at least one of the at least three primer sets is selected from the group consisting of: a forward primer comprising at least 15 contiguous nucleotides of SEQ ID NO. 15 and a reverse primer comprising at least 15 contiguous nucleotides of SEQ ID NO. 16, a forward primer comprising at least 15 contiguous nucleotides of SEQ ID NO. 17 and a reverse primer comprising at least 15 contiguous nucleotides of SEQ ID NO. 18, and a forward primer comprising at least 15 contiguous nucleotides of SEQ ID NO. 19 and a reverse primer comprising at least 15 contiguous nucleotides of SEQ ID NO. 20, and wherein at least one of the at least three primer sets is selected from the group consisting of: a forward primer comprising at least 15 contiguous nucleotides of SEQ ID NO. 1 and a reverse primer comprising at least 15 contiguous nucleotides of SEQ ID NO. 2; a forward primer comprising at least 15 contiguous nucleotides of SEQ ID NO. 3 and a reverse primer comprising at least 15 contiguous nucleotides of SEQ ID NO. 4, and a forward primer comprising at least 15 contiguous nucleotides of SEQ ID NO. 5 and a reverse primer comprising at least 15 contiguous nucleotides of SEQ ID NO. 6.

In various embodiments, at least one of the at least three primer sets is selected from the group consisting of: a forward primer comprising SEQ ID NO. 7 and a reverse primer comprising SEQ ID NO. 8, a forward primer comprising SEQ ID NO. 9 and a reverse primer comprising SEQ ID NO. 10, a forward primer comprising SEQ ID NO. 11 and a reverse primer comprising SEQ ID NO. 12, and a forward primer comprising SEQ ID NO. 13 and a reverse primer comprising SEQ ID NO. 14, wherein at least one of the at least three primer sets is selected from the group consisting of: a forward primer comprising SEQ ID NO. 15 and a reverse primer comprising SEQ ID NO. 16, a forward primer comprising SEQ ID NO. 17 and a reverse primer comprising SEQ ID NO. 18, and a forward primer comprising SEQ ID NO. 19 and a reverse primer comprising SEQ ID NO. 20, and wherein at least one of the at least three primer sets is selected from the group consisting of: a forward primer comprising SEQ ID NO. 1 and a reverse primer comprising SEQ ID NO. 2; a forward primer comprising SEQ ID NO. 3 and a reverse primer comprising SEQ ID NO. 4, and a forward primer comprising SEQ ID NO. 5 and a reverse primer comprising SEQ ID NO. 6.

In various embodiments, the at least one of the at least three primer sets is selected from the group consisting of: a forward primer comprising at least 15 contiguous nucleotides of SEQ ID NO. 21 and a reverse primer comprising at least 15 contiguous nucleotides of SEQ ID NO. 22, and a forward primer comprising at least 15 contiguous nucleotides of SEQ ID NO. 23 and a reverse primer comprising at least 15 contiguous nucleotides of SEQ ID NO. 24, wherein at least one of the at least three primer sets is selected from the group consisting of: a forward primer comprising at least 15 contiguous nucleotides of SEQ ID NO. 25 and a reverse primer comprising at least 15 contiguous nucleotides of SEQ ID NO. 26, and a forward primer comprising at least 15 contiguous nucleotides of SEQ ID NO. 29 and a reverse primer comprising at least 15 contiguous nucleotides of SEQ ID NO. 30, and wherein at least one of the at least three primer sets is selected from the group consisting of: a forward primer comprising at least 15 contiguous nucleotides of SEQ ID NO. 25 and a reverse primer comprising at least 15 contiguous nucleotides of SEQ ID NO. 26, and a forward primer comprising at least 15 contiguous nucleotides of SEQ ID NO. 27 and a reverse primer comprising at least 15 contiguous nucleotides of SEQ ID NO. 28.

In various embodiments, at least one of the at least three primer sets is selected from the group consisting of: a forward primer comprising SEQ ID NO. 21 and a reverse primer comprising SEQ ID NO. 22, and a forward primer comprising SEQ ID NO. 23 and a reverse primer comprising SEQ ID NO. 24, wherein at least one of the at least three primer sets is selected from the group consisting of: a forward primer comprising SEQ ID NO. 25 and a reverse primer comprising SEQ ID NO. 26, and a forward primer comprising SEQ ID NO. 29 and a reverse primer comprising SEQ ID NO. 30, and wherein at least one of the at least three primer sets is selected from the group consisting of: a forward primer comprising SEQ ID NO. 25 and a reverse primer comprising SEQ ID NO. 26, and a forward primer comprising SEQ ID NO. 27 and a reverse primer comprising SEQ ID NO. 28.

In various embodiments, the set of reagents comprises at least three primer sets for amplifying at least three biomarkers, wherein each primer set of the at least three primer sets comprises a forward outer primer, a backward outer primer, a forward inner primer, a backward inner primer, a forward loop primer, and a backward loop primer for amplifying one of the at least three biomarkers, and wherein at least one of the at least three biomarkers is selected from the group consisting of EVL, BTN3A2, HLA-DPA1, IDH3A, ACBD3, EXOSC10, SNRK, MMP8, ZNF831, MME, CD3G, STOM, C14orf159, PUM2, MSH2, DCTD, BNT3A2, or HLA-DPA1, at least one biomarker of the at least three biomarkers is selected from the group consisting of SERPINB1, GSPT1, ECSIT, LAT, NCOA4, EPB42, RPS6KA5, HK3, UCP2, or NUP88, and at least one biomarker of the at least three biomarkers is selected from the group consisting of MPP1, HMBS, TAL1, C9orf78, POLR2L, SLC27A3, BTN3A2, DDX50, FCHSD2, GSTK1, UBE2E1, PRPF3, TOMM70A, EPB42, GABARAPL2, CASP4, SLC1A5, IGF2BP2, ANXA3, GBP2, TNFRSF1, OR TNFRSF1A.

In various embodiments, at least one of the at least three primer sets is selected from the group consisting of: a forward outer primer, a backward outer primer, a forward inner primer, a backward inner primer, a forward loop primer, and a backward loop primer, each of which is configured to enable amplification of at least one biomarker selected from the group consisting of: EVL, BTN3A2, HLA-DPA1, IDH3A, ACBD3, EXOSC10, SNRK, MMP8, ZNF831, MME, CD3G, STOM, C14orf159, PUM2, MSH2, DCTD, BNT3A2, or HLA-DPA1, a forward outer primer, a backward outer primer, a forward inner primer, a backward inner primer, a forward loop primer, and a backward loop primer, each of which is configured to enable amplification of at least one biomarker selected from the group consisting of: SERPINB1, GSPT1, ECSIT, LAT, NCOA4, EPB42, RPS6KA5, HK3, UCP2, or NUP88, and a forward outer primer, a backward outer primer, a forward inner primer, a backward inner primer, a forward loop primer, and a backward loop primer, each of which is configured to enable amplification of at least one biomarker selected from the group consisting of: MPP1, HMBS, TAL1, C9orf78, POLR2L, SLC27A3, BTN3A2, DDX50, FCHSD2, GSTK1, UBE2E1, PRPF3, TOMM70A, EPB42, GABARAPL2, CASP4, SLC1A5, IGF2BP2, ANXA3, GBP2, TNFRSF1, OR TNFRSF1A.

### BRIEF DESCRIPTION OF THE DRAWINGS

These and other features, aspects, and advantages of the present disclosure will become better understood with regard to the following description, and accompanying drawings, where:

FIG. 1A is a block diagram of a process for identifying subtypes of dysregulated host response patients, building a patient subtype classifier, and evaluating efficacy of corticosteroid therapy for dysregulated host response patients based on subtype classifications identified using the patient subtype classifier, in accordance with an embodiment.

FIG. 1B is a system environment overview for determining a therapy recommendation for a patient, in accordance with an embodiment.

FIG. 2 is a graph of the individual accuracies determined for each combination of three biomarkers, with one biomarker from each subtype, for the Full Model.

FIG. 3 is a graph of the individual accuracies determined for each combination of three biomarkers, with one biomarker from each subtype, for the SS Model.

FIG. 4 is a graph of the individual accuracies determined for each combination of three biomarkers, with one biomarker from each subtype, for the S Model.

FIG. 5 is a graph of the individual accuracies determined for each combination of three biomarkers, with one biomarker from each subtype, for the P Model.

FIGS. 6A-6D are graphs of individual accuracies determined for each combination of three biomarkers, with one biomarker from each subtype, for the SS.B1, SS.B2, SS.B3, and SS.B4 models, respectively.

FIG. 7 is an example flow process for determining therapeutic hypotheses for patient subtypes, in accordance with an embodiment.

FIG. 8 depicts the conclusions of the further analysis of Tables 6 and 7, in accordance with an embodiment.

FIG. 9 depicts a heat map depicting differential expression of genes from Table 6 for dysregulated host response patients having subtypes A, B, and C, and for healthy subjects without dysregulated host response, in accordance with an embodiment.

FIG. 10 depicts risk of morality for dysregulated host response patients having subtypes A, B, and C, in accordance with an embodiment.

FIG. 11 depicts differential expression of the genes of Table 7 that are associated with pharmacology of hydrocortisone therapy (e.g., regulation of the glucocorticoid receptor signaling pathway) for the subtypes A, B, and C, in accordance with an embodiment.

FIG. 12 provides support for a hypothesis of differential response to checkpoint inhibition therapy between the subtypes A, B, and C, by depicting differential expression of genes of Table 7 that are associated with pharmacology of checkpoint inhibition therapy (e.g., regulation of immune checkpoints and related immune functions mediated by cytokines) for subtypes A, B, and C, in accordance with an embodiment.

FIG. 13 depicts an example of a precision platform clinical trial design, in accordance with an embodiment.

FIG. 14 depicts an example workflow for the use of the patient subtype classifiers discussed throughout this disclosure, in targeting therapies for septic shock patients, in accordance with an embodiment.

FIG. 15 depicts an example dysregulated host response patient subtyping test that employs an FDA-cleared patient sample collection system (e.g., PAXgene Blood RNA System), and an FDA-cleared Real Time PCR system (e.g. the Thermo Fisher Quantstudio Dx System), in accordance with an embodiment.

FIG. 16 illustrates an example computer for implementing the methods described herein, in accordance with an embodiment.

The figures depict various embodiments of the present disclosure for purposes of illustration only. One skilled in the art will readily recognize from the following discussion that alternative embodiments of the structures and methods illustrated herein can be employed without departing from the principles of the disclosure described herein.

### DETAILED DESCRIPTION

### I. Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

The term “patient” or “subject” encompasses or organism, mammals including humans or non-humans (e.g., non-human primates, canines, felines, murines, bovines, equines, and porcines), whether in vivo, ex vivo, or in vitro, male or female.

The term “sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art. Examples of an aliquot of body fluid include amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humour.

The terms “marker,” “markers,” “biomarker,” and “biomarkers” encompass, without limitation, lipids, lipoproteins, proteins, cytokines, chemokines, growth factors, peptides, nucleic acids, genes, and oligonucleotides, together with their related complexes, metabolites, mutations, variants, polymorphisms, modifications, fragments, subunits, degradation products, elements, and other analytes or sample-derived measures. In particular embodiments discussed herein, the biomarkers are genes. However, in alternative embodiments, the biomarkers can include any other measurable substance in a sample from a subject. A marker can also include mutated proteins, mutated nucleic acids, variations in copy numbers, and/or transcript variants, in circumstances in which such mutations, variations in copy number and/or transcript variants are useful for generating a predictive model, or are useful in predictive models developed using related markers (e.g., non-mutated versions of the proteins or nucleic acids, alternative transcripts, etc.). In some embodiments, the biomarkers discussed throughout this disclosure can include a nucleic acid, including DNA, modified (e.g., methylated) DNA, cDNA, and RNA, including coding (e.g., mRNAs) and non-coding RNA (e.g., sncRNAs), a protein, including a post-transcriptionally modified protein (e.g., phosphorylated, glycosylated, myristilated, etc. proteins), a nucleotide (e.g., adenosine triphosphate (ATP), adenosine diphosphate (ADP), and adenosine monophosphate (AMP)), including cyclic nucleotides such as cyclic adenosine monophosphate (cAMP) and cyclic guanosine monophosphate (cGMP), a biologic, an ADC, a small molecule, such as oxidized and reduced forms of nicotinamide adenine dinucleotide (NADP/NADPH), a volatile compound, and any combination thereof.

The term “antibody” is used in the broadest sense and specifically covers monoclonal antibodies (including full length monoclonal antibodies), polyclonal antibodies, multi specific antibodies (e.g., bispecific antibodies), and antibody fragments that are antigen-binding so long as they exhibit the desired biological activity, e.g., an antibody or an antigen-binding fragment thereof.

“Antibody fragment,” and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”).

The term “obtaining or having obtained quantitative data” encompasses obtaining a set of data determined from at least one sample. Obtaining a dataset encompasses obtaining a sample and processing the sample to experimentally determine the data. The phrase also encompasses receiving a set of data, e.g., from a third party that has processed the sample to experimentally determine the dataset. Additionally, the phrase encompasses mining data from at least one database or at least one publication or a combination of databases and publications. A dataset can be obtained by one of skill in the art via a variety of known ways including stored on a storage memory.

Any terms not directly defined herein shall be understood to have the meanings commonly associated with them as understood within the art of the disclosure. Certain terms are discussed herein to provide additional guidance to the practitioner in describing the compositions, devices, methods and the like of aspects of the disclosure, and how to make or use them. It will be appreciated that the same thing can be said in more than one way. Consequently, alternative language and synonyms can be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the disclosure herein.

Additionally, as used in the specification, the singular forms “a,” “an” and “the” include plural referents unless the context clearly dictates otherwise.

### II. Overview: Biomarker Panels for Guiding Dysregulated Host Response Therapy

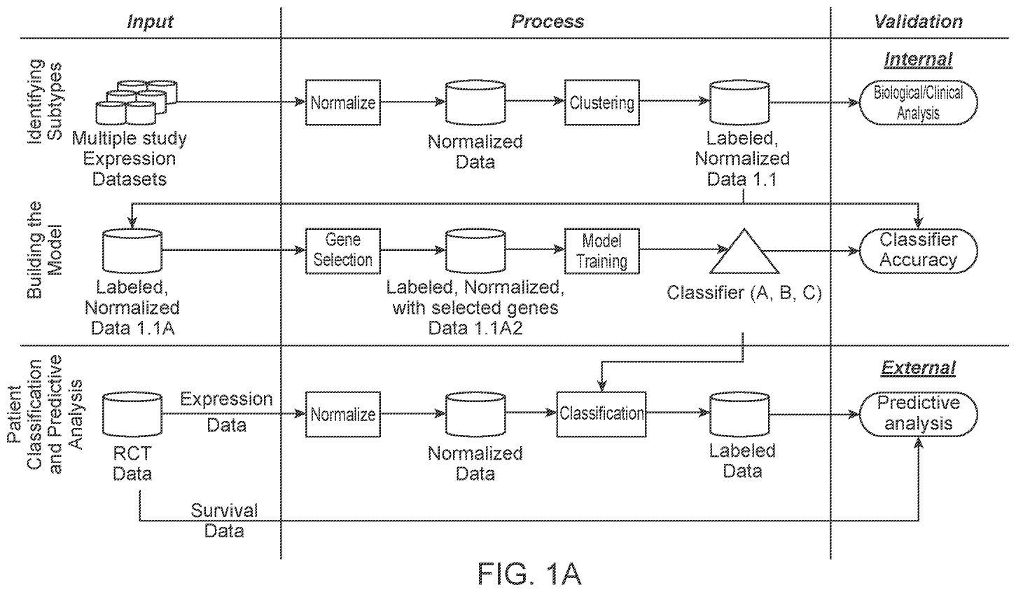

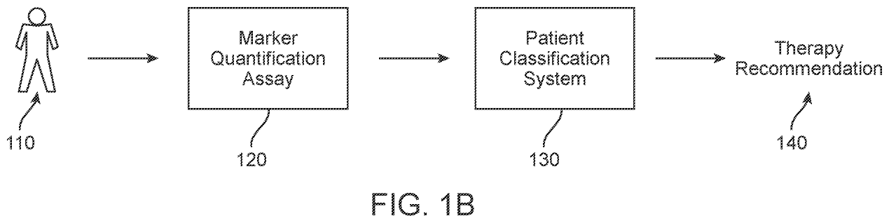

FIG. 1A is a block diagram of a process for identifying subtypes of dysregulated host response patients (row 1), building patient subtype classifiers (row 2), and evaluating efficacy of therapies for dysregulated host response patients based on subtype classifications identified using the patient subtype classifiers (row 3), in accordance with an embodiment. To identify subtypes of dysregulated host response patients, working datasets compiled from historical transcriptomic data from sepsis patients were created as described in further detail below. Then, clustering analysis was performed on the working dataset to identify subtypes of dysregulated host response patients based on differential biomarker expression. These clusters are labeled (e.g., subtype A, subtype B, subtype C, etc.) such that the data can be used for training and building a model (second row).

In various embodiments, the process of building a model that predicts patient subtypes, hereafter referred to as a patient subtype classifier, involves using the labeled data. The labeled data is analyzed to select biomarkers (e.g., “gene selection” as shown in FIG. 1A) that are informative for predicting certain patient subtypes. In various embodiments, patient subtype classifiers were trained using the labeled training data using. As depicted in the embodiment in FIG. 1A, the patient subtype classifier (depicted as a triangle) can be trained to classify a patient into one of three subtypes (e.g., subtype A, subtype B, and subtype C). In some embodiments, fewer (e.g., two subtypes) or additional (e.g., more than three) subtypes can be predicted by the patient subtype classifier. The patient subtype classifier can undergo validation using a test dataset (e.g., dataset other than the labeled training data) to ensure sufficient classifier performance

The trained patient subtype classifiers can be deployed to classify specific patients. In one embodiment, the patient subtype classifier analyzes data derived from randomized controlled trial (RCT) data pertaining to one or more patients and outputs predictions for the patients. For example, the patient subtype classifier analyzes quantitative biomarker expression data for patients that have been involved in a randomized controlled trial and classifies the patients in one of the different subtypes.

IIA. System Environment Overview

FIG. 1B depicts an overview of a system environment for determining a therapy recommendation 140 for a patient 110, in accordance with an embodiment. The system environment 100 provides context in order to introduce a marker quantification assay 120 and a patient classification system 130.

In various embodiments, a test sample is obtained from the subject 110. The test sample is analyzed to determine quantitative values of one or more biomarkers by performing the marker quantification assay 120. The marker quantification assay 120 may be a quantitative reverse transcription polymerase chain reaction (RT-PCR) assay, a microarray, a sequencing assay, or an immunoassay, examples of which are described in further detail below. The quantitative values of biomarkers can be quantified RT-PCR data, transcriptomics data, and/or RNA-seq data. The quantified expression values of the biomarkers are provided to the patient classification system 130.

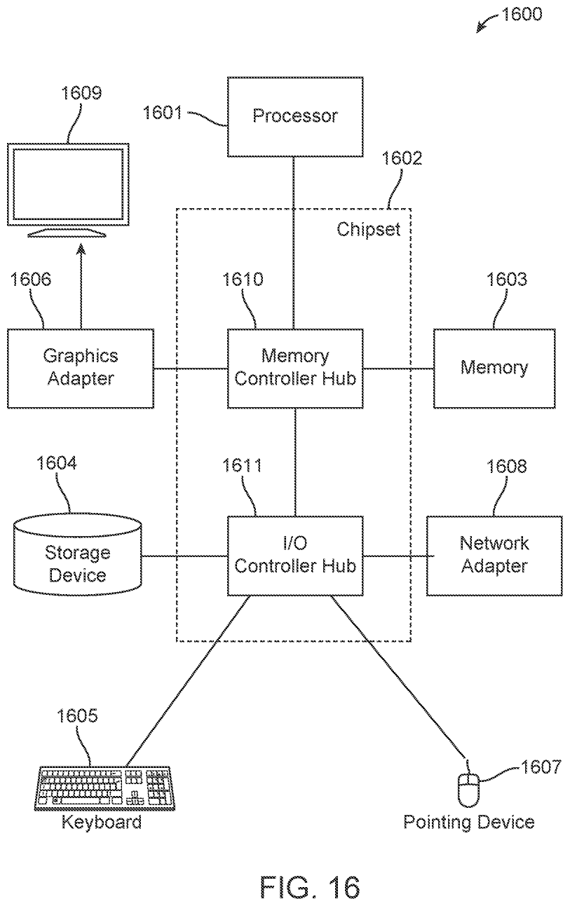

Generally, the patient classification system 130 includes one or more computers, such as example computer 1600 as discussed below with respect to FIG. 16. Therefore, in various embodiments, the steps described in reference to the patient classification system 130 are performed in silico. The patient classification system 130 analyzes the received biomarker expression values from the marker quantification assay 120. In various embodiments, the patient classification system 130 determines a classification for the patient 110. For example, a classification for the patient 110 can be one of multiple subtypes characterized by the quantitative biomarkers of the patient 110. In various embodiments, the patient classification system 130 determines a therapy recommendation 140 for the patient 110. In such embodiments, the patient classification system 130 determines a therapy recommendation 140 for the patient 110 based on a classification of the patient 110.

In various embodiments, the patient classification system 130 applies a patient subtype classifier to predict a classification for patient 110. In various embodiments, a patient subtype classifier can be a machine-learned model. In such embodiments, the patient classification system 130 can train the patient subtype classifier using training data and/or deploy the patient subtype classifier to analyze the quantitative expression values of biomarkers of the patient 110.

In various embodiments, the marker quantification assay 120 and the patient classification system 130 can be employed by different parties. For example, a first party performs the marker quantification assay 120 which then provides the results to a second party which implements the patient classification system 130. For example, the first party may be a clinical laboratory that obtains test samples from subjects 110 and performs the assay 120 on the test samples. The second party receives the expression values of biomarkers resulting from the performed assay 120 analyzes the expression values using the patient classification system 130.

In various embodiments, the patient classification system 130 can be a distributed computing system implemented in a cloud computing environment. For example, steps performed by the patient classification system 130 can be performed using systems in geographically different locations. In particular embodiments, the patient classification system 130 receives quantitative biomarker data from the marker quantification assay 120 at a first location. The patient classification system 130 transmits the quantitative biomarker data and analyzes the quantitative biomarker data to predict a classification using a patient subtype classifier at a second location (e.g., cloud computing). The patient classification system 130 can further transmit the classification back to the first location for subsequent use.

Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

In various embodiments, the marker quantification assay 120 and patient classification system 130 are implemented in a critical care setting such that a therapy recommendation is to be generated for a patient 110 within a maximum amount of time. In various embodiments, the maximum amount of time is 30 minutes. In various embodiments, the maximum amount of time is 1 hour, 2 hours, 3 hours, 4 hours, 5 hours, 6 hours, 7 hours, 8 hours, 9 hours, 10 hours, 11 hours, or 12 hours. In other embodiments, the marker quantification assay 120 and patient classification system 130 are not implemented in a critical care setting.

IIB. Methods for Determining a Therapy Recommendation

In various embodiments, the patient classification system 130 (as described above in reference to FIG. 1B) analyzes quantitative data for a biomarker set, the quantitative data derived from a patient (e.g., patient 110 in FIG. 1B), and determines a therapy recommendation for the patient. Generally, the patient classification system 130 applies a patient subtype classifier that analyzes the quantitative data for the biomarker set and classifies the patient in a classification. The patient classification system 130 can determine a therapy recommendation for the patient based on the classification of the patient.

The patient classification system 130 receives quantitative data from the marker quantification assay 120. Here, the quantitative data from the marker quantification assay 120 can include quantitative levels of one or more biomarkers that were determined from a sample obtained from a patient. In various embodiments, the patient classification system 130 normalizes the quantitative data. For example the patient classification system 130 can normalize the quantitative data based on study-specific parameters (such that data is normalized for a study) and/or based on parameters specific for a particular assay or platform used to generate the quantitative data. In various embodiments, the patient classification system 130 can normalize the quantitative data according to normalization parameters derived the healthy samples. In such embodiments, the resulting quantitative data are normalized across patients and studies at the end of the normalization process. Such embodiments that involve normalizing quantitative data can be implemented during research settings (non-critical care settings). In some embodiments, the patient classification system 130 need not normalize the quantitative data prior to analysis by the patient subtype classifier. Such embodiments that do not involve normalizing the quantitative data can be implemented in critical care settings where a rapid analysis and classification is needed for a patient 110. The patient classification system 130 analyzes the quantitative data, which hereafter also encompasses normalized quantitative data.

As one example, the patient classification system 130 analyzes quantitative data for a biomarker set derived from a microarray analysis. The patient classification system 130 applies a patient subtype classifier that analyzes the quantitative microarray data and classifies the patient, which can later be used to determine a therapy recommendation. As another example, the patient classification system 130 analyzes qPCR data, which measures the relative or absolute expression level of biomarkers. In various embodiments, normalization or calibration processes are implemented. The quantitative data of the biomarker set are used to calculate the scores for different classifications (e.g., subtypes), which then will be used for subtype assignment by a patient subtype classifier. As another example, the patient classification system 130 analyzes RNA sequencing data, which includes relative expression levels of model genes and their transcripts. Using sequencing reads alignment methods (e.g. Hisat2, and Bowtie2), expression estimation methods (e.g. StringTie, Salmon) and normalization processes (e.g. quantile normalization), the estimated expression of model genes can be used to calculate classification-specific scores for downstream classification by a patient subtype classifier. In various embodiments, the patient classification system 130 can convert quantitative data derived from a first type of assay to quantitative data of a second type of assay using normalization factors. For example, the patient classification system 130 can convert quantitative data derived from microarray data to either qPCR data or RNA sequencing data. The conversion can entail one or more normalization factors involving normalization or calibration processes for qPCR data or normalization processes (e.g., quantile normalization) for RNA sequencing data. Thus, the patient classification system 130 can apply different patient subtype classifiers to analyze different types of quantitative data.

The patient classification system 130 implements the patient subtype classifier to analyze quantitative data for biomarkers. In one embodiment, the patient subtype classifier is a trained machine-learned model. Thus, the patient subtype classifier can be trained to receive, as input, quantitative data of a biomarker set, and analyze the input to output a classification for the patient. In some embodiments, the patient subtype classifier is not a machine-learned model. In various embodiments, patient subtype classifier outputs a prediction of one classification for the patient out of X possible classifications. For example, the patient subtype classifier can output a prediction of a patient subtype for the patient out of a possible X patient subtypes. In various embodiments, X may be two possible classifications. In various embodiments, X may be more than two possible classifications. In various embodiments, X may be three, four, five, six, seven, eight, nine, or ten possible classifications. In various embodiments, X may be more than ten possible classifications.

In some embodiments, the patient classification system 130 calculates scores from the quantitative data and then provides the calculated scores as input to the patient subtype classifier. Thus, the patient subtype classifier determines a classification for the patient based on the calculated scores.

In various embodiments, the patient classification system 130 calculates multiple scores, each score corresponding to a patient subtype (e.g., classification). For example, if the goal is to classify the patient in a classification out of X possible classifications, the patient classification system 130 calculates X scores. The X scores are then provided as input to the patient subtype classifier to predict the classification. These scores are hereafter referred to as classification-specific scores.

In various embodiments, to calculate a classification-specific score, the patient classification system 130 determines subscores derived from quantitative data of one or more biomarkers in the biomarker set and uses the subscores to determine the classification-specific score. In one embodiment, a subscore is calculated from one or more biomarkers that are differentially expressed in the patient in comparison to a control value. In various embodiments, the control value may be a value derived from a different set of patients, such as healthy patients. In various embodiments, the control value may be a baseline value derived from the same patient (e.g., a baseline value corresponding to when the same patient was previously healthy).

In various embodiments, the patient classification system 130 determines a subscore determined from quantitative data of one or more biomarkers that are upregulated in the patient in comparison to the control value. In various embodiments, the patient classification system 130 determines a subscore determined from quantitative data of one or more biomarkers that are downregulated in the patient in comparison to the control value. In various embodiments, the patient classification system 130 determines a first subscore determined from quantitative data of one or more biomarkers that are upregulated in the patient in comparison to the control value and further determines a second subscore determined from quantitative data of one or more biomarkers that are downregulated in the patient in comparison to a control value. In various embodiments, a subscore can be an aggregation of the quantitative data of the one or more biomarkers. For example, a subscore can be a mean, a median, or a geometric mean of quantitative data of the one or more biomarkers. In various embodiments, the patient classification system 130 can further scale the sub scores.

In various embodiments, the quantitative data of one or more biomarkers that are analyzed refer to biomarkers that have been previously categorized as influencing the particular subtype that the classification-specific score is being calculated for. For example, if the patient classification system 130 is determining a classification-specific for subtype A, the patient classification system 130 determines subscores using quantitative data of biomarkers that are categorized as influencing the subtype A. Examples of biomarkers that are categorized with certain subtypes are shown below in Tables 1, 2A-2B, 3, and 4A-4D. Specifically, row number 1 in each of Tables 1, 2A-2B, 3, and 4A-4D show biomarkers that are categorized with subtype A, row number 2 in each of Tables 1, 2A-2B, 3, and 4A-4D show biomarkers that are categorized with subtype B, and row number 3 in each of Tables 1, 2A-2B, 3, and 4A-4D show biomarkers that are categorized with subtype C.

In various embodiments, the patient classification system 130 combines one or more subscores to determine the classification-specific score. For example, the patient classification system 130 can determine a difference between a first subscore and a second subscore. The difference can represent the classification-specific score.

As a specific example, the patient classification system 130 can determine a classification-specific score using the following steps: the patient classification system 130 determines a first geometric mean of the quantitative expression data for the subject for one or more biomarkers of the candidate classification, wherein the quantitative expression data for the subject for the one or more biomarkers of the candidate classification are increased relative to the quantitative expression data for the one or more biomarkers for one or more control subjects. The patient classification system 130 determines a second geometric mean of the quantitative expression for the subject for one or more additional biomarkers of the candidate classification, wherein the quantitative expression data for the subject for the one or more additional biomarkers of the candidate classification are decreased relative to the quantitative expression data for the one or more additional biomarkers for the one or more control subjects. The patient classification system 130 determines a difference between the first geometric mean and the second geometric mean, the first and second geometric means optionally subject to scaling. Here, the difference can represent the classification-specific score.

In various embodiments, the patient classification system 130 determines multiple classification-specific scores and provides them as input to the patient subtype classifier. The patient subtype classifier analyzes the classification-specific scores and outputs a classification for the patient. Embodiments of the patient subtype classifier are described in further detail below.

In various embodiments, based on the classification-specific scores, the patient subtype classifier outputs a classification. For example, the patient subtype classifier may analyze X classification-specific scores and outputs a prediction for one class out of Xpossible classifications. As another, the patient subtype classifier may analyze X classification-specific scores and outputs a prediction for one class out of two possible classifications. As a specific example, the patient subtype classifier may analyze 3 classification-specific scores (e.g., specific for subtype A, subtype B, and subtype C), and outputs a prediction for a class out of two possible classifications (e.g., subtype A v. not subtype A, subtype B v. not subtype B, or subtype C v. not subtype C).

Generally, the classification determined by the patient subtype classifier guides the selection of a therapy recommendation. In various embodiments, the therapy recommendation refers to whether a therapy is likely to be beneficial to a patient. In particular embodiments, the disease of interest is sepsis and therefore, the therapy recommendation pertain to whether a corticosteroid therapy, such as hydrocortisone, is likely to be of benefit to a patient. In one embodiment, the therapy recommendation can indicate whether the patient is likely to be “favorably responsive” or “non-responsive” to a therapy. In one embodiment, the therapy recommendation can indicate whether the patient is likely to be “favorably responsive”, “adversely responsive”, or “non-responsive” to a therapy.

Examples of a therapy include: immune stimulation therapy, suppression of immune regulation therapy, blocking of immune suppression therapy, blocking of complement activity therapy, anti-inflammatory therapy, a checkpoint inhibitor, a blocker of complement components, a blocker of complement component receptors, a blocker of a pro-inflammatory cytokine, modulators of coagulation therapy, and modulators of vascular permeability therapy. Additional examples of a therapy include: GM-CSF, anti-PD-1, anti-PD-L1, anti-CLTA-4, anti-CEACAM-1, anti-TIM-3, anti-BTLA, IL-7, INF-gamma, IFN-beta 1a regulator, IL-22 agonist, IFN-alpha regulator, IFN-lambda regulator, IFN-alpha 2b stimulant, anti-C5a, anti-C3a, anti-C5aR, anti-C3aR, anti-TNF-alpha, and anti-IL-6, Anti-HMGB1, ST2 antibody, IL-33 antibody, activated protein C, antithrombin, and thrombomodulin.

Additional examples of a therapy and corresponding therapy recommendations for different patient subtypes (e.g., subtype A, subtype B, and subtype C) are shown below in Table 8. Specifically, the therapy recommendations are shown in the column titled “Subtype Hypothesis” and support for that hypothesis is found in the column titled “Evidence.” Altogether the therapy recommendation determined by the patient classification system 130 can be provided to guide therapy for the patient.

The impact of a particular therapy and a patient subtype, such as those hypothesized in Table 8, may have been previously determined by analyzing patient cohorts who have received the particular therapy. For example, such patient cohorts may have been involved in a clinical trial. Thus, the patients may be exhibiting dysregulated host responses and therefore, were enrolled in the trial. Therefore, patients in the clinical trial are classified with a patient subtype (e.g., using the methods described above) and their responses to the therapy (e.g., favorably responsive, adversely responsive, non-responsive) are tracked and recorded. For each subtype, the responses of patients receiving the therapy are compared to control patients. If the comparison yields a statistically significant difference patients of the subtype are labeled as favorably responsive or adversely responsive to the therapy. If the comparison does not yield a statistically significant difference (e.g., p-value not greater than a threshold value), patients of the subtype are labeled as non-responsive to the therapy. In various embodiments, the statistical significance threshold is a p-value, where the p-value is any one of 0.01, 0.0.5, or 0.1.

In particular embodiments, the compared measurable on which statistical significance is determined is patient mortality. Therefore, the mortality of patients who receive a therapy is compared to mortality of control patients to determine whether there is statistical significance indicating an effect due to the therapy. For example, if the patients of a subtype who receive a therapy exhibit a statistically significantly increased survival time in comparison to control patients who did not receive the therapy, then patients of this subtype can be identified as favorably responsive to the therapy. As another example, if patients of a subtype who receive a therapy exhibit a statistically significantly decreased survival time in comparison to control patients who did not receive the therapy, then patients of this subtype can be identified as adversely responsive to the therapy. As yet another example, if patients of a subtype who receive a therapy do not exhibit a statistically significantly increased or a statistically significantly decreased survival time in comparison to control patients who did not receive the therapy, then patients of this subtype can be identified as not responsive to the therapy.

IIB. Methods for Determining a Therapy Hypothesis

In various embodiments, methods disclosed herein involve the identification of therapeutic hypotheses for different patient subtypes. In various embodiments, the process of identifying a therapeutic hypothesis is performed by the patient classification system 130. In some embodiments, the process of identifying a therapeutic hypothesis is performed by third party system which provides a therapeutic hypothesis to the patient classification system 130. In various embodiments, a therapy hypothesis is specific for a patient subtype. Therefore, a therapy hypothesis is useful for identifying a therapy recommendation, as discussed above in reference to FIG. 1B.

Generally, a therapeutic hypothesis involves analyzing genes that are differentially expressed across different subtypes. By identifying patterns of differentially expressed genes that are implicated in certain known biological pathways, certain patient subtypes can be associated with particular dysregulated pathways. A therapeutic hypothesis comprising a candidate therapeutic can be selected. Here, a candidate therapeutic can modulate parts of the dysregulated pathways, thereby representing a possible avenue of therapy for treating particular patient subtypes.

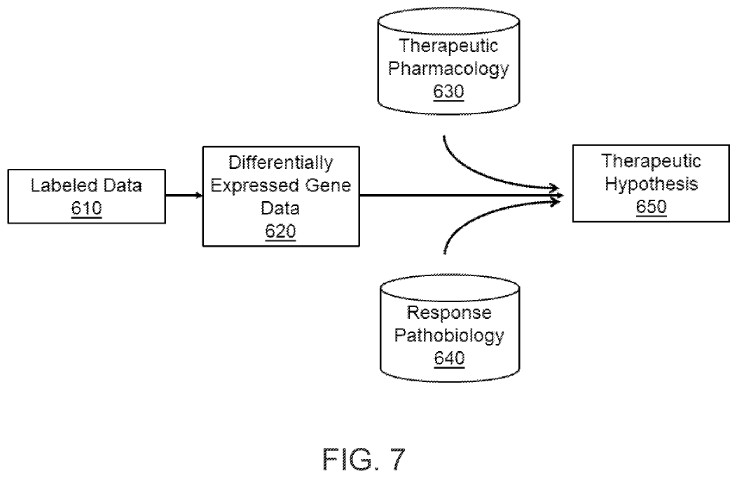

Reference is now made to FIG. 7, which depicts an example flow process for determining therapeutic hypotheses for patient subtypes, in accordance with an embodiment. Generally, FIG. 7 depicts the use of labeled data 610 to generate differentially expressed gene data 620. The differentially expressed gene data 620 can be used to identify a therapeutic hypothesis 650. In some embodiments, the differentially expressed gene data 620 is analyzed together with therapeutic pharmacology data 630 and response pathobiology data 640 to determine the therapeutic hypothesis. In some embodiments, the differentially expressed gene data 620 is analyzed with one of therapeutic pharmacology data 630 or respond pathobiology data 640 to determine the therapeutic hypothesis 650. In some embodiments, only the differentially expressed gene data 620 is analyzed to determine the therapeutic hypothesis 650.

The labeled data 610 represents patient data that have been labeled with one or more classifications. For example, the labeled data 610 can be labeled with patient subtypes (e.g., subtype A, subtype B, subtype C, etc.). In various embodiments, the patient data comprises quantitative data of one or more biomarkers of patients. In various embodiments, the patient data is clinical trial data and therefore, the quantitative data of one or more biomarkers can be data obtained from patients enrolled in the clinical trial.

The labels of the labeled data can be previously generated through various means. In various embodiments, the labels of the data can be generated using a model, such as a patient subtype classifier described herein. For example, the quantitative data of biomarkers from patients are analyzed using the patient subtype classifier to predict a classification for patients. Thus, the predicted classification for each patient can serve as a label for the labeled data. In various embodiments, the labels of the data can be generated through a clustering analysis. For example, the quantitative data of biomarkers can be analyzed through unsupervised clustering, thereby generating clusters of patients that have similar expression of various biomarkers. Each cluster of patients can be labeled. In various embodiments, a cluster can be labeled based on outcomes of patients in the clinical trials. For example, if a majority of patients in a cluster exhibited prolonged survival time in response to a therapy, the cluster can be labeled as a subtype that is responsive to the therapy.

The differentially expressed gene data 620 comprises gene level fold changes of biomarker expression between patients of different subtypes. Using the labeled data 610, gene expression from patients of individual subtypes are aggregated and compared across subtypes. For example, a statistical measure of gene expression for patients of a subtype can be determined (e.g., a mean, a median, a mode, a geometric mean). The statistical measure of gene expression for patients of a first subtype are compared to a statistical measure of gene expression for patients of a second subtype. This can be performed across the different patient subtypes and across various genes. Thus, the differentially expressed gene data 620 includes gene level fold changes of different biomarkers across different patient subtypes.

In various embodiments, the differentially expressed gene data 620 includes gene level fold changes for at least twenty biomarkers. In various embodiments, the differentially expressed gene data 620 includes gene level fold changes for at least fifty biomarkers. In various embodiments, the differentially expressed gene data 620 includes gene level fold changes for at least 100 biomarkers, at least 200 biomarkers, at least 300 biomarkers, at least 400 biomarkers, at least 500 biomarkers, at least 1000 biomarkers, at least 2000 biomarkers, at least 3000 biomarkers, at least 4000 biomarkers, at least 5000 biomarkers, at least 10,000 biomarkers, at least 50,000 biomarkers, or at least 100,000 biomarkers.

In various embodiments, the differentially expressed gene data 620 can be represented as a database or a table that documents gene level fold changes between patients of different subtypes. An example of such a gene level fold changes between patient subtypes is shown below in Table 7. Specifically, for each gene, a gene level fold change (e.g., ratio) between different subtypes (e.g., subtype A/subtype B denoted as “A/B”) is shown.

To determine a therapeutic hypothesis 650, patterns of gene level fold changes are identified across the differentially expressed gene data 620. In various embodiments, patterns of gene level fold changes refer to at least a threshold number of genes that are differentially expressed in a first patient subtype in comparison to a second patient subtype. In various embodiments, patterns of gene level fold changes refer to at least a threshold number of genes that are overexpressed in a first patient subtype in comparison to a second patient subtype. In various embodiments, patterns of gene level fold changes refer to at least a threshold number of genes that are underexpressed in a first patient subtype in comparison to a second patient subtype.

In various embodiments, the threshold number of genes include genes that are involved in a common biological pathway. Example biological pathways include, but are not limited to: innate immune pathways, chronic inflammation pathways, acute inflammation pathways, coagulation pathways, complement pathways, signaling pathways (e.g., TLR signaling pathway or glucocorticoid receptor signaling pathway), and the like. In various embodiments, the involvement of genes in certain biological pathways is curated from publicly available databases such as the Reactome Pathway Database or the KEGG Pathway database.

In various embodiments, the threshold number of genes involved in a common biological pathway is at least 2 genes. In various embodiments, the threshold number of genes is at least 3 genes, at least 4 genes, at least 5 genes, at least 6 genes, at least 7 genes, at least 8 genes, at least 9 genes, at least 10 genes, at least 15 genes, at least 20 genes, at least 25 genes, at least 50 genes, at least 75 genes, at least 100 genes, at least 200 genes, at least 300 genes, at least 400 genes, at least 500 genes, or at least 1000 genes. In various embodiments, the threshold number of genes involved in a common biological pathway is 2 genes. In various embodiments, the threshold number of genes involved in a common biological pathway is 3 genes, 4 genes, 5 genes, 6 genes, 7 genes, 8 genes, 9 genes, 10 genes, 11 genes, 12 genes, 13 genes, 14 genes, 15 genes, 16 genes, 17 genes, 18 genes, 19 genes, 20 genes, 25 genes, 50 genes, 75 genes, 100 genes, 200 genes, 300 genes, 400 genes, 500 genes, 600 genes, 700 genes, 800 genes, 900 genes, or 1000 genes.

Altogether, patterns of gene level fold changes, as indicated by a threshold number of genes involved in a common biological pathway, are useful for understanding the underlying biology that may be involved in a patient subtype. For example, genes involved in inflammation may be differentially expressed in subtype A in comparison to those genes in subtype B. Thus, subtype A can be associated or characterized by inflammation based processes.

The patterns of gene level fold changes between subtypes is analyzed to determine a therapeutic hypothesis 650 which, in some scenarios, includes a class of a candidate therapeutic of a candidate therapeutic itself (e.g., including but not limited to a drug therapy or a gene therapy). For example, given the characterization that a particular patient subtype is associated with an underlying biological pathway or process, a target involved in the biological pathway or process can serve as a druggable target. Thus, a class of a candidate therapeutic or a candidate therapeutic that modulates the target involved in the biological pathway can be promising as a therapeutic hypothesis 650. Examples of a class of a therapy include, but are not limited to: immune stimulation therapy, suppression of immune regulation therapy, blocking of immune suppression therapy, blocking of complement activity therapy, anti-inflammatory therapy, a checkpoint inhibitor, a blocker of complement components, a blocker of complement component receptors, a blocker of a pro-inflammatory cytokine, modulators of coagulation therapy, and modulators of vascular permeability therapy. Examples of a candidate therapy include but are not limited to: GM-CSF, anti-PD-1, anti-PD-L1, anti-CLTA-4, anti-CEACAM-1, anti-TIM-3, anti-BTLA, IL-7, INF-gamma, IFN-beta 1a regulator, IL-22 agonist, IFN-alpha regulator, IFN-lambda regulator, IFN-alpha 2b stimulant, anti-C5a, anti-C3a, anti-C5aR, anti-C3aR, anti-TNF-alpha, and anti-IL-6, Anti-HMGB1, ST2 antibody, IL-33 antibody, activated protein C, antithrombin, and thrombomodulin.

The therapeutic pharmacology data 630 is useful for developing a therapeutic hypothesis for a particular class of therapy or for a particular candidate therapy. Generally, therapeutic pharmacology data 630 is useful for understanding what therapeutic effects, if any, can be imparted by a class of therapy or candidate therapy. For example, therapeutic pharmacology data 630 can include molecular data of therapeutics, clinical pharmacology data of therapeutics (e.g., pharmacokinetics and pharmacodynamics data), and/or data identifying therapeutics that are useful for modulating activity in particular biological pathways. For example, for a given candidate therapeutic (e.g., an anti-PD-1 inhibitor), the therapeutic pharmacology data 630 is useful for understanding how different patients respond to the anti-PD-1 inhibitor.

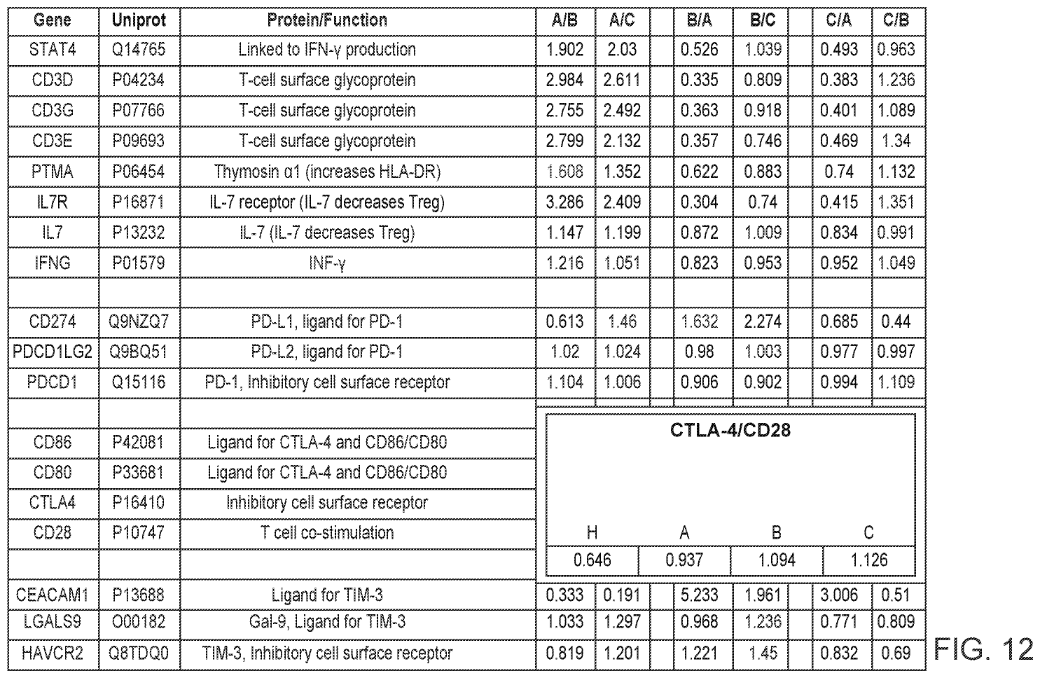

Examples of therapeutic pharmacology data 630 is shown in FIG. 12. For example, PD-1 blockade is expected to up-regulate IL-7 and CTLA-4 blockade is expected to up-regulate INF-gamma and to stimulate immune activity more broadly. In patients with down-regulated immune activity, PD-L1 and CTLA-4 is up-regulated, while IL-7 and INF-gamma are down-regulated. Therefore, blockade of PD-1/PD-L1 will likely result in up-regulation of IL-7 and blockade of CTLA-4 upregulation of INF-gamma, and stimulation of immune activity more broadly.

The response pathobiology data 640 is useful for developing a hypothesis as to therapeutic effects, independent of a particular candidate therapeutic, that may benefit a particular patient subtype. In various embodiments, response pathobiology data 640 can include patient data corresponding to patients that responded favorably. In various embodiments, response pathobiology data 640 includes patient data of patient subtypes that indicate differential expression of biomarkers associated with certain biological activity. The differentially expressed biomarkers can be promising targets for modulation. For example, dysregulated host response patients of subtype A exhibit up-regulation of biomarkers associated with innate immune activity involved in pathogen recognition (e.g., via recognition of pathogen-associated molecular patterns (PAMPs)), up-regulation of biomarkers associated with innate immune regulation, and up-regulation of biomarkers associated with adaptive immune activity. As another example, dysregulated host response patients of subtype B exhibit up-regulation of biomarkers associated with innate immune activity involved in recognition of damage-associated molecular patterns (DAMPs), up-regulation of biomarkers associated with DAMPs, up-regulation of biomarkers associated with inflammation (e.g. TNF-alpha), up-regulation of biomarkers associated with complement activity, down-regulation of biomarkers associated with adaptive immune activity, up-regulation of biomarkers associated with adaptive immune suppression, and up-regulation of markers associated with increased risk of acute kidney injury. As another example, subtype C patients exhibit down-regulation of biomarkers associated with innate and adaptive immune activity, up-regulation of biomarkers associated with DAMPs, up-regulation of biomarkers associated with cellular recruitment (e.g. G-CSF and GM-CSF), up-regulation of biomarkers associated with increased risk of thrombosis, and up-regulation of biomarkers associated with coagulation.

The therapeutic hypothesis 650 for a patient subtype can be subsequently tested and validated. For example, the therapeutic hypothesis 650 can be tested in pre-clinical or clinical studies and trials (e.g., a randomized controlled trial) by providing subjects or patients of the subtype a candidate therapeutic and monitoring their response.

IIC. Patient Subtype Classifier

In various embodiments, the patient subtype classifier is a machine-learned model that analyzes quantitative data of biomarkers or classification-specific scores derived from quantitative data of biomarkers and predicts a classification. In various embodiments, the patient subtype classifier is any one of a regression model (e.g., linear regression, logistic regression, or polynomial regression), decision tree, random forest, support vector machine, Naïve Bayes model, k-means cluster, or neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks), or any combination thereof.

The patient subtype classifier can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In various embodiments, the patient subtype classifier is trained using supervised learning algorithms, unsupervised learning algorithms, semi-supervised learning algorithms (e.g., partial supervision), weak supervision, transfer, multi-task learning, or any combination thereof.

In various embodiments, the patient subtype classifier has one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, and coefficients in a regression model. The model parameters of the patient subtype classifier are trained (e.g., adjusted) using the training data to improve the predictive capacity of the patient subtype classifier.

In some embodiments, the patient subtype classifier is a regression, such as a logistic regression. Parameters of the logistic regression are trained using the training data such that when the logistic regression is applied, it outputs a classification based on the different classification-specific scores. The parameters of the logistic regression can be trained to maximize the differences between the different classifications (e.g., subtype A, subtype B, and subtype C).

In some embodiments, the patient subtype classifier is a support vector machine. The support vector machine is trained with a single or a set of hyperplanes that maximizes the differences among the X different classifications. In one embodiment, the support vector machine is trained with single or a set of hyperplanes that maximizes the differences among 3 different classifications (e.g., subtype A, subtype B, and subtype C). As a specific example, the support vector machine is trained with a set of hyperplanes that maximizes the differences among the 3 different classification-specific scores (e.g., scores for each of subtype A, subtype B, and subtype C). Therefore, the trained support vector machine can use the hyperplanes to output a prediction of a classification when provided quantitative data of biomarkers or classification-specific scores derived from quantitative data of biomarkers.

In some embodiments, the patient subtype classifier may be a non-machine learned model. The patient subtype classifier may employ one or more threshold values for comparison against the classification-specific scores. Depending on the comparison between the threshold values and the classification-specific scores, the patient subtype classifier outputs a predicted classification. In various embodiments, a threshold value is specific for a classification. Therefore, there may be X threshold values to be compared against X classification-specific scores.

In some embodiments, a threshold value may be a fixed value (e.g., fixed value=0). Here, the classification-specific scores are compared to the fixed threshold value and patient subtype classifier determines the classification based on the comparison. For example, assuming there are two classification-specific scores, the patient subtype classifier may compare each of the first classification-specific score and the second classification-specific score to the fixed threshold. In one embodiment, if the first classification-specific score is greater than the fixed threshold value and the second classification-specific score is less than a fixed threshold value, then the patient subtype classifier can output a particular classification. Similar logic can be applied for determining classifications using more than two classification-specific scores.

In some embodiments, a threshold value may be determined from training samples including data from patients who have been classified (e.g., classified as subtype A, subtype B, and/or subtype C). Such a threshold value may derived from a receiver operating curve (ROC) demonstrating the sensitivity/specificity of a model that classified the patients of the training samples. For example, for patients in the training sample classified as subtype A, a receiver operating curve is generated that demonstrates the sensitivity and specificity of the classifier. The threshold value can be the top-left part of the plot, representing the closest point in the ROC to perfect sensitivity or specificity.

The classification-specific scores are compared to corresponding threshold values, and based on the comparison, the patient subtype classifier determines the classification. For example, assuming there are two classification-specific scores for subtype A and subtype B, the patient subtype classifier may compare the subtype A classification-specific score to a subtype A threshold value and may further compare the subtype B classification-specific score to a subtype B threshold value. Thus, depending on the two comparisons, the patient subtype classifier determines the classification. In one embodiment, if the first classification-specific score is greater than the first threshold value and the second classification-specific score is less than a second threshold value, then the patient subtype classifier can output a particular classification. Similar logic can be applied for determining classifications using more than two classification-specific scores and/or more than two threshold values. Examples of subtype specific threshold values that are derived from training samples are described below in Table 18.

IID. Biomarker Panel

Embodiments described herein involve the analysis of biomarkers. As described herein, a biomarker panel, also referred to as a biomarker set, can be implemented to analyze values of biomarkers for a patient. In various embodiments, a biomarker panel can be a multivariate biomarker panel. In such embodiments, the multivariate biomarker panel includes more than one biomarker. In various embodiments, the multivariate biomarker panel includes two biomarkers. In various embodiments, the multivariate biomarker panel includes 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 biomarkers. In particular embodiments, the multivariate biomarker panel includes 3 biomarkers. In particular embodiments, the multivariate biomarker panel includes 4 biomarkers. In particular embodiments, the multivariate biomarker panel includes 5 biomarkers. In particular embodiments, the multivariate biomarker panel includes 6 biomarkers. In particular embodiments, the multivariate biomarker panel includes 8 biomarkers. In particular embodiments, the multivariate biomarker panel includes 10 biomarkers. In particular embodiments, the multivariate biomarker panel includes 15 biomarkers. In particular embodiments, the multivariate biomarker panel includes 16 biomarkers. In particular embodiments, the multivariate biomarker panel includes 24 biomarkers.

In various embodiments, the multivariate biomarker panel includes biomarkers selected from the following markers: EVL, BTN3A2, HLA-DPA1, IDH3A, ACBD3, EXOSC10, SNRK, MMP8, SERPINB1, GSPT1, MPP1, HMBS, TAL1, C9orf78, POLR2L, SLC27A3, DDX50, FCHSD2, GSTK1, UBE2E1, TNFRSF1A, PRPF3, TOMM70A, ZNF831, MME, CD3G, STOM, ECSIT, LAT, NCOA4, SLC1A5, IGF2BP2, ANXA3, C14orf159, PUM2, EPB42, RPS6KA5, GBP2, MSH2, DCTD, HK3, UCP2, NUP88, GABARAPL2, and CASP4.

In various embodiments, the multivariate biomarker panel includes at least two biomarkers selected from the following markers: EVL, BTN3A2, HLA-DPA1, IDH3A, ACBD3, EXOSC10, SNRK, MMP8, SERPINB1, GSPT1, MPP1, HMBS, TAL1, C9orf78, POLR2L, SLC27A3, DDX50, FCHSD2, GSTK1, UBE2E1, TNFRSF1A, PRPF3, TOMM70A, ZNF831, MME, CD3G, STOM, ECSIT, LAT, NCOA4, SLC1A5, IGF2BP2, ANXA3, C14orf159, PUM2, EPB42, RPS6KA5, GBP2, MSH2, DCTD, HK3, UCP2, NUP88, GABARAPL2, and CASP4.

In various embodiments, the multivariate biomarker panel include X number of biomarkers, where X is the number of possible classifications that the patient subtype classifier can predict. For example, for a patient subtype classifier that predicts three different subtypes (e.g., subtype A, subtype B, and subtype C), the multivariate biomarker panel can include three different biomarkers.

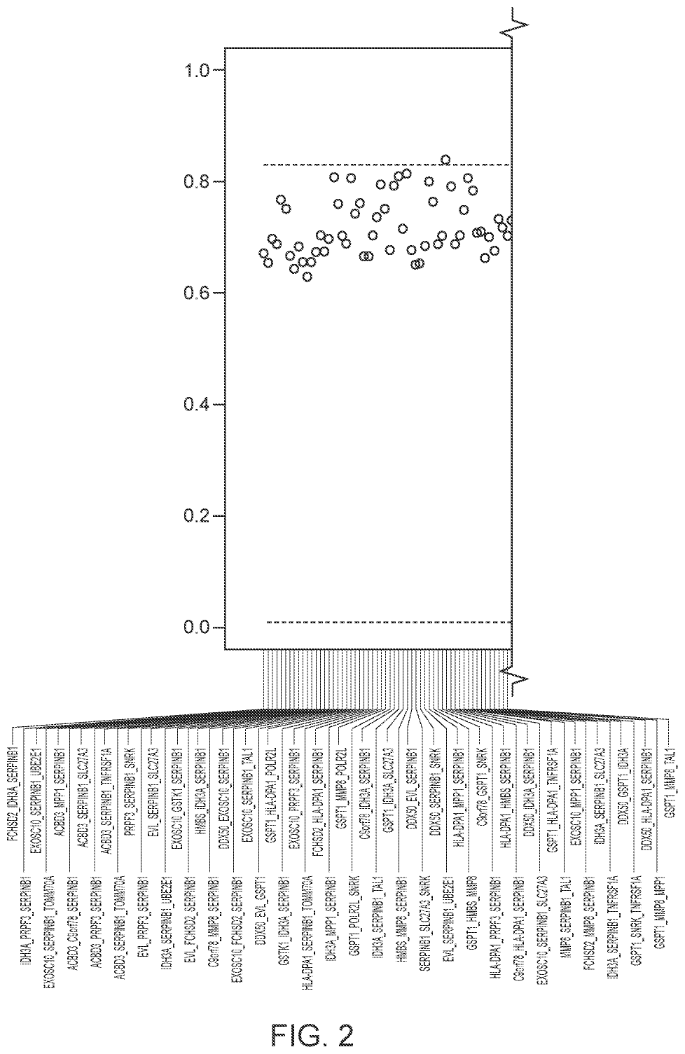

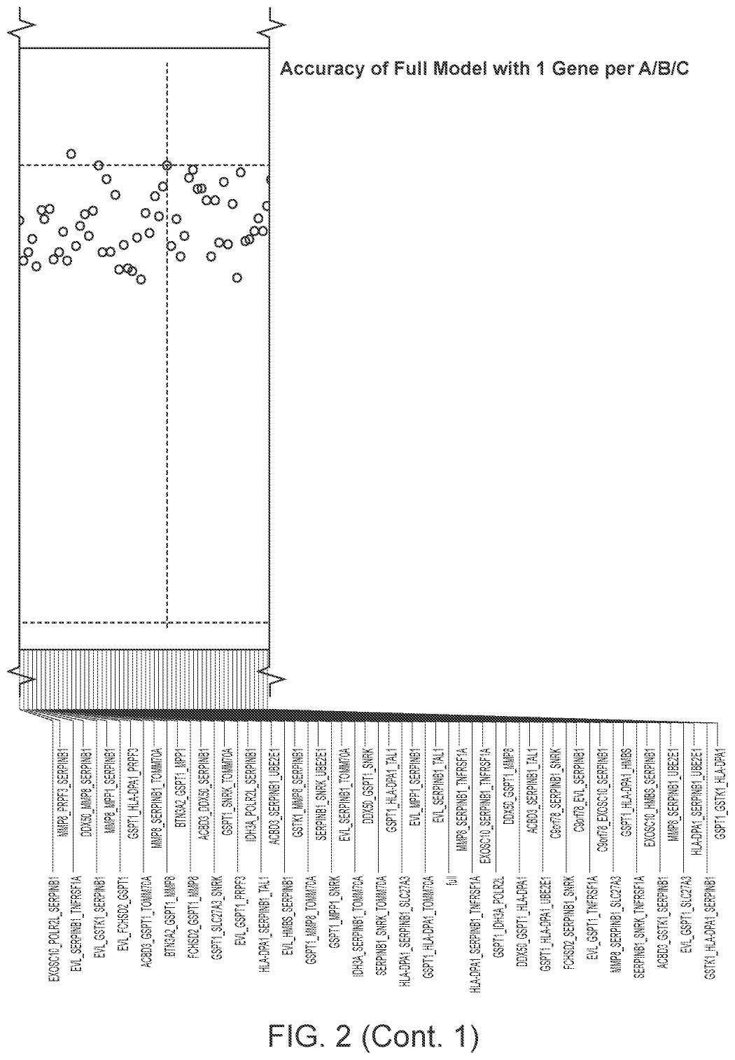

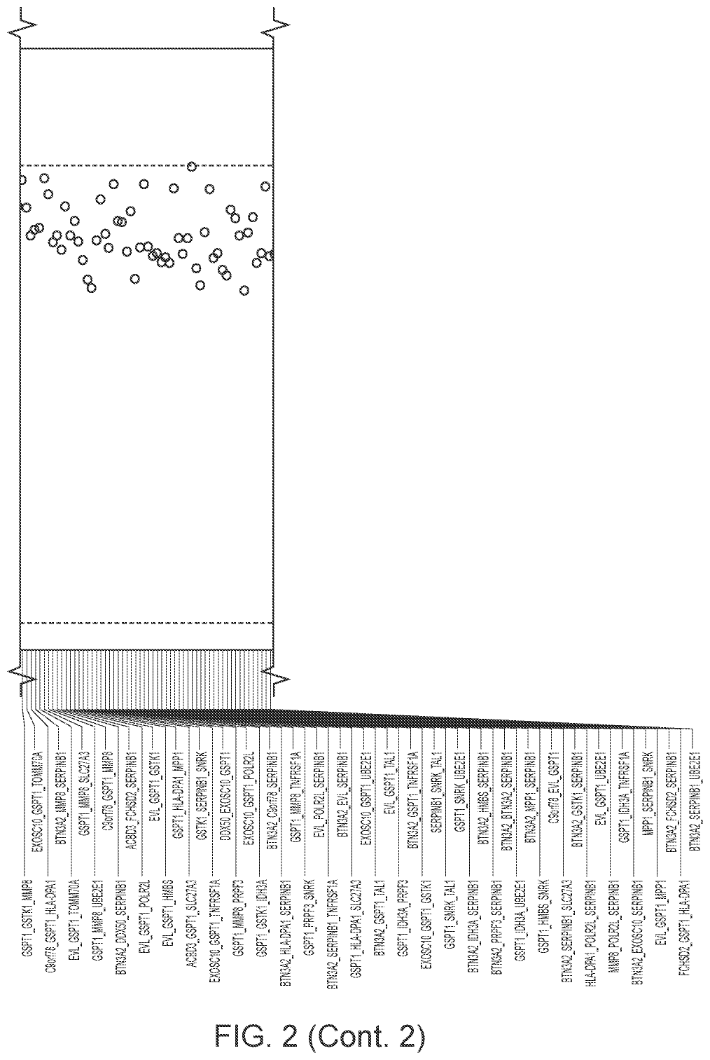

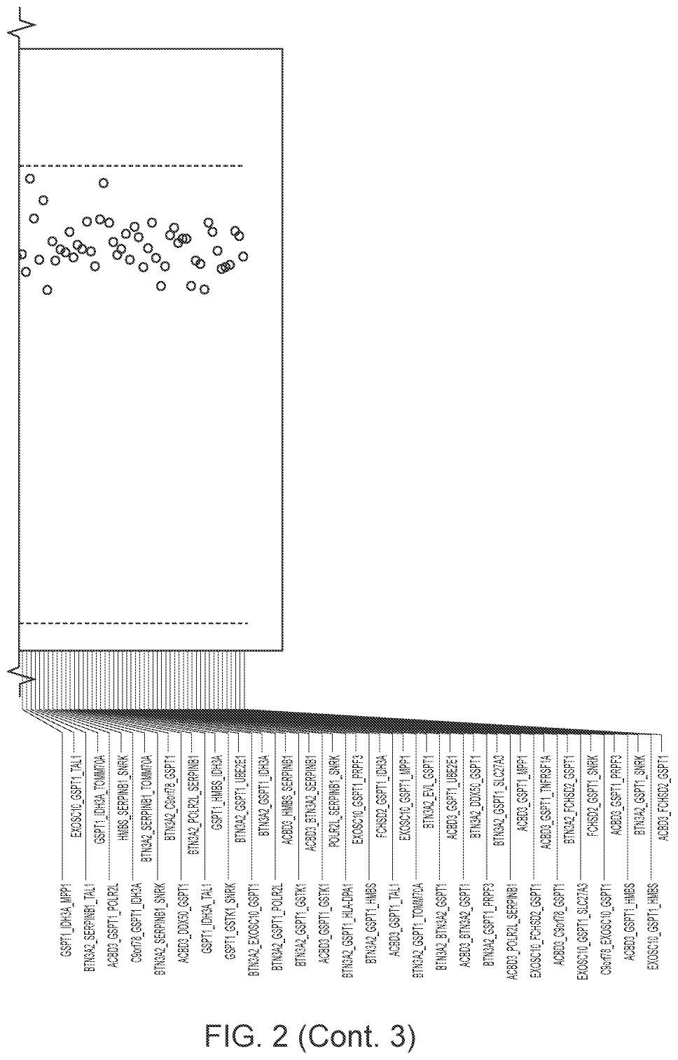

In various embodiments, the multivariate biomarker panel includes a first biomarker selected from EVL, BTN3A2, HLA-DPA1, IDH3A, ACBD3, EXOSC10, SNRK, MMP8, a second biomarker selected from SERPINB1 and GSPT1, and a third biomarker selected from MPP1, HMBS, TAL1, C9orf78, POLR2L, SLC27A3, BTN3A2, DDX50, FCHSD2, GSTK1, UBE2E1, TNFRSF1A, PRPF3, and TOMM70A. Example accuracies of a multivariate biomarker panel implementing combinations of three biomarkers described above is shown in FIG. 2.

In various embodiments, the multivariate biomarker panel includes one or more biomarkers selected from EVL, BTN3A2, HLA-DPA1, IDH3A, ACBD3, EXOSC10, SNRK, MMP8, one or more biomarkers selected from SERPINB1 and GSPT1, and one or more biomarkers selected from MPP1, HMBS, TAL1, C9orf78, POLR2L, SLC27A3, BTN3A2, DDX50, FCHSD2, GSTK1, UBE2E1, TNFRSF1A, PRPF3, and TOMM70A.

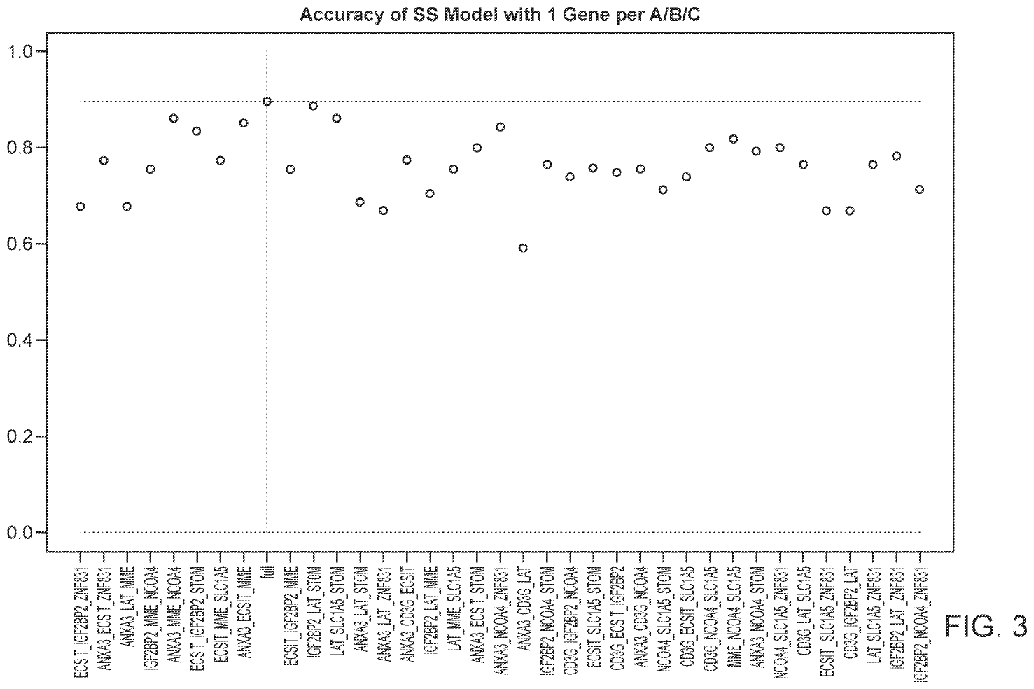

In one embodiment, the multivariate biomarker panel includes a first biomarker selected from ZNF831, MME, CD3G, and STOM, a second biomarker selected from ECSIT, LAT, and NCOA4, and a third biomarker selected from SLC1A5, IGF2BP2, and ANXA3. Example accuracies of a multivariate biomarker panel implementing combinations of three biomarkers described above is shown in FIG. 3.

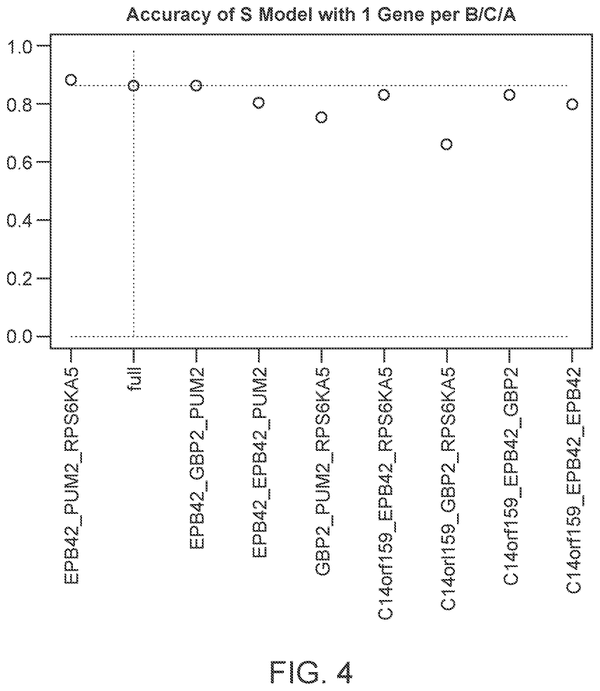

In one embodiment, the multivariate biomarker panel includes a first biomarker selected from C14orf159 and PUM2, a second biomarker selected from EPB42 and RPS6KA5, and a third biomarker selected from GBP2. Example accuracies of a multivariate biomarker panel implementing combinations of three biomarkers described above is shown in FIG. 4.

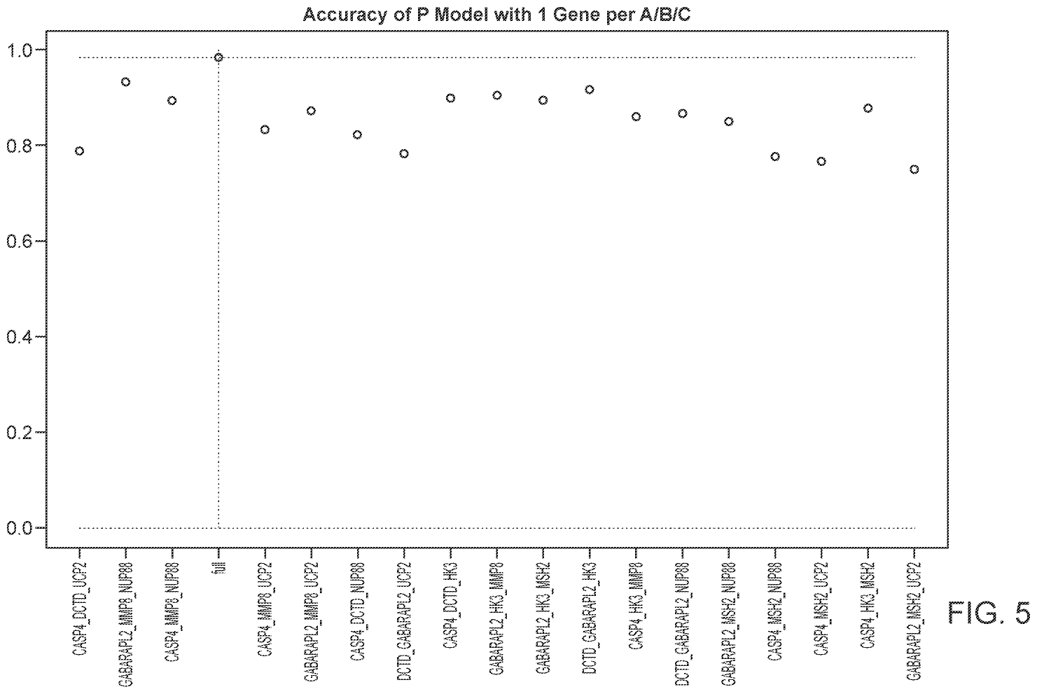

In one embodiment, the multivariate biomarker panel includes a first biomarker selected from MSH2, DCTD, and MMP8, a second biomarker selected from HK3, UCP2, and NUP88, and a third biomarker selected from GABARAPL2 and CASP4. Example accuracies of a multivariate biomarker panel implementing combinations of three biomarkers described above is shown in FIG. 5.

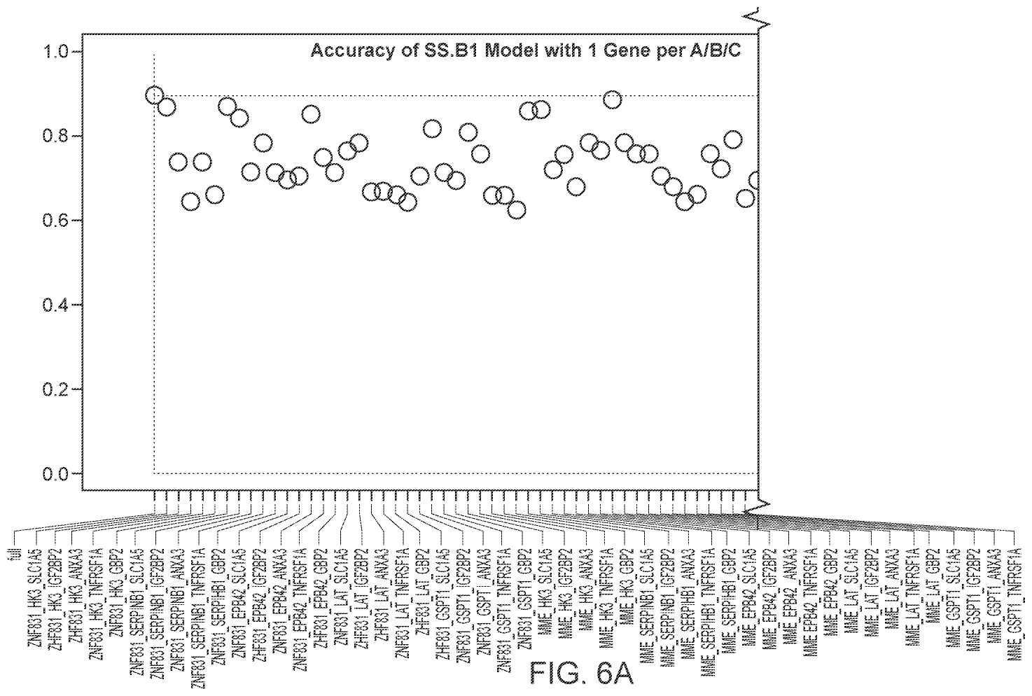

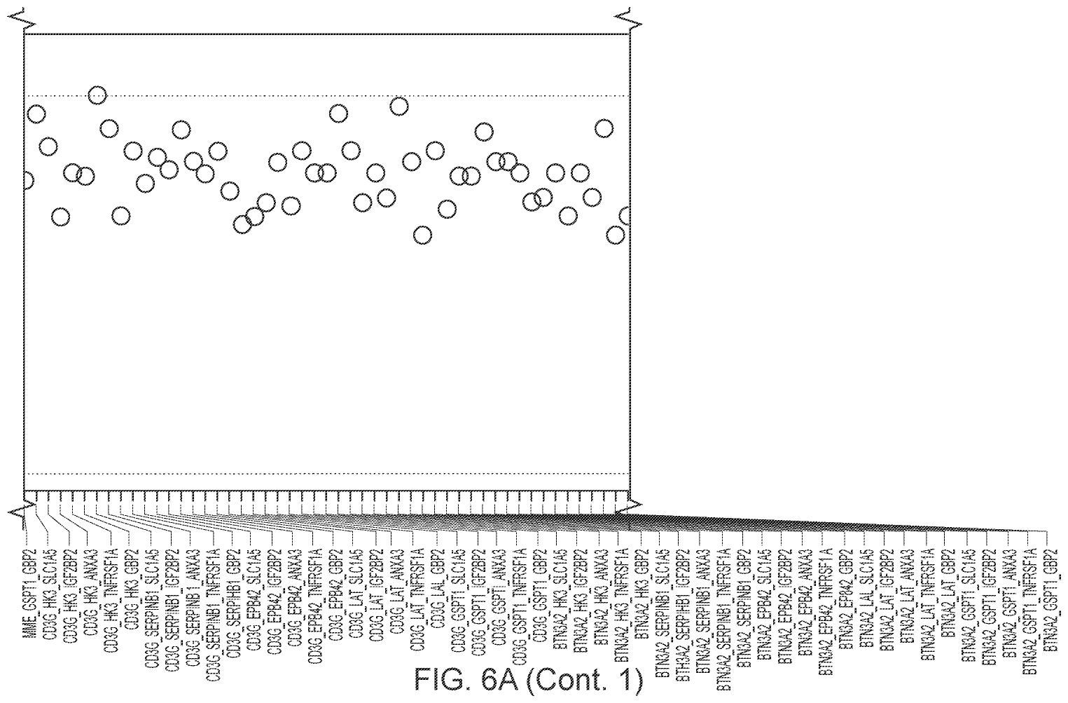

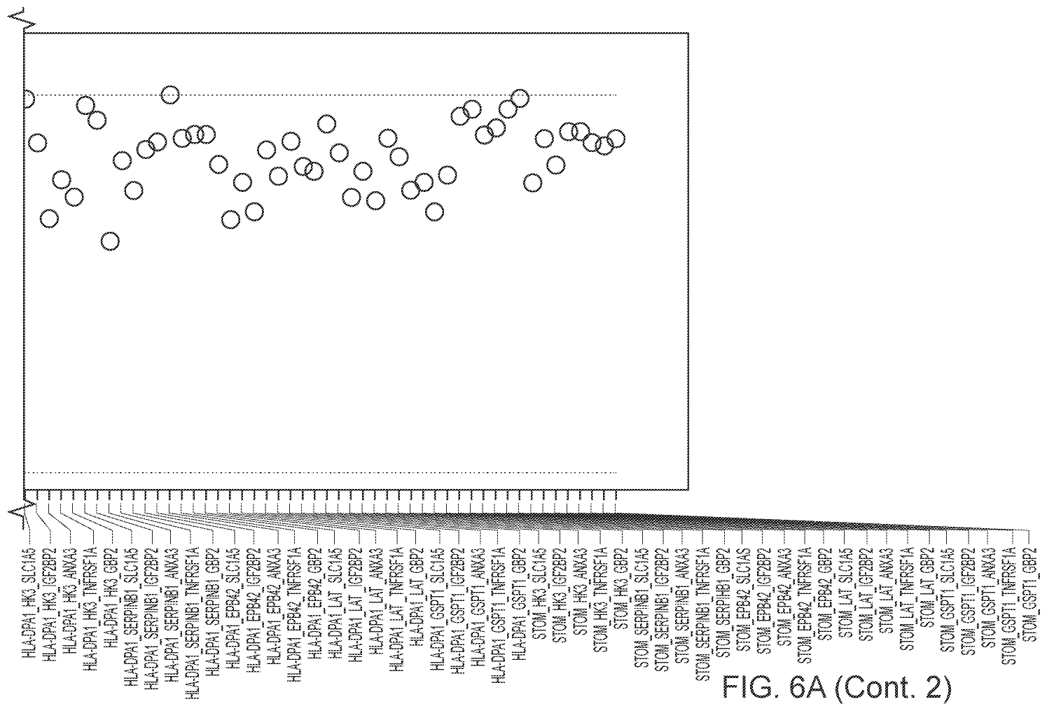

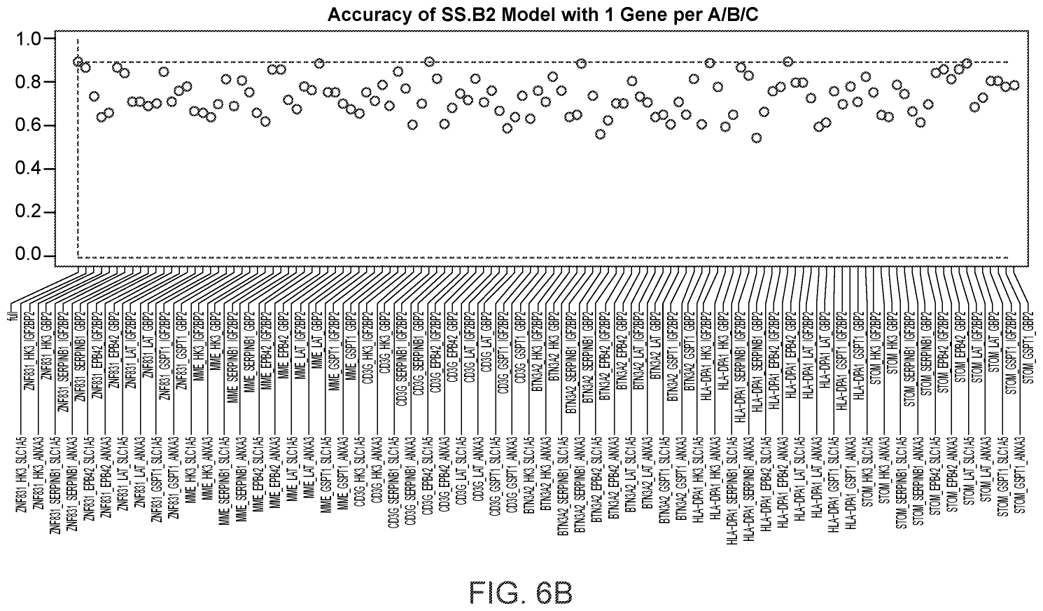

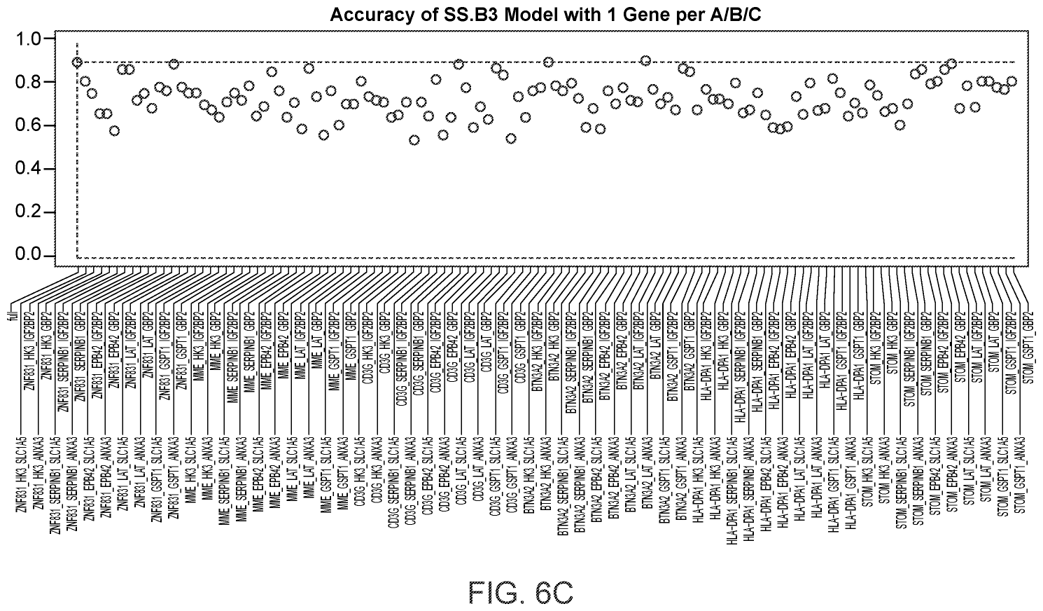

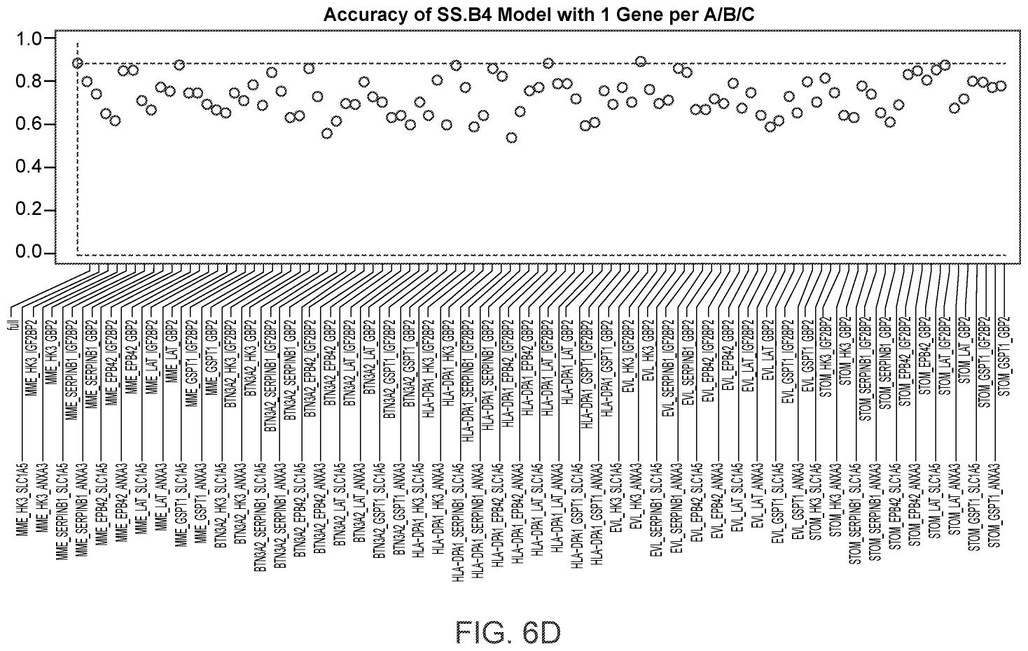

In one embodiment, the multivariate biomarker panel includes a first biomarker selected from STOM, ZNF831, CD3G, MME, BTN3A2, and HLA-DPA1, a second biomarker selected from EPB42, GSPT1, LAT, HK3, and SERPINB1, and a third biomarker selected from GBP2, TNFRSF1A, SLC1A5, IGF2BP2, and ANXA3. Example accuracies of a multivariate biomarker panel implementing combinations of three biomarkers described above is shown in FIG. 6A.

In one embodiment, the multivariate biomarker panel includes a first biomarker selected from STOM, ZNF831, CD3G, MME, BTN3A2, and HLA-DPA1, a second biomarker selected from EPB42, GSPT1, LAT, HK3, and SERPINB1, and a third biomarker selected from GBP2, SLC1A5, IGF2BP2, and ANXA3. Example accuracies of a multivariate biomarker panel implementing combinations of three biomarkers described above is shown in FIG. 6B.

In one embodiment, the multivariate biomarker panel includes a first biomarker selected from STOM, MME, BTN3A2, HLA-DPA1, and EVL, a second biomarker selected from EPB42, GSPT1, LAT, HK3, and SERPINB1, and a third biomarker selected from BTN3A2, TNFRSF1A, SLC1A5, IGF2BP2, and ANXA3. Example accuracies of a multivariate biomarker panel implementing combinations of three biomarkers described above is shown in FIG. 6C.

In one embodiment, the multivariate biomarker panel includes a first biomarker selected from STOM, MME, BTN3A2, HLA-DPA1, and EVL, a second biomarker selected from EPB42, GSPT1, LAT, HK3, and SERPINB1, and a third biomarker selected from GBP2, SLC1A5, IGF2BP2, and ANXA3. Example accuracies of a multivariate biomarker panel implementing combinations of three biomarkers described above is shown in FIG. 6D.

Although the embodiments described above may refer to a “first biomarker,” “second biomarker,” and/or “third biomarker,” the terms “first biomarker,” “second biomarker,” and/or “third biomarker,” each encompass one or more biomarkers. For example, a “first biomarker” can refer to one or more biomarkers selected from EVL, BTN3A2, HLA-DPA1, IDH3A, ACBD3, EXOSC10, SNRK, MMP8. A “second biomarker” can refer to one or more biomarkers selected from SERPINB1 and GSPT1. A “third biomarker” can refer to one or more biomarkers selected from MPP1, HMBS, TAL1, C9orf78, POLR2L, SLC27A3, BTN3A2, DDX50, FCHSD2, GSTK1, UBE2E1, TNFRSF1A, PRPF3, and TOMM70A.

In one embodiment, the multivariate biomarker panel includes four, five, six, seven, eight, nine, ten, eleven, twelve, thirteen, fourteen, fifteen, sixteen, seventeen, eighteen, nineteen, twenty, twenty one, twenty two, twenty three, or twenty four biomarkers selected from EVL, BTN3A2, HLA-DPA1, IDH3A, ACBD3, EXOSC10, SNRK, MMP8, SERPINB1, GSPT1, MPP1, HMBS, TAL1, C9orf78, POLR2L, SLC27A3, BTN3A2, DDX50, FCHSD2, GSTK1, UBE2E1, TNFRSF1A, PRPF3, and TOMM70A.

In one embodiment, the multivariate biomarker panel includes four, five, six, seven, eight, nine, or ten biomarkers selected from ZNF831, MME, CD3G, STOM, ECSIT, LAT, NCOA4, SLC1A5, IGF2BP2, and ANXA3.

In one embodiment, the multivariate biomarker panel includes four or five biomarkers selected from C14orf159, PUM2, EPB42, RPS6KA5, and GBP2.

In one embodiment, the multivariate biomarker panel includes four, five, six, seven, or eight biomarkers selected from MSH2, DCTD, MMP8, HK3, UCP2, NUP88, GABARAPL2 and CASP4.

In one embodiment, the multivariate biomarker panel includes four, five, six, seven, eight, nine, ten, eleven, twelve, thirteen, fourteen, fifteen or sixteen biomarkers selected from STOM, ZNF831, CD3G, MME, BTN3A2, HLA-DPA1, EPB42, GSPT1, LAT, HK3, SERPINB1, GBP2, TNFRSF1A, SLC1A5, IGF2BP2, and ANXA3.

In one embodiment, the multivariate biomarker panel includes four, five, six, seven, eight, nine, ten, eleven, twelve, thirteen, fourteen, or fifteen biomarkers selected from STOM, ZNF831, CD3G, MME, BTN3A2, HLA-DPA1, EPB42, GSPT1, LAT, HK3, SERPINB1, GBP2, SLC1A5, IGF2BP2, and ANXA3.

In one embodiment, the multivariate biomarker panel includes four, five, six, seven, eight, nine, ten, eleven, twelve, thirteen, fourteen, or fifteen biomarkers selected from STOM, MME, BTN3A2, HLA-DPA1, EVL, EPB42, GSPT1, LAT, HK3, SERPINB1, BTN3A2, TNFRSF1A, SLC1A5, IGF2BP2, and ANXA3.

In one embodiment, the multivariate biomarker panel includes four, five, six, seven, eight, nine, ten, eleven, twelve, thirteen, fourteen, or fifteen biomarkers selected from STOM, MME, BTN3A2, HLA-DPA1, EVL, EPB42, GSPT1, LAT, HK3, SERPINB1, GBP2, SLC1A5, IGF2BP2, and ANXA3.

IIE. Assays

As shown in FIG. 1B, the system environment 100 involves implementing a marker quantification assay 120 for determining quantitative data for one or more biomarkers. Examples of an assay (e.g., marker quantification assay 120) for one or more markers include DNA assays, microarrays, polymerase chain reaction (PCR), RT-PCR, Southern blots, Northern blots, antibody-binding assays, enzyme-linked immunosorbent assays (ELISAs), flow cytometry, protein assays, Western blots, nephelometry, turbidimetry, chromatography, mass spectrometry, immunoassays, including, by way of example, but not limitation, RIA, immunofluorescence, immunochemiluminescence, immunoelectrochemiluminescence, or competitive immunoassays, immunoprecipitation. The information from the assay can be quantitative and sent to a computer system as described in further detail in reference to FIG. 16. The information can also be qualitative, such as observing patterns or fluorescence, which can be translated into a quantitative measure by a user or automatically by a reader or computer system.

In various embodiments, the assay can be any one of RT-qPCR (quantitative reverse transcription polymerase chain reaction), qPCR (quantitative polymerase chain reaction), PCR (polymerase chain reaction), RT-PCR (reverse transcription polymerase chain reaction), SDA (strand displacement amplification), RPA (recombinase polymerase amplification), MDA (multiple displacement amplification), HDA (helicase dependent amplification), LAMP (loop-mediated isothermal amplification), RCA (rolling circle amplification), NASBA (nucleic acid-sequence-based amplification), and any other isothermal or thermocycled amplification reaction. In particular embodiments, the assay is a RT-qPCR assay or a LAMP assay. For example, in a critical care setting where a classification and therapy recommendation is to be rapidly developed for a patient (e.g., within 30 minutes or within 2 hours), assay can be RT-qPCR or a LAMP assay that enables rapid quantification of the biomarkers in a sample obtained from the patient.

In various embodiments, the marker quantification assay 120 involves performing sequencing to obtain sequence reads (e.g., sequence reads for generating a sequencing library). The sequence reads can be quantified to determine quantitative data of biomarkers. Sequence reads can be achieved with commercially available next generation sequencing (NGS) platforms, including platforms that perform any of sequencing by synthesis, sequencing by ligation, pyrosequencing, using reversible terminator chemistry, using phospholinked fluorescent nucleotides, or real-time sequencing. As an example, amplified nucleic acids may be sequenced on an Illumina MiSeq platform.

When pyrosequencing, libraries of NGS fragments are cloned in-situ amplified by capture of one matrix molecule using granules coated with oligonucleotides complementary to adapters. Each granule containing a matrix of the same type is placed in a microbubble of the “water in oil” type and the matrix is cloned amplified using a method called emulsion PCR. After amplification, the emulsion is destroyed and the granules are stacked in separate wells of a titration picoplate acting as a flow cell during sequencing reactions. The ordered multiple administration of each of the four dNTP reagents into the flow cell occurs in the presence of sequencing enzymes and a luminescent reporter, such as luciferase. In the case where a suitable dNTP is added to the 3′ end of the sequencing primer, the resulting ATP produces a flash of luminescence within the well, which is recorded using a CCD camera. It is possible to achieve a read length of more than or equal to 400 bases, and it is possible to obtain 10⁶ readings of the sequence, resulting in up to 500 million base pairs (megabytes) of the sequence. Additional details for pyrosequencing is described in Voelkerding et al., Clinical Chem., 55: 641-658, 2009; MacLean et al., Nature Rev. Microbiol., 7: 287-296; U.S. Pat. Nos. 6,210,891; 6,258,568; each of which is hereby incorporated by reference in its entirety.

On the Solexa/Illumina platform, sequencing data is produced in the form of short readings. In this method, fragments of a library of NGS fragments are captured on the surface of a flow cell that is coated with oligonucleotide anchor molecules. An anchor molecule is used as a PCR primer, but due to the length of the matrix and its proximity to other nearby anchor oligonucleotides, elongation by PCR leads to the formation of a “vault” of the molecule with its hybridization with the neighboring anchor oligonucleotide and the formation of a bridging structure on the surface of the flow cell. These DNA loops are denatured and cleaved. Straight chains are then sequenced using reversibly stained terminators. The nucleotides included in the sequence are determined by detecting fluorescence after inclusion, where each fluorescent and blocking agent is removed prior to the next dNTP addition cycle. Additional details for sequencing using the Illumina platform is found in Voelkerding et al., Clinical Chem., 55: 641-658, 2009; MacLean et al., Nature Rev. Microbiol., 7: 287-296; U.S. Pat. Nos. 6,833,246; 7,115,400; 6,969,488; each of which is hereby incorporated by reference in its entirety.

Sequencing of nucleic acid molecules using SOLiD technology includes clonal amplification of the library of NGS fragments using emulsion PCR. After that, the granules containing the matrix are immobilized on the derivatized surface of the glass flow cell and annealed with a primer complementary to the adapter oligonucleotide. However, instead of using the indicated primer for 3′ extension, it is used to obtain a 5′ phosphate group for ligation for test probes containing two probe-specific bases followed by 6 degenerate bases and one of four fluorescent labels. In the SOLiD system, test probes have 16 possible combinations of two bases at the 3′ end of each probe and one of four fluorescent dyes at the 5′ end. The color of the fluorescent dye and, thus, the identity of each probe, corresponds to a certain color space coding scheme. After many cycles of alignment of the probe, ligation of the probe and detection of a fluorescent signal, denaturation followed by a second sequencing cycle using a primer that is shifted by one base compared to the original primer. In this way, the sequence of the matrix can be reconstructed by calculation; matrix bases are checked twice, which leads to increased accuracy. Additional details for sequencing using SOLiD technology is found in Voelkerding et al., Clinical Chem., 55: 641-658, 2009; MacLean et al., Nature Rev. Microbiol., 7: 287-296; U.S. Pat. Nos. 5,912,148; 6,130,073; each of which is incorporated by reference in its entirety.

In particular embodiments, HeliScope from Helicos BioSciences is used. Sequencing is achieved by the addition of polymerase and serial additions of fluorescently-labeled dNTP reagents. Switching on leads to the appearance of a fluorescent signal corresponding to dNTP, and the specified signal is captured by the CCD camera before each dNTP addition cycle. The reading length of the sequence varies from 25-50 nucleotides with a total yield exceeding 1 billion nucleotide pairs per analytical work cycle. Additional details for performing sequencing using HeliScope is found in Voelkerding et al., Clinical Chem., 55: 641-658, 2009; MacLean et al., Nature Rev. Microbiol., 7: 287-296; U.S. Pat. Nos. 7,169,560; 7,282,337; 7,482,120; 7,501,245; 6,818,395; 6,911,345; 7,501,245; each of which is incorporated by reference in its entirety.

In some embodiments, a Roche sequencing system 454 is used. Sequencing 454 involves two steps. In the first step, DNA is cut into fragments of approximately 300-800 base pairs, and these fragments have blunt ends. Oligonucleotide adapters are then ligated to the ends of the fragments. The adapter serve as primers for amplification and sequencing of fragments. Fragments can be attached to DNA-capture beads, for example, streptavidin-coated beads, using, for example, an adapter that contains a 5′-biotin tag. Fragments attached to the granules are amplified by PCR within the droplets of an oil-water emulsion. The result is multiple copies of cloned amplified DNA fragments on each bead. At the second stage, the granules are captured in wells (several picoliters in volume). Pyrosequencing is carried out on each DNA fragment in parallel. Adding one or more nucleotides leads to the generation of a light signal, which is recorded on the CCD camera of the sequencing instrument. The signal intensity is proportional to the number of nucleotides included. Pyrosequencing uses pyrophosphate (PPi), which is released upon the addition of a nucleotide. PPi is converted to ATP using ATP sulfurylase in the presence of adenosine 5′phosphosulfate. Luciferase uses ATP to convert luciferin to oxyluciferin, and as a result of this reaction, light is generated that is detected and analyzed. Additional details for performing sequencing 454 is found in Margulies et al. (2005) Nature 437: 376-380, which is hereby incorporated by reference in its entirety.

Ion Torrent technology is a DNA sequencing method based on the detection of hydrogen ions that are released during DNA polymerization. The microwell contains a fragment of a library of NGS fragments to be sequenced. Under the microwell layer is the hypersensitive ion sensor ISFET. All layers are contained within a semiconductor CMOS chip, similar to the chip used in the electronics industry. When dNTP is incorporated into a growing complementary chain, a hydrogen ion is released that excites a hypersensitive ion sensor. If homopolymer repeats are present in the sequence of the template, multiple dNTP molecules will be included in one cycle. This results in a corresponding amount of hydrogen atoms being released and in proportion to a higher electrical signal. This technology is different from other sequencing technologies that do not use modified nucleotides or optical devices. Additional details for Ion Torrent Technology is found in Science 327 (5970): 1190 (2010); US Patent Application Publication Nos. 20090026082, 20090127589, 20100301398, 20100197507, 20100188073, and 20100137143, each of which is incorporated by reference in its entirety.

In various embodiments, immunoassays designed to quantitate markers can be used in screening including multiplex assays. Measuring the concentration of a target marker in a sample or fraction thereof can be accomplished by a variety of specific assays. For example, a conventional sandwich type assay can be used in an array, ELISA, RIA, etc. format. Other immunoassays include Ouchterlony plates that provide a simple determination of antibody binding. Additionally, Western blots can be performed on protein gels or protein spots on filters, using a detection system specific for the markers as desired, conveniently using a labeling method.

Protein based analysis, using an antibody that specifically binds to a polypeptide (e.g. marker), can be used to quantify the marker level in a test sample obtained from a subject. In various embodiments, an antibody that binds to a marker can be a monoclonal antibody. In various embodiments, an antibody that binds to a marker can be a polyclonal antibody. For multiplex analysis of markers, arrays containing one or more marker affinity reagents, e.g. antibodies can be generated. Such an array can be constructed comprising antibodies against markers. Detection can utilize one or a panel of marker affinity reagents, e.g. a panel or cocktail of affinity reagents specific for one, two, three, four, five, six, seven, eight, nine, ten, eleven, twelve, thirteen, fourteen, fifteen, sixteen, seventeen, eighteen, nineteen, twenty, twenty one, twenty two, twenty three, twenty four or more markers.

In various embodiments, determining the quantitative expression data for each of the at least three biomarkers comprises: contacting the sample with a reagent; generating a plurality of complexes between the reagent and the plurality of biomarkers in the sample; and detecting the plurality of complexes to obtain a dataset associated with the sample, wherein the dataset comprises the quantitative expression data for the biomarker.

### EXAMPLES

### III. Dsyregulated Host Response Patient Subtypes

Custom processing of 14 datasets from sepsis studies from the literature was performed to identify dysregulated host response subtypes.⁴⁶ For each study, patients were classified as either adult or pediatric. To distinguish between pediatric and adult patients, manual literature review was performed. Then, adult patients were classified as either sepsis (S) or septic shock (SS), septic shock being a subset of sepsis. To distinguish between adult sepsis and adult septic shock patients, the rate of patient vasopressor use reported in the literature (normally at the first day) was used. If the rate of patient vasopressor use was more than 50%, the whole study cohort was classified as septic shock. In contrast, if the rate of patient vasopressor use was less than 50%, the whole study cohort was classified as sepsis. Based on these classifications of adult or pediatric and sepsis or septic shock, patient samples were classified as Full samples (including adult, pediatric, sepsis, and septic shock patient samples), SS samples (including only adult septic shock patient samples), S samples (including only adult sepsis patient samples), and P samples (including only pediatric sepsis and septic shock patient samples).

Following classification of the patient samples from each literature study, for each study, biomarker expression data were normalized within the study and curated with methodologies specific to the study's array platform technology and to the study's available data format. Healthy control samples and patient samples were processed by the COCONUT framework,⁴⁷ which normalized the samples with the same array platform and transformed patient expression data according to normalization parameters derived from the healthy samples. The resulting expression data were quantile normalized across patients and studies at the end of the normalization process.

The COINCIDE algorithm was then used to rank the genes based on the expression data.⁴⁷ Then, for each set of classified patient samples (e.g., Full samples, SS samples, S samples, and P samples), for each subset of genes ranked (i.e. 100, 250, 500, 1000, 1500 genes, so on and so forth), the COMMUNAL clustering algorithm was used to identify the optimal number of clusters,^{47, 49} as well as to label each patient sample.⁴⁶ COMMUNAL maps of cluster optimality were generated for each set of classified patient samples, in which the X-axis is the number of clusters, the Y-axis is the number of included genes, and the Z-axis is the mean validity score.

The COMMUNAL map of cluster optimality of a Full Model (including adult, pediatric, sepsis, and septic shock patient samples), exhibited three clusters for 574 out of 700 training samples. The remaining training samples were reported as inconclusive.

The COMMUNAL map of cluster optimality for a SS Model (including only adult septic shock patient samples), exhibited three clusters for 115 out of 165 training samples.

The COMMUNAL map of cluster optimality for a S Model (including only adult septic patient samples), exhibited four clusters for 153 out of 308 training samples. However, the fourth cluster did not reveal consistent results among different clustering algorithms.

The COMMUNAL map of cluster optimality for a P Model (including only pediatric sepsis and septic shock patient samples), exhibited three clusters for 180 out of 227 training samples.

Stable optima were consistently observed at K=3 clusters. Using the patient expression data and cluster labels, Gene Ontology (GO) analysis was performed to characterize the nature and functionality of each cluster, hereinafter referred to as a “subtype”. Subtypes were named as A (lower mortality, adaptive immune activation), B (higher mortality, innate immune activation), and C (higher mortality, older, and with clinical and molecular evidence of coagulopathy).⁴⁶ The biological functions indicated in the GO analysis demonstrated distinct characteristics among the different subtypes, indicating high potential for guided treatment.

### IV. Dysregulated Host Response Patient Subtype Classifiers

Eight classification Models, including the Full Model based on the Full samples, the SS Model based on the SS samples, the S Model based on the S samples, the P Model based on the P samples, as well as the SS.B1, SS.B2, SS.B3, and SS.B4 models were developed. As detailed below, to train the classification Models based on the associated samples, training labels for each training sample were determined using unsupervised clustering procedures including, normalization, the COCONUT method, the COINCIDE method, and the COMMUNAL method.

The methodology of building the classifiers was guided by a number of considerations, particularly in data transformation and normalization, which impact classifier performance the most. Specifically, the classifiers were built based on the following considerations. First, the classification is time-sensitive because dysregulated host response progression is dynamic (e.g., patients can transition from one subtype to another over time). Therefore, time matched data were analyzed. The time matched data analyzed included data from blood collected from patients within 24 hours of sepsis diagnosis. In cases in which time series data existed, data from the first time point was used. Second, the final classification was envisioned to be a measure of a few biomarkers selected from tens of thousands of biomarkers, so down-selection of the most important biomarkers was implemented. Third, the training sets for the subtype classifiers did not have any outcome labels based on a randomized placebo-controlled trial design, so the trial datasets were selected exclusively as a test set for classifier performance evaluation. Fourth, the VANISH trial raw expression data were measured with the Illumina platform and reported in a different format than the format of expression data of the training set, so the normalization used in the clustering process required special consideration. Fifth, the classification process applied similar data transformation to the training set and the test set to achieve the best performance. Finally, the transformation and normalization strategies that worked best for the clustering process and even the training process may not necessarily perform well in classifying subtypes to differentiate corticosteroid response because the training set did not involve outcome data.

Based on these six considerations, the classifiers were built with a normalization scheme for both the training and test expression data. A platform normalization matrix was built out of all genes of all healthy and sepsis samples. As the number of samples in the matrix was large, individual samples' expression data were quantile normalized against the matrix as a perturbation. To train the classifiers, the expression data from the training set was batch normalized and curated, and then normalized by the platform normalization matrix, as described in detail below.

Sets of potentially significant biomarkers were identified by Significance Analysis of Microarrays (SAM).⁴⁸ As another example, sets of potentially significant biomarkers are identifiable using qPCR or RNA sequencing data. qPCR measures the relative or absolute expression level of biomarkers. Normalization or calibration processes are implemented. RNA sequencing data measures relative expression levels of model genes and their transcripts. Using sequencing reads alignment methods (e.g. Hisat2, and Bowtie2), expression estimation methods (e.g. StringTie, Salmon) and normalization processes (e.g. quantile normalization), the estimated expression of model genes are quantified.

These sets of potentially significant biomarker sets were down-selected by at least 2-fold change, and forward-search methodology was used to identify a small set of biomarkers for feature calculation.⁵¹ The calculated features (e.g., summarized differential gene expression)⁵¹ and clustering label of each sample were finally used to train the multi-class classifiers, implemented as e1071::svm with radial kernel, 0.1 gamma, and 10 cost. Tables 1, 2A-2B, and 3 below depict the genes identified for each subtype (e.g., A, B, and C) for each classifier (e.g., the Full Model, the SS Model, the S Model, and the P Model). Specifically, Table 1 depicts the genes for each subtype (e.g., A, B, and C) for the Full Model, Table 2A depicts the genes for each subtype (e.g., A, B, and C) for the SS Model, Table 2B depicts the genes for each subtype (e.g., A, B, and C) for the S Model, and Table 3 depicts the genes for each subtype (e.g., A, B, and C) for the P Model. Note that in certain embodiments, the entire set of genes for a given Model is used to train and/or test the Model. However, in alternative embodiments, only a subset of the set of genes for a given Model is used to train and/or test the Model. For example, in some embodiments, at least one gene from each subtype A, B, and C (e.g., at least one gene from each row 1, 2, and 3 in one of the below Tables 1, 2A, 2B, and 3) may be used to train and/or test a model.

TABLE 1
Full Model Biomarkers

| Row Number | Subtype | Role | Biomarkers |
|---|---|---|---|
| 1 | A | up | EVL, BTN3A2, HLA-DPA1, IDH3A, |
| | | | ACBD3, EXOSC10, SNRK |
| | | down | MMP8 |
| 2 | B | up | SERPINB1 |
| | | down | GSPT1 |
| 3 | C | up | MPP1, HMBS, TAL1, C9orf78, |
| | | | POLR2L |
| | | down | SLC27A3, BTN3A2, DDX50, |
| | | | FCHSD2, GSTK1, UBE2E1, |
| | | | TNFRSF1A, PRPF3, TOMM70A |

TABLE 2A
SS Model Biomarkers

| Row Number | Subtype | Role | Biomarkers |
|---|---|---|---|
| 1 | A | up | ZNF831, MME, CD3G |
| | | down | STOM |
| 2 | B | up | |
| | | down | ECSIT, LAT, NCOA4 |
| 3 | C | up | SLC1A5, IGF2BP2, ANXA3 |
| | | down | |

TABLE 2B
S Model Biomarkers

| Row Number | Subtype | Role | Biomarkers |
|---|---|---|---|
| 1 | A | up | C14orf159, PUM2 |
| | | down | |
| 2 | B | up | |
| | | down | EPB42, RPS6KA5 |
| 3 | C | up | EPB42 |
| | | down | GBP2 |

TABLE 3
P Model Biomarkers

| Row Number | Subtype | Role | Biomarkers |
|---|---|---|---|
| 1 | A | up | MSH2, DCTD |
| | | down | MMP8 |
| 2 | B | up | HK3 |
| | | down | UCP2, NUP88 |
| 3 | C | up | GABARAPL2 |
| | | down | CASP4 |

Additional models were created in order to include at least one up- and one down-gene in the model to enable the calculation of scores in an assay based on relative gene expression. Two methods were applied based on forward selection and backward elimination. Forward selection is an iterative method that starts with no genes in the model. In each iteration, features are added that improves the model until the addition of a new variable does not improve the performance of the model. In backward elimination, all the genes are included and then the least significant feature is removed at each iteration if there is improvement in the performance of the model. This is repeated until no improvement is observed from the removal of features. As an example, the SS model was taken as a starting point for the creation of an alternative model. The metric used for evaluating model performance was leave-one-out accuracy and the model's similarity in labeling patients when compared to the Full model.

In this exercise, the backward elimination method produced superior results. Tables 4A-4D depicts four additional models generated by this method named SS.B1, SS.B2, SS.B3, and SS.B4.

TABLE 4A
SS.B1

| Row Number | Subtype | Role | Biomarkers |
|---|---|---|---|
| 1 | A | down | STOM |
| | | up | ZNF831, CD3G, MME, |
| | | | BTN3A2, HLA-DPA1 |
| 2 | B | down | EPB42, GSPT1, LAT |
| | | up | HK3, SERPINB1 |
| 3 | C | down | GBP2, TNFRSF1A |
| | | up | SLC1A5, IGF2BP2, ANXA3 |

TABLE 4B
SS.B2

| Row Number | Subtype | Role | Biomarkers |
|---|---|---|---|
| 1 | A | down | STOM |
| | | up | ZNF831, CD3G, MME, |
| | | | BTN3A2, HLA-DPA1 |
| 2 | B | down | EPB42, GSPT1, LAT |
| | | up | HK3, SERPINB1 |
| 3 | C | down | GBP2 |
| | | up | SLC1A5, IGF2BP2, ANXA3 |

TABLE 4C
SS.B3

| Row Number | Subtype | Role | Biomarkers |
|---|---|---|---|
| 1 | A | down | STOM |
| | | up | MME, BTN3A2, HLA-DPA1, EVL |
| 2 | B | down | EPB42, GSPT1, LAT |
| | | up | HK3, SERPINB1 |
| 3 | C | down | BTN3A2, TNFRSF1A |
| | | up | SLC1A5, IGF2BP2, ANXA3 |

TABLE 4D
SS.B4

| Row Number | Subtype | Role | Biomarkers |
|---|---|---|---|
| 1 | A | down | STOM |
| | | up | MME, BTN3A2, HLA-DPA1, EVL |
| 2 | B | down | EPB42, GSPT1, LAT |
| | | up | HK3, SERPINB1 |
| 3 | C | down | GBP2 |
| | | up | SLC1A5, IGF2BP2, ANXA3 |

TABLE 4E
Identification of biomarkers included in each of
the Full model, SS model, S model, P model, SS.B1
model, SS.B2 model, SS.B3 model, and SS.B4 model.

| Gene | Alias | Uniprot ID |
|---|---|---|
| EVL | Enah/Vasp-like | Q9UI08 |
| BTN3A2 | Butyrophilin Subfamily 3 Member | P78410 |
| | A2 | |
| HLA-DPA1 | Major Histocompatibility Complex, | P20036 |
| | Class II, DP Alpha 1 | |
| IDH3A | Isocitrate Dehydrogenase (NAD(+)) | P50213 |
| | 3 Catalytic Subunit Alpha | |
| ACBD3 | Acyl-CoA Binding Domain | Q9H3P7 |
| | Containing 3 | |
| EXOSC10 | Exosome Component 10 | Q01780 |
| SNRK | SNF Related Kinase | Q9NRH2 |
| MMP8 | Matrix Metallopeptidase 8 | P22894 |
| SERPINB1 | Serpin Family B Member 1 | P30740 |
| GSPT1 | G1 To S Phase Transition 1 | P15170 |
| MPP1 | Membrane Palmitoylated Protein 1 | Q00013 |
| HMBS | Hydroxymethylbilane Synthase | P08397 |
| TAL1 | TAL BHLH Transcription Factor 1, | P17542 |
| | Erythroid Differentiation Factor | |
| C9orf78 | Chromosome 9 Open Reading Frame | Q9NZ63 |
| | 78 | |
| POLR2L | RNA Polymerase II, I And III | P62875 |
| | Subunit L | |
| SLC27A3 | Solute Carrier Family 27 Member 3 | Q5K4L6 |
| DDX50 | DExD-Box Helicase 50 | Q9BQ39 |
| FCHSD2 | FCH And Double SH3 Domains 2 | O94868 |
| GSTK1 | Glutathione S-Transferase Kappa 1 | Q9Y2Q3 |
| UBE2E1 | Ubiquitin Conjugating Enzyme E2 | P51965 |
| | E1 | |
| TNFRSF1A | TNF Receptor Superfamily Member | P19438 |
| | 1A | |
| PRPF3 | Pre-MRNA Processing Factor 3 | O43395 |
| TOMM70A | Translocase Of Outer Mitochondrial | O94826 |
| | Membrane 70 | |
| ZNF831 | Zinc Finger Protein 831 | Q5JPB2 |
| MME | Membrane Metalloendopeptidase | P08473 |
| CD3G | CD3g Molecule | P09693 |
| STOM | Stomatin | P27105 |
| ECSIT | ECSIT Signaling Integrator | Q9BQ95 |
| LAT | Linker For Activation Of T Cells | O43561 |
| NCOA4 | Nuclear Receptor Coactivator 4 | Q13772 |
| SLC1A5 | Solute Carrier Family 1 Member 5 | Q15758 |
| IGF2BP2 | Insulin lake Growth Factor 2 | Q9Y6M1 |
| | MRNA Binding Protein 2 | |
| ANXA3 | Annexin A3 | P12429 |
| C14orf159 | D-Glutamate Cyclase | Q7Z3D6 |
| PUM2 | Pumilio RNA Binding Family | Q8TB72 |
| | Member 2 | |
| EPB42 | Erythrocyte Membrane Protein Band | P16452 |
| | 4.2 | |
| RPS6KA5 | Ribosomal Protein S6 Kinase A5 | O75582 |
| GBP2 | Guanylate Binding Protein 2 | P32456 |
| MSH2 | MutS Homolog 2 | P43246 |
| DCTD | DCMP Deaminase | P32321 |
| HK3 | Hexokinase 3 | P52790 |
| UCP2 | Uncoupling Protein 2 | P55851 |
| NUP88 | Nucleoporin 88 | Q99567 |
| GABARAPL2 | GABA Type A Receptor Associated | P60520 |
| | Protein Like 2 | |
| CASP4 | Caspase 4 | P49662 |

Table 5 depicts primer sets for amplifying genes identified by the SS Model and depicted above in Table 2A, primer sets for amplifying genes identified by the S Model and depicted above in Table 3B, and primer sets for amplifying genes identified by the SS.B2 Model and depicted above in Table 4B. Each primer set includes a pair of single-stranded DNA primers (i.e., a forward primer and a reverse primer) for amplifying one gene by, for example, RT-qPCR. In some embodiments the entire sequence of a primer may be used in amplification of the associated gene. In alternative embodiments, at least 15 contiguous nucleotides of a primer sequence may be used in amplification of the associated gene. In certain embodiments, primer sequences other than those mentioned provided in Table 5 can be used to amplify one or more of the genes from Tables 1, 2A, 2B, 3, and 4A-4D.

TABLE 5
RT-qPCR Primer Sequences

| | | | | | Forward | Reverse |
|---|---|---|---|---|---|---|
| | | | | | Primer | Primer |
| | | | Forward | Reverse | SEQ ID | SEQ ID |
| Gene | Model | Subtype | Primer | Primer | NO. | NO. |
| SLC1A5 | SS/SS.B2 | C | ATCACCATC | CCACAGCCA | 1 | 2 |
| | | | CTGGTCACG | GGATCAAGG | | |
| | | | G | AG | | |
| IGF2BP2 | SS | C | AAGACCGTG | TTTCCCTGAT | 3 | 4 |
| | | | AACGAACT | CTTGCGCTG | | |
| | | | GCA | T | | |
| ANXA3 | SS/SS.B2 | C | CGAGCCTTG | TGTTCGAAT | 5 | 6 |
| | | | AAGGGTATT | GTCCAAAAG | | |
| | | | GG | GTCA | | |
| ZNF831 | SS/SS.B2 | A | ACCTGGGTG | GGTGATTCT | 7 | 8 |
| | | | CGAAGAAG | GAGGTGGCA | | |
| | | | AAG | CA | | |
| MME | SS/SS.B2 | A | AACTTTGCA | GCAGAGTTC | 9 | 10 |
| | | | CAGGTGTGG | TGCAAAGTC | | |
| | | | TG | CC | | |
| CD3G | SS/SS.B2 | A | GCCCCTCAA | AGGAGGAGA | 11 | 12 |
| | | | GGATCGAG | ACACCTGGA | | |
| | | | AAG | CT | | |
| STOM | SS/SS.B2 | A | AAAGGTGG | AAGGGCTGC | 13 | 14 |
| | | | AGCGTGTGG | AGGAGATTC | | |
| | | | AAA | AG | | |
| ECSIT | SS | B | CCGGAGGA | CATGCACAT | 15 | 16 |
| | | | GTGGAACCT | GGCGAAGAC | | |
| | | | CTA | AG | | |
| LAT | SS/SS.B2 | B | TGTGTCCCA | CAGCTCCTG | 17 | 18 |
| | | | GGAACTGC | CAGATTCTC | | |
| | | | ATC | GT | | |
| NCOA4 | SS | B | GGGCAACCT | CAAACTGCA | 19 | 20 |
| | | | CAGCCAGTT | GGGAGGCCA | | |
| | | | AT | TA | | |
| C14orf159 | S | A | CCCTCCCGT | TTCTGGATC | 21 | 22 |
| | | | CGGTCATTA | ATCTCGGCG | | |
| | | | AG | TG | | |
| PUM2 | S | A | TGCACAAGA | GGTGGTCCT | 23 | 24 |
| | | | TTCGACCTC | CCAATAGGT | | |
| | | | ACA | CC | | |
| EPB42 | S/SS.B2 | B, C | TGCCATCAA | CTCTCTGTGA | 25 | 26 |
| | | | GATGCCAG | ATGAGCCCC | | |
| | | | AGAA | C | | |
| GBP2 | S/SS.B2 | C | CAGGGCCCA | GGCTCCAAT | 27 | 28 |
| | | | GTTAATGGC | GATTTGCTTC | | |
| | | | A | TCA | | |
| RPS6KA5 | S | B | AGCAACCTT | ACTCTCACT | 29 | 30 |
| | | | CCACGCCTT | GGAACTGCT | | |
| | | | TA | GC | | |
| GSPT1 | SS.B2 | | GACTTCCCT | TCACAGTAT | 31 | 32 |
| | | | CAGATGGGT | TGTGCAGGG | | |
| | | | CG | TCA | | |
| IGFBP2 | SS.B2 | | AAGACCGTG | TTTCCCTGAT | 33 | 34 |
| | | | AACGAACT | CTTGCGCTG | | |
| | | | GCA | T | | |
| HK3 | SS.B2 | | GAACGCTCT | CTCTGACTG | 35 | 36 |
| | | | ACAAGCTGC | CAGGAACGT | | |
| | | | AC | GA | | |
| SERPINB1 | SS.B2 | | TCCTGCTGC | GTCCACTCA | 37 | 38 |
| | | | CGGATGAC | TGCAACTTTT | | |
| | | | ATT | CCA | | |
| BTN3A2 | SS.B2 | | GCTGACTTA | CAGAGCGGG | 39 | 40 |
| | | | TTGGTATCG | AAATAAGCC | | |
| | | | GACG | TAAGA | | |
| HLA-DPA1 | SS.B2 | | CCAGGGGA | AGAGCTTGA | 41 | 42 |
| | | | CCCTGTGAA | AGGGTCAGC | | |
| | | | ATA | AAT | | |

In certain embodiments, genes may be amplified by methods other than RT-qPCR. For example, in some embodiments, genes may be amplified via LAMP (loop-mediated isothermal amplification). In such embodiments in which a gene is amplified via LAMP, a primer set for amplifying the gene includes a forward outer primer, a backward outer primer, a forward inner primer, a backward inner primer, a forward loop primer, and a backward loop primer.

Sensitivity analysis was performed for each classifier, for each combination of three genes, with one gene selected from each subtype. Specifically, the accuracies of the classifiers were measured to demonstrate that the accuracy of each classifier in identifying a subject's subtype using any combination of three genes, with one gene from each subtype, is greater than 50% (e.g., greater than random chance).

To calculate accuracy for a given classifier for a given combination of three genes, leave-one-out accuracy of the training samples of the training dataset on which the classifier was trained was implemented. The training dataset included N training samples, each training sample including a label y and features x. The leave-one-out accuracy for the classifier for the combination of three genes was calculated based on N calculations. The Ni calculation leaves out the training sample i during training of the classifier. Then, the trained classifier is used to make a prediction zᵢ for the features xᵢ that corresponds to the training sample i that was left out of the training data set. The prediction zᵢ is then compared to the label yᵢ to determine the accuracy of the prediction. The leave-one-out accuracy for the classifier for the combination of three genes was calculated as the number of correct predictions z, divided by N.

FIGS. 2-5 depict the individual accuracies determined for each combination of three genes, with one gene from each subtype, for the Full, SS, S, and P Models, respectively. Specifically, FIG. 2 is a graph of the individual accuracies determined for each combination of three genes, with one gene from each subtype, for the Full Model. FIG. 3 is a graph of the individual accuracies determined for each combination of three genes, with one gene from each subtype, for the SS Model. FIG. 4 is a graph of the individual accuracies determined for each combination of three genes, with one gene from each subtype, for the S Model. FIG. 5 is a graph of the individual accuracies determined for each combination of three genes, with one gene from each subtype, for the P Model. As shown in FIGS. 2-5, each classifier, for each combination of three genes, with one gene from each subtype, demonstrated an accuracy of greater than 50% (e.g., greater than random chance). Furthermore, the average accuracies of the Full, SS, S, and P Models were 82.93%, 89.6%, 86.3%, and 98.3%, respectively. Therefore, each classifier demonstrated an average accuracy of greater than 50% (e.g., greater than random chance). Included in each of FIGS. 2-5 is the accuracy of a model that incorporates all of the genes for a particular model (denoted as “full” in each respective figure). For example, for the Full model, incorporating all of the genes refers to a Full model that analyzes all the biomarkers shown in Table 1. For the SS model, incorporating all of the genes refers to the SS model that analyzes all the biomarkers shown in Table 2A. For the S model, incorporating all of the genes refers to the S model that analyzes all the biomarkers shown in Table 2B. For the P model, incorporating all of the genes refers to the SS model that analyzes all the biomarkers shown in Table 3.

FIGS. 6A-6D are graphs of individual accuracies determined for each combination of three biomarkers, with one biomarker from each subtype, for the SS.B1, SS.B2, SS.B3, and SS.B4 models, respectively. These models exhibit accuracies of 89.57%. Included in each of FIGS. 6A-6D is the accuracy of each model that incorporates all of the genes for a particular model (denoted as “full” in each respective figure). For example, for the SS.B1 model, incorporating all of the genes refers to a SS.B1 model that analyzes all the biomarkers shown in Table 4A. For example, for the SS.B2 model, incorporating all of the genes refers to a SS.B2 model that analyzes all the biomarkers shown in Table 4B. For example, for the SS.B3 model, incorporating all of the genes refers to a SS.B3 model that analyzes all the biomarkers shown in Table 4C. For example, for the SS.B4 model, incorporating all of the genes refers to a SS.B4 model that analyzes all the biomarkers shown in Table D.

### V. Identification of Therapeutics for Treatment of Dysregulated Host Response Patient Subtypes

Based on the differential biomarker expression determined for each dysregulated host response subtype, an immune state was determined for each subtype. Specifically, subtype A was determined to be associated with the adaptive immune state, subtype B was determined to be associated with the innate immune state and the complement immune state, and subtype C was determined to be associated with the coagulopathic immune state. Then, biomarkers indicated as related to dysregulated host response, immune state, and the pharmacology of existing therapeutics were identified in the literature. Table 6 below depicts a representative list of genes associated with dysregulated host response, immune state, and the pharmacology of existing therapeutics that were identified from the literature.

TABLE 6
Representative Examples of Genes Associated with Dysregulated host
response, Immune State, and Pharmacology of Existing Therapeutics

| Gene | Uniprot | Immune state | | Protein/Function | Effect |
|---|---|---|---|---|---|
| TREM1 | Q9NP99 | Innate | PAMP | Triggering receptor | Pro- |
| | | | | expressed by myeloid | inflammatory |
| | | | | cells-1 | |
| CD180 | Q99467 | Innate | PAMP | Controls B cell | Pro- |
| | | | | recognition and signaling | inflammatory |
| | | | | of LPS via TLR4 | |
| MIF | P14174 | Innate | PAMP | Stimulated by bacterial | Pro- |
| | | | | antigens (and | inflammatory |
| | | | | glucocorticoids thus | |
| | | | | counteracting it's effects) | |
| CD14 | P08571 | Innate | PAMP | PAMP recognition | Pro- |
| | | | | | inflammatory |
| IL15 | P40933 | Innate | PAMP | IL-15. Secreted following | Pro- |
| | | | | viral infection. Induces | inflammatory |
| | | | | the proliferation of natural | |
| | | | | killer cells. | |
| IL6 | P05231 | Innate | PAMP | IL-6 | Pro and Anti- |
| | | | | | inflammatory |
| TLR2 | O60603 | Innate | PAMP/DAMP | Recognizes bacterial, | Pro- |
| | | | | fungal, viral, and certain | inflammatory |
| | | | | endogenous substances | |
| TLR6 | Q9Y2C9 | Innate | PAMP | Recognizes lipopeptides | Pro- |
| | | | | derived from gram- | inflammatory |
| | | | | positive bacteria and | |
| | | | | mycoplasma and several | |
| | | | | fungal cell wall | |
| | | | | saccharides | |
| NLRP1 | Q9C000 | Innate | PAMP | Notch-like receptor. | Pro- |
| | | | | activates an antibacterial | inflammatory |
| | | | | immune response | |
| CASP1 | P29466 | Innate | PAMP/DAMP | Interleukin-1 converting | Pro- |
| | | | | enzyme | inflammatory |
| IL1B | P01584 | Innate | PAMP/DAMP | IL-1β | Pro- |
| | | | | | inflammatory |
| IL18 | Q14116 | Innate | PAMP/DAMP | IL-18 | Pro- |
| | | | | | inflammatory |
| PYCARD | Q9ULZ3 | Innate | PAMP/DAMP | ASC (PRR), activates | Pro- |
| | | | | caspsase 1 and pro- | inflammatory |
| | | | | inflammatory cytokines | |
| TLR4 | O00206 | Innate | PAMP/DAMP | PRR that activates innate | Pro- |
| | | | | immunity via NF-kB | inflammatory |
| TNF | P01375 | Innate | PAMP/DAMP | TNF-α expressed by | Pro- |
| | | | | macrophages | inflammatory |
| EBI3 | Q14213 | Innate | PAMP/DAMP | IL-27B, Expressed by | Adaptive |
| | | | | APC via TLR4 activation, | function |
| | | | | activates Th1, Tr1, inhibits | |
| | | | | Th2, Th17, Treg | |
| IL27 | Q8NEV9 | Innate | PAMP/DAMP | IL-27, Expressed by APC | Adaptive |
| | | | | via TLR4 activation, | function |
| | | | | activates Th1, Tr1, inhibits | |
| | | | | Th2, Th17, Treg | |
| IL1RN | P18510 | Innate | PAMP/DAMP | IL-1 receptor antagonist, | Anti- |
| | | | | prevents IL-1A and B | inflammatory |
| | | | | from binding | |
| HSPD1 | P10809 | Innate | DAMP | HSP60 | Pro- |
| | | | | | inflammatory |
| IL1RL1 | Q01638 | Innate | DAMP | ST2 (receptor of IL-33 | Pro- |
| | | | | which is upregulated by | inflammatory |
| | | | | DAMPs) | |
| S100A9 | P06702 | Innate | DAMP | Heat shock protein | Pro- |
| | | | | (DAMP trigger) | inflammatory |
| HSPA1B | P0DMV8 | Innate | DAMP | Heat shock 70 kDa protein | Pro- |
| | | | | 1B (DAMP trigger) | inflammatory |
| HSPA1A | P0DMV9 | Innate | DAMP | Heat shock 70 kDa protein | Pro- |
| | | | | 1A (DAMP trigger) | inflammatory |
| MPO | P05164 | Innate | DAMP | Myeloperoxidase (DAMP | Pro- |
| | | | | trigger) | inflammatory |
| ELANE | P08246 | Innate | DAMP | Neutrophil elastase | Pro- |
| | | | | (DAMP trigger) | inflammatory |
| CTSG | P08311 | Innate | DAMP | Cathepsin G (DAMP | Pro- |
| | | | | trigger) | inflammatory |
| HMGB1 | P09429 | Innate | DAMP | d HMG-1 (DAMP trigger) | Pro- |
| | | | | | inflammatory |
| CD24 | P25063 | Innate | DAMP | In association with | Pro- |
| | | | | SIGLEC10 may be | inflammatory |
| | | | | involved in the selective | |
| | | | | suppression of the immune | |
| | | | | response to danger- | |
| | | | | associated molecular | |
| | | | | patterns (DAMPs) such as | |
| | | | | HMGB1, HSP70 and | |
| | | | | HSP90. | |
| SIGIRR | Q6IA17 | Innate | | Single Ig IL-1-related | Pro- |
| | | | | receptor, attenuates TLR4 | inflammatory |
| | | | | activity | |
| CSF3 | P09919 | Innate | Cell | G-CSF (pro and anti- | Pro- |
| | | | recruitment | inflammatory), expression | inflammatory |
| | | | | triggered by IL-17 | |
| CSF2 | P04141 | Innate | Cell | GM-CSF, encoded in Th2, | Pro- |
| | | | recruitment, | stimulates stem cells to | inflammatory |
| | | | Innate | produce granulocytes | |
| | | | immune | (neutrophils, eosinophils, | |
| | | | stimlation | and basophils) and | |
| | | | | monocytes via STAT5 | |
| C3AR1 | Q16581 | Complement | Complement | C3a receptor | Complement |
| | | | | | activity |
| C5AR1 | P21730 | Complement | Complement | C5a receptor | Complement |
| | | | | | activity |
| C5AR2 | Q9P296 | Complement | Complement | C5a receptor | Complement |
| | | | | | activity |
| STAT1 | P42224 | Adaptive | | Activated by IFNa, IFNg, | Pro- |
| | | | | EGF, PDGF, IL-6. | inflammatory |
| | | | | Activates Th1, inhibits | |
| | | | | Th17, Treg | |
| IFNG | P01579 | Adaptive | | INF-γ (innate and | Pro- |
| | | | | adaptive) | inflammatory |
| LTA | P01374 | Adaptive | | TNF-β, Lymphotoxin- | Pro- |
| | | | | alpha (LT-α), expressed | inflammatory |
| | | | | by lymphocytes activates | |
| | | | | innate immunity via NF- | |
| | | | | kB | |
| STAT4 | Q14765 | Adaptive | | IFN-γ production triggered | Pro- |
| | | | | by IL-12 | inflammatory |
| CD28 | P10747 | Adaptive | | T cell co-stimulation, IL-6 | Pro- |
| | | | | stimulation, IL-10 | inflammatory |
| | | | | stimulation | |
| CD3D | P04234 | Adaptive | | T-cell surface glycoprotein | Pro- |
| | | | | CD3 gamma chain | inflammatory |
| CD3G | P07766 | Adaptive | | T-cell surface glycoprotein | Pro- |
| | | | | CD3 gamma chain | inflammatory |
| CD3E | P09693 | Adaptive | | T-cell surface glycoprotein | Pro- |
| | | | | CD3 gamma chain | inflammatory |
| PTMA | P06454 | Adaptive | | Thymosin al (increases | Pro- |
| | | | | HLA-DR) | inflammatory |
| IL7R | P16871 | Adaptive | | IL-7 receptor (IL-7 | Pro- |
| | | | | decreases Treg) | inflammatory |
| IL7 | P13232 | Adaptive | | IL-7 (IL-7 decreases Treg) | Pro- |
| | | | | | inflammatory |
| TNFRSF18 | Q9Y5U5 | Adaptive | | Glucocorticoid-induced | Pro- |
| | | | | tumor necrosis factor | inflammatory |
| | | | | receptor family-related | |
| | | | | gene (GITR) involved in | |
| | | | | inhibiting the suppressive | |
| | | | | activity of T-regulatory | |
| | | | | cells and extending the | |
| | | | | survival of T-effector | |
| | | | | cells, decreases IL-10 | |
| IL13 | P35225 | Adaptive | Pro and anti- | IL-13 (Th2 cytokine) | Anti- |
| | | | inflammatory | mediator of allergic | inflammatory |
| | | | | inflammatory response | |
| GZMB | P10144 | Adaptive | | Granzyme-B, expressed | Anti- |
| | | | | by Treg to lyse T cells | inflammatory |
| TNFRSF14 | Q92956 | Adaptive | | a APC HVEM (suppresses | Anti- |
| | | | | adaptive and activates | inflammatory |
| | | | | innate) | |
| BTLA | Q7Z6A9 | Adaptive | | a Tcell BTLA (Inhibitory | Anti- |
| | | | | cell surface receptor) | inflammatory |
| PDCD1 | Q15116 | Adaptive | | b APC PD-1 (Inhibitory | Anti- |
| | | | | cell surface receptor) | inflammatory |
| CD274 | Q9NZQ7 | Adaptive | | b Tcell PD-L1 (Inhibitory | Anti- |
| | | | | cell surface receptor) | inflammatory |
| HLA-DRA | P01903 | Adaptive | | c APC Peptide | Anti- |
| | | | | presentation | inflammatory |
| LAG3 | P18627 | Adaptive | | c Tcell LAG-3 (Inhibitory | Anti- |
| | | | | cell surface receptor) | inflammatory |
| CEACAM1 | P13688 | Adaptive | | d APC ligand for TIM-3 | Anti- |
| | | | | | inflammatory |
| HAVCR2 | Q8TDQ0 | Adaptive | | d Tcell TIM-3 (Inhibitory | Anti- |
| | | | | cell surface receptor) | inflammatory |
| CD86 | P42081 | Adaptive | | e APC Ligand for CTLA-4 | Anti- |
| | | | | | inflammatory |
| CTLA4 | P16410 | Adaptive | | e Tcell CTLA-4 | Anti- |
| | | | | (Inhibitory cell surface | inflammatory |
| | | | | receptor) | |
| IL10 | P22301 | Adaptive | | IL-10, expressed by Th2 | Anti- |
| | | | | | inflammatory |
| TGFB1 | P01137 | Adaptive | | TGF-β (inhinits Th and | Anti- |
| | | | | cytokines) | inflammatory |
| IL2RA | P01589 | Adaptive | | Treg activity | Anti- |
| | | | | | inflammatory |
| FOXP3 | Q9BZS1 | Adaptive | | FoxP3 (Treg) | Anti- |
| | | | | | inflammatory |
| SERPINE1 | P05121 | Coagulation | | Plasminogen activator | Pro- |
| | | | | inhibitor-1 (PAI-1), | coagulant |
| | | | | elevated risk of | |
| | | | | thrombosis | |
| F2 | P00734 | Coagulation | | Thrombin | Pro- |
| | | | | | coagulant |
| F3 | P13726 | Coagulation | | Tissue factor | Pro- |
| | | | | | coagulant |
| F5 | P12259 | Coagulation | | Factor V | Pro- |
| | | | | | coagulant |
| F7 | P08709 | Coagulation | | Factor VII | Pro- |
| | | | | | coagulant |
| F8 | P00451 | Coagulation | | Factor VIII | Pro- |
| | | | | | coagulant |
| F10 | P00742 | Coagulation | | Factor X | Pro- |
| | | | | | coagulant |
| F12 | P00748 | Coagulation | | Factor XII | Pro- |
| | | | | | coagulant |
| F13A1 | P00488 | Coagulation | | Factor XIII, A1 | Pro- |
| | | | | polypeptide | coagulant |
| ITGA2B | P08514 | Coagulation | | Integrin alpha 2b. | Pro- |
| | | | | Following activation | coagulant |
| | | | | integrin alpha-IIb/beta-3 | |
| | | | | brings about | |
| | | | | platelet/platelet interaction | |
| | | | | through binding of soluble | |
| | | | | fibrinogen. This step leads | |
| | | | | to rapid platelet | |
| | | | | aggregation which | |
| | | | | physically plugs ruptured | |
| | | | | endothelial cell surface. | |
| ITGB3 | P05106 | Coagulation | | Integrin beta 3. The | Pro- |
| | | | | ITGB3 protein product is | coagulant |
| | | | | the integrin beta chain beta | |
| | | | | 3. Integrins are integral | |
| | | | | cell-surface proteins | |
| | | | | composed of an alpha | |
| | | | | chain and a beta chain. A | |
| | | | | given chain may combine | |
| | | | | with multiple partners | |
| | | | | resulting in different | |
| | | | | integrins. Integrin beta 3 is | |
| | | | | found along with the alpha | |
| | | | | IIb chain in platelets. | |
| | | | | Integrins are known to | |
| | | | | participate in cell adhesion | |
| | | | | as well as cell-surface- | |
| | | | | mediated signaling. | |
| FGA | P02671 | Coagulation | | Fibrinogen alpha chain | Pro- |
| | | | | | coagulant |
| FGB | | Coagulation | | Fibrinogen beta chain | Pro- |
| | | | | | coagulant |
| FIBCD1 | Q8N539 | Coagulation | | Fibrinogen c domain | Pro- |
| | | | | containing 1 | coagulant |
| PTAFR | P25105 | Coagulation | Platelet | Platelet-activating factor | Pro- |
| | | | Activation | receptor | coagulant |
| THBD | P07204 | Coagulation | | Thrombomodulin | Anti- |
| | | | | | coagulant |
| TFPI | P10646 | Coagulation | | Tissue factor pathway | Anti- |
| | | | | inhibitor | coagulant |
| SERPINC1 | P01008 | Coagulation | | Antithrombin | Anti- |
| | | | | | coagulant |
| PROS1 | P07225 | Coagulation | | Protein S | Anti- |
| | | | | | coagulant |
| PROC | | Coagulation | | Protein C | Anti- |
| | | | | | coagulant |
| S1PR3 | Q99500 | Vascular | | Maintaining vascular | Decreased |
| | | Permeability | | integrity | permeability |
| S1PR1 | P21453 | Vascular | | Sphingosine-1-phosphate | Decreased |
| | | Permeability | | receptor 1, T cell | permeability |
| | | | | suppression | |
| ANGPT1 | Q15389 | Vascular | | Angiopoietin 1 | Decreased |
| | | Permeability | | | permeability |
| ANGPT2 | O15123 | Vascular | | angiopoietin 2 | Increased |
| | | Permeability | | | permeability |

For each gene in Table 6, the fold-change in gene expression was calculated between subtypes. Specifically, for each subtyping Model (Full/S/SS/P), linear regression was used to compare each gene expression among A/B/C subtypes. In order to adjust batch effects of microarray dataset from different studies, study IDs were included in the linear regression model. From the linear regression model, the coefficients of subtypes were used to calculate gene expression fold changes and Benjamini-Hochberg (BH)⁵³ adjusted p-values of subtypes were used to indicate if expression differences were statistically significant. Table 7 below depicts a representative dataset for subtype fold-changes in expression of the genes in Table 6. The fold-changes in gene expression between subtypes (e.g. fold change “A/B”=2\hat{ }(A−B) where A and B are the log 2 mean expression for the listed gene for the given subtype A and B) are listed as the numerical values in the table. Bold or underlined indicates a statistically significant fold-change as determined by BH. Bold indicates up-regulation and underlined indicates down-regulation. This dataset was then used to identify therapeutic candidates for the treatment of dysregulated host response taking into account whether the gene is expected to be appreciably expressed in blood.

TABLE 7
Representative Examples of Fold-Changes in Gene Expression Between A/B/C Subtypes

| Gene | A/B | A/C | B/A | B/C | C/A | C/B | Examples of Related Therapeutics |
|---|---|---|---|---|---|---|---|
| TREM1 | 1.382 | 1.786 | 0.724 | 1.2 | 0.56 | 0.833 | nangibotide (MOTREM), TREM-1 inhibitor |
| CD180 | 1.612 | 1.635 | 0.62 | 0.935 | 0.612 | 1.069 | |
| MIF | 1.348 | 1.281 | 0.742 | 0.988 | 0.781 | 1.012 | |
| CD14 | 0.934 | 1.698 | 1.071 | 1.724 | 0.589 | 0.58 | |
| IL15 | 1.07 | 1.758 | 0.934 | 1.547 | 0.569 | 0.646 | IL-15, NIZ985 |
| IL6 | 1.019 | 0.963 | 0.981 | 0.949 | 1.038 | 1.054 | Tocilizumab/anti-IL-6R |
| NLRP1 | 1.56 | 1.706 | 0.641 | 1.025 | 0.586 | 0.975 | |
| CASP1 | 0.916 | 1.692 | 1.091 | 1.81 | 0.591 | 0.552 | Emricasan (Novartis), pan-caspsase inhibitor |
| IL1B | 0.965 | 2.017 | 1.036 | 1.903 | 0.496 | 0.525 | IL1R1 (Amgen) |
| IL18 | 0.824 | 1.059 | 1.214 | 1.35 | 0.944 | 0.741 | |
| CXCL8 | 1.03 | 1.016 | 0.97 | 0.966 | 0.984 | 1.035 | |
| PYCARD | 0.832 | 1.496 | 1.202 | 1.722 | 0.669 | 0.581 | Emricasan, pan-caspase inhibitor |
| TLR4 | 0.577 | 1.1 | 1.733 | 1.87 | 0.909 | 0.535 | Resatorvid (Takeda), Eritoran (Eisai), HU-003 |
| | | | | | | | (Huons), NI-0101 (NovImmune) |
| TNF | 0.785 | 1.091 | 1.274 | 1.433 | 0.917 | 0.698 | CytoFab (anti-TNF-α AstraZeneca), |
| | | | | | | | Adalimumab/Humira, Infliximab/Remicade, |
| | | | | | | | Nerelimomab, Humicade, Afelimomab, |
| | | | | | | | rhTNFbp (TNF binding protein) |
| EBI3 | 0.555 | 0.711 | 1.801 | 1.198 | 1.406 | 0.834 | |
| IL27 | 0.794 | 0.985 | 1.26 | 1.18 | 1.015 | 0.848 | |
| IL1RL1 | 0.902 | 0.906 | 1.109 | 1.029 | 1.104 | 0.972 | |
| MPO | 0.547 | 0.407 | 1.828 | 0.669 | 2.459 | 1.494 | |
| S100A9 | 0.803 | 0.938 | 1.245 | 1.165 | 1.066 | 0.858 | |
| HSPA1B | 0.594 | 0.632 | 1.683 | 1.224 | 1.582 | 0.817 | |
| HSPA1A | 0.686 | 0.723 | 1.457 | 1.113 | 1.383 | 0.899 | |
| ELANE | 0.553 | 0.356 | 1.807 | 0.529 | 2.811 | 1.891 | |
| CTSG | 0.738 | 0.507 | 1.355 | 0.588 | 1.973 | 1.702 | |
| HMGB1 | 0.92 | 1.069 | 1.088 | 1.222 | 0.935 | 0.818 | |
| IL33 | 0.998 | 0.988 | 1.002 | 0.983 | 1.012 | 1.017 | |
| HSP90B1 | 1.112 | 1.108 | 0.899 | 1.033 | 0.902 | 0.968 | |
| HSPD1 | 1.19 | 1.161 | 0.84 | 1.04 | 0.861 | 0.962 | |
| S100A8 | 1.099 | 0.925 | 0.91 | 0.909 | 1.081 | 1.1 | |
| IL1A | 0.929 | 1.053 | 1.076 | 1.128 | 0.949 | 0.886 | IL1R1 (Amgen) |
| C3AR1 | 0.48 | 1.034 | 2.084 | 2.021 | 0.967 | 0.495 | |
| C5AR1 | 0.728 | 1.262 | 1.373 | 1.611 | 0.792 | 0.621 | IFX-1 (anti-C5a InflaRx), Soliris, Ultomiris |
| | | | | | | | (anti-C5a Alexion), Avacopan (anti-C5aR |
| C5AR2 | 0.835 | 1.046 | 1.198 | 1.234 | 0.956 | 0.81 | ChemoCentryx), C5a inhibitor/CaCP 29 |
| | | | | | | | (InflaRx) |
| C2 | 1.071 | 1.134 | 0.934 | 1.1 | 0.882 | 0.909 | |
| C4B | 0.718 | 0.494 | 1.393 | 0.544 | 2.024 | 1.838 | |
| SIGIRR | 1.682 | 1.668 | 0.594 | 0.99 | 0.599 | 1.01 | |
| CPB2 | 1.011 | 0.978 | 0.989 | 0.966 | 1.022 | 1.036 | |
| IL12A | 1.039 | 1.007 | 0.962 | 0.989 | 0.993 | 1.011 | |
| IL12B | 1.006 | 0.987 | 0.994 | 0.992 | 1.013 | 1.008 | |
| IL4 | 1.035 | 1.004 | 0.966 | 0.975 | 0.996 | 1.026 | |
| IL5 | 1.012 | 0.966 | 0.988 | 0.96 | 1.036 | 1.042 | |
| IL13 | 1.032 | 0.936 | 0.969 | 0.936 | 1.069 | 1.068 | |
| STAT1 | 1.093 | 2.243 | 0.915 | 1.924 | 0.446 | 0.52 | |
| IFNG | 1.216 | 1.051 | 0.823 | 0.953 | 0.952 | 1.049 | INF-gama, Actimmune, Recombinant protein, |
| | | | | | | | Genentech |
| LTA | 1.235 | 0.951 | 0.81 | 0.668 | 1.052 | 1.497 | |
| STAT4 | 1.902 | 2.03 | 0.526 | 1.039 | 0.493 | 0.963 | |
| HLA-DRA | 2.62 | 2.47 | 0.382 | 0.906 | 0.405 | 1.104 | |
| CD28 | 1.338 | 1.41 | 0.748 | 1.006 | 0.709 | 0.994 | AB103, Atox Bio (peptide CD28 Antagonist) |
| | | | | | | | (contraindicated) |
| CD3D | 2.984 | 2.611 | 0.335 | 0.809 | 0.383 | 1.236 | |
| CD3G | 2.755 | 2.492 | 0.363 | 0.918 | 0.401 | 1.089 | |
| CD3E | 2.799 | 2.132 | 0.357 | 0.746 | 0.469 | 1.34 | |
| PTMA | 1.608 | 1.352 | 0.622 | 0.883 | 0.74 | 1.132 | Thymosin alpha I (Roche), Thymalfasin |
| | | | | | | | peptide, T-lymphocyte subset modulators; Th1 |
| | | | | | | | cell stimulants; Th2-cell-inhibitors |
| | | | | | | | (immunostimulant) (SciClone Pharmaceuticals) |
| IL7R | 3.286 | 2.409 | 0.304 | 0.74 | 0.415 | 1.351 | |
| IL7 | 1.147 | 1.199 | 0.872 | 1.009 | 0.834 | 0.991 | CYT-107 (IL-7 Revnimmune) |
| IL17A | 1 | 0.986 | 1 | 0.968 | 1.014 | 1.033 | |
| IL3 | 1.01 | 0.991 | 0.99 | 1.006 | 1.009 | 0.994 | |
| GZMB | 2.204 | 2.453 | 0.454 | 1.152 | 0.408 | 0.868 | |
| BTLA | 1.93 | 1.708 | 0.518 | 0.913 | 0.585 | 1.095 | |
| TNFRSF14 | 1.119 | 1.752 | 0.894 | 1.472 | 0.571 | 0.679 | |
| LAG3 | 1.465 | 1.272 | 0.683 | 0.972 | 0.786 | 1.029 | |
| PDCD1 | 1.104 | 1.006 | 0.906 | 0.902 | 0.994 | 1.109 | Nivolumab, anti-PD-1 monoclonal antibody, |
| | | | | | | | pembrolizumab/Keytruda |
| CD274 | 0.613 | 1.46 | 1.632 | 2.274 | 0.685 | 0.44 | Anti-PD-L1 (BMS-936559) |
| CD86 | 2.177 | 1.939 | 0.459 | 0.89 | 0.516 | 1.124 | |
| HAVCR2 | 0.819 | 1.201 | 1.221 | 1.45 | 0.832 | 0.69 | |
| CTLA4 | 1.158 | 1.044 | 0.863 | 0.975 | 0.958 | 1.026 | anti-CTLA-4 monoclonal antibody, |
| | | | | | | | Ipilimumab, Medarex |
| IL10 | 0.639 | 0.783 | 1.566 | 1.233 | 1.277 | 0.811 | |
| FOXP3 | 1.036 | 0.921 | 0.965 | 0.903 | 1.085 | 1.107 | |
| IL2 | 1.017 | 0.985 | 0.983 | 0.975 | 1.016 | 1.025 | IL-2 Roncoleukin |
| IL2RA | 0.964 | 1.024 | 1.038 | 1.114 | 0.977 | 0.897 | |
| TNFRSF18 | 1.057 | 0.954 | 0.946 | 0.938 | 1.048 | 1.066 | |
| TGFB1 | 0.875 | 0.996 | 1.143 | 1.156 | 1.004 | 0.865 | |
| SERPINE1 | 1.007 | 0.904 | 0.993 | 0.887 | 1.106 | 1.127 | defibrotide |
| F2 | 1.005 | 0.934 | 0.995 | 0.928 | 1.07 | 1.077 | Antithrombin (CSL Behring), tanogitran |
| | | | | | | | (antagonizes Factors Xa and IIa), Boehringer |
| | | | | | | | Ingelheim |
| F3 | 0.981 | 0.89 | 1.02 | 0.933 | 1.124 | 1.072 | |
| F5 | 0.603 | 1.077 | 1.658 | 1.603 | 0.929 | 0.624 | |
| F7 | 1.017 | 0.887 | 0.983 | 0.893 | 1.127 | 1.12 | |
| F8 | 0.599 | 0.959 | 1.67 | 1.516 | 1.042 | 0.66 | |
| F9 | 0.997 | 0.978 | 1.003 | 0.979 | 1.022 | 1.021 | TNX-832, Sunol cH36, mAb, Factor IX |
| | | | | | | | inhibitors; Factor X inhibitors |
| F10 | 1.017 | 0.939 | 0.984 | 0.937 | 1.065 | 1.068 | TNX-832, Sunol cH36, mAb, Factor IX |
| | | | | | | | inhibitors; Factor X inhibitors, tanogitran |
| | | | | | | | (antagonizes Factors Xa and IIa), Boehringer |
| | | | | | | | Ingelheim |
| F11 | 1.008 | 0.976 | 0.992 | 0.98 | 1.024 | 1.021 | |
| F12 | 0.656 | 0.762 | 1.526 | 1.163 | 1.313 | 0.86 | |
| F13A1 | 1.67 | 0.763 | 0.599 | 0.463 | 1.311 | 2.158 | |
| ITGA2B | 0.977 | 0.513 | 1.023 | 0.528 | 1.948 | 1.892 | |
| ITGB3 | 0.918 | 0.607 | 1.09 | 0.623 | 1.647 | 1.605 | |
| FGA | 1.011 | 0.937 | 0.989 | 0.947 | 1.067 | 1.055 | |
| FGB | 0.997 | 0.944 | 1.003 | 0.955 | 1.059 | 1.047 | |
| FGG | 0.995 | 0.982 | 1.005 | 0.975 | 1.018 | 1.026 | |
| FIBCD1 | 1.046 | 0.864 | 0.956 | 0.751 | 1.157 | 1.332 | |
| PTAFR | 0.722 | 1.21 | 1.384 | 1.403 | 0.827 | 0.713 | Minopafant (PAF antagonist), Pafase |
| | | | | | | | (inactivates PAF), TCV-309 (PAF antagonist), |
| | | | | | | | YM-264 (PAF antagonist), SM-12502 (PAF |
| | | | | | | | antagonist), UK-74505 (PAF receptor |
| | | | | | | | antagonist), Ginkgolide B (PAF inhinbitor), |
| | | | | | | | Epafipase (Recombinant Human Platelet- |
| | | | | | | | Activating Factor Acetylhydrolase) |
| CSF3 | 1.04 | 0.895 | 0.962 | 0.875 | 1.117 | 1.142 | G-CSF, Filgrastim, Recombinant protein, |
| | | | | | | | Immunostimulants, Amgen (contraindicsted) |
| CSF2 | 1.015 | 0.879 | 0.985 | 0.882 | 1.138 | 1.133 | Sargramostim (Genzyme, etc.) |
| PROS1 | 0.784 | 0.447 | 1.275 | 0.581 | 2.238 | 1.722 | |
| PROC | 1.051 | 0.973 | 0.952 | 0.927 | 1.028 | 1.079 | |
| THBD | 0.666 | 1.231 | 1.501 | 1.769 | 0.812 | 0.565 | Thrombomodulin, ART-123 (Asahi), Protein C |
| | | | | | | | Stimulant |
| TFPI | 0.908 | 0.645 | 1.101 | 0.74 | 1.55 | 1.351 | tifacogin (recombinant TFPI) |
| SERPINC1 | 1.019 | 0.95 | 0.981 | 0.958 | 1.052 | 1.044 | |
| PROCR | 1.023 | 1.009 | 0.977 | 1.009 | 0.991 | 0.991 | |
| S1PR3 | 1.58 | 1.639 | 0.633 | 1.013 | 0.61 | 0.988 | |
| S1PR1 | 0.579 | 1.035 | 1.727 | 1.687 | 0.966 | 0.593 | |
| ANGPT1 | 1.026 | 0.929 | 0.975 | 0.906 | 1.076 | 1.104 | |
| ANGPT2 | 1.021 | 0.964 | 0.98 | 0.948 | 1.037 | 1.055 | |
| TEK | 1.013 | 0.966 | 0.987 | 0.971 | 1.035 | 1.029 | |
| AGT | 1.036 | 0.935 | 0.966 | 0.909 | 1.07 | 1.1 | GIAPREZA (La Jolla) |
| ACE | 1.008 | 0.956 | 0.992 | 0.892 | 1.046 | 1.122 | GIAPREZA (La Jolla) |
| REN | 1.02 | 0.947 | 0.98 | 0.931 | 1.056 | 1.074 | GIAPREZA (La Jolla) |
| ACE2 | 1.012 | 0.981 | 0.988 | 0.974 | 1.019 | 1.027 | GIAPREZA (La Jolla) |
| AGTR1 | 1.022 | 0.989 | 0.979 | 0.981 | 1.011 | 1.019 | GIAPREZA (La Jolla) |
| GLP1R | 1.021 | 0.911 | 0.979 | 0.917 | 1.098 | 1.09 | Exenatide, Byetta, Bydureon, GLP-1 receptor |
| | | | | | | | agonist (Amylin Pharmaceuticals) |
| TNFRSF1B | 1.044 | 1.719 | 0.958 | 1.628 | 0.582 | 0.614 | p75 TNF receptor, Recombinant protein, TNF |
| | | | | | | | blocker, Amgen |
| IL11 | 1.036 | 0.932 | 0.965 | 0.913 | 1.073 | 1.096 | Oprelvekin, Thrombocytopenia |
| TNFRSF1A | 0.773 | 1.435 | 1.293 | 1.75 | 0.697 | 0.571 | Lenercept (Roche) |
| LTF | 0.13 | 0.129 | 7.68 | 1.141 | 7.752 | 0.876 | Talactoferrin alfa, Apolactoferrin, recombinant |
| | | | | | | | lactoferrin (Agennix) |
| IL3RA | 0.796 | 0.776 | 1.256 | 1.111 | 1.289 | 0.9 | Interleukin-3-receptor-alpha-subunit- |
| | | | | | | | antagonists, Talacotuzumab |
| FLT3 | 0.971 | 1.041 | 1.03 | 1.062 | 0.961 | 0.942 | Flt3 ligand, Mobista, Fms-like tyrosine kinase |
| | | | | | | | 3 stimulants, increases Treg proliferation, |
| | | | | | | | Amgen |
| TLR3 | 1.033 | 1.017 | 0.968 | 0.997 | 0.984 | 1.003 | Poly-ICLC, TLR3 agonist, Janssen, Peptide P7 |
| MS4A1 | 2.023 | 1.954 | 0.494 | 0.862 | 0.512 | 1.16 | Rituximab, destroys B cells expressing CD20 |
| CASP2 | 0.975 | 1.101 | 1.026 | 1.119 | 0.908 | 0.894 | Emricasan |
| CASP3 | 0.774 | 1.177 | 1.292 | 1.442 | 0.85 | 0.694 | Emricasan |
| CASP4 | 0.9 | 1.642 | 1.111 | 1.791 | 0.609 | 0.558 | Emricasan |
| CASP5 | 0.744 | 2.056 | 1.343 | 2.593 | 0.486 | 0.386 | Emricasan |
| CASP6 | 1.205 | 1.362 | 0.83 | 1.118 | 0.734 | 0.894 | Emricasan |
| CASP7 | 0.976 | 1.233 | 1.025 | 1.221 | 0.811 | 0.819 | Emricasan |
| CASP8 | 0.992 | 1.307 | 1.008 | 1.303 | 0.765 | 0.767 | Emricasan |
| CASP9 | 0.81 | 0.965 | 1.235 | 1.142 | 1.037 | 0.876 | Emricasan |
| CASP10 | 0.975 | 1.047 | 1.026 | 1.1 | 0.955 | 0.909 | Emricasan |
| CASP12 | 1.003 | 0.985 | 0.997 | 0.985 | 1.015 | 1.015 | Emricasan |
| CASP14 | 0.996 | 0.985 | 1.004 | 0.929 | 1.015 | 1.077 | Emricasan |
| BDKRB2 | 1.035 | 0.973 | 0.966 | 0.942 | 1.028 | 1.062 | deltibant (bradykinin-2 (BK-2) receptor |
| | | | | | | | antagonist), NPC-17761 (bradykinin-2 (BK-2) |
| | | | | | | | receptor antagonist) |
| KNG1 | 1.01 | 0.985 | 0.99 | 0.979 | 1.015 | 1.021 | deltibant (bradykinin-2 (BK-2) receptor |
| | | | | | | | antagonist), NPC-17761 (bradykinin-2 (BK-2) |
| | | | | | | | receptor antagonist) |
| PLA2G3 | 0.987 | 0.966 | 1.013 | 0.964 | 1.036 | 1.037 | varespladib (sPLA2 inhibitor), IPP-201007 |
| | | | | | | | (sPLA2 inhibitor) |
| PLAT | 1.019 | 0.991 | 0.982 | 0.979 | 1.009 | 1.022 | defibrotide |
| PDYN | 1.002 | 0.972 | 0.998 | 0.976 | 1.029 | 1.025 | naloxone |
| PENK | 0.985 | 0.983 | 1.015 | 0.993 | 1.018 | 1.007 | naloxone |
| OPRM1 | 1.008 | 0.99 | 0.992 | 0.99 | 1.01 | 1.01 | naloxone |
| POMC | 1.093 | 0.971 | 0.915 | 0.971 | 0.971 | 0.971 | naloxone |
| PNOC | 1.697 | 1.367 | 0.589 | 0.826 | 0.732 | 1.21 | naloxone |
| NOS2 | 0.971 | 0.961 | 0.971 | 0.971 | 1.041 | 0.971 | GW-274150 (NOS inhibitor), hemoximer (NO |
| | | | | | | | scavenger), nebacumab (NO scavenger), ONO- |
| | | | | | | | 1714 (iNOS inhibitor), Tilarginine (NO |
| | | | | | | | synthase inhibitor), Norathiol (NO-inhibitor), |
| | | | | | | | targinine (NOS inhibitor), aSeptiMab (anti- |
| | | | | | | | NOS) |
| ADM | 0.565 | 1.263 | 1.771 | 2.185 | 0.792 | 0.458 | adrecizumab (stabilizes/increases |
| | | | | | | | adrenomedullin and reverses vascular |
| | | | | | | | permeability) |
| RAMP2 | 0.971 | 0.9 | 0.971 | 0.903 | 1.111 | 1.108 | adrecizumab (stabilizes/increases |
| | | | | | | | adrenomedullin and reverses vascular |
| | | | | | | | permeability) |
| RAMP3 | 0.971 | 0.971 | 0.971 | 0.952 | 0.971 | 1.05 | adrecizumab (stabilizes/increases |
| | | | | | | | adrenomedullin and reverses vascular |
| | | | | | | | permeability) |
| CALCRL | 1.016 | 1.006 | 0.984 | 0.996 | 0.994 | 1.004 | adrecizumab (stabilizes/increases |
| | | | | | | | adrenomedullin and reverses vascular |
| | | | | | | | permeability) |
| FAS | 0.668 | 0.971 | 1.497 | 1.718 | 0.971 | 0.582 | asunercept (blocks CD95 ligand) |
| FASLG | 1.3 | 1.159 | 0.769 | 0.952 | 0.863 | 0.971 | asunercept (blocks CD95 ligand) |
| ADRA2A | 0.971 | 0.971 | 0.971 | 0.901 | 0.971 | 1.11 | centhaquin, Alpha-2A adrenergic receptor |
| | | | | | | | agonist and Alpha-1 adrenergic receptor |
| | | | | | | | antagonist: reduces blood lactate and increase |
| | | | | | | | blood pressure |
| ADRA1A | 0.971 | 0.971 | 0.971 | 0.911 | 0.971 | 1.098 | centhaquin, Alpha-2A adrenergic receptor |
| | | | | | | | agonist and Alpha-1 adrenergic receptor |
| | | | | | | | antagonist: reduces blood lactate and increase |
| | | | | | | | blood pressure |
| TMSB4X | 0.971 | 0.971 | 0.971 | 1.106 | 0.971 | 0.905 | timbetasin (synthetic TB4) |
| ACTA1 | 0.971 | 0.937 | 0.971 | 0.971 | 1.067 | 0.971 | timbetasin |
| ACTA2 | 1.01 | 1.017 | 0.99 | 1.056 | 0.983 | 0.947 | timbetasin |
| ACTC1 | 1.005 | 0.966 | 0.995 | 0.971 | 1.035 | 1.03 | timbetasin |
| ACTB | 0.819 | 0.971 | 1.22 | 1.132 | 0.971 | 0.883 | timbetasin |
| ACTG1 | 0.878 | 0.971 | 1.139 | 1.193 | 0.971 | 0.838 | timbetasin |
| ACTG2 | 0.971 | 0.941 | 0.971 | 0.94 | 1.062 | 1.064 | timbetasin |
| GPX1 | 1.618 | 0.741 | 0.618 | 0.45 | 1.35 | 2.22 | Rexis (enhances Glutathione peroxidase) |
| GPX2 | 0.971 | 0.971 | 0.971 | 0.848 | 0.971 | 1.18 | Rexis (enhances Glutathione peroxidase) |
| GPX3 | 0.971 | 0.848 | 0.971 | 0.847 | 1.18 | 1.181 | Rexis (enhances Glutathione peroxidase) |
| GPX4 | 1.776 | 0.858 | 0.563 | 0.487 | 1.166 | 2.055 | Rexis (enhances Glutathione peroxidase) |
| GPX5 | 0.971 | 0.971 | 0.971 | 0.894 | 0.971 | 1.119 | Rexis (enhances Glutathione peroxidase) |
| GPX6 | 1.037 | 0.999 | 0.965 | 0.962 | 1.001 | 1.039 | Rexis (enhances Glutathione peroxidase) |
| GPX7 | 0.886 | 1.057 | 1.128 | 1.192 | 0.947 | 0.839 | Rexis (enhances Glutathione peroxidase) |
| GPX8 | 0.967 | 0.965 | 1.034 | 0.981 | 1.036 | 1.019 | Rexis (enhances Glutathione peroxidase) |
| Cxcl9 | 1.019 | 1.022 | 0.981 | 1.035 | 0.979 | 0.966 | ISU201 |
| Cxcl10 | 1.167 | 1.467 | 0.857 | 1.301 | 0.681 | 0.768 | ISU201 |
| Icam1 | 0.789 | 1.148 | 1.268 | 1.455 | 0.871 | 0.687 | ISU201 |
| Vcam1 | 1.035 | 0.965 | 0.966 | 0.943 | 1.037 | 1.06 | ISU201 |
| IL12B | 1.006 | 0.987 | 0.994 | 0.992 | 1.013 | 1.008 | ISU201 |
| Csf1 | 1.017 | 0.955 | 0.983 | 0.894 | 1.047 | 1.118 | ISU201 |
| PCSK9 | 0.76 | 0.838 | 1.316 | 1.212 | 1.194 | 0.825 | LGT-209, anti-PCSK9 antibody |
| TLR2 | 0.563 | 1.181 | 1.775 | 2.055 | 0.847 | 0.487 | Peptide P13 |
| TLR9 | 1.067 | 1.013 | 0.937 | 0.958 | 0.987 | 1.044 | Peptide P13, Peptide P16 |
| TLR6 | 0.796 | 1.548 | 1.256 | 1.779 | 0.646 | 0.562 | Tinospora cordifolia derivative |
| ALOX5AP | 0.528 | 0.701 | 1.893 | 1.422 | 1.427 | 0.703 | AKI: montelukast |
| PLA2G4A | 0.721 | 1.068 | 1.387 | 1.467 | 0.936 | 0.682 | AKI: montelukast |
| MGST2 | 0.945 | 1.209 | 1.059 | 1.292 | 0.827 | 0.774 | AKI: montelukast |
| CYSLTR1 | 1.058 | 1.847 | 0.945 | 1.682 | 0.541 | 0.595 | AKI: montelukast |
| CYSLTR2 | 1.098 | 0.866 | 0.911 | 0.831 | 1.155 | 1.204 | AKI: montelukast |
| LTB4R2 | 0.988 | 0.942 | 1.012 | 0.917 | 1.062 | 1.09 | AKI: montelukast |
| LCN2 | 0.073 | 0.09 | 13.764 | 1.458 | 11.128 | 0.686 | AKI: montelukast |
| Bdnf | 1.007 | 0.983 | 0.993 | 0.989 | 1.018 | 1.011 | Hydrocortisone |
| Ncoa2 | 0.943 | 1.107 | 1.061 | 1.162 | 0.903 | 0.861 | Hydrocortisone |
| Nr3c1 | 0.712 | 1.121 | 1.404 | 1.564 | 0.892 | 0.64 | Hydrocortisone |
| Ntrk2 | 1.015 | 0.979 | 0.986 | 0.962 | 1.021 | 1.04 | Hydrocortisone |
| Ppp5c | 1.031 | 1.007 | 0.97 | 0.984 | 0.993 | 1.016 | Hydrocortisone |
| Arntl | 0.857 | 1.446 | 1.166 | 1.634 | 0.691 | 0.612 | Hydrocortisone |
| Clock | 1.091 | 1.317 | 0.916 | 1.141 | 0.759 | 0.876 | Hydrocortisone |
| Cry1 | 1.372 | 1.206 | 0.729 | 0.866 | 0.829 | 1.155 | Hydrocortisone |
| Cry2 | 1.198 | 1.127 | 0.835 | 0.99 | 0.887 | 1.01 | Hydrocortisone |
| Phb | 1.517 | 1.357 | 0.659 | 0.926 | 0.737 | 1.08 | Hydrocortisone |
| Per1 | 0.891 | 0.796 | 1.122 | 0.904 | 1.257 | 1.106 | Hydrocortisone |
| Arid1a | 0.97 | 1.24 | 1.031 | 1.226 | 0.807 | 0.815 | Hydrocortisone |
| Ptges3 | 1.189 | 1.044 | 0.841 | 0.875 | 0.958 | 1.143 | Hydrocortisone |
| Ywhah | 0.778 | 0.784 | 1.285 | 0.991 | 1.276 | 1.01 | Hydrocortisone |

FIG. 8 depicts the conclusions of this further analysis of Tables 6 and 7, in accordance with an embodiment. Dysregulated host response patients of subtype A exhibit up-regulation of biomarkers associated with innate immune activity involved in pathogen recognition (e.g., via recognition of pathogen-associated molecular patterns (PAMPs)), up-regulation of biomarkers associated with innate immune regulation, and up-regulation of biomarkers associated with adaptive immune activity. Dysregulated host response patients of subtype B exhibit up-regulation of biomarkers associated with innate immune activity involved in recognition of damage-associated molecular patterns (DAMPs), up-regulation of biomarkers associated with DAMPs, up-regulation of biomarkers associated with inflammation (e.g. TNF-alpha), up-regulation of biomarkers associated with complement activity, down-regulation of biomarkers associated with adaptive immune activity, up-regulation of biomarkers associated with adaptive immune suppression, and up-regulation of markers associated with increased risk of acute kidney injury. Subtype C patients exhibit down-regulation of biomarkers associated with innate and adaptive immune activity, up-regulation of biomarkers associated with DAMPs, up-regulation of biomarkers associated with cellular recruitment (e.g. G-CSF and GM-CSF), up-regulation of biomarkers associated with increased risk of thrombosis, and up-regulation of biomarkers associated with coagulation.

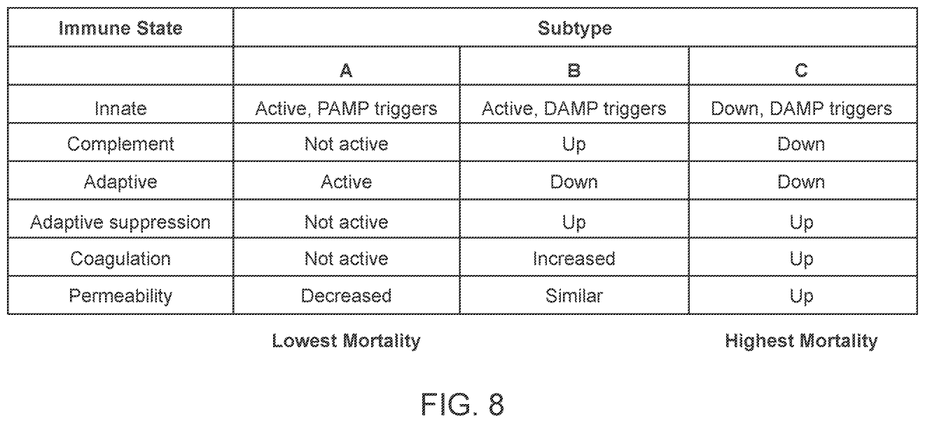

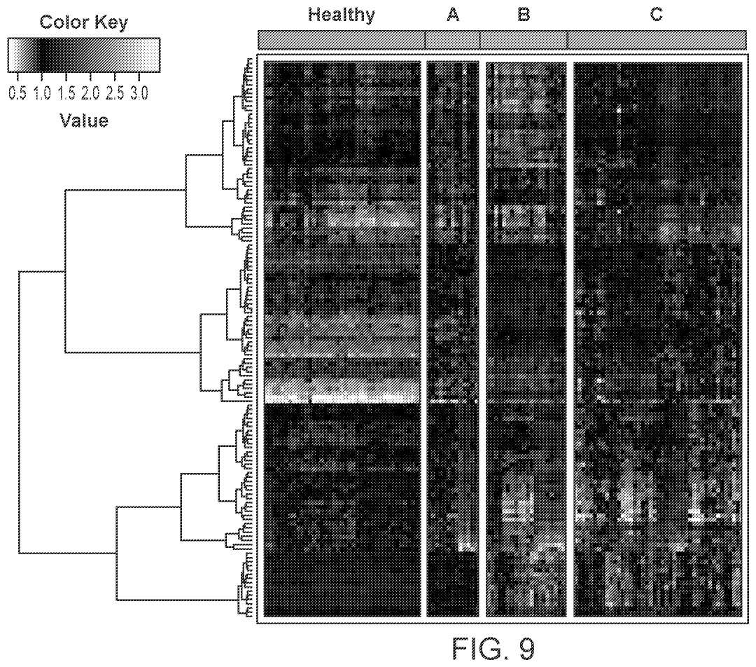

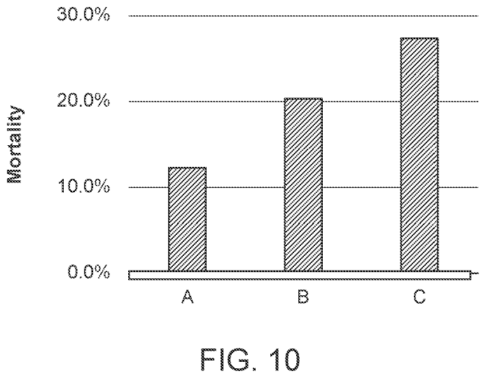

These findings of differential biomarker expression between subtypes A, B, and C inform general therapeutic strategies. FIG. 9 depicts a heat map depicting differential expression of genes from Table 6 for dysregulated host response patients having subtypes A, B, and C, and for healthy subjects without dysregulated host response, in accordance with an embodiment. As discussed below with regard to FIG. 10, subtype A patients exhibit relatively low mortality, which may be attributable to relatively beneficial host response. In fact, as shown in FIG. 9, differential expression of genes for dysregulated host response patients having subtype A most closely resembles differential expression of genes for healthy subjects without dysregulated host response. Thus, in subtype A patients, it may be beneficial to avoid immunomodulatory agents exhibiting immunosuppressive effects that suppress the beneficial host response. In subtype B patients, it may be beneficial to stimulate adaptive immune activity, attenuate innate immune stimulants (e.g. TNF-α), attenuate complement immune activity, attenuate DAMPs and/or block DAMP receptors, and activate PAMP receptors. In subtype C patients, it may be beneficial to simulate adaptive immune activity, administer anticoagulants or agents that indirectly attenuate pro-coagulation factors, decrease vascular permeability, attenuate DAMPs and/or block DAMP receptors, and activate PAMP receptors.

FIG. 10 depicts risk of mortality for dysregulated host response patients having subtypes A, B, and C, in accordance with an embodiment. As mentioned above, subtype A patients exhibit a low risk of morality, relative to subtype B and C patients. Furthermore, subtype C patients exhibit a high risk of morality, relative to subtype A and B patients. Therefore, the subtyping Models may be used as a prognostic to assess the risk of mortality of a dysregulated host response patient.

### VI. Evaluation of Therapeutics for Dysregulated Host Response Patient Subtypes

As discussed above, the genes of Tables 6 and 7 are associated with pharmacology of existing therapeutics. For instance, examples of existing therapeutics that are associated with certain genes are indicated in Table 7. Analysis of these genes of Tables 6 and 7 according to subtype, informs the use of the existing therapeutics associated with these genes for treating dysregulated host response patients of the subtype. Specifically, Table 8 depicts therapeutic hypotheses for systemic immune patients having subtypes A, B, and C, determined based on the analysis of differential gene expression of Table 7, in accordance with an embodiment.

As a specific example, while anti-TNF-alpha has failed to show benefit in past sepsis clinical trials, analysis of differential gene expression according to subtype can inform which specific subtype of patients may respond to anti-TNF-alpha. In this example, the TNF gene is seen to be up-regulated in patients having subtype B and thus, subtype B patients may specifically respond to anti-TNF-alpha therapy.

Table 8 below summarizes an analysis of existing therapeutics that are anticipated to provide the desired therapeutic effects for subtypes A, B, and C mentioned above.

TABLE 8
Representative Examples of Therapeutic Hypotheses for Dysregulated host response Patient Subtypes

| Genetic | Trade | | Sepsis | Anticipated | Subtype | |
|---|---|---|---|---|---|---|
| Name | Name | Description | Hypothesis | Effect | Hypothesis | Evidence |
| Anti- | BMS- | Anti- | Blocks upregulation | Increase | May benefit | Type B and C |
| PD-L1 | 936559 | PD-L1 | of PD-1/PD-L1 to | adaptive | subtype B | patients have a |
| | | | restore immune cell | immune | and C | suppressed |
| | | | function | activity | | adaptive |
| | | | | | | immune |
| | | | | | | response and |
| | | | | | | Type B up- |
| | | | | | | regulated PD- |
| | | | | | | L1 and down- |
| | | | | | | regulated |
| | | | | | | INF-g |
| PD-L1 | BMS- | Peptide that | Blocks upregulation | Increase | May benefit | Type B and C |
| blocker | 986189 | blocks | of PD-1/PD-L1 to | adaptive | subtype B | patients have a |
| | | PD/PD-L1 | restore immune cell | immune | and C | suppressed |
| | | | function | activity | | adaptive |
| | | | | | | immune |
| | | | | | | response and |
| | | | | | | Type B up- |
| | | | | | | regulated PD- |
| | | | | | | L1 and down- |
| | | | | | | regulated |
| | | | | | | INF-g |
| Anti- | CM-24 | anti- | | Increase | May benefit | Type B and C |
| CEACAM1 | | CEACAM1 | | adaptive | subtype B | patients have a |
| | | | | immune | and C | suppressed |
| | | | | activity | | adaptive |
| | | | | | | immune |
| | | | | | | response and |
| | | | | | | up-regulated |
| | | | | | | CEACAM1 |
| | | | | | | and TIM-3 |
| Anti- | MK-1966 | anti-IL-10 | | Increase | May benefit | Type B patients |
| IL-10R | | receptor | | adaptive | subtype B | have a |
| | | | | immune | | suppressed |
| | | | | activity | | adaptive |
| | | | | | | immune |
| | | | | | | response and |
| | | | | | | up-regulated |
| | | | | | | IL-10 |
| TNF | JTE 607 | Reduces | These results suggest | Decrease | May benefit | Type B |
| inhibitor | | TNF-α, | that JTE-607 can | inflammation, | subtype B | exhibits |
| | | IL-1β, IL-6, | inhibit the production | increase | | relative high |
| | | IL-8, IL-10 | of inflammatory | adaptive | | gene |
| | | | cytokines such as | | | expression of |
| | | | tumor necrosis factor- | | | TNF-a (pro- |
| | | | alpha, interleukin-6 | | | inflammatory |
| | | | and cytokine-induced | | | cytokine) and |
| | | | neutrophil | | | IL-10 (adaptive |
| | | | chemoattractant and | | | immune |
| | | | attenuate acid- | | | suppressant) |
| | | | induced lung injury in | | | |
| | | | rats. This agent might | | | |
| | | | be therapeutically | | | |
| | | | useful for lung injury | | | |
| IL-7 | CYT-107 | IL-7, | A defining | Increase | May benefit | IL-7 gene |
| | | immune | pathophysiologic | adaptive | subtype B | expression is |
| | | stimulant | feature of sepsis is | immune | and C | relatively low |
| | | | profound apoptosis- | activity | | in subtype B |
| | | | induced death and | | | and C and these |
| | | | depletion of CD4+ | | | Types exhibit |
| | | | and CD8+ T cells. | | | down- |
| | | | Interleukin-7 (IL-7) is | | | regulation of |
| | | | an antiapoptotic | | | genes |
| | | | common γ-chain | | | associated with |
| | | | cytokine that is | | | immune |
| | | | essential for | | | activity. |
| | | | lymphocyte | | | |
| | | | proliferation and | | | |
| | | | survival. Clinical | | | |
| | | | trials of IL-7 in over | | | |
| | | | 390 oncologic and | | | |
| | | | lymphopenic patients | | | |
| | | | showed that IL-7 was | | | |
| | | | safe, invariably | | | |
| | | | increased CD4+ and | | | |
| | | | CD8+ lymphocyte | | | |
| | | | counts, and improved | | | |
| | | | immunity. | | | |
| | tanogitran | Antithrombin: | | anti- | May benefit | Type C patients |
| | | antagonizes | | coagulant | subtype C | have |
| | | Factors Xa | | | | upregulated |
| | | and IIa | | | | genes related to |
| | | | | | | coagulation |
| | TNX-832, | mAb Factor IX | Tissue factor (TF) is a | anti- | May benefit | Type C patients |
| | Sunol | inhibitors; | transmembrane | coagulant | subtype C | have |
| | cH36 | Factor X | glycoprotein that acts | | | upregulated |
| | | inhibitors | as the principal | | | genes related to |
| | | | initiator of the | | | coagulation |
| | | | extrinsic coagulation | | | |
| | | | pathway. TF is a key | | | |
| | | | mediator between the | | | |
| | | | immune system and | | | |
| | | | coagulation and is the | | | |
| | | | principal activator of | | | |
| | | | coagulation. Vessel | | | |
| | | | injury or pathological | | | |
| | | | conditions leading to | | | |
| | | | the exposure TF in the | | | |
| | | | vascular adventitia | | | |
| | | | layer or induction of | | | |
| | | | TF expression on | | | |
| | | | endothelial cells and | | | |
| | | | monocytes permits | | | |
| | | | interactions between | | | |
| | | | TF and coagulation | | | |
| | | | factor VIIa (FVIIa) | | | |
| | | | resulting in the | | | |
| | | | formation of the high | | | |
| | | | affinity TF-FVIIa | | | |
| | | | complex. TNX-832 | | | |
| | | | (formerly known as | | | |
| | | | Sunol-cH36), directed | | | |
| | | | against human TF, | | | |
| | | | which can block the | | | |
| | | | pathological | | | |
| | | | complications of TF- | | | |
| | | | dependent thrombus | | | |
| | | | formation. The | | | |
| | | | blockage by TNX-832 | | | |
| | | | of initiating events in | | | |
| | | | the extrinsic | | | |
| | | | coagulation pathway | | | |
| | | | may attenuate the | | | |
| | | | effects on pro- | | | |
| | | | inflammatory events | | | |
| | tifacogin | TFPI: anti- | Systemic activation of | anti- | May benefit | Type C patients |
| | | coaggulant | coagulation and | coagulant | subtype C | have |
| | | | thrombus formation in | | | upregulated |
| | | | the microvasculature | | | genes related to |
| | | | accompanies organ | | | coagulation |
| | | | dysfunction and | | | |
| | | | excess mortality in | | | |
| | | | severe sepsis. Tissue | | | |
| | | | factor | | | |
| | | | (thromboplastin) is a | | | |
| | | | major initiator of the | | | |
| | | | blood coagulation | | | |
| | | | process. Endothelial | | | |
| | | | damage is common in | | | |
| | | | severe sepsis, as | | | |
| | | | shown by elevations | | | |
| | | | in endothelial derived | | | |
| | | | factors, such as von | | | |
| | | | Willebrand factor, | | | |
| | | | and by the presence of | | | |
| | | | coagulation | | | |
| | | | abnormalities, | | | |
| | | | including | | | |
| | | | prolongation of | | | |
| | | | prothrombin time, in | | | |
| | | | more than 90% of | | | |
| | | | patients who are | | | |
| | | | severely ill and | | | |
| | | | infected. It is | | | |
| | | | hypothesized that in | | | |
| | | | patients with severe | | | |
| | | | sepsis, TFPI may | | | |
| | | | protect the | | | |
| | | | microvasculature | | | |
| | | | endothelium from | | | |
| | | | coagulation and | | | |
| | | | sepsis-induced injury. | | | |
| | | | This hypothesis is | | | |
| | | | supported by several | | | |
| | | | preclinical studies in | | | |
| | | | which exogenous | | | |
| | | | TFPI expressed in | | | |
| | | | mammalian cells | | | |
| | | | and/or Escherichia | | | |
| | | | coli improved | | | |
| | | | outcome in septic | | | |
| | | | animals | | | |
| | iloprost | Anti- | combination therapy | anti- | May benefit | Coagulation in |
| | trometamol + | coagulant | in septic shock | coagulant | subtype C | subtype C |
| | eptifibatide | | patients is expected to | | | |
| | | | deactivate the | | | |
| | | | endothelium and | | | |
| | | | restore vascular | | | |
| | | | integrity, reduce | | | |
| | | | formation of | | | |
| | | | microvascular | | | |
| | | | thrombosis and | | | |
| | | | dissolve existing clots | | | |
| | | | in the | | | |
| | | | microcirculation and | | | |
| | | | maintain platelet | | | |
| | | | counts, thereby | | | |
| | | | improving platelet- | | | |
| | | | mediated immune | | | |
| | | | function and reducing | | | |
| | | | the risk of bleeding. | | | |
| | | | Together this is | | | |
| | | | expected to translate | | | |
| | | | into reduced organ | | | |
| | | | failure and improved | | | |
| | | | outcome in patients | | | |
| | | | with septic shock. | | | |
| | Pafase, | PAF | BN 52021 is an | anti- | May benefit | PAF receptor |
| | Ginkgolide B | inhinbitor | effective and specific | coagulant | subtype B | upregulated in |
| | | | PAF receptor | | | subtype B |
| | | | antagonist (PAFra) | | | |
| | | | with proven inhibiting | | | |
| | | | effects on PAF- | | | |
| | | | induced events, i.e. in | | | |
| | | | vitro on platelet Study | | | |
| | | | Design aggregation, | | | |
| | | | and in animals on | | | |
| | | | shock events induced | | | |
| | | | by endotoxin | | | |
| | | | (hypotension, | | | |
| | | | gastrointestinal | | | |
| | | | disorders and | | | |
| | | | bronchial spasm). [7] | | | |
| | | | It has also been | | | |
| | | | demonstrated that | | | |
| | | | preventive | | | |
| | | | administration of | | | |
| | | | BN 52021 in rats | | | |
| | | | attenuated the | | | |
| | | | reactions to injected | | | |
| | | | endotoxin: the | | | |
| | | | mortality rate was | | | |
| | | | decreased and the | | | |
| | | | release of | | | |
| | | | thromboxane and | | | |
| | | | prostaglandin factor | | | |
| | | | 1-α | | | |
| | | | (PGF1-α) was | | | |
| | | | reduced. | | | |
| | Epafipase or | Recombinant | The therapeutic | anti- | May benefit | PAF receptor |
| | Pafase | Human | rationale for the | coagulant | subtype B | upregulated in |
| | | Platelet- | administration of | | | subtype B |
| | | Activating | rPAF-AH in | | | |
| | | Factor | severe sepsis is to | | | |
| | | Acetylhydrolase | increase PAF-AH | | | |
| | | | activity in the | | | |
| | | | presence of | | | |
| | | | generalized | | | |
| | | | inflammation and | | | |
| | | | coagulation. The | | | |
| | | | therapeutic | | | |
| | | | potential for this | | | |
| | | | strategy was | | | |
| | | | supported | | | |
| | | | by the results from a | | | |
| | | | phase II trial of | | | |
| | | | rPAF-AH in 127 | | | |
| | | | patients with severe | | | |
| | | | sepsis (36). A phase | | | |
| | | | III trial was | | | |
| | | | undertaken | | | |
| | | | to confirm these | | | |
| | | | results in patients at | | | |
| | | | risk | | | |
| | | | for ARDS and | | | |
| | | | mortality from severe | | | |
| | | | sepsis. | | | |
| | Minopafant, | PAF | | anti- | May benefit | PAF receptor |
| | TCV-309, | antagonist | | coagulant | subtype B | upregulated in |
| | YM-264, | | | | | subtype B |
| | SM-12502, | | | | | |
| | UK-74505 | | | | | |
| | NI-0101 | Blocks | Toll-Like Receptor 4 | Decrease | May benefit | TLR4 gene |
| | | TLR4 | (TLR4) signal | inflammation | subtype B | upregulated in |
| | | | pathway plays an | | | subtype B vs. |
| | | | important role in | | | subtype A |
| | | | initiating the innate | | | |
| | | | immune response and | | | |
| | | | its activation by | | | |
| | | | bacterial endotoxin is | | | |
| | | | responsible for | | | |
| | | | chronic and acute | | | |
| | | | inflammatory | | | |
| | | | disorders that are | | | |
| | | | becoming more and | | | |
| | | | more frequent in | | | |
| | | | developed countries. | | | |
| | | | Modulation of the | | | |
| | | | TLR4 pathway is a | | | |
| | | | potential strategy to | | | |
| | | | specifically target | | | |
| | | | these pathologies. | | | |
| | HU-003 | Blocks | Toll-Like Receptor 4 | Decrease | May benefit | TLR4 gene |
| | | TLR4 | (TLR4) signal | inflammation | subtype B | upregulated in |
| | | | pathway plays an | | | subtype B vs. |
| | | | important role in | | | subtype A |
| | | | initiating the innate | | | |
| | | | immune response and | | | |
| | | | its activation by | | | |
| | | | bacterial endotoxin is | | | |
| | | | responsible for | | | |
| | | | chronic and acute | | | |
| | | | inflammatory | | | |
| | | | disorders that are | | | |
| | | | becoming more and | | | |
| | | | more frequent in | | | |
| | | | developed countries. | | | |
| | | | Modulation of the | | | |
| | | | TLR4 pathway is a | | | |
| | | | potential strategy to | | | |
| | | | specifically target | | | |
| | | | these pathologies. | | | |
| | Eritoran | Blocks | Toll-Like Receptor 4 | Decrease | May benefit | TLR4 gene |
| | | TLR4 | (TLR4) signal | inflammation | subtype B | upregulated in |
| | | | pathway plays an | | | subtype B vs. |
| | | | important role in | | | subtype A |
| | | | initiating the innate | | | |
| | | | immune response and | | | |
| | | | its activation by | | | |
| | | | bacterial endotoxin is | | | |
| | | | responsible for | | | |
| | | | chronic and acute | | | |
| | | | inflammatory | | | |
| | | | disorders that are | | | |
| | | | becoming more and | | | |
| | | | more frequent in | | | |
| | | | developed countries. | | | |
| | | | Modulation of the | | | |
| | | | TLR4 pathway is a | | | |
| | | | potential strategy to | | | |
| | | | specifically target | | | |
| | | | these pathologies. | | | |
| | Resatorvid | Blocks | Toll-Like Receptor 4 | Decrease | May benefit | TLR4 gene |
| | | TLR4 | (TLR4) signal | inflammation | subtype B | upregulated in |
| | | | pathway plays an | | | subtype B vs. |
| | | | important role in | | | subtype A |
| | | | initiating the innate | | | |
| | | | immune response and | | | |
| | | | its activation by | | | |
| | | | bacterial endotoxin is | | | |
| | | | responsible for | | | |
| | | | chronic and acute | | | |
| | | | inflammatory | | | |
| | | | disorders that are | | | |
| | | | becoming more and | | | |
| | | | more frequent in | | | |
| | | | developed countries. | | | |
| | | | Modulation of the | | | |
| | | | TLR4 pathway is a | | | |
| | | | potential strategy to | | | |
| | | | specifically target | | | |
| | | | these pathologies. | | | |
| | CytoFab | anti- | CytoFab is a | Decrease | May benefit | TNF-α gene |
| | | TNF-α | polyclonal antibody | inflammation | subtype B | upregulated in |
| | | | against tumor necrosis | | | subtype B vs. |
| | | | factor alpha, which is | | | subtype A |
| | | | produced in vast | | | |
| | | | quantities in sepsis | | | |
| | | | patients and | | | |
| | | | contributes to the | | | |
| | | | symptoms and organ | | | |
| | | | dysfunctions that | | | |
| | | | eventually kill the | | | |
| | | | patient. Phase IIb | | | |
| | | | results showed that | | | |
| | | | CytoFab significantly | | | |
| | | | reduced TNF-alpha in | | | |
| | | | the blood and lung | | | |
| | | | tissues of sepsis | | | |
| | | | patients, and patients | | | |
| | | | required five days' | | | |
| | | | less mechanical | | | |
| | | | ventilation than when | | | |
| | | | treated with placebo. | | | |
| | | | There was also a trend | | | |
| | | | towards improved | | | |
| | | | survival; | | | |
| | | | approximately one | | | |
| | | | third of patients with | | | |
| | | | severe sepsis die from | | | |
| | | | major organ failure at | | | |
| | | | present. | | | |
| Nerelimomab | Nerelimomab | anti- | | Decrease | May benefit | TNF-α gene |
| | | TNF-α | | inflammation | subtype B | upregulated in |
| | | | | | | subtype B vs. |
| | | | | | | subtype A |
| | Humicade | anti- | | Decrease | May benefit | TNF-α gene |
| | | TNF-α | | inflammation | subtype B | upregulated in |
| | | | | | | subtype B vs. |
| | | | | | | subtype A |
| | rhTNFbP | TNF binding | | Decrease | May benefit | TNF-α gene |
| | | protein | | inflammation | subtype B | upregulated in |
| | | | | | | subtype B vs. |
| | | | | | | subtype A |
| p55 TNF | Lenercept | recombinant | Lenercept is a | Decrease | May benefit | TNF-α gene |
| receptor | | TNF receptor | recombinant protein | inflammation | subtype B | upregulated |
| | | p55, binds | that is constructed by | | | and p55 TNF |
| | | TNF-a | fusing human soluble | | | receptor up- |
| | | | p55 TNF receptors | | | regulated in |
| | | | (extracellular domain) | | | subtype B vs. |
| | | | to an immunoglobulin | | | subtype A |
| | | | G1 heavy chain | | | |
| | | | fragment and is | | | |
| | | | expressed as a | | | |
| | | | dimeric molecule in | | | |
| | | | Chinese hamster | | | |
| | | | ovary cells. | | | |
| | | | Preclinical studies | | | |
| | | | demonstrated that | | | |
| | | | lenercept binds to and | | | |
| | | | neutralizes TNF and | | | |
| | | | prevents death in a | | | |
| | | | variety of animal | | | |
| | | | models of sepsis and | | | |
| | | | septic shock | | | |
| | Bicizar | Complement | C1-esterase inhibitor | Decrease | May benefit | Complement |
| | | C1 inhibitor | (C1 INH) is an alpha- | inflammation | subtype B | system in |
| | | | globulin controlling | | | highly |
| | | | the first part of the | | | activated in |
| | | | classic complement | | | subtype B vs. |
| | | | pathway and is a | | | subtype A |
| | | | natural inhibitor of | | | |
| | | | complement and | | | |
| | | | contact system | | | |
| | | | proteases and a major | | | |
| | | | downregulator of | | | |
| | | | inflammatory | | | |
| | | | processes in blood. | | | |
| | | | During sepsis, an | | | |
| | | | overactive | | | |
| | | | complement system | | | |
| | | | may compromise the | | | |
| | | | eff ectiveness of | | | |
| | | | innate immunity. | | | |
| anti-C5a | IFX-1/ | anti-C5a | Given the strong pro- | Decrease | May benefit | Complement |
| | CaCP29 | | inflammatory and | inflammation | subtype B | system in |
| | | | modulatory activities | | | highly |
| | | | of C5a signaling, | | | activated in |
| | | | therapeutic | | | subtype B vs. |
| | | | intervention at the | | | subtype A |
| | | | level of C5a or the | | | |
| | | | C5a receptor (C5aR; | | | |
| | | | CD88) remains a | | | |
| | | | focal area. | | | |
| | | | Neutralizing | | | |
| | | | antibodies against | | | |
| | | | C5a have | | | |
| | | | demonstrated | | | |
| | | | protective effects in | | | |
| | | | experimental sepsis. | | | |
| anti-C5aR | Avacopan | anti-C5aR | | Decrease | May benefit | Complement |
| | | (will be | | inflammation | subtype B | system in |
| | | approved in | | | | highly |
| | | 2020) | | | | activated in |
| | | | | | | subtype B vs. |
| | | | | | | subtype A |
| | ISU201 | suppressed | | Decrease | May benefit | TNF-a and |
| | | accumulation of | | inflammation | subtype B | Icam1 are up- |
| | | pulmonary | | | and C | regulated in |
| | | neutrophils | | | | subtype B and |
| | | and | | | | and vcam1 and |
| | | eosinophils, | | | | Csf1 genes are |
| | | while | | | | up-regulated in |
| | | accelerating | | | | subtype C |
| | | the decline | | | | |
| | | in CXCL1, | | | | |
| | | TNF-α, and | | | | |
| | | IL-6 in | | | | |
| | | lavage fluid | | | | |
| | | and lung | | | | |
| | | tissue. | | | | |
| | | ISU201 | | | | |
| | | significantly | | | | |
| | | reduced | | | | |
| | | peak | | | | |
| | | expression | | | | |
| | | of mRNA | | | | |
| | | for the | | | | |
| | | chemokines | | | | |
| | | Cxcl9 and | | | | |
| | | Cxcl10, the | | | | |
| | | adhesion | | | | |
| | | molecules | | | | |
| | | Icam1 and | | | | |
| | | Vcam1, and the | | | | |
| | | proinflammatory | | | | |
| | | cytokines | | | | |
| | | Il1b, | | | | |
| | | Il12p40, | | | | |
| | | and Csf1. | | | | |
| | PGX-100 | Reduces | | Decrease | May benefit | TNF-a and |
| | (modified | IL-6, IL-8, | | inflammation | subtype B | complement |
| | heparin) | TNF-a, | | | | system up- |
| | | CRP. CRP | | | | regulated in |
| | | activates the | | | | subtype B vs. |
| | | complement | | | | subtype A |
| | | system | | | | |
| | LGT-209 | anti-PCSK9 | anti-PCSK9 antibody: | Decrease | May benefit | PCSK9 gene is |
| | | antibody | LGT-209 as a novel | inflammation | subtype B | up-regulated in |
| | | | means to clear | | | subtype B |
| | | | endotoxin and other | | | |
| | | | bacterial toxins out of | | | |
| | | | a patient's system | | | |
| | centhaquin | Alpha-2A | | Increase | May benefit | Both receptors |
| | | adrenergic | | blood | subtype C | are up- |
| | | receptor | | pressure | | regulated in |
| | | agonist and | | | | subtype C |
| | | Alpha-1 | | | | |
| | | adrenergic | | | | |
| | | receptor | | | | |
| | | antagonist: | | | | |
| | | reduces | | | | |
| | | blood lactate | | | | |
| | | and increase | | | | |
| | | blood | | | | |
| | | pressure | | | | |
| Thrombomodulin | ART-123/ | Protein C | Thrombomodulin is | anti- | May benefit | Type C patients |
| | REMODULIN/ | Stimulant, | an endothelial cell | coagulant | subtype C | have |
| | treprostinil | thrombomodulin | surface | | | upregulated |
| | | | transmembrane | | | genes related to |
| | | | protein critical to the | | | coagulation. |
| | | | regulation of | | | |
| | | | intravascular | | | |
| | | | coagulation. rhTM | | | |
| | | | was approved and | | | |
| | | | now is being used | | | |
| | | | clinically for the | | | |
| | | | treatment of | | | |
| | | | disseminated | | | |
| | | | intravascular | | | |
| | | | coagulation (DIC) in | | | |
| | | | Japan. As its | | | |
| | | | mechanism of action, | | | |
| | | | thrombin-rhTM | | | |
| | | | complex catalyzes the | | | |
| | | | activation of protein | | | |
| | | | C. Activated protein | | | |
| | | | C proteolytically | | | |
| | | | inactivates | | | |
| | | | coagulation co-factors | | | |
| | | | Va and VIIIa, thereby | | | |
| | | | inhibiting | | | |
| | | | amplification of the | | | |
| | | | coagulation system | | | |
| | asunercept | blocks CD95 | | Reduces | May benefit | CD95 is |
| | | ligand | | tissue | subtype B | uptregulated in |
| | | receptor | | damage | | subtype B |
| | | antagonist | | | | |
| | Rexis | enhances | | Reduces | May benefit | Glutathione |
| | | Glutathione | | tissue | subtype C | peroxidase |
| | | peroxidase | | damage | | genes up- |
| | | | | | | regulated in |
| | | | | | | subtype C |
| | IL-15, | Pro- | | Immune | May benefit | IL-15 gene |
| | NIZ985 | inflammatory | | stimulant | subtype C | expression is |
| | | cytokine | | | | low in subtype |
| | | | | | | C relative to |
| | | | | | | Typa A |
| anti- | Keytruda/ | anti- | Blocks upregulation | Increase | May benefit | Type B and C |
| PD-1 | pembrolizumab | PD-1 | of PD-1/PD-L1 to | adaptive | subtype B | patients have a |
| | | | restore immune cell | immune | and C | suppressed |
| | | | function | activity | | adaptive |
| | | | | | | immune |
| | | | | | | response |
| anti- | Nivolumab | anti- | Blocks upregulation | Increase | May benefit | Type B and C |
| PD-1 | | PD-1 | of PD-1/PD-L1 to | adaptive | subtype B | patients have a |
| | | | restore immune cell | immune | and C | suppressed |
| | | | function | activity | | adaptive |
| | | | | | | immune |
| | | | | | | response |
| anti- | Ipilimumab/ | anti- | CTLA-4 is a negative | Increase | May benefit | Type B and C |
| CTLA-4 | YERVOY | CTLA-4 | co-stimulatory | adaptive | subtype B | patients have a |
| | | | molecule that acts in a | immune | and C | suppressed |
| | | | fashion similar to PD- | activity | | adaptive |
| | | | 1 to induce | | | immune |
| | | | suppression of T cell | | | response, IL-2 |
| | | | function. | | | is also down- |
| | | | | | | regulated in |
| | | | | | | subtype B |
| | | | | | | patients |
| Ulinastatin | Ulinastatin | inactivates | The exact mechanism | anti- | May benefit | Type C patients |
| | | many serine | of action of | coagulant | subtype C | have |
| | | proteases, | ulinastatin in sepsis is | immune | | upregulated |
| | | including | not clear, it is likely | activity | | genes related to |
| | | trypsin, | that it may attenuate | | | coagulation |
| | | chymotrypsin, | the inflammatory | | | |
| | | kallikrein, | response by acting at | | | |
| | | plasmin, | multiple sites. Many | | | |
| | | granulocyte | of the intermediaries | | | |
| | | elastase, | in the systemic | | | |
| | | cathepsin, | inflammatory | | | |
| | | thrombin, | processes are serine | | | |
| | | and factors | proteases. These | | | |
| | | IXa, Xa, | include trypsin, | | | |
| | | XIa, and | thrombin, | | | |
| | | XlIa | chymotrypsin, | | | |
| | | | kallikrein, plasmin, | | | |
| | | | neutrophil elastase, | | | |
| | | | cathepsin, neutrophil | | | |
| | | | protease-3, and | | | |
| | | | coagulation factors | | | |
| | | | IXa, Xa, XIa, and | | | |
| | | | XlIa. It is now being | | | |
| | | | recognized that | | | |
| | | | besides their | | | |
| | | | proteolytic activity, | | | |
| | | | these proteases have | | | |
| | | | an important role in | | | |
| | | | regulation of | | | |
| | | | inflammation through | | | |
| | | | inter- and intracellular | | | |
| | | | signaling pathways. | | | |
| | | | To counter-regulate | | | |
| | | | the effect of these | | | |
| | | | proteases, several | | | |
| | | | protease inhibitors are | | | |
| | | | produced by the liver | | | |
| | | | in the presence of | | | |
| | | | inflammation; these | | | |
| | | | include acute phase | | | |
| | | | reactants such as α1- | | | |
| | | | antitrypsin and | | | |
| | | | proteins of the inter- | | | |
| | | | α-inhibitor family. | | | |
| | | | Urinary trypsin | | | |
| | | | inhibitor is one such | | | |
| | | | important protease | | | |
| | | | inhibitor found in | | | |
| | | | human blood and | | | |
| | | | urine; it has been also | | | |
| | | | referred to in the | | | |
| | | | literature as | | | |
| | | | ulinastatin or bikunin | | | |
| Adalimumab | Humira | anti- | | Decrease | May benefit | TNF-α gene |
| | | TNF-α | | inflammation | subtype B | upregulated in |
| | | | | | | subtype B vs. |
| | | | | | | subtype A |
| Infliximab | Remicade | anti- | | Decrease | May benefit | TNF-α gene |
| | | TNF-α | | inflammation | subtype B | upregulated in |
| | | | | | | subtype B vs. |
| | | | | | | subtype A |
| p75 TNF | Adalimumab | p75 TNF | | Decrease | May benefit | TNF-α gene |
| receptor | | receptor, | | inflammation | subtype B | upregulated |
| | | Binds TNF-a | | | | subtype B vs. |
| | | | | | | subtype A |
| anti-C5a | Ultomiris | anti-C5a | | Decrease | May benefit | Complement |
| | | | | inflammation | subtype B | system in |
| | | | | | | highly |
| | | | | | | activated in |
| | | | | | | subtype B vs. |
| | | | | | | subtype A |
| anti-C5a | Soliris | anti-C5a | | Decrease | May benefit | Complement |
| | | | | inflammation | subtype B | system in |
| | | | | | | highly |
| | | | | | | activated in |
| | | | | | | subtype B vs. |
| | | | | | | subtype A |
| IL1R1 | Kineret/ | INF-gama, | TNF-α and IL-1 (a | Immune | May benefit | INF-gamma |
| | anakinra | immune | term used for a family | stimulant | subtype B | gene is less |
| | | stimulant | of proteins, including | | | expressed in |
| | | | IL-1α and IL-1β) are | | | subtype B vs. |
| | | | powerful | | | subtype A |
| | | | proinflammatory | | | |
| | | | cytokines that have | | | |
| | | | been implicated in a | | | |
| | | | large number of | | | |
| | | | infectious and | | | |
| | | | noninfectious | | | |
| | | | inflammatory | | | |
| | | | diseases. The release | | | |
| | | | of TNF-α from | | | |
| | | | macrophages begins | | | |
| | | | within 30 minutes | | | |
| | | | after the inciting | | | |
| | | | event, following gene | | | |
| | | | transcription and | | | |
| | | | RNA translation, | | | |
| | | | which established this | | | |
| | | | mediator to be an | | | |
| | | | early regulator of the | | | |
| | | | immune response. | | | |
| | | | TNF-α acts via | | | |
| | | | specific | | | |
| | | | transmembrane | | | |
| | | | receptors, TNF | | | |
| | | | receptor (TNFR)1, | | | |
| | | | and TNFR2, leading | | | |
| | | | to the activation of | | | |
| | | | immune cells and the | | | |
| | | | release of an array of | | | |
| | | | downstream | | | |
| | | | immunoregulatory | | | |
| | | | mediators. Likewise, | | | |
| | | | IL-1 is released | | | |
| | | | primarily from | | | |
| | | | activated | | | |
| | | | macrophages in a | | | |
| | | | timely manner similar | | | |
| | | | to TNF-α, signals | | | |
| | | | through two distinct | | | |
| | | | receptors, termed IL-1 | | | |
| | | | receptor type I (IL- | | | |
| | | | 1R1) and IL-1R2, and | | | |
| | | | has comparable | | | |
| | | | downstream effects | | | |
| | | | on immune cells | | | |
| | progesterone | reduces IL-6 | | Decrease | May benefit | Type B |
| | | and TNF-a | | inflammation | subtype B | exhibits |
| | | | | | | relative high |
| | | | | | | gene |
| | | | | | | expression of |
| | | | | | | TNF-a |
| | Thymosin | Thymalfasin | Thymosin alpha 1 | Immune | May benefit | Thymosin |
| | alpha I | peptide, | (Ta1) is a naturally | stimulant | subtype B | alpha I gene |
| | (SciClone | T-lymphocyte | occurring thymic | | | highly |
| | Pharmaceuticals, | subset | peptide. It acts as an | | | expressed in |
| | Roche) | modulators; | endogenous regulator | | | subtype A vs. |
| | | Th1 cell | of both the innate and | | | subtype B and |
| | | stimulants; | adaptive immune | | | drug could |
| | | Th2-cell- | systems. It is used | | | increase |
| | | inhibitors | worldwide for treating | | | adaptive |
| | | | diseases associated | | | immune |
| | | | with immune | | | activity |
| | | | dysfunction including | | | |
| | | | viral infections such | | | |
| | | | as hepatitis B and C, | | | |
| | | | certain cancers, and | | | |
| | | | for vaccine | | | |
| | | | enhancement | | | |
| | Actimmune | INF-gama, | TNF-α and IL-1 (a | Immune | May benefit | INF-gamma |
| | | immune | term used for a family | stimulant | subtype B | gene is less |
| | | stimulant | of proteins, including | | | expressed in |
| | | | IL-1α and IL-1β) are | | | subtype B vs. |
| | | | powerful | | | subtype A |
| | | | proinflammatory | | | |
| | | | cytokines that have | | | |
| | | | been implicated in a | | | |
| | | | large number of | | | |
| | | | infectious and | | | |
| | | | noninfectious | | | |
| | | | inflammatory | | | |
| | | | diseases. The release | | | |
| | | | of TNF-α from | | | |
| | | | macrophages begins | | | |
| | | | within 30 minutes | | | |
| | | | after the inciting | | | |
| | | | event, following gene | | | |
| | | | transcription and | | | |
| | | | RNA translation, | | | |
| | | | which established this | | | |
| | | | mediator to be an | | | |
| | | | early regulator of the | | | |
| | | | immune response. | | | |
| | | | TNF-α acts via | | | |
| | | | specific | | | |
| | | | transmembrane | | | |
| | | | receptors, TNF | | | |
| | | | receptor (TNFR)1, | | | |
| | | | and TNFR2, leading | | | |
| | | | to the activation of | | | |
| | | | immune cells and the | | | |
| | | | release of an array of | | | |
| | | | downstream | | | |
| | | | immunoregulatory | | | |
| | | | mediators. Likewise, | | | |
| | | | IL-1 is released | | | |
| | | | primarily from | | | |
| | | | activated | | | |
| | | | macrophages in a | | | |
| | | | timely manner similar | | | |
| | | | to TNF-α, signals | | | |
| | | | through two distinct | | | |
| | | | receptors, termed IL-1 | | | |
| | | | receptor type I | | | |
| | | | (IL-1R1) and IL-1R2, | | | |
| | | | and has comparable | | | |
| | | | downstream effects | | | |
| | | | on immune cells | | | |
| | defibrotide | protects the | | anti- | May benefit | Type C has |
| | | cells lining | | coagulant | subtype C | Plasminogen |
| | | bloods | | | | activator |
| | | vessels and | | | | inhibitor-1 |
| | | preventing | | | | upregulated |
| | | blood | | | | |
| | | clotting. | | | | |
| | | mixture of | | | | |
| | | single- | | | | |
| | | stranded | | | | |
| | | oligonucleotides | | | | |
| | | that is | | | | |
| | | purified | | | | |
| | | from the | | | | |
| | | intestinal | | | | |
| | | mucosa of | | | | |
| | | pigs | | | | |
| | nangibotide | Anti-TREM-1, | | Anti- | May benefit | TREM-1 gene |
| | (MOTREM) | blocks | | inflammatory | subtype B | is highly |
| | | TREM-1 | | | | expressed in |
| | | which is a | | | | subtype A |
| | | trigger of | | | | patients but |
| | | pathogen- | | | | these patients |
| | | induced | | | | already exhibit |
| | | inflammation | | | | relatively low |
| | | | | | | mortality |
| | EA-230 | Reduces | | Anti- | May benefit | Reducing IL-10 |
| | | IL-6, IL-10, | | inflammatory | subtype B | and TNF-a |
| | | INF-g, TNF-a, | | | | whose genes |
| | | E-Selectin | | | | are highly |
| | | | | | | expressed in B |
| | | | | | | may be |
| | | | | | | beneficial but |
| | | | | | | reducing INF-g |
| | | | | | | whose genes |
| | | | | | | are highly |
| | | | | | | expressed in |
| | | | | | | subtype A may |
| | | | | | | not be |
| | | | | | | beneficial |
| | curcumin | NF-kB | | Anti- | May benefit | Type A may |
| | | inhibitor | | inflammatory | subtype B | benefit from |
| | | | | | | pathogen- |
| | | | | | | mediated |
| | | | | | | inflammation |
| | | | | | | that required |
| | | | | | | NF-kB |
| | Emricasan | pan-caspsase | | Anti- | May benefit | Up-regulated in |
| | | inhibitor | | inflammatory | subtype B | subtype B vs. C |
| | | | | | | and A |
| | IL1R1 | Inhinits | | Anti- | May benefit | May benefit |
| | | IL-1A, IL-1B, | | inflammatory | subtype B | subtype B since |
| | | and IL-1 | | | | IL-1 receptor |
| | | receptor | | | | antagonist gene |
| | | antagonist | | | | is highly |
| | | | | | | expressed in |
| | | | | | | subtype B vs. |
| | | | | | | subtype A |
| | AB103 | peptide | | Immune | May be | CD28 gene is |
| | | CD28 | | suppressant | contraindicated | highly |
| | | Antagonist | | | | expressed in |
| | | | | | | subtype A |
| | | | | | | patients and |
| | | | | | | these patients |
| | | | | | | already exhibit |
| | | | | | | relatively low |
| | | | | | | mortality. |
| | | | | | | AB103 would |
| | | | | | | suppress |
| | | | | | | adaptive |
| | | | | | | immune |
| | | | | | | activity. |
| | Filgrastim | G-CSF, | | Immune | May benefit | G-CSF is |
| | | immune | | stimulant | subtype C | highly |
| | | stimulant | | | | expressed in |
| | | | | | | subtype C |
| | | | | | | which has the |
| | | | | | | worst outcomes |
| | Sagramostim | GM-CSF, | | Immune | May benefit | GM-CSF may |
| | | immune | | stimulant | subtype B | increase innate |
| | | stimulant | | | and C | activity |
| | | | | | | associated with |
| | | | | | | pathogen |
| | | | | | | recognition and |
| | | | | | | subtype B and |
| | | | | | | C exhibit |
| | | | | | | down- |
| | | | | | | regulation of |
| | | | | | | immune |
| | | | | | | activity |
| | | | | | | associated with |
| | | | | | | pathogen |
| | | | | | | clearance. |
| | Roncoleukin | IL-2, | | Immune | May be | Would |
| | | promotes | | suppressant | contraindicated | suppress |
| | | T-reg | | | | immune |
| | | | | | | activity |
| | adrecizumab | stabilizes/increases | | Decrease | May benefit | Stabilizes a |
| | | adrenomedullin and | | vascular | subtype C | vasodilator that |
| | | reverses | | permeability | | is already |
| | | vascular | | | | down-regulated |
| | | permeability | | | | in subtype C |
| | | | | | | while it's |
| | | | | | | receptors are |
| | | | | | | up-regulated in |
| | | | | | | subtype C |
| | Talacotuzumab | Interleukin- | | Immune | Type B and | IL-3 receptor |
| | | 3-receptor- | | stimulant | C may | up-regulated in |
| | | alpha- | | | benefit | subtype B and C |
| | | subunit- | | | | |
| | | antagonists | | | | |
| | Mobista | Flt3 ligand, | | Immune | May be | Would |
| | | Fms-like | | suppressant | contraindicated | suppress |
| | | tyrosine | | | | adaptive |
| | | kinase 3 | | | | immune |
| | | stimulants, | | | | activity |
| | | increases | | | | |
| | | Treg | | | | |
| | | proliferation | | | | |
| | Rituximab | Destroys B | | Immune | May be | CD20 gene |
| | | cells | | suppressant | contraindicated | expression is |
| | | expressing | | | | up-regulated in |
| | | CD20 | | | | subtype A |
| | GW-274150, | NOS | | | May benefit | INOS up- |
| | Tilarginine, | inhibitor | | | subtype C | regulated in |
| | Norathiol, | | | | | subtype C |
| | targinine | | | | | |
| | timbetasin | synthetic | | | May benefit | TB4 up- |
| | | TB4 | | | subtype C | regulated in |
| | | | | | | subtype B vs C |
| | Peptide | TLR2 | | Anti- | May benefit | TLR2 is up- |
| | P13 | inhibitor | | inflammatory | subtype B | regulated in |
| | | | | | | subtype B |
| | Tinospora | TRL6 | | Anti- | May benefit | TLR6 is up- |
| | cordifolia | inhibitor | | inflammatory | subtype B | regulated in |
| | derivative | | | | | subtype B |
| Tocilizumab | ACTE | Anti- | | Anti- | May benefit | Type B patients |
| | MRA | IL-6 | | inflammatory | subtype B | are inflammed |
| | abatacept | Fc region | Suppression of | Immune | May be | Patients |
| | | of the | adaptive immune | suppression | contraindicated | exhibiting |
| | | immunoglobulin | activity. Abatacept | | | adaptive |
| | | IgG1 | binds to the CD80 and | | | immune activity |
| | | fused to the | CD86 molecules, and | | | exhibit lower |
| | | extracellular | prevents co- | | | mortality |
| | | domain of | stimulation for T cell | | | |
| | | CTLA-4 | activation. | | | |
| | Abetimus | Made of four | | Anti- | Type B and | Type B and C |
| | | double- | | inflammatory | C patients | patients exhibit |
| | | stranded | | | may benefit | up-regulation of |
| | | oligodeoxyri | | | | DAMP-mediated innate |
| | | bonucleotides | | | | immune activity |
| | | that are | | | | relative to |
| | | attached to a | | | | subtype A |
| | | carrier | | | | patients |
| | | platform and | | | | |
| | | are designed | | | | |
| | | to block | | | | |
| | | specific B- | | | | |
| | | cell anti | | | | |
| | | double | | | | |
| | | stranded | | | | |
| | | DNA | | | | |
| | | antibodies | | | | |
| | Abrilumab | Anti-α4β7 | α4β7 integrin is a | Anti- | Type B | Type B patients |
| | | antibody | validated target in | inflammatory | patients may | exhibit up- |
| | | | inflammatory bowel | | benefit | regulated |
| | | | disease. Gut-specific | | | expression of |
| | | | homing is the | | | TNF-alpha gene |
| | | | mechanism by which | | | |
| | | | activated T cells and | | | |
| | | | antibody-secreting | | | |
| | | | cells (ASCs) are | | | |
| | | | targeted to both | | | |
| | | | inflamed and non- | | | |
| | | | inflamed regions of | | | |
| | | | the gut in order to | | | |
| | | | provide an effective | | | |
| | | | immune response. | | | |
| | | | This process relies on | | | |
| | | | the key interaction | | | |
| | | | between the integrin | | | |
| | | | α4β7 and the | | | |
| | | | addressin MadCAM-1 | | | |
| | | | on the surfaces of the | | | |
| | | | appropriate cells. | | | |
| | | | Additionally, this | | | |
| | | | interaction is | | | |
| | | | strengthened by the | | | |
| | | | presence of CCR9, a | | | |
| | | | chemokine receptor, | | | |
| | | | which interacts with | | | |
| | | | TECK. | | | |
| | adalimumab | Anti-TNF- | Attenuation of pro- | Anti- | Type B | Type B patients |
| | | alpha | inflammatory | inflammatory | patients may | exhibit up- |
| | | | cytokines TNF-alpha | | benefit | regulated |
| | | | and IL-6 | | | expression of |
| | | | | | | TNF-alpha gene |
| | Afelimomab | Anti-TNF- | Attenuation of pro- | Anti- | Type B | Type B patients |
| | | alpha | inflammatory | inflammatory | patients may | exhibit up- |
| | | | cytokines TNF-alpha | | benefit | regulated |
| | | | and IL-6 | | | expression of |
| | | | | | | TNF-alpha gene |
| | Alefacept | Fusion | Suppression of | Immune | May be | Patients |
| | | protein | adaptive immune | suppressant | contraindicated | exhibiting |
| | | combining | activity. Inhibits the | | | adaptive |
| | | part of an | activation of CD4+ | | | immune activity |
| | | antibody with | and CD8+ T cells by | | | exhibit lower |
| | | a protein that | interfering with CD2 | | | mortality |
| | | blocks the | on the T cell | | | |
| | | growth of | membrane thereby | | | |
| | | some types of | blocking the | | | |
| | | T cells | costimulatory | | | |
| | | | molecule LFA-3/CD2 | | | |
| | | | interaction and | | | |
| | | | induces apoptosis of | | | |
| | | | memory-effector T | | | |
| | | | lymphocytes. | | | |
| | anakinra | Recombinant | | Immune | Type B and | INF-gama gene |
| | | human | | stimulant | C patients | is less expressed |
| | | interleukin-1 | | | may benefit | in subtype B |
| | | receptor | | | | and C vs. |
| | | antagonist | | | | subtype A |
| | Andecaliximab | Recombinant | | Anti- | Type B and | Type B and C |
| | | chimeric | | inflammatory | C patients | patients exhibit |
| | | IgG4 | | | may benefit | up-regulation of |
| | | monoclonal | | | | MMP9 and |
| | | antibody | | | | DAMP- |
| | | against | | | | mediated innate |
| | | metalloproteinase-9 | | | | immune activity |
| | | (MMP9) | | | | relative to |
| | | | | | | subtype A |
| | | | | | | patients |
| | Anrukinzumab | Anti- | IL-13 is a mediator of | Anti- | Type C | Type C patients |
| | | interleukin 13 | allergic inflammatory | inflammatory | patients may | have IL-13 up- |
| | | monoclonal | response | | benefit | regulated |
| | | antibody | | | | relative to |
| | | | | | | subtype A |
| | Anti- | Infusion of | | Immune | May be | Patients |
| | lymphocyte | animal- | | suppressant | contraindicated | exhibiting |
| | globulin | antibodies | | | | adaptive |
| | | against | | | | immune activity |
| | | human T | | | | exhibit lower |
| | | cells | | | | mortality |
| | Anti | Infusion of | | Immune | May be | Patients |
| | thymocyte | horse or | | suppressant | contraindicated | exhibiting |
| | globulin | rabbit- | | | | adaptive |
| | | derived | | | | immune activity |
| | | antibodies | | | | exhibit lower |
| | | against | | | | mortality |
| | | human T | | | | |
| | | cells | | | | |
| | antifolate | Class of | Interferes with cell- | Immune | May be | Patients |
| | | antimetabolite | mediated immune | suppressant | contraindicated | exhibiting |
| | | medications | response. Antifolates | | | pathogen- |
| | | that | act specifically during | | | specific innate |
| | | antagonise | DNA and RNA | | | and adaptive |
| | | the actions of | synthesis, and thus are | | | immune activity |
| | | folic acid | cytotoxic during the | | | exhibit lower |
| | | (vitamin B9), | S-phase of the cell | | | mortality |
| | | typically via | cycle, exhibiting a | | | |
| | | inhibiting | greater toxic effect on | | | |
| | | dihydrofolate | rapidly dividing cells | | | |
| | | reductase | such as malignant | | | |
| | | (DHFR) | cells, myeloid cells, | | | |
| | | | as well | | | |
| | | | gastrointestinal and | | | |
| | | | oral mucosa. | | | |
| | Apolizumab | Humanized | | Immune | May be | Patients |
| | | monoclonal | | suppressant | contraindicated | exhibiting |
| | | antibody | | | | pathogen- |
| | | against HLA- | | | | specific innate |
| | | DR beta | | | | and adaptive |
| | | | | | | immune activity |
| | | | | | | exhibit lower |
| | | | | | | mortality |
| | Apremilast | Small | Down-regulation of | Anti- | May benefit | Type B patients |
| | | molecule | pro-inflammatory | inflammatory | subtype B but | exhibit up- |
| | | inhibitor of | cytokines (e.g. TNF- | | risk of | regulated |
| | | the enzyme | alpha) and up- | | contraindication | expression of |
| | | phosphodiest | regulation of adaptive | | | TNF-alpha gene |
| | | erase 4 | immune suppression | | | and up- |
| | | (PDE4) | (IL-10) | | | regulation of IL- |
| | | (enzyme that | | | | 10 which may |
| | | breaks down | | | | suppress |
| | | cyclic | | | | beneficial |
| | | adenosine | | | | adaptive |
| | | monophosph | | | | immune activity |
| | | ate (cAMP)) | | | | |
| | | resulting in | | | | |
| | | down- | | | | |
| | | regulation if | | | | |
| | | TNF-alpha, | | | | |
| | | IL-17, and | | | | |
| | | IL-23, and | | | | |
| | | up-regulation | | | | |
| | | of IL-10 | | | | |
| | Aselizumab | Humanized | Interferes with | Immune | May be | Patients |
| | | monoclonal | leukocyte function | suppressant | contraindicated | exhibiting |
| | | antibody | | | | adaptive |
| | | against | | | | immune activity |
| | | CD62L | | | | exhibit lower |
| | | | | | | mortality |
| | Atezolizumab | Humanized, | Interferes with | Immune | Type B and | Type B and C |
| | | engineered | adaptive immune | stimulant | C patients | patients exhibit |
| | | monoclonal | suppression | | may benefit | adaptive |
| | | antibody of | | | | immune |
| | | IgG1 isotype | | | | suppression, |
| | | against the | | | | subtype B |
| | | protein | | | | patients exhibit |
| | | programmed | | | | up-regulation of |
| | | cell death- | | | | PD-L1 gene |
| | | ligand 1 | | | | relative to other |
| | | | | | | types, subtype C |
| | | | | | | patients exhibit |
| | | | | | | up-regulation of |
| | | | | | | PD-1 gene, and |
| | | | | | | patients |
| | | | | | | exhibiting |
| | | | | | | adaptive |
| | | | | | | immune activity |
| | | | | | | exhibit lower |
| | | | | | | mortality |
| | Avelumab | Whole | Interruption of | Immune | Type B and | Type B and C |
| | | human | adaptive immune | stimulant | C patients | patients exhibit |
| | | monoclonal | suppression to | | may benefit | adaptive |
| | | antibody of | increase adaptive | | | immune |
| | | isotype IgG1 | immune activity. | | | suppression, |
| | | that binds | Formation of a PD- | | | subtype B |
| | | to the | 1/PD-L1 | | | patients exhibit |
| | | programmed | receptor/ligand | | | up-regulation of |
| | | death-ligand | complex leads to | | | PD-L1 gene |
| | | 1 (PD-L1) | inhibition of CD8+ | | | relative to other |
| | | | T cells, and therefore | | | types, subtype C |
| | | | inhibition of an | | | patients exhibit |
| | | | immune reaction. | | | up-regulation of |
| | | | Avelumab blocks the | | | PD-1 gene, and |
| | | | formation of PD- | | | patients |
| | | | 1/PDL1 ligand pairs | | | exhibiting |
| | | | is blocked and CD8+ | | | adaptive |
| | | | T cell immune | | | immune activity |
| | | | response should be | | | exhibit lower |
| | | | increased. | | | mortality |
| | azathioprine | Azathioprine | By inhibiting purine | Immune | May be | Patients |
| | | inhibits | synthesis, less DNA | suppressant | contraindicated | exhibiting |
| | | purine | and RNA are | | | adaptive |
| | | synthesis. | produced for the | | | immune activity |
| | | Purines are | synthesis of white | | | exhibit lower |
| | | needed to | blood cells, thus | | | mortality |
| | | produce | causing | | | |
| | | DNA and | immunosuppression. | | | |
| | | RNA. | | | | |
| | Basiliximab | Chimeric | Prevents T cells from | Immune | May be | Patients |
| | | mouse- | replicating and from | suppressant | contraindicated | exhibiting |
| | | human | activating B cells and | | | adaptive |
| | | monoclonal | thus production of | | | immune activity |
| | | antibody to | antibodies | | | exhibit lower |
| | | the α chain | | | | mortality |
| | | (CD25) of | | | | |
| | | the IL-2 | | | | |
| | | receptor of T | | | | |
| | | cells | | | | |
| | Belatacept | Fusion | Suppression of | Immune | May be | Patients |
| | | protein | adaptive immune | suppressant | contraindicated | exhibiting |
| | | composed of | activity. Prevents co- | | | adaptive |
| | | the Fc | stimulation for T cell | | | immune activity |
| | | fragment of a | activation. | | | exhibit lower |
| | | human IgG1 | | | | mortality |
| | | immunoglobulin | | | | |
| | | linked to the | | | | |
| | | extracellular | | | | |
| | | domain of | | | | |
| | | CTLA-4 | | | | |
| | Belimumab | Human | Belimumab reduces | Immune | May be | Patients |
| | | monoclonal | the number of | suppressant | contraindicated | exhibiting |
| | | antibody that | circulating B cells | | | adaptive |
| | | inhibits B- | | | | immune activity |
| | | cell | | | | exhibit lower |
| | | activating | | | | mortality |
| | | factor | | | | |
| | | (BAFF) | | | | |
| | Benralizumab | Murine | Binds to IL-5R via its | Immune | May be | Patients |
| | | humanized | Fab domain, blocking | suppressant | contraindicated | exhibiting |
| | | monocolonal | the binding of IL-5 to | | | adaptive |
| | | antibody | its receptor and | | | immune activity |
| | | against the | resulting in inhibition | | | exhibit lower |
| | | alpha-chain | of eosinophil | | | mortality |
| | | of the | differentiation and | | | |
| | | interleukin-5 | maturation in bone | | | |
| | | receptor | marrow. In addition, | | | |
| | | (CD125) | this antibody is able | | | |
| | | | to bind through its | | | |
| | | | afucosylated Fc | | | |
| | | | domain to the RIIIa | | | |
| | | | region of the Fcy | | | |
| | | | receptor on NK cells, | | | |
| | | | macrophages, and | | | |
| | | | neutrophils, thus | | | |
| | | | strongly inducing | | | |
| | | | antibody-dependent, | | | |
| | | | cell-mediated | | | |
| | | | cytotoxicity in both | | | |
| | | | circulating and tissue- | | | |
| | | | resident eosinophils. | | | |
| | Bertilimumab | Human | CCL11 selectively | Immune | May be | Patients |
| | | monoclonal | recruits eosinophils | suppressant | contraindicated | exhibiting |
| | | antibody that | by inducing their | | | adaptive |
| | | binds to | chemotaxis, and | | | immune activity |
| | | eotaxin-1 | therefore, is | | | exhibit lower |
| | | | implicated in allergic | | | mortality |
| | | | responses. | | | |
| | Besilesomab | Mouse | Diagnostic use only | Immune | May be | Diagnostic use |
| | | monoclonal | | suppressant | contraindicated | only |
| | | antibody | | | | |
| | | labelled | | | | |
| | | with the | | | | |
| | | radioactive | | | | |
| | | isotope | | | | |
| | | technetium- | | | | |
| | | 99m. It is | | | | |
| | | used to detect | | | | |
| | | inflammatory | | | | |
| | | lesions and | | | | |
| | | metastases. It | | | | |
| | | binds to an | | | | |
| | | immunoglobulin, | | | | |
| | | IgG1 | | | | |
| | | isotype. | | | | |
| | Bleselumab | Anti-CD40 | CD40 is a | Immune | May be | Patients |
| | | monoclonal | costimulatory protein | suppressant | contraindicated | exhibiting |
| | | antibody | found on antigen- | | | adaptive |
| | | | presenting cells and is | | | immune activity |
| | | | required for their | | | exhibit lower |
| | | | activation | | | mortality |
| | Blisibimod | Tetrameric | Antagonist of B -cell | Immune | May be | Patients |
| | | BAFF | activating factor | suppressant | contraindicated | exhibiting |
| | | binding | (BAFF) | | | adaptive |
| | | domain fused | | | | immune activity |
| | | to a human | | | | exhibit lower |
| | | IgG1 Fc | | | | mortality |
| | | region | | | | |
| | Brazikumab | Monoclonal | Inhibits Th17 function | Immune | May be | Patients |
| | | antibody that | | suppressant | contraindicated | exhibiting |
| | | binds to the | | | | adaptive |
| | | IL23 receptor | | | | immune activity |
| | | | | | | exhibit lower |
| | | | | | | mortality |
| | Briakinumab | Human | IL-12 is involved in | Immune | May be | Patients |
| | | monoclonal | the differentiation of | suppressant | contraindicated | exhibiting |
| | | antibody | naive T cells into Th1 | | | adaptive |
| | | targetting | cells | | | immune activity |
| | | IL-12 and | | | | exhibit lower |
| | | IL-23 | | | | mortality |
| | Brodalumab | Human | Blocks recruitment of | Immune | May be | Patients |
| | | monoclonal | immune cells, such as | suppressant | contraindicated | exhibiting |
| | | antibody | monocytes and | | | adaptive |
| | | targetting | neutrophils to the site | | | immune activity |
| | | interleukin | of inflammation. | | | exhibit lower |
| | | 17 receptor A | | | | mortality |
| | Canakinumab | Human | Attenuates IL-1 beta | Anti- | Type B | Type B patients |
| | | monoclonal | | inflammatory | patients may | exhibit up- |
| | | antibody | | | benefit | regulation of |
| | | targeted at | | | | inflammatory |
| | | interleukin-1 | | | | cytokines |
| | | beta | | | | |
| | Carlumab | Human | CCL2 recruits | Immune | May be | Patients |
| | | recombinant | monocytes, memory | suppressant | contraindicated | exhibiting |
| | | monoclonal | T cells, and dendritic | | | adaptive |
| | | antibody | cells to the sites of | | | immune activity |
| | | (type IgG1 | inflammation | | | exhibit lower |
| | | kappa) that | produced by either | | | mortality |
| | | targets | tissue injury or | | | |
| | | human CC | infection | | | |
| | | chemokine | | | | |
| | | ligand 2 | | | | |
| | | (CCL2) | | | | |
| | Cedelizumab | Murine | CD4+ T helper cells | Immune | May be | Patients |
| | | humanized | are white blood cells | suppressant | contraindicated | exhibiting |
| | | monocolonal | that are an essential | | | adaptive |
| | | antibody | part of the human | | | immune activity |
| | | against CD4 | immune system. | | | exhibit lower |
| | | | Depletion impairs | | | mortality |
| | | | immune activity. | | | |
| | Certolizumab | Fragment of | Attenuates TNF-alpha | Anti- | Type B | Type B patients |
| | pegol | a monoclonal | | inflammatory | patients may | have up- |
| | | antibody | | | benefit | regulated TNF- |
| | | specific to | | | | alpha gene |
| | | tumor | | | | expression |
| | | necrosis | | | | relastive to |
| | | factor alpha | | | | subtype A |
| | | | | | | patients |
| | chloroquine | Antimalarial | Against rheumatoid | Immune | May be | Patients |
| | | drug | arthritis, it operates by | suppressant | contraindicated | exhibiting |
| | | | inhibiting lymphocyte | | | adaptive |
| | | | proliferation, | | | immune activity |
| | | | phospholipase A2, | | | exhibit lower |
| | | | antigen presentation | | | mortality thus |
| | | | in dendritic cells, | | | inhibition of |
| | | | release of enzymes | | | lymphocyte |
| | | | from lysosomes, | | | proliferation and |
| | | | release of reactive | | | antigen |
| | | | oxygen species from | | | presentation |
| | | | macrophages, and | | | could be |
| | | | production of IL-1. | | | detrimental, |
| | | | | | | subtype B |
| | | | | | | patients have |
| | | | | | | up-regulation of |
| | | | | | | pro- |
| | | | | | | inflammatory |
| | | | | | | cytokines |
| | | | | | | including |
| | | | | | | phospholipase |
| | | | | | | A2 activity thus |
| | | | | | | inhibition of |
| | | | | | | phospholipase |
| | | | | | | A2, release of |
| | | | | | | enzymes from |
| | | | | | | lysosomes, |
| | | | | | | release of |
| | | | | | | reactive oxygen |
| | | | | | | species from |
| | | | | | | macrophages, |
| | | | | | | and production |
| | | | | | | of IL-1 could be |
| | | | | | | beneficial, and |
| | | | | | | subtype C |
| | | | | | | patients |
| | | | | | | similarly exhibit |
| | | | | | | inflammation |
| | | | | | | from cell and |
| | | | | | | tissue damage |
| | | | | | | and thus |
| | | | | | | inhibition of |
| | | | | | | enzyme release |
| | | | | | | and reactive |
| | | | | | | oxygen species |
| | | | | | | may be |
| | | | | | | beneficial in |
| | | | | | | these patients. |
| | Clazakizumab | Aglycosylated, | Attenuation of pro- | Anti- | Type B | Type B patients |
| | | humanized | inflammatory | inflammatory | patients may | have up- |
| | | rabbit | cytokine IL-6 | | benefit | regulated pro- |
| | | monoclonal | | | | inflammatory |
| | | antibody | | | | cytokines |
| | | against | | | | |
| | | interleukin-6 | | | | |
| | Clenoliximab | Chimeric | CD4+ T helper cells | Immune | May be | Patients |
| | | Macacairus/ | are white blood cells | suppressant | contraindicated | exhibiting |
| | | Homo | that are an essential | | | adaptive |
| | | sapiens | part of the human | | | immune activity |
| | | monoclonal | immune system. | | | exhibit lower |
| | | antibody | Depletion impairs | | | mortality |
| | | against CD4 | immune activity. | | | |
| | corticosteroids | Class of | Anti-inflammatory, | Anti- | Type B and | Immunosupressive |
| | | steroid | immunosuppressive, | inflammatory | C patients | effects may |
| | | hormones | anti-proliferative, and | | may benefit | harm subtype A |
| | | that are | vasoconstrictive | | | patients, anti- |
| | | produced in | effects | | | inflammatory |
| | | the adrenal | | | | effects may |
| | | cortex of | | | | benefit subtype |
| | | vertebrates, | | | | B patients, |
| | | as well as the | | | | vasoconstrictive |
| | | synthetic | | | | effects may |
| | | analogues of | | | | benefit subtype |
| | | these | | | | C patients. |
| | | hormones | | | | |
| | cyclosporine | Immunosuppressant | Lower the activity of | Immune | May be | Patients |
| | | medication | T-cells | suppressant | contraindicated | exhibiting |
| | | and natural | | | | adaptive |
| | | product | | | | immune activity |
| | | | | | | exhibit lower |
| | | | | | | mortality |
| | Daclizumab | Humanized | Reduction of T-cell | Immune | May be | Patients |
| | | monoclonal | responses and | suppressant | contraindicated | exhibiting |
| | | antibody that | expansion of CD56 | | | adaptive |
| | | binds to | bright natural killer | | | immune activity |
| | | CD25, the | cells | | | exhibit lower |
| | | alpha subunit | | | | mortality |
| | | of the IL-2 | | | | |
| | | receptor of | | | | |
| | | T-cells | | | | |
| | Hydroxychloroquine | Antimalarial | Against rheumatoid | Immune | May be | Type A |
| | | amyloquilone | arthritis, it operates by | suppressant | contraindicated | patients exhibit |
| | | drug | inhibiting lymphocyte | | | lower mortality |
| | | | proliferation, | | | and thus |
| | | | phospholipase A2, | | | inhibition of |
| | | | antigen presentation | | | lymphocyte |
| | | | in dendritic cells, | | | proliferation |
| | | | release of enzymes | | | and antigen |
| | | | from lysosomes, | | | presentation |
| | | | release of reactive | | | could prolong |
| | | | oxygen species from | | | viral clearance. |
| | | | macrophages, and | | | subtype B |
| | | | production of IL-1 | | | patients exhibit |
| | | | | | | up-regulation |
| | | | | | | of pro- |
| | | | | | | inflammatory |
| | | | | | | cytokines and |
| | | | | | | thus the anti- |
| | | | | | | inflammatory |
| | | | | | | properties of |
| | | | | | | hydroxychloro |
| | | | | | | quine may be |
| | | | | | | beneficial to |
| | | | | | | these patients. |
| | Azithromycin | Macrolide | Exhibit anti- | Anti- | Type B | Type B patients |
| | | antibiotic | inflammatory | inflammatory | patients may | exhibit up- |
| | | | properties via | | benefit | regulation of |
| | | | suppression of pro- | | | pro- |
| | | | inflammatory host | | | inflammatory |
| | | | response that may | | | cytokines and |
| | | | contribute to | | | thus the anti- |
| | | | inflammation of the | | | inflammatory |
| | | | airways | | | properties of |
| | | | | | | azithromycin |
| | | | | | | may be |
| | | | | | | beneficial to |
| | | | | | | these patients. |
| | Anti-GM- | | | Immune | May be | GM-CSF may |
| | CSF | | | suppresant | contraindicated | increase innate |
| | | | | | | activity |
| | | | | | | associated with |
| | | | | | | pathogen |
| | | | | | | recognition and |
| | | | | | | subtype B and |
| | | | | | | C exhibit |
| | | | | | | down- |
| | | | | | | regulation of |
| | | | | | | immune |
| | | | | | | activity |
| | | | | | | associated with |
| | | | | | | pathogen |
| | | | | | | clearance. |
| | CD24Fc | DAMP | | Anti- | Type B and | Type B and C |
| | | receptor | | inflammtory | C patients | patients exhibit |
| | | blocker | | | may benefit | up-regulation |
| | | | | | | of DAMPs |
| | | | | | | which may |
| | | | | | | contribute to |
| | | | | | | inflammation |

VI.A. Dysregulated Host Response Patient Subtype A

VI.A.1. Corticosteroids

As discussed in detail above, septic patients that remain hypotensive and require vasopressors to maintain a mean arterial pressure ≥65 mmHg are characterized as having septic shock—a condition that exhibits a hospital mortality in excess of 40%. Septic shock patients that show no clinical improvement (defined as having a systolic blood pressure <90 mmHg for more than one hour following both adequate fluid resuscitation and vasopressor therapy) are deemed refractory to vasopressor therapy and are thus characterized as refractory septic shock patients. In many cases, refractory septic shock patients are given corticosteroid therapy, such as hydrocortisone, based on rationale that the therapy may enable vasopressor responsiveness.

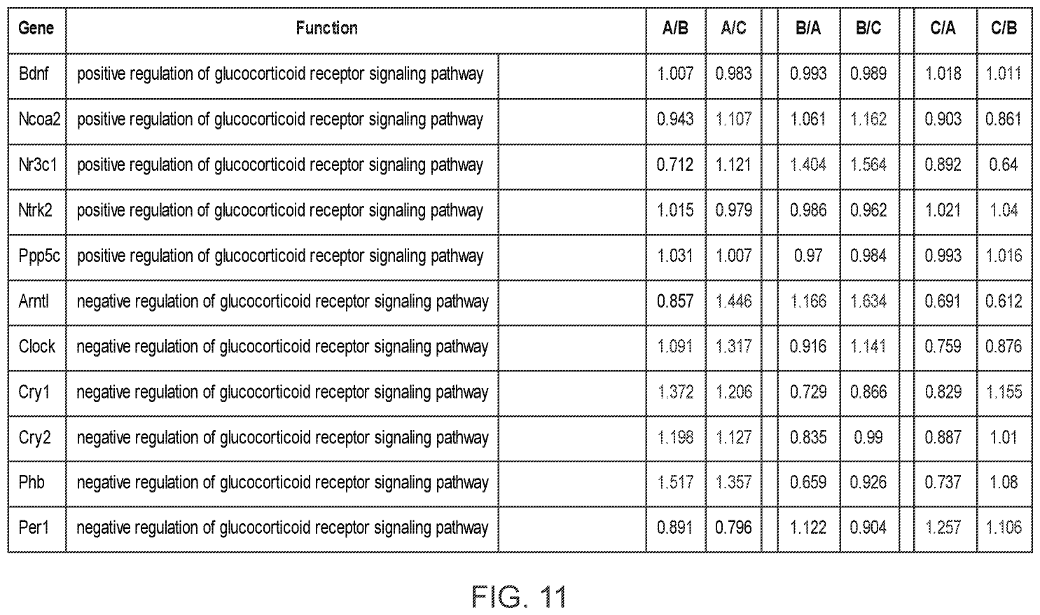

To evaluate the efficacy of hydrocortisone therapy in sepsis patients having subtypes A, B, and C, differential expression of the genes of Table 7 that are associated with pharmacology of hydrocortisone therapy were evaluated for the subtypes A, B, and C. Specifically, FIG. 11 depicts differential expression of the genes of Table 7 that are associated with pharmacology of hydrocortisone therapy (e.g., regulation of the glucocorticoid receptor signaling pathway) for the subtypes A, B, and C, in accordance with an embodiment. As shown in FIG. 11, subtype A patients exhibit differential expression of genes associated with glucocorticoid receptor signaling than subtype B patients. Specifically, relative to subtype B patients, subtype A patients exhibit down-regulation of genes associated with positive regulation of the glucocorticoid receptor signaling pathway, but up-regulation of genes associated with negative regulation of the glucocorticoid receptor signaling pathway. In other words, relative to subtype A patients, subtype B patients exhibit up-regulation of genes associated with positive regulation of the glucocorticoid receptor signaling pathway, but down-regulation of genes associated with negative regulation of the glucocorticoid receptor signaling pathway.

Due to this differential expression of genes associated with glucocorticoid receptor signaling between patients of subtypes A, B, and C, it was hypothesized that hydrocortisone therapy is differentially effective for the different subtypes. To test this hypothesis, multiple cohort datasets were analyzed for differential expressions and survival rates to evaluate the effect of hydrocortisone among different dysregulated host response subtypes. Specifically, the constructed classifiers discussed above. were applied to two placebo-controlled trials: the VANISH trial and a Burn-Induced SIRS trial to evaluate the survival rate of the patients that received hydrocortisone therapy.^{13, 50}

To evaluate hydrocortisone therapy response in dysregulated host response patients of the identified dysregulated host response patient subtypes, as discussed in detail below³¹ the patient subtype classifiers were applied to a transcriptomic dataset from a placebo-controlled hydrocortisone clinical trials in sepsis patients and burn-induced SIRS patients that failed to show a difference in mortality between the treatment and placebo arms of the trial. Differential responses to hydrocortisone therapy were identified for the different patient subtypes. Specifically, one patient subtype is shown to benefit from hydrocortisone, and one or both of the other patient subtypes are shown to worsen with hydrocortisone.

The test expression data from each trial were normalized by the platform normalization matrix described above¹³ so that the test data were more consistent with the training data. The classifiers (e.g., the Full Model, the SS Model, the S Model, and the P Model) were then applied to the normalized data such that the patients were classified into A, B, and C subtypes. In contrast to the COCONUT method, the normalization approach described herein is simpler because it does not use controls and instead employs a platform normalization matrix, and then selects all of the samples from the matrix used by the target platform of the target sample and then co-normalizes them together. Therefore, each sample in the target samples was normalized independently with the normalization matrix of the sample array platform.

Survival and mortality rates were calculated at day 28 because survival and mortality labels at other time points were not available. A single-time-point survival analysis was performed to observe the difference of survival rate between the hydrocortisone therapy group and the placebo group in each subtype. Binomial and Chi-squared with continuity correction tests were used to test for the significance of these differences. Mortality reduction when ruling out hydrocortisone was calculated as: 1−(Placebo's mortality rate/Hydrocortisone's mortality rate). Conversely, mortality reduction when ruling in hydrocortisone was calculated as: 1−(Hydrocortisone's mortality rate/Placebo's mortality rate), whichever denominator was larger.

Tables 9-16 below depict the survival analyses for each subtype (e.g., A, B, and C) for each classifier (e.g., the Full Model, the SS Model, the S Model, and the P Model) for each trial. Specifically, Tables 9 and 13 depicts survival analysis for each subtype (e.g., A, B, and C) for the Full Model, Tables 10 and 14 depicts survival analysis for each subtype (e.g., A, B, and C) for the SS Model, Tables 11 and 15 depicts survival analysis for each subtype (e.g., A, B, and C) for the S Model, and Tables 12 and 16 depicts survival analysis for each subtype (e.g., A, B, and C) for the P Model.

TABLE 9
Full Model VANISH Trial Survival Analysis

| Hydro | B | C | A | CA | Total |
|---|---|---|---|---|---|
| Alive | 16 | 11 | 9 | 20 | 36 |
| Dead | 6 | 8 | 8 | 16 | 22 |
| Total | 22 | 19 | 17 | 36 | 58 |
| Survival rate | 72.7% | 57.9% | 52.9% | 55.6% | 62.1% |
| Mortality rate | 27.3% | 42.1% | 47.1% | 44.4% | 37.9% |
| Placebo | | | | | |
| Alive | 11 | 18 | 15 | 33 | 44 |
| Dead | 8 | 4 | 3 | 7 | 15 |
| Total | 19 | 22 | 18 | 40 | 59 |
| Survival rate | 57.9% | 81.8% | 83.3% | 82.5% | 74.6% |
| Mortality rate | 42.1% | 18.2% | 16.7% | 17.5% | 25.4% |
| Grand total | 41 | 41 | 35 | 76 | 117 |
| Mortality Reduction | Hydro | Placebo | Placebo | Placebo | Placebo |
| Group | | | | | |
| Binomial P-value | 1E−01 | 1E−02 | 3E−03 | 2E−04 | 2E−02 |
| Chi-squared P-value | 5E−01 | 2E−01 | 1E−01 | 2E−02 | 2E−01 |
| Mortality reduction | 35.2% | 56.8% | 64.6% | 60.6% | 33.0% |
| Overall Mortality | 34.1% | 29.3% | 31.4% | 30.3% | 31.6% |

TABLE 10
SS Model VANISH Trial Survival Analysis

| Hydro | B | C | A | CA | Total |
|---|---|---|---|---|---|
| Alive | 5 | 16 | 15 | 31 | 36 |
| Dead | 5 | 8 | 9 | 17 | 22 |
| Total | 10 | 24 | 24 | 48 | 58 |
| Survival rate | 50.0% | 66.7% | 62.5% | 64.6% | 62.1% |
| Mortality rate | 50.0% | 33.3% | 37.5% | 35.4% | 37.9% |
| Placebo | | | | | |
| Alive | 9 | 17 | 18 | 35 | 44 |
| Dead | 6 | 8 | 1 | 9 | 15 |
| Total | 15 | 25 | 19 | 44 | 59 |
| Survival rate | 60.0% | 68.0% | 94.7% | 79.5% | 74.6% |
| Mortality rate | 40.0% | 32.0% | 5.3% | 20.5% | 25.4% |
| Grand total | 25 | 49 | 43 | 92 | 117 |
| Mortality Reduction | Placebo | Placebo | Placebo | Placebo | Placebo |
| Group | | | | | |
| Binomial P-value | 4E−01 | 5E−01 | 2E−06 | 1E−02 | 2E−02 |
| Chi-squared P-value | 9E−01 | 8E−01 | 3E−02 | 2E−01 | 2E−01 |
| Mortality reduction | 20.0% | 4.0% | 86.0% | 42.2% | 33.0% |
| Overall Mortality | 44.0% | 32.7% | 23.3% | 28.3% | 31.6% |

TABLE 11
S Model VANISH Trial Survival Analysis

| Hydro | B | C | A | CA | Total |
|---|---|---|---|---|---|
| Alive | 12 | 8 | 16 | 8 | 20 |
| Dead | 9 | 4 | 9 | 4 | 13 |
| Total | 21 | 12 | 25 | 12 | 33 |
| Survival rate | 57.1% | 66.7% | 64.0% | 66.7% | 60.6% |
| Mortality rate | 42.9% | 33.3% | 36.0% | 33.3% | 39.4% |
| Placebo | | | | | |
| Alive | 17 | 8 | 19 | 8 | 25 |
| Dead | 9 | 5 | 1 | 5 | 14 |
| Total | 26 | 13 | 20 | 13 | 39 |
| Survival rate | 65.4% | 61.5% | 95.0% | 61.5% | 64.1% |
| Mortality rate | 34.6% | 38.5% | 5.0% | 38.5% | 35.9% |
| Grand total | 47 | 25 | 45 | 25 | 72 |
| Mortality Reduction | 29 | 16 | 35 | 16 | 45 |
| Group | | | | | |
| Binomial P-value | 18 | 9 | 10 | 9 | 27 |
| Chi-squared P-value | 0.0762 | 0.0225 | 4.5145 | 0.0225 | 0.0037 |
| Mortality reduction | Placebo | Hydro | Placebo | Hydro | Placebo |
| Overall Mortality | 0.28 | 0.48 | 0.000002 | 5E−01 | 4E−01 |

TABLE 12
P Model VANISH Trial Survival Analysis

| Hydro | B | C | A | CA | Total |
|---|---|---|---|---|---|
| Alive | 23 | 4 | 9 | 13 | 36 |
| Dead | 8 | 4 | 10 | 14 | 22 |
| Total | 31 | 8 | 19 | 27 | 58 |
| Survival rate | 74.2% | 50.0% | 47.4% | 48.1% | 62.1% |
| Mortality rate | 25.8% | 50.0% | 52.6% | 51.9% | 37.9% |
| Placebo | | | | | |
| Alive | 20 | 9 | 15 | 24 | 44 |
| Dead | 8 | 5 | 2 | 7 | 15 |
| Total | 28 | 14 | 17 | 31 | 59 |
| Survival rate | 71.4% | 64.3% | 88.2% | 77.4% | 74.6% |
| Mortality rate | 28.6% | 35.7% | 11.8% | 22.6% | 25.4% |
| Grand total | 59 | 22 | 36 | 58 | 117 |
| Mortality Reduction | Hydro | Placebo | Placebo | Placebo | Placebo |
| Group | | | | | |
| Binomial P-value | 5E−01 | 3E−01 | 2E−05 | 9E−04 | 2E−02 |
| Chi-squared P-value | 1E+00 | 8E−01 | 2E−02 | 4E−02 | 2E−01 |
| Mortality reduction | 9.7% | 28.6% | 77.6% | 56.5% | 33.0% |
| Overall Mortality | 27.1% | 40.9% | 33.3% | 36.2% | 31.6% |

TABLE 13
Full Model Burn-Induced SIRS Trial Survival Analysis

| Hydro | B | C | A | CA | Total |
|---|---|---|---|---|---|
| Alive | 6 | 1 | 2 | 3 | 9 |
| Dead | 1 | 4 | 1 | 5 | 6 |
| Total | 7 | 5 | 3 | 8 | 15 |
| Survival rate | 85.7% | 20.0% | 66.7% | 37.5% | 60.0% |
| Mortality rate | 14.3% | 80.0% | 33.3% | 62.5% | 40.0% |
| Placebo | | | | | |
| Alive | 3 | 5 | 5 | 10 | 13 |
| Dead | 2 | 0 | 0 | 0 | 2 |
| Total | 5 | 5 | 5 | 10 | 15 |
| Survival rate | 60.0% | 100.0% | 100.0% | 100.0% | 86.7% |
| Mortality rate | 40.0% | 0.0% | 0.0% | 0.0% | 13.3% |
| Grand total | 12 | 10 | 8 | 18 | 30 |
| Mortality | Hydro | Placebo | Placebo | Placebo | Placebo |
| Reduction | | | | | |
| Group | | | | | |
| Binomial | 2E−01 | 0E+00 | 0E+00 | 0E+00 | 1E−02 |
| P-value | | | | | |
| Chi-squared | 7E−01 | 5E−02 | 8E−01 | 2E−02 | 2E−01 |
| P-value | | | | | |
| Mortality | 64.3% | 100.0% | 100.0% | 100.0% | 66.7% |
| reduction | | | | | |
| Overall | 25.0% | 40.0% | 12.5% | 27.8% | 26.7% |
| Mortality | | | | | |

TABLE 14
SS Model Burn-Induced SIRS Trial Survival Analysis

| Hydro | B | C | A | CA | Total |
|---|---|---|---|---|---|
| Alive | 2 | 3 | 4 | 7 | 9 |
| Dead | 0 | 4 | 2 | 6 | 6 |
| Total | 2 | 7 | 6 | 13 | 15 |
| Survival rate | 100.0% | 42.9% | 66.7% | 53.8% | 60.0% |
| Mortality rate | 0.0% | 57.1% | 33.3% | 46.2% | 40.0% |
| Placebo | | | | | |
| Alive | 5 | 4 | 4 | 8 | 13 |
| Dead | 0 | 1 | 1 | 2 | 2 |
| Total | 5 | 5 | 5 | 10 | 15 |
| Survival rate | 100.0% | 80.0% | 80.0% | 80.0% | 86.7% |
| Mortality rate | 0.0% | 20.0% | 20.0% | 20.0% | 13.3% |
| Grand total | 7 | 12 | 11 | 23 | 30 |
| Mortality Reduction | Placebo | Placebo | Placebo | Placebo | Placebo |
| Group | | | | | |
| Binomial P-value | 1E+00 | 3E−02 | 3E−01 | 3E−02 | 1E−02 |
| Chi-squared P-value | — | 5E−01 | 9E−01 | 4E−01 | 2E−01 |
| Mortality reduction | — | 65.0% | 40.0% | 56.7% | 66.7% |
| Overall Mortality | 0.0% | 41.7% | 27.3% | 34.8% | 26.7% |

TABLE 15
S Model Burn-Induced SIRS Trial Survival Analysis

| Hydro | B | C | A | CA | Total |
|---|---|---|---|---|---|
| Alive | 6 | 0 | 3 | 3 | 9 |
| Dead | 1 | 3 | 2 | 5 | 6 |
| Total | 7 | 3 | 5 | 8 | 15 |
| Survival rate | 85.7% | 0.0% | 60.0% | 37.5% | 60.0% |
| Mortality rate | 14.3% | 100.0% | 40.0% | 62.5% | 40.0% |
| Placebo | | | | | |
| Alive | 4 | 4 | 5 | 9 | 13 |
| Dead | 1 | 0 | 1 | 1 | 2 |
| Total | 5 | 4 | 6 | 10 | 15 |
| Survival rate | 80.0% | 100.0% | 83.3% | 90.0% | 86.7% |
| Mortality rate | 20.0% | 0.0% | 16.7% | 10.0% | 13.3% |
| Grand total | 12 | 7 | 11 | 18 | 30 |
| Mortality Reduction | Hydro | Placebo | Placebo | Placebo | Placebo |
| Group | | | | | |
| Binomial P-value | 6E−01 | 0E+00 | 2E−01 | 4E−04 | 1E−02 |
| Chi-squared P-value | 6E−01 | 6E−02 | 9E−01 | 7E−02 | 2E−01 |
| Mortality reduction | 28.6% | 100.0% | 58.3% | 84.0% | 66.7% |
| Overall Mortality | 16.7% | 42.9% | 27.3% | 33.3% | 26.7% |

TABLE 16
P Model Burn-Induced SIRS Trial Survival Analysis

| Hydro | B | C | A | CA | Total |
|---|---|---|---|---|---|
| Alive | 5 | 0 | 4 | 4 | 9 |
| Dead | 1 | 4 | 1 | 5 | 6 |
| Total | 6 | 4 | 5 | 9 | 15 |
| Survival rate | 83.3% | 0.0% | 80.0% | 44.4% | 60.0% |
| Mortality rate | 16.7% | 100.0% | 20.0% | 55.6% | 40.0% |
| Placebo | | | | | |
| Alive | 4 | 3 | 6 | 9 | 13 |
| Dead | 2 | 0 | 0 | 0 | 2 |
| Total | 6 | 3 | 6 | 9 | 15 |
| Survival rate | 66.7% | 100.0% | 100.0% | 100.0% | 86.7% |
| Mortality rate | 33.3% | 0.0% | 0.0% | 0.0% | 13.3% |
| Grand total | 12 | 7 | 11 | 18 | 30 |
| Mortality | Hydro | Placebo | Placebo | Placebo | Placebo |
| Reduction | | | | | |
| Group | | | | | |
| Binomial | 4E−01 | 0E+00 | 0E+00 | 0E+00 | 1E−02 |
| P-value | | | | | |
| Chi-squared | 1E+00 | 6E−02 | 9E−01 | 4E−02 | 2E−01 |
| P-value | | | | | |
| Mortality | 50.0% | 100.0% | 100.0% | 100.0% | 66.7% |
| reduction | | | | | |
| Overall | 25.0% | 57.1% | 9.1% | 27.8% | 26.7% |
| Mortality | | | | | |

An alternative method for identifying patients that may be harmed by immunosuppressive effects of hydrocortisone is based on employing A and B scores to identify patients expected to exhibit increased immune activity and lower inflammation. In simple terms, this method is based on a classifying patients with a high A score and low B score.

In one example, previously identified subtypes of sepsis patients were used to tune the model to identify these type A and B patients. Two distinct sepsis response signatures (SRS1 and SRS2) were identified in five public studies (E-MTAB-4421, E-MTAB-4451, E-MTAB-5273, E-MTAB-5274, and E-MTAB-7581), where HumanHT-12 v4 BeadChip were used to generate the gene expression profiles of the patient samples. The processed data of those five studies were downloaded and processed using R programming language and software environment for statistical analysis (version 3.6.3). The Bioconductor annotation package, illuminaHumanv4.db (version 1.26.0), was used to annotate microarray probes and expression levels of genes were determined by each individual probe or mean of probes belonging to the same gene. In order to remove cohort biases, the Bioconductor package, limma (version 3.42.2), was used to remove batch effects. Using ss.b2 panel genes, subtype A, B, and C scores were calculated by geometric mean of up/down genes.

To build the classifier, we defined E-MTAB-4421, E-MTAB-4451, E-MTAB-5273, and E-MTAB-5274 as the training dataset and VANISH (E-MTAB-7581) as the testing dataset. We used features (subtype A, B, and C scores) and class labels (SRS1 vs SRS2) in the training dataset to build a machine-learned classifier based on support-vector machine (SVM) method. SVM is a supervised machine learning method for classification analysis. The algorithm finds a single or a set of hyperplanes that maximize the margin among subtype A, B, and C scores. In order to capture non-linear data, the kernel function was used. R package e1071 was used to build the SVM classifier with following parameters: method=“C-classification”, kernal=“radial”, gamma=0.1, and cost=10.

The accuracy of the classifiers was evaluated by Leave-One-Out (LOO) cross-validation over the training dataset. Also the classifier was applied to 117 controlled samples from VANISH trial. The patients predicted as Type-A (SRS2-like) exhibited significant 28-day mortality reduction when hydrocortisone was applied in comparison to placebo. These Type-A exhibited 75.5% mortality reduction in the placebo group in comparison to the hydrocortisone group (Fisher exact test p-value 0.0093). The Type-A (SRS2-like) and Type-B (SRS1-like) classifier exhibited an accuracy of 88.6%. Table 17 below depict the survival analyses for each subtype for the SS.B2 model.

TABLE 17
SS.B2 Model VANISH Trial Survival Analysis

| Hydro | A | B | Total |
|---|---|---|---|
| Alive | 22 | 14 | 36 |
| Dead | 14 | 8 | 22 |
| Total | 36 | 22 | 58 |
| Survival rate | 61.1% | 63.6% | 62.1% |
| Mortality rate | 38.9% | 36.4% | 37.9% |
| Placebo | | | |
| Alive | 31 | 13 | 44 |
| Dead | 3 | 12 | 15 |
| Total | 34 | 25 | 59 |
| Survival rate | 91.2% | 52.0% | 74.6% |
| Mortality rate | 8.8% | 48.0% | 25.4% |
| Grand total | 70 | 47 | 117 |
| Mortality Reduction | Placebo | Hydro | Placebo |
| Group | | | |
| Fisher exact test | 5E−03 | 6E−01 | 2E−01 |
| Binomial P-value | 1E−06 | 2E−01 | 2E−02 |
| Chi-squared P-value | 8E−03 | 6E−01 | 2E−01 |
| Mortality reduction | 77.3% | 24.2% | 33.0% |
| Overall Mortality | 24.3% | 42.6% | 31.6% |

In addition to the SVM-method, thresholds can be employed to scores in order to define A vs. B labels. We discovered that subtype A and B scores play an important role in subtype SRS1 and SRS2 classification. Therefore we applied a heuristic threshold (threshold=0) to subtype A and B scores to classify SRS1-like and SRS2-like in VANISH: SRS2-like label was assigned to samples with subtype A score >0 and subtype B score <0 and SRS1-like label was assigned to the rest of samples. With the simple heuristic threshold (threshold=0), the patients predicted as SRS2-like exhibited 85.2% 28-day mortality reduction in the placebo group in comparison to the hydrocortisone group (Fisher exact test p-value 0.0159).

Besides the heuristic threshold, we also derived the thresholds for subtype A and B scores using the training dataset. Same as the SVM method, we defined E-MTAB-4421, E-MTAB-4451, E-MTAB-5273, and E-MTAB-5274 as the training dataset and VANISH (E-MTAB-7581) as the testing dataset. To identify the best threshold of subtype A score to classify subtype SRS1 and SRS2 in training dataset, we fitted subtype A scores and SRS subtype labels into ROC curve (receiver operating characteristic curve) and identified the threshold for subtype A score (A threshold=−0.2664) which is the closest point to the top-left part of the plot with perfect sensitivity or specificity. With the same method, the optimal B score threshold (B threshold=0.3179) was selected to classify subtype SRS1 and SRS2 in the training set. We applied the defined optimal thresholds for subtype A and B scores to the VANISH trail: the samples whose subtype A scores are above the A threshold and subtype B scores are below the B threshold were labeled as SRS2-like and the rest VANISH trail samples were labeled as SRS1-like. With such classification, the patients with the SRS2-like label showed 81.7% 28-day mortality reduction in the placebo group in comparison to the hydrocortisone group (Fisher exact test p-value 0.0065).

Various thresholds can be employed in order to optimize for mortality reduction (mr) and for the number of patients who may benefit (percentage of patients that are B). Table 18 below depict the survival analyses for each subtype for the SS.B2 model.

TABLE 18
SS.B2 Model VANISH Trial Survival Analysis

| | | | | | | | hydro_alive, |
|---|---|---|---|---|---|---|---|
| | | | Accuracy | % of | | Fisher | hydro_dead, |
| | A score | B score | in training | patients | | exact | placebo_alive, |
| class | cutoff | cutoff | set | that are B | mr | test | placebo_dead |
| B | 0.0000 | 0.0000 | 0.74830 | 23.08% | 83.8% | 0.01831837 | 7, 9, 10, 1 |
| A | 0.0000 | 0.0000 | 0.74830 | 76.92% | 5.8% | 1 | 29, 13, 34, 14 |
| B | −0.2664 | 0.3179 | 0.82483 | 41.88% | 82.5% | 0.00361372 | 13, 11, 23, 2 |
| A | −0.2664 | 0.3179 | 0.82483 | 58.12% | 15.4% | 0.80002799 | 23, 11, 21, 13 |
| B | 0.0000 | 0.0000 | 0.75283 | 23.08% | 83.8% | 0.01831837 | 7, 9, 10, 1 |
| A | 0.0000 | 0.0000 | 0.75283 | 76.92% | 5.8% | 1 | 29, 13, 34, 14 |
| B | −0.1277 | 0.3630 | 0.83673 | 39.32% | 83.3% | 0.00267971 | 11, 11, 22, 2 |
| A | −0.1277 | 0.3630 | 0.83673 | 60.68% | 17.7% | 0.62103399 | 25, 11, 22, 13 |
| B | 0.0000 | 0.0000 | 0.76871 | 23.08% | 83.8% | 0.01831837 | 7, 9, 10, 1 |
| A | 0.0000 | 0.0000 | 0.76871 | 76.92% | 5.8% | 1 | 29, 13, 34, 14 |
| B | −0.0406 | 0.1067 | 0.79592 | 25.64% | 87.3% | 0.00669665 | 7, 9, 13, 1 |
| A | −0.0406 | 0.1067 | 0.79592 | 74.36% | 0.5% | 1 | 29, 13, 31, 14 |
| B | 0.0000 | 0.0000 | 0.74830 | 23.93% | 85.2% | 0.01587078 | 7, 9, 11, 1 |
| A | 0.0000 | 0.0000 | 0.74830 | 76.07% | 3.8% | 1 | 29, 13, 33, 14 |
| B | −0.2664 | 0.3179 | 0.82483 | 39.32% | 81.7% | 0.00651932 | 12, 10, 22, 2 |
| A | −0.2664 | 0.3179 | 0.82483 | 60.68% | 10.3% | 0.80648544 | 24, 12, 22, 13 |
| B | 0.0000 | 0.0000 | 0.75283 | 17.09% | 82.5% | 0.02810193 | 4, 7, 8, 1 |
| A | 0.0000 | 0.0000 | 0.75283 | 82.91% | 12.3% | 0.82470462 | 32, 15, 36, 14 |
| B | −0.1277 | 0.3630 | 0.83673 | 29.91% | 79.0% | 0.01164257 | 8, 9, 16, 2 |
| A | −0.1277 | 0.3630 | 0.83673 | 70.09% | 0.0% | 1 | 28, 13, 28, 13 |
| B | 0.0000 | 0.0000 | 0.76871 | 37.61% | 86.8% | 0.01260373 | 15, 10, 18, 1 |
| A | 0.0000 | 0.0000 | 0.76871 | 62.39% | 3.8% | 1 | 21, 12, 26, 14 |
| B | −0.0406 | 0.1067 | 0.79592 | 41.88% | 79.2% | 0.01807688 | 15, 10, 22, 2 |
| A | −0.0406 | 0.1067 | 0.79592 | 58.12% | 2.1% | 1 | 21, 12, 22, 13 |

Response to hydrocortisone therapy for each subtype of patients identified by each Model for each of the sepsis and burn-induced SIRS patient studies, was evaluated based on a p-values and mortality reduction for the subtype. To evaluate possible adverse response to hydrocortisone therapy for a subtype, a binomial p-value was calculated as the probability of achieving a random survival rate of less than or equal to the survival rate P(X<=x) observed in the hydrocortisone therapy group for the subtype, assuming that the survival rate observed in the placebo therapy group for the subtype was an expected survival rate for patients receiving no hydrocortisone therapy. To evaluate possible favorable response to hydrocortisone therapy for a subtype, a chi-squared p-value was calculated for the survival rate P(X>=x) observed in the hydrocortisone therapy group for the subtype. The chi-squared p-value was calculated with continuity correction when computed for 2-by-2 tables.

As an example, assuming a chosen, statistically significant p-value of at least 0.1, hydrocortisone therapy response was evaluated based on mortality reduction, as well as binomial and chi-squared p-values, for sepsis and SIRS patients, for each subtype, and for each Model, as follows.

First, assuming the chosen statistically significant p-value of at least 0.1, hydrocortisone therapy response was evaluated for sepsis patients. As shown in Tables 9-12, sepsis patients assigned to the A subtype by the Full, SS, S, and P Models exhibited statistically significant 28-day mortality reduction when placebo was applied in comparison to hydrocortisone therapy. Specifically, the Full, SS, S, and P Models identified a subtype, A, exhibiting 64.6%, 86.0%, 86.1%, and 77.6%, respectively, lower mortality in the placebo group when compared to the hydrocortisone therapy group. As shown in Table 9, sepsis patients assigned to the B subtype by the Full Model exhibited statistically significant 28-day mortality reduction when hydrocortisone was applied in comparison to placebo. Specifically, the Full Model identified a subtype, B, exhibiting 35.2%, lower mortality in the hydrocortisone group when compared to the placebo group. As shown in Table 9, sepsis patients assigned to the C subtype by the Full Model exhibited statistically significant 28-day mortality reduction when placebo was applied in comparison to hydrocortisone therapy. Specifically, the Full Model identified a subtype, C, exhibiting 56.8% lower mortality in the placebo group when compared to the hydrocortisone therapy group.

Additionally, assuming the chosen statistically significant p-value of at least 0.1, hydrocortisone therapy response was evaluated for SIRS patients. As shown in Tables 13, 15, and 16, SIRS patients assigned to the A subtype by the Full, S, and P Models exhibited statistically significant 28-day mortality reduction when placebo was applied in comparison to hydrocortisone therapy. Specifically, the Full, S, and P Models identified a subtype, A, exhibiting 100% lower mortality in the placebo group when compared to the hydrocortisone therapy group. As shown in Table 15, SIRS patients assigned to the B subtype by the S Model exhibited statistically significant 28-day mortality reduction when hydrocortisone was applied in comparison to placebo. Specifically, the S Model identified a subtype, B, exhibiting 28.6%, lower mortality in the hydrocortisone group when compared to the placebo group. As shown in Tables 13-16, SIRS patients assigned to the C subtype by the Full, SS, S, and P Models exhibited statistically significant 28-day mortality reduction when placebo was applied in comparison to hydrocortisone therapy. Specifically, the Full, SS, S, and P Models identified a subtype, A, exhibiting 100%, 65%, 100%, and 100%, respectively, lower mortality in the placebo group when compared to the hydrocortisone therapy group.

Based on these observations of differential mortality reduction as a result of hydrocortisone therapy or placebo therapy between the subtypes A, B, and C identified for both sepsis and SIRS patients by the Full, SS, S, and P Models, the subtypes can be assigned more descriptive titles such as “favorably responsive”, “adversely responsive”, and “non-responsive” to corticosteroid therapy. For example, assuming the chosen statistically significant p-value of at least 0.1, subtypes can be assigned titles as follows.

First, assuming the chosen statistically significant p-value of at least 0.1, subtypes A, B, and C identified for sepsis patients by the Full, SS, S, and P Models can be assigned titles as follows. Because mortality reduction was statistically significant in the placebo group compared to the hydrocortisone therapy group for sepsis patients assigned to subtype A by the Full, SS, S, and P Models, sepsis patients assigned to subtype A by at least one of the Full, SS, S, and P Models can be colloquially referred to as “adversely responsive” to corticosteroid therapy. Similarly, because mortality reduction was statistically significant in the placebo group compared to the hydrocortisone therapy group for sepsis patients assigned to subtype C by the Full Model, sepsis patients assigned to subtype C by the Full Model can be colloquially referred to as “adversely responsive” to corticosteroid therapy. Conversely, because mortality reduction was statistically significant in the hydrocortisone therapy group compared to the placebo group for sepsis patients assigned to subtype B by the Full Model, sepsis patients assigned to subtype B by the Full Model can be colloquially referred to as “favorably responsive” to corticosteroid therapy. Finally, because correlation between therapy group and mortality reduction was not statistically significant for sepsis patients assigned to subtypes B and C by the SS, S, and P Models, sepsis patients assigned to subtype B or C by at least one of the SS, S, and P Models can colloquially referred to as “non-responsive” to corticosteroid therapy.

Additionally, assuming the chosen statistically significant p-value of at least 0.1, subtypes A, B, and C identified for SIRS patients by the Full, SS, S, and P Models can be assigned titles as follows. Because mortality reduction was statistically significant in the placebo group compared to the hydrocortisone therapy group for SIRS patients assigned to subtype A by the Full, S, and P Models, SIRS patients assigned to subtype A by at least one of the Full, S, and P Models can be colloquially referred to as “adversely responsive” to corticosteroid therapy. Similarly, because mortality reduction was statistically significant in the placebo group compared to the hydrocortisone therapy group for SIRS patients assigned to subtype C by the Full, SS, S, and P Models, SIRS patients assigned to subtype C by at least one of the Full, SS, S, and P Models can be colloquially referred to as “adversely responsive” to corticosteroid therapy. Conversely, because mortality reduction was statistically significant in the hydrocortisone therapy group compared to the placebo group for SIRS patients assigned to subtype B by the S Model, SIRS patients assigned to subtype B by the S Model one can be colloquially referred to as “favorably responsive” to corticosteroid therapy. Because correlation between therapy group and mortality reduction was not statistically significant for SIRS patients assigned to subtype B by the Full, SS, and P Models, SIRS patients assigned to subtype B by at least one of the Full, SS, and P Models can be colloquially referred to as “non-responsive” to corticosteroid therapy. Finally, because correlation between therapy group and mortality reduction was not statistically significant for SIRS patients assigned to subtype A by the SS Model, SIRS patients assigned to subtype A by the SS Model can be colloquially referred to as “non-responsive” to corticosteroid therapy.

Further based on these observations of mortality reduction in sepsis and SIRS patients assigned to subtypes A, B, and C by the Full, SS, S, and P Models, subtyped sepsis and SIRS patients may be provided treatment recommendations accordingly. For instance, in one embodiment, patients subtyped as “favorably responsive” to corticosteroid therapy can be recommended treatment with corticosteroids, while patients subtyped as “adversely responsive” to corticosteroid therapy can be recommended no corticosteroid therapy, and while patients subtyped as “non-responsive” to corticosteroid therapy can be provided with no therapy recommendation.

Discrepancies between treatment recommendations for a given subtype (e.g., subtype A, B, or C) across models (e.g., the Full, SS, S, and P Model) and types of dysregulated host response (e.g., sepsis or SIRS) are due to the fact that statistical significance of mortality reduction for a subtype varies according to the model used to assign the subtype, as well as the type of dysregulated host response. For example, as discussed above, for a statistically significant p-value of at least 0.1, sepsis patients that are determined to be of subtype C by the Full Model may be subtyped as “adversely responsive” to corticosteroid therapy, and thus recommended no corticosteroid therapy. Conversely, for a statistically significant p-value of at least 0.1, sepsis patients that are determined to be of subtype C by the SS Model may be subtyped as “non-responsive” to corticosteroid therapy, and thus may not be provided with a therapy recommendation, while SIRS patients that are determined to be of subtype C by the SS Model may be subtyped as “adversely responsive” to corticosteroid therapy, and thus recommended no corticosteroid therapy. Therefore, the titles assigned to subtypes A, B, and C for each model and for each type of dysregulated host response, and thus the therapy recommendations, are dependent upon the chosen statistically significant p-value. In alternative embodiments, the statistically significant p-value may be adjusted, and thus the titles assigned to subtypes A, B, and C, as well as the therapy recommendations, may be adjusted. For example, in some embodiments, the statistically significant p-value may be less than 0.1.

Furthermore, as described above, survival analyses were performed independently from classifier training, which prevented the training from overfitting issues. Thus, the observations of differential response to corticosteroid therapy among the three different subtypes can likely be attributed to the fundamental link between therapy and the biological nature of each subtype. For instance, the most significant molecular functions from the GO analysis of the A subtype were antigen binding, MEW protein complex binding, and cytokine binding, which are strong indicators for adaptive immune response. In the survival analysis results for the A subtype, significant mortality reduction was observed in the placebo group compared to the corticosteroid therapy group, inferring that the corticosteroid therapy might be potentially disturbing the already working adaptive immune response of the A subtype patients. On the contrary, according to the GO analysis of the B subtype, the B subtype was significantly enriched with interleukin (IL)-1 receptor and complement component Cl, indicating a more likely innate immune response. Indeed, for the B subtype, instead of a mortality reduction in the placebo group, a mortality reduction was observed with corticosteroid therapy.

VI.B. Dysregulated Host Response Patient Subtypes B and C

VI.B.1. Immune Stimulants: Checkpoint Inhibitors, Interleukins, and Mediators of T-Cell Regulation Attenuation

As discussed in detail above, subtype B and C patients may benefit from immune stimulants. Examples of therapies for stimulating the immune system include checkpoint inhibitors, interleukins such as IL-7, and therapies that attenuate the regulation and suppression of T-cell function such as blockers of IL-10, and TGF-β.

FIG. 12 provides support for a hypothesis of differential response to checkpoint inhibition therapy between the subtypes A, B, and C, by depicting differential expression of genes of Table 7 that are associated with pharmacology of checkpoint inhibition therapy (e.g., regulation of immune checkpoints and related immune functions mediated by cytokines) for subtypes A, B, and C, in accordance with an embodiment.

As shown in FIG. 12, subtypes B and C exhibit down-regulation of immune markers including IL-7 and INF-γ. Conversely, subtype A exhibits up-regulation of immune markers including IL-7 and INF-γ. PD-1 and PD-L1 are receptor/ligand immune inhibitory cell surface markers. Checkpoint inhibition of PD-1/PD-L1 interaction results in upregulation of IL-7. As shown in FIG. 12, subtype B patients exhibit up-regulation of PD-L1 and down-regulation of IL-7. Thus subtype B patients may benefit from anti-PD-1 and anti-PD-L1 therapy.

CD28 interacts with CD86 and CD80 to mediate stimulation of T-cell function. CTLA-4 interacts with CD86 and CD80 to mediate inhibition of T-cell function. Checkpoint inhibition of CTLA-4 causes upregulation of INF-γ. As shown in FIG. 12, subtype B and C patients exhibit an increased ratio of CTLA-4/CD28 and decreased expression of INF-γ. Therefore, subtype B and C patients may benefit from anti-CTLA-4 therapy.

TIM-3 interacts with CEACAM-1 to mediate inhibition of T cell function. As shown in FIG. 12, subtype B and C patients exhibit up-regulation of CEACAM-1 and TIM-3. Therefore, subtype B and C patients may benefit from anti-CEACAM-1 and anti-TIM-3 therapy.

VI.C. Dysregulated Host Response Patient Subtype C

VI.C.1. Modulators of Coagulation and Modulators of Vascular Permeability

As discussed in detail above, subtype C patients exhibit coagulopathy and may benefit from modulators of coagulation such as anticoagulants and modulators of vascular permeability. Specifically, therapies that indirectly modulate coagulation factors, such as activated protein C and antithrombin, may be of particular benefit to subtype C patients due to the complexity of the coagulation system and difficulty of managing coagulation by targeting specific coagulation factors directly.

### VII. Benefits Conferred by Systemic Immume Response Patient Subtype Classifiers

VII.A. Improvement of Acute Care

Syndromes caused by dysregulated host response, such as sepsis, are not single diseases, but rather are heterogeneous processes. As a result, evaluation of effective therapies has been hampered by limitations in the ability to classify patients into homogeneous subtypes based on pathogenesis. The improved ability to subtype patients exhibiting dysregulated host response can therefore enable identification and evaluation of effective new therapies for treating dysregulated host response syndromes such as sepsis.

VII.B. Precision Clinical Trials

The improved ability to subtype patients exhibiting dysregulated host response also enables the design and execution of precision clinical trials and the ability to test effectiveness potential new therapies by targeting the therapies to specific subtypes of patients. The improved ability to subtype patients exhibiting dysregulated host response also allows for predictive therapy enrichment in positively-responsive patients and avoiding the use of therapies in non-responsive or adversely-responsive patients.

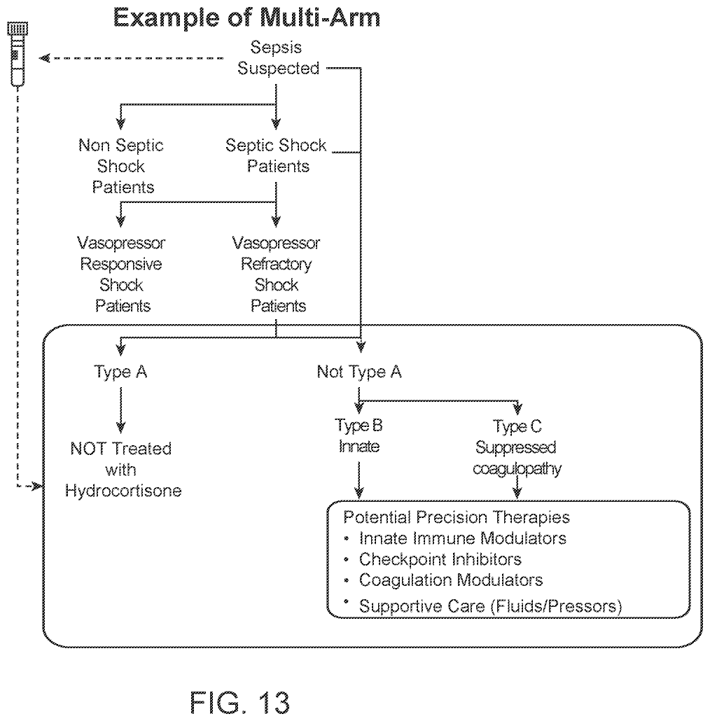

FIG. 13 depicts an example of a precision clinical trial design, in accordance with an embodiment. FIG. 13 depicts an example of a precision platform clinical trial design, in accordance with an embodiment.

VII.C. Precision Care

The improved ability to subtype patients exhibiting dysregulated host response also enables the delivery of precision care. The patient subtype classifiers discussed throughout this disclosure allow for the development of tests for guiding dysregulated host response therapy, and in particular for guiding dysregulated host response therapy in acute care. Specifically, the patient subtype classifiers discussed throughout this disclosure can serve as a companion diagnostic to enable the safe and effective use of dysregulated host response therapy.

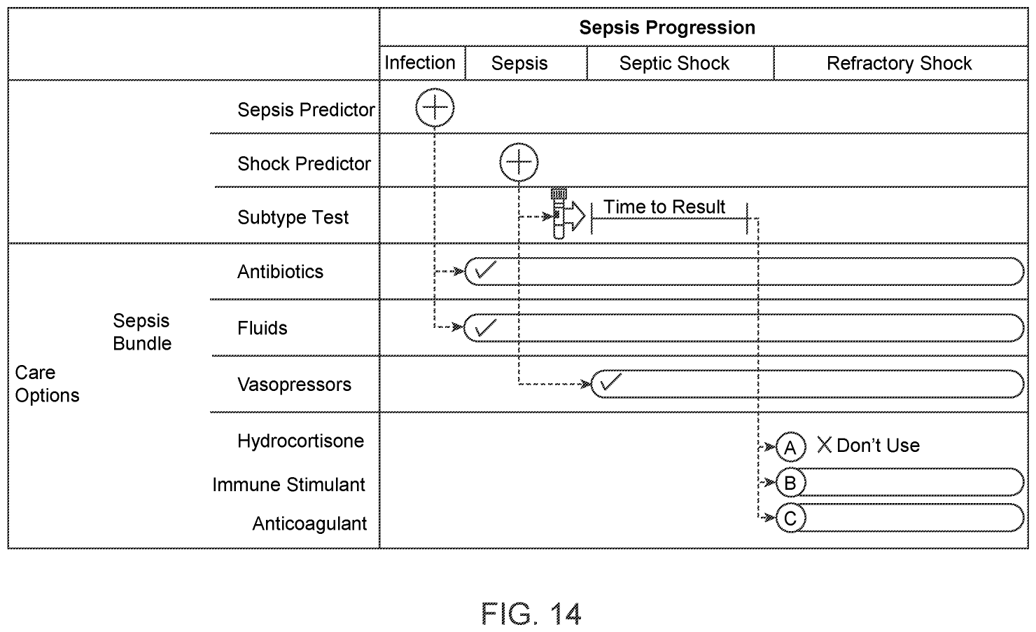

FIG. 14 depicts an example workflow for the use of the patient subtype classifiers discussed throughout this disclosure, in targeting therapies for septic shock patients, in accordance with an embodiment. The same approach can similarly be used to target therapies for patients exhibiting sepsis other than septic shock, as well as other dysregulated host response syndromes resulting from insults other than infection, such as burns, acute respiratory distress syndrome, acute kidney injury, and/or any other insults.

VII.D. Patient Subtyping Test

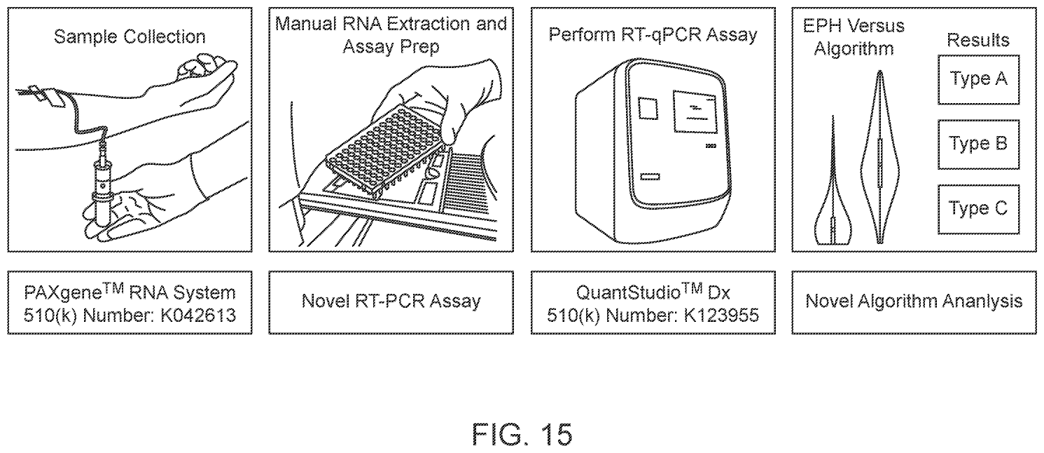

FIG. 15 depicts an example dysregulated host response patient subtyping test that employs an FDA-cleared patient sample collection system (e.g., PAXgene Blood RNA System), and an FDA-cleared Real Time PCR system (e.g. the Thermo Fisher Quantstudio Dx System), in accordance with an embodiment. An RT-qPCR test that quantifies the absolute and/or relative expression levels of genes that enable patient subtyping may be run using a testing system such as the one depicted in FIG. 15. This test can then be used in precision trials and in precision care as discussed above.

In some embodiments, the subtyping test can be differently configured. For example, the subtyping test need not employ the manual RNA extraction and assay preparation step shown in FIG. 15. In such embodiments, the sample can be directly added to a system for performing RT-qPCR and the extraction and PCR analysis can be performed all in one.

### VIII. Example Computer

FIG. 16 illustrates an example computer 1600 for implementing the methods described herein, in accordance with an embodiment. The computer 1600 includes at least one processor 1601 coupled to a chipset 1602. The chipset 1602 includes a memory controller hub 1610 and an input/output (I/O) controller hub 1611. A memory 1603 and a graphics adapter 1606 are coupled to the memory controller hub 1610, and a display 1609 is coupled to the graphics adapter 1606. A storage device 1604, an input device 1607, and network adapter 1608 are coupled to the I/O controller hub 1611. Other embodiments of the computer 1600 have different architectures.

The storage device 1604 is a non-transitory computer-readable storage medium such as a hard drive, compact disk read-only memory (CD-ROM), DVD, or a solid-state memory device. The memory 1603 holds instructions and data used by the processor 1601. The input interface 1607 is a touch-screen interface, a mouse, track ball, or other type of pointing device, a keyboard, or some combination thereof, and is used to input data into the computer 1600. In some embodiments, the computer 1600 can be configured to receive input (e.g., commands) from the input interface 1607 via gestures from the user. The graphics adapter 1606 displays images and other information on the display 1609. The network adapter 1608 couples the computer 1600 to one or more computer networks.

The computer 1600 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 1604, loaded into the memory 1603, and executed by the processor 1601.

The types of computers 1600 used to implement the methods described herein can vary depending upon the embodiment and the processing power required by the entity. For example, the diagnostic/treatment system can run in a single computer 1600 or multiple computers 1600 communicating with each other through a network such as in a server farm. The computers 1600 can lack some of the components described above, such as graphics adapters 1606, and displays 1609.

### IX. Example Kit Implementation

Also disclosed herein are kits for determining a therapy recommendation for an individual. Such kits can include reagents for detecting expression levels of one or biomarkers and instructions for classifying based on the detected expression levels and selecting a therapy recommendation based on the classification.

The detection reagents can be provided as part of a kit. Thus, the invention further provides kits for detecting the presence of a panel of biomarkers of interest in a biological test sample. A kit can comprise a set of reagents for generating a dataset via at least one protein detection assay (e.g., immunoassay or RT-PCR assay) that analyzes the test sample from the subject. In various embodiments, the set of reagents enable detection of quantitative expression levels of biomarkers described in any of Tables 1, 2A-2B, 3, and 4A-4D. In certain aspects, the reagents include one or more antibodies that bind to one or more of the markers. The antibodies may be monoclonal antibodies or polyclonal antibodies. In some aspects, the reagents can include reagents for performing ELISA including buffers and detection agents. In some aspects, the reagents include primers that are designed to hybridize with nucleic acids transcribed from genes identified in any of Tables 1, 2A-2B, 3, and 4A-4D.

A kit can include instructions for use of a set of reagents. For example, a kit can include instructions for performing at least one biomarker detection assay such as an immunoassay, a protein-binding assay, an antibody-based assay, an antigen-binding protein-based assay, a protein-based array, an enzyme-linked immunosorbent assay (ELISA), flow cytometry, a protein array, a blot, a Western blot, nephelometry, turbidimetry, chromatography, mass spectrometry, enzymatic activity, proximity extension assay, and an immunoassay selected from RIA, immunofluorescence, immunochemiluminescence, immunoelectrochemiluminescence, immunoelectrophoretic, a competitive immunoassay, and immunoprecipitation.

In various embodiments, a kit can include instructions for performing at least one of RT-qPCR (quantitative reverse transcription polymerase chain reaction), qPCR (quantitative polymerase chain reaction), PCR (polymerase chain reaction), RT-PCR (reverse transcription polymerase chain reaction), SDA (strand displacement amplification), RPA (recombinase polymerase amplification), MDA (multiple displacement amplification), HDA (helicase dependent amplification), LAMP (loop-mediated isothermal amplification), RCA (rolling circle amplification), NASBA (nucleic acid-sequence-based amplification), and any other isothermal or thermocycled amplification reaction.

In various embodiments, the kit includes instructions for determining quantitative expression data for three biomarkers, the instructions including: contacting the sample with a reagent; generating a plurality of complexes between the reagent and the plurality of biomarkers in the sample; and detecting the plurality of complexes to obtain a dataset associated with the sample, wherein the dataset comprises the quantitative expression data for the biomarker.

In various embodiments, the kits include instructions for practicing the methods disclosed herein (e.g., methods for training and/or implementing a patient subtype classifier). These instructions can be present in the subject kits in a variety of forms, one or more of which can be present in the kit. One form in which these instructions can be present is as printed information on a suitable medium or substrate, e.g., a piece or pieces of paper on which the information is printed, in the packaging of the kit, in a package insert, etc. Yet another means would be a computer readable medium, e.g., diskette, CD, hard-drive, network data storage, etc., on which the information has been recorded. Yet another means that can be present is a website address which can be used via the internet to access the information at a removed site. Any convenient means can be present in the kits.

### X. Additional Considerations

All references, issued patents and patent applications cited within the body of the specification are hereby incorporated by reference in their entirety, for all purposes.

The foregoing description of the embodiments of the disclosure has been presented for the purpose of illustration; it is not intended to be exhaustive or to limit the disclosure to the precise forms disclosed. Persons skilled in the relevant art can appreciate that many modifications and variations are possible in light of the above disclosure.

Some portions of this description describe the embodiments of the disclosure in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like.

Any of the steps, operations, or processes described herein can be performed or implemented with one or more hardware or software modules, alone or in combination with other devices. In one embodiment, a software module is implemented with a computer program product including a computer-readable non-transitory medium containing computer program code, which can be executed by a computer processor for performing any or all of the steps, operations, or processes described.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments of the disclosure may also relate to a product that is produced by a computing process described herein. Such a product may include information resulting from a computing process, where the information is stored on a non-transitory, tangible computer-readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the disclosure be limited not by this detailed description, but rather by any claims that issue on an application based hereon. Accordingly, the disclosure of the embodiments of the disclosure is intended to be illustrative, but not limiting, of the scope of the disclosure.

### XI. Additional Embodiments

Disclosed herein is a method comprising: obtaining a sample from a subject exhibiting dysregulated host response, wherein the sample comprises a plurality of biomarkers; determining quantitative expression data for at least one biomarker selected from the group consisting of the biomarkers listed in Row 1 of one of Tables 1, 2A, 2B, and 3, at least one biomarker selected from the group consisting of the biomarkers listed in Row 2 of the one of Tables 1, 2A, 2B, and 3, and at least one biomarker selected from the group consisting of the biomarkers listed in Row 3 of the one of Tables 1, 2A, 2B, and 3; and determining a classification of the subject based on the quantitative expression data using a patient subtype classifier, the classification of the subject comprising one of subtype A, subtype B, or subtype C.

Additionally disclosed herein is a method comprising: obtaining a classification of a subject exhibiting dysregulated host response, the classification of the subject comprising one of subtype A, subtype B, or subtype C; and identifying a therapy recommendation for the subject based at least in part on the classification, wherein responsive to the classification of the subject comprising subtype A, the therapy recommendation identified for the subject comprises at least no immunosuppressive therapy, wherein responsive to the classification of the subject comprising subtype B, the therapy recommendation identified for the subject comprises at least one of no therapy recommendation, immune stimulation therapy, suppression of immune regulation therapy, blocking of immune suppression therapy, blocking of complement activity therapy, and anti-inflammatory therapy, and wherein responsive to the classification of the subject comprising subtype C, the therapy recommendation identified for the subject comprises at least one of no therapy recommendation, immune stimulation therapy, suppression of immune regulation therapy, blocking of immune suppression therapy, modulators of coagulation therapy, and modulators of vascular permeability therapy.

Additionally disclosed herein is a computer-implemented method comprising: obtaining quantitative expression data from a sample from a subject exhibiting dysregulated host response for at least one biomarker selected from the group consisting of the biomarkers listed in Row 1 of one of Tables 1, 2A, 2B, and 3, at least one biomarker selected from the group consisting of the biomarkers listed in Row 2 of the one of Tables 1, 2A, 2B, and 3, and at least one biomarker selected from the group consisting of the biomarkers listed in Row 3 of the one of Tables 1, 2A, 2B, and 3; and determining, by a computer processor, a classification of the subject based on the quantitative expression data using a patient subtype classifier, the classification of the subject comprising one of subtype A, subtype B, or subtype C.

Additionally disclosed herein is a computer-implemented method comprising: obtaining, by a computer processor, a classification of a subject exhibiting dysregulated host response, the classification of the subject comprising one of subtype A, subtype B, or subtype C; and identifying a therapy recommendation for the subject based at least in part on the classification, wherein responsive to the classification of the subject comprising subtype A, the therapy recommendation identified for the subject comprises at least no immunosuppressive therapy, wherein responsive to the classification of the subject comprising subtype B, the therapy recommendation identified for the subject comprises at least one of no therapy recommendation, immune stimulation therapy, suppression of immune regulation therapy, blocking of immune suppression therapy, blocking of complement activity therapy, and anti-inflammatory therapy, and wherein responsive to the classification of the subject comprising subtype C, the therapy recommendation identified for the subject comprises at least one of no therapy recommendation, immune stimulation therapy, suppression of immune regulation therapy, blocking of immune suppression therapy, modulators of coagulation therapy, and modulators of vascular permeability therapy.

Additionally disclosed herein is a non-transitory computer-readable storage medium storing computer program instructions that when executed by a computer processor, cause the computer processor to: store quantitative expression data from a sample from a subject exhibiting dysregulated host response, the quantitative expression data for at least one biomarker selected from the group consisting of the biomarkers listed in Row 1 of one of Tables 1, 2A, 2B, and 3, at least one biomarker selected from the group consisting of the biomarkers listed in Row 2 of the one of Tables 1, 2A, 2B, and 3, and at least one biomarker selected from the group consisting of the biomarkers listed in Row 3 of the one of Tables 1, 2A, 2B, and 3; and determine a classification of the subject based on the quantitative expression data using a patient subtype classifier, the classification of the subject comprising one of subtype A, subtype B, or subtype C.

Additionally disclosed herein is a non-transitory computer-readable storage medium storing computer program instructions that when executed by a computer processor, cause the computer processor to: obtain a classification of a subject exhibiting dysregulated host response, the classification of the subject comprising one of subtype A, subtype B, or subtype C; and identify a therapy recommendation for the subject based at least in part on the classification, wherein responsive to the classification of the subject comprising subtype A, the therapy recommendation identified for the subject comprises at least no immunosuppressive therapy, wherein responsive to the classification of the subject comprising subtype B, the therapy recommendation identified for the subject comprises at least one of no therapy recommendation, immune stimulation therapy, suppression of immune regulation therapy, blocking of immune suppression therapy, blocking of complement activity therapy, and anti-inflammatory therapy, and wherein responsive to the classification of the subject comprising subtype C, the therapy recommendation identified for the subject comprises at least one of no therapy recommendation, immune stimulation therapy, suppression of immune regulation therapy, blocking of immune suppression therapy, modulators of coagulation therapy, and modulators of vascular permeability therapy.

Additionally disclosed herein is a system comprising: a storage memory for storing quantitative expression data from a sample from a subject exhibiting dysregulated host response, the quantitative expression data for at least one biomarker selected from the group consisting of the biomarkers listed in Row 1 of one of Tables 1, 2A, 2B, and 3, at least one biomarker selected from the group consisting of the biomarkers listed in Row 2 of the one of Tables 1, 2A, 2B, and 3, and at least one biomarker selected from the group consisting of the biomarkers listed in Row 3 of the one of Tables 1, 2A, 2B, and 3; and a processor communicatively coupled to the storage memory for determining a classification of the subject based on the quantitative expression data using a patient subtype classifier, the classification of the subject comprising one of subtype A, subtype B, or subtype C.

Additionally disclosed herein is a system comprising: a processor for: obtaining a classification of a subject exhibiting dysregulated host response, the classification of the subject comprising one of subtype A, subtype B, or subtype C; and identifying a therapy recommendation for the subject based at least in part on the classification, wherein responsive to the classification of the subject comprising subtype A, the therapy recommendation identified for the subject comprises at least no immunosuppressive therapy, wherein responsive to the classification of the subject comprising subtype B, the therapy recommendation identified for the subject comprises at least one of no therapy recommendation, immune stimulation therapy, suppression of immune regulation therapy, blocking of immune suppression therapy, blocking of complement activity therapy, and anti-inflammatory therapy, and wherein responsive to the classification of the subject comprising subtype C, the therapy recommendation identified for the subject comprises at least one of no therapy recommendation, immune stimulation therapy, suppression of immune regulation therapy, blocking of immune suppression therapy, modulators of coagulation therapy, and modulators of vascular permeability therapy.

Additionally disclosed herein is a kit comprising: a plurality of reagents for determining, from a sample obtained from a subject exhibiting dysregulated host response, quantitative expression data for at least one biomarker selected from the group consisting of the biomarkers listed in Row 1 of one of Tables 1, 2A, 2B, and 3, at least one biomarker selected from the group consisting of the biomarkers listed in Row 2 of the one of Tables 1, 2A, 2B, and 3, and at least one biomarker selected from the group consisting of the biomarkers listed in Row 3 of the one of Tables 1, 2A, 2B, and 3; and instructions for using the plurality of reagents to determine the quantitative expression data from the sample from the subject.

Additionally disclosed herein is a composition comprising at least three primer sets for amplifying at least three biomarkers, wherein each primer set of the at least three primer sets comprises a pair of single-stranded DNA primers for amplifying one of the at least three biomarkers, and wherein at least one of the at least three biomarkers is selected from the group consisting of the biomarkers listed in Row 1 of one of Tables 1, 2A, 2B, and 3, at least one biomarker of the at least three biomarkers is selected from the group consisting of the biomarkers listed in Row 2 of the one of Tables 1, 2A, 2B, and 3, and at least one biomarker of the at least three biomarkers is selected from the group consisting of the biomarkers listed in Row 3 of the one of Tables 1, 2A, 2B, and 3.

In various embodiments, the one of Tables 1, 2A, 2B, and 3 comprises Table 1, wherein the at least one of the at least three primer sets is selected from the group consisting of: a forward primer comprising at least 15 contiguous nucleotides of SEQ ID NO. 7 and a reverse primer comprising at least 15 contiguous nucleotides of SEQ ID NO. 8, a forward primer comprising at least 15 contiguous nucleotides of SEQ ID NO. 9 and a reverse primer comprising at least 15 contiguous nucleotides of SEQ ID NO. 10, a forward primer comprising at least 15 contiguous nucleotides of SEQ ID NO. 11 and a reverse primer comprising at least 15 contiguous nucleotides of SEQ ID NO. 12, and a forward primer comprising at least 15 contiguous nucleotides of SEQ ID NO. 13 and a reverse primer comprising at least 15 contiguous nucleotides of SEQ ID NO. 14, wherein at least one of the at least three primer sets is selected from the group consisting of: a forward primer comprising at least 15 contiguous nucleotides of SEQ ID NO. 15 and a reverse primer comprising at least 15 contiguous nucleotides of SEQ ID NO. 16, a forward primer comprising at least 15 contiguous nucleotides of SEQ ID NO. 17 and a reverse primer comprising at least 15 contiguous nucleotides of SEQ ID NO. 18, and a forward primer comprising at least 15 contiguous nucleotides of SEQ ID NO. 19 and a reverse primer comprising at least 15 contiguous nucleotides of SEQ ID NO. 20, and wherein at least one of the at least three primer sets is selected from the group consisting of: a forward primer comprising at least 15 contiguous nucleotides of SEQ ID NO. 1 and a reverse primer comprising at least 15 contiguous nucleotides of SEQ ID NO. 2; a forward primer comprising at least 15 contiguous nucleotides of SEQ ID NO. 3 and a reverse primer comprising at least 15 contiguous nucleotides of SEQ ID NO. 4, and a forward primer comprising at least 15 contiguous nucleotides of SEQ ID NO. 5 and a reverse primer comprising at least 15 contiguous nucleotides of SEQ ID NO. 6.

In various embodiments, the one of Tables 1, 2A, 2B, and 3 comprises Table 1, wherein at least one of the at least three primer sets is selected from the group consisting of: a forward primer comprising SEQ ID NO. 7 and a reverse primer comprising SEQ ID NO. 8, a forward primer comprising SEQ ID NO. 9 and a reverse primer comprising SEQ ID NO. 10, a forward primer comprising SEQ ID NO. 11 and a reverse primer comprising SEQ ID NO. 12, and a forward primer comprising SEQ ID NO. 13 and a reverse primer comprising SEQ ID NO. 14, wherein at least one of the at least three primer sets is selected from the group consisting of: a forward primer comprising SEQ ID NO. 15 and a reverse primer comprising SEQ ID NO. 16, a forward primer comprising SEQ ID NO. 17 and a reverse primer comprising SEQ ID NO. 18, and a forward primer comprising SEQ ID NO. 19 and a reverse primer comprising SEQ ID NO. 20, and wherein at least one of the at least three primer sets is selected from the group consisting of: a forward primer comprising SEQ ID NO. 1 and a reverse primer comprising SEQ ID NO. 2; a forward primer comprising SEQ ID NO. 3 and a reverse primer comprising SEQ ID NO. 4, and a forward primer comprising SEQ ID NO. 5 and a reverse primer comprising SEQ ID NO. 6.

In various embodiments, the one of Tables 1, 2A, 2B, and 3 comprises Table 2B, wherein the at least one of the at least three primer sets is selected from the group consisting of: a forward primer comprising at least 15 contiguous nucleotides of SEQ ID NO. 21 and a reverse primer comprising at least 15 contiguous nucleotides of SEQ ID NO. 22, and a forward primer comprising at least 15 contiguous nucleotides of SEQ ID NO. 23 and a reverse primer comprising at least 15 contiguous nucleotides of SEQ ID NO. 24, wherein at least one of the at least three primer sets is selected from the group consisting of: a forward primer comprising at least 15 contiguous nucleotides of SEQ ID NO. 25 and a reverse primer comprising at least 15 contiguous nucleotides of SEQ ID NO. 26, and a forward primer comprising at least 15 contiguous nucleotides of SEQ ID NO. 29 and a reverse primer comprising at least 15 contiguous nucleotides of SEQ ID NO. 30, and wherein at least one of the at least three primer sets is selected from the group consisting of: a forward primer comprising at least 15 contiguous nucleotides of SEQ ID NO. 25 and a reverse primer comprising at least 15 contiguous nucleotides of SEQ ID NO. 26, and a forward primer comprising at least 15 contiguous nucleotides of SEQ ID NO. 27 and a reverse primer comprising at least 15 contiguous nucleotides of SEQ ID NO. 28.

In various embodiments, the one of Tables 1, 2A, 2B, and 3 comprises Table 2B, wherein at least one of the at least three primer sets is selected from the group consisting of: a forward primer comprising SEQ ID NO. 21 and a reverse primer comprising SEQ ID NO. 22, and a forward primer comprising SEQ ID NO. 23 and a reverse primer comprising SEQ ID NO. 24, wherein at least one of the at least three primer sets is selected from the group consisting of: a forward primer comprising SEQ ID NO. 25 and a reverse primer comprising SEQ ID NO. 26, and a forward primer comprising SEQ ID NO. 29 and a reverse primer comprising SEQ ID NO. 30, and wherein at least one of the at least three primer sets is selected from the group consisting of: a forward primer comprising SEQ ID NO. 25 and a reverse primer comprising SEQ ID NO. 26, and a forward primer comprising SEQ ID NO. 27 and a reverse primer comprising SEQ ID NO. 28.

Additionally disclosed herein is a composition comprising at least three primer sets for amplifying at least three biomarkers, wherein each primer set of the at least three primer sets comprises a forward outer primer, a backward outer primer, a forward inner primer, a backward inner primer, a forward loop primer, and a backward loop primer for amplifying one of the at least three biomarkers, and wherein at least one of the at least three biomarkers is selected from the group consisting of the biomarkers listed in Row 1 of one of Tables 1, 2A, 2B, and 3, at least one biomarker of the at least three biomarkers is selected from the group consisting of the biomarkers listed in Row 2 of the one of Tables 1, 2A, 2B, and 3, and at least one biomarker of the at least three biomarkers is selected from the group consisting of the biomarkers listed in Row 3 of the one of Tables 1, 2A, 2B, and 3.

In various embodiments, at least one of the at least three primer sets is selected from the group consisting of: a forward outer primer configured to enable amplification of the at least one biomarker listed in Row 1 of Tables 1, 2A, 2B, and 3, a backward outer primer configured to enable amplification of the at least one biomarker listed in Row 1 of Tables 1, 2A, 2B, and 3, a forward inner primer configured to enable amplification of the at least one biomarker listed in Row 1 of Tables 1, 2A, 2B, and 3, a backward inner primer configured to enable amplification of the at least one biomarker listed in Row 1 of Tables 1, 2A, 2B, and 3, a forward loop primer configured to enable amplification of the at least one biomarker listed in Row 1 of Tables 1, 2A, 2B, and 3, and a backward loop primer configured to enable amplification of the at least one biomarker listed in Row 1 of Tables 1, 2A, 2B, and 3, wherein at least one of the at least three primer sets is selected from the group consisting of: a forward outer primer configured to enable amplification of the at least one biomarker listed in Row 2 of Tables 1, 2A, 2B, and 3, a backward outer primer configured to enable amplification of the at least one biomarker listed in Row 2 of Tables 1, 2A, 2B, and 3, a forward inner primer configured to enable amplification of the at least one biomarker listed in Row 2 of Tables 1, 2A, 2B, and 3, a backward inner primer configured to enable amplification of the at least one biomarker listed in Row 2 of Tables 1, 2A, 2B, and 3, a forward loop primer configured to enable amplification of the at least one biomarker listed in Row 2 of Tables 1, 2A, 2B, and 3, and a backward loop primer configured to enable amplification of the at least one biomarker listed in Row 2 of Tables 1, 2A, 2B, and 3, and wherein at least one of the at least three primer sets is selected from the group consisting of: a forward outer primer configured to enable amplification of the at least one biomarker listed in Row 3 of Tables 1, 2A, 2B, and 3, a backward outer primer configured to enable amplification of the at least one biomarker listed in Row 3 of Tables 1, 2A, 2B, and 3, a forward inner primer configured to enable amplification of the at least one biomarker listed in Row 3 of Tables 1, 2A, 2B, and 3, a backward inner primer configured to enable amplification of the at least one biomarker listed in Row 3 of Tables 1, 2A, 2B, and 3, a forward loop primer configured to enable amplification of the at least one biomarker listed in Row 3 of Tables 1, 2A, 2B, and 3, and a backward loop primer configured to enable amplification of the at least one biomarker listed in Row 3 of Tables 1, 2A, 2B, and 3.

In various embodiments, the dysregulated host response comprises one of sepsis and dysregulated host response not caused by infection. In various embodiments, methods described above further comprise administering or having administered therapy to the subject based on the therapy recommendation. In various embodiments, responsive to the classification of the subject comprising subtype A, the therapy recommendation identified for the subject further comprises at least no corticosteroid therapy. In various embodiments, responsive to the classification of the subject comprising subtype B, the therapy recommendation identified for the subject further comprises at least one of a checkpoint inhibitor, a blocker of complement components, a blocker of complement component receptors, and a blocker of a pro-inflammatory cytokine. In various embodiments, responsive to the classification of the subject comprising subtype C, the therapy recommendation identified for the subject further comprises at least one of a checkpoint inhibitor and an anticoagulant. In various embodiments, responsive to the classification of the subject comprising subtype A, the therapy recommendation identified for the subject further comprises no hydrocortisone.

In various embodiments, responsive to the classification of the subject comprising subtype B, the therapy recommendation identified for the subject further comprises at least one of anti-PD-1, anti-PD-L1, anti-CLTA-4, anti-CEACAM-1, anti-TIM-3, IL-7, anti-C5a, anti-C3a, anti-C5aR, anti-C3aR, anti-TNF-alpha, and anti-IL-6. In various embodiments, responsive to the classification of the subject comprising subtype C, the therapy recommendation identified for the subject further comprises at least one of anti-PD-1, anti-PD-L1, anti-CLTA-4, anti-CEACAM-1, anti-TIM-3, IL-7, activated protein C, antithrombin, and thrombomodulin. In various embodiments, the classification is pre-determined. In various embodiments, the method further comprises determining the classification, and wherein determining the classification comprises: obtaining a sample from the subject exhibiting dysregulated host response, wherein the sample comprises a plurality of biomarkers; determining quantitative expression data for at least three biomarkers; and determining the classification of the subject based on the quantitative expression data using a patient subtype classifier.

In various embodiments, the at least three biomarkers comprise at least one biomarker selected from the group consisting of the biomarkers listed in Row 1 of one of Tables 1, 2A, 2B, and 3, at least one biomarker selected from the group consisting of the biomarkers listed in Row 2 of the one of Tables 1, 2A, 2B, and 3, and at least one biomarker selected from the group consisting of the biomarkers listed in Row 3 of the one of Tables 1, 2A, 2B, and 3. In various embodiments, the obtained sample comprises a blood sample from the subject. In various embodiments, the method further comprises determining that the subject exhibiting dysregulated host response does not exhibit shock, and wherein the one of Tables 1, 2A, 2B, and 3 comprises one of Tables 1, 2B, and 4. In various embodiments, the method further comprises determining that the subject exhibiting dysregulated host response is further exhibiting shock, and wherein the one of Tables 1, 2A, 2B, and 3 comprises one of Tables 1, 2A, and 3. In various embodiments, the method further comprises determining that the subject exhibiting dysregulated host response is an adult subject, and wherein the one of Tables 1, 2A, 2B, and 3 comprises one of Tables 1, 2A, and 2B. In various embodiments, the method further comprises determining that the subject exhibiting dysregulated host response is a pediatric subject, and wherein the one of Tables 1, 2A, 2B, and 3 comprises one of Tables 1 and 3.

In various embodiments, the quantitative expression data for at least one of the at least three biomarkers is determined by one of RT-qPCR (quantitative reverse transcription polymerase chain reaction), qPCR (quantitative polymerase chain reaction), PCR (polymerase chain reaction), RT-PCR (reverse transcription polymerase chain reaction), SDA (strand displacement amplification), RPA (recombinase polymerase amplification), MDA (multiple displacement amplification), HDA (helicase dependent amplification), LAMP (loop-mediated isothermal amplification), RCA (rolling circle amplification), NASBA (nucleic acid-sequence-based amplification), and any other isothermal or thermocycled amplification reaction.

In various embodiments, determining the quantitative expression data for each of the at least three biomarkers comprises: contacting the sample with a reagent; generating a plurality of complexes between the reagent and the plurality of biomarkers in the sample; and detecting the plurality of complexes to obtain a dataset associated with the sample, wherein the dataset comprises the quantitative expression data for the biomarker.

In various embodiments, determining a classification of the subject based on the quantitative expression data using a patient subtype classifier comprises: determining, by the patient subtype classifier, for each candidate classification of the subject, a classification-specific score for the subject by: determining a first geometric mean of the quantitative expression data for the subject for one or more biomarkers of the candidate classification, wherein the quantitative expression data for the subject for the one or more biomarkers of the candidate classification are increased relative to the quantitative expression data for the one or more biomarkers for one or more control subjects; determining a second geometric mean of the quantitative expression for the subject for one or more additional biomarkers of the candidate classification, wherein the quantitative expression data for the subject for the one or more additional biomarkers of the candidate classification are decreased relative to the quantitative expression data for the one or more additional biomarkers for the one or more control subjects; and determining a difference between the first geometric mean and the second geometric mean, the first and second geometric means optionally subject to scaling, and the difference comprising the classification-specific score for the subject; and determining, by the patient subtype classifier, using a multi-class regression model, based on the classification-specific score for each candidate classification of the subject, the classification of the subject, wherein the candidate classifications of the subject comprise subtype A, subtype B, and subtype C.

In various embodiments, the method further comprises prior to determining a classification of the subject based on the quantitative expression data using a patient subtype classifier, normalizing the quantitative expression data based on quantitative expression data for one or more housekeeping genes.

In various embodiments, the patient subtype classifier is a machine-learned model. In various embodiments, the one of Tables 1, 2A, 2B, and 3 comprises Table 1, and wherein the patient subtype classifier has an average accuracy of at least 82.93%. In various embodiments, the one of Tables 1, 2A, 2B, and 3 comprises Table 2A, and wherein the patient subtype classifier has an average accuracy of at least 89.6%. In various embodiments, the one of Tables 1, 2A, 2B, and 3 comprises Table 2B, and wherein the patient subtype classifier has an average accuracy of at least 86.3%. In various embodiments, the one of Tables 1, 2A, 2B, and 3 comprises Table 3, and wherein the patient subtype classifier has an average accuracy of at least 98.3%.

In various embodiments, the therapy recommendation identified for the subject further comprises corticosteroid therapy, no corticosteroid therapy, or no therapy recommendation, identifying that the therapy recommendation for the subject comprises at least no corticosteroid therapy comprises determining that a statistical significance of an average day 28 reduction in mortality of subjects exhibiting dysregulated host response, determined based on the one of Tables 1, 2A, 2B, and 3 to be of the determined classification of the subject, and not provided corticosteroid therapy, is greater than or equal to a threshold statistical significance, identifying that the therapy recommendation for the subject comprises corticosteroid therapy comprises determining that a statistical significance of an average day 28 reduction in mortality of subjects exhibiting dysregulated host response, determined based on the one of Tables 1, 2A, 2B, and 3 to be of the determined classification of the subject, and provided corticosteroid therapy, is greater than or equal to a threshold statistical significance, and identifying that the therapy recommendation for the subject comprises no therapy recommendation comprises: determining that a statistical significance of an average day 28 reduction in mortality of subjects exhibiting dysregulated host response, determined based on the one of Tables 1, 2A, 2B, and 3 to be of the determined classification of the subject, and not provided corticosteroid therapy, is less than a threshold statistical significance; and determining that a statistical significance of an average day 28 reduction in mortality of subjects exhibiting dysregulated host response, determined based on the one of Tables 1, 2A, 2B, and 3 to be of the determined classification of the subject, and provided corticosteroid therapy, is less than a threshold statistical significance.

In various embodiments, a statistical significance comprises a p-value, and wherein the threshold statistical significance comprises at least 0.1. In various embodiments, the therapy recommendation identified for the subject further comprises corticosteroid therapy, no corticosteroid therapy, or no therapy recommendation, wherein the dysregulated host response comprises sepsis, wherein the one of Tables 1, 2A, 2B, and 3 comprises Table 1, wherein identifying that the therapy recommendation for the subject comprises no corticosteroid therapy comprises determining that the classification of the subject comprises subtype A or subtype C, and wherein identifying that the therapy recommendation for the subject comprises corticosteroid therapy comprises determining that the classification of the subject comprises subtype B.

In various embodiments, the therapy recommendation identified for the subject further comprises corticosteroid therapy, no corticosteroid therapy, or no therapy recommendation, wherein the dysregulated host response comprises dysregulated host response not caused by infection, wherein the one of Tables 1, 2A, 2B, and 3 comprises Table 1 or Table 3, wherein identifying that the therapy recommendation for the subject comprises no corticosteroid therapy comprises determining that the classification of the subject comprises subtype A or subtype C, and wherein identifying that the therapy recommendation for the subject comprises no therapy recommendation comprises determining that the classification of the subject comprises subtype B.

In various embodiments, the therapy recommendation identified for the subject further comprises corticosteroid therapy, no corticosteroid therapy, or no therapy recommendation, wherein the dysregulated host response comprises sepsis, wherein the one of Tables 1, 2A, 2B, and 3 comprises Table 2A, Table 2B or Table 3, wherein identifying that the therapy recommendation for the subject comprises no corticosteroid therapy comprises determining that the classification of the subject comprises subtype A, and wherein identifying that the therapy recommendation for the subject comprises no therapy recommendation comprises determining that the classification of the subject comprises subtype B or subtype C.

In various embodiments, the therapy recommendation identified for the subject further comprises corticosteroid therapy, no corticosteroid therapy, or no therapy recommendation, wherein the dysregulated host response comprises dysregulated host response not caused by infection, wherein the one of Tables 1, 2A, 2B, and 3 comprises Table 2A, wherein identifying that the therapy recommendation for the subject comprises no corticosteroid therapy comprises determining that the classification of the subject comprises subtype C, and wherein identifying that the therapy recommendation for the subject comprises no therapy recommendation comprises determining that the classification of the subject comprises subtype A or subtype B.

In various embodiments, the therapy recommendation identified for the subject further comprises corticosteroid therapy, no corticosteroid therapy, or no therapy recommendation, wherein the dysregulated host response comprises dysregulated host response not caused by infection, wherein the one of Tables 1, 2A, 2B, and 3 comprises Table 2B, wherein identifying that the therapy recommendation for the subject comprises no corticosteroid therapy comprises determining that the classification of the subject comprises subtype A or subtype C, and wherein identifying that the therapy recommendation for the subject comprises corticosteroid therapy comprises determining that the classification of the subject comprises subtype B.

In various embodiments, an average day 28 reduction in mortality of subjects exhibiting dysregulated host response that comprises sepsis, determined to be of subtype A based on Table 1, and not provided corticosteroid therapy, is between 5.0%-64.6%, compared to subjects exhibiting dysregulated host response that comprises sepsis, determined to be of subtype A based on Table 1, and provided corticosteroid therapy. In various embodiments, an average day 28 reduction in mortality of subjects exhibiting dysregulated host response that comprises sepsis, determined to be of subtype A based on Table 2A, and not provided corticosteroid therapy, is between 5.0%-86.0%, compared to subjects exhibiting dysregulated host response that comprises sepsis, determined to be of subtype A based on Table 2A, and provided corticosteroid therapy. In various embodiments, an average day 28 reduction in mortality of subjects exhibiting dysregulated host response that comprises sepsis, determined to be of subtype A based on Table 2B, and not provided corticosteroid therapy, is between 5.0%-86.1%, compared to subjects exhibiting dysregulated host response that comprises sepsis, determined to be of subtype A based on Table 2B, and provided corticosteroid therapy.

In various embodiments, an average day 28 reduction in mortality of subjects exhibiting dysregulated host response that comprises sepsis, determined to be of subtype A based on Table 3, and not provided corticosteroid therapy, is between 5.0%-77.6%, compared to subjects exhibiting dysregulated host response that comprises sepsis, determined to be of subtype A based on Table 3, and provided corticosteroid therapy. In various embodiments, an average day 28 reduction in mortality of subjects exhibiting dysregulated host response that comprises sepsis, determined to be of subtype B based on Table 1, and provided corticosteroid therapy, is between 5.0%-35.2%, compared to subjects exhibiting dysregulated host response that comprises sepsis, determined to be of subtype B based on Table 1, and not provided corticosteroid therapy. In various embodiments, an average day 28 reduction in mortality of subjects exhibiting dysregulated host response that comprises dysregulated host response not caused by infection, determined to be of subtype A or subtype C based on Table 1, and not provided corticosteroid therapy, is between 5.0%-100.0%, compared to subjects exhibiting dysregulated host response that comprises dysregulated host response not caused by infection, determined to be of subtype A or subtype C based on Table 1, and provided corticosteroid therapy. In various embodiments, an average day 28 reduction in mortality of subjects exhibiting dysregulated host response that comprises dysregulated host response not caused by infection, determined to be of subtype A or subtype C based on Table 2A, and not provided corticosteroid therapy, is between 5.0%-56.7%, compared to subjects exhibiting dysregulated host response that comprises dysregulated host response not caused by infection, determined to be of subtype A or subtype C based on Table 2A, and provided corticosteroid therapy.

In various embodiments, an average day 28 reduction in mortality of subjects exhibiting dysregulated host response that comprises dysregulated host response not caused by infection, determined to be of subtype A or subtype C based on Table 2B, and not provided corticosteroid therapy, is between 5.0%-100.0%, compared to subjects exhibiting dysregulated host response that comprises dysregulated host response not caused by infection, determined to be of subtype A or subtype C based on Table 2B, and provided corticosteroid therapy. In various embodiments, an average day 28 reduction in mortality of subjects exhibiting dysregulated host response that comprises dysregulated host response not caused by infection, determined to be of subtype A or C based on Table 3, and not provided corticosteroid therapy, is between 5.0%-100.0%, compared to subjects exhibiting dysregulated host response that comprises dysregulated host response not caused by infection, determined to be of subtype A or C based on Table 3, and provided corticosteroid therapy.

### XII. References

1. Rather A R, Kasana B. The Third International Consensus Definitions for Sepsis and Septic Shock (Sepsis-3) [Internet]. JMS SKIMS. 2015. pp. 162-164. doi:10.33883/jms.v18i2.269
2. Bellani G, Laffey J G, Pham T, Fan E, Brochard L, Esteban A, et al. Epidemiology, Patterns of Care, and Mortality for Patients With Acute Respiratory Distress Syndrome in Intensive Care Units in 50 Countries. JAMA. 2016; 315: 788-800.
3. Liu V, Escobar G J, Greene J D, Soule J, Whippy A, Angus D C, et al. Hospital deaths in patients with sepsis from 2 independent cohorts. JAMA. 2014; 312: 90-92.
4. Marshall J C. Why have clinical trials in sepsis failed? Trends Mol Med. 2014; 20: 195-203.
5. Vincent J-L, Mongkolpun W. Non-antibiotic therapies for sepsis: an update. Expert Rev Anti Infect Ther. 2019; 17: 169-175.
6. Young D, Lamb S E, Shah S, MacKenzie I, Tunnicliffe W, Lall R, et al. High-Frequency Oscillation for Acute Respiratory Distress Syndrome [Internet]. New England Journal of Medicine. 2013. pp. 806-813. doi:10.1056/nejmoa1215716
7. Ferguson N D, Cook D J, Guyatt G H, Mehta S, Hand L, Austin P, et al. High-Frequency Oscillation in Early Acute Respiratory Distress Syndrome [Internet]. New England Journal of Medicine. 2013. pp. 795-805. doi:10.1056/nejmoa1215554
8. Meade M O, Young D, Hanna S, Zhou Q, Bachman T E, Bollen C, et al. Severity of Hypoxemia and Effect of High-Frequency Oscillatory Ventilation in Acute Respiratory Distress Syndrome [Internet]. American Journal of Respiratory and Critical Care Medicine. 2017. pp. 727-733. doi:10.1164/rccm.201609-1938oc
9. Meade M O, Cook D J, Guyatt G H, Slutsky A S, Arabi Y M, James Cooper D, et al. Ventilation Strategy Using Low Tidal Volumes, Recruitment Maneuvers, and High Positive End-Expiratory Pressure for Acute Lung Injury and Acute Respiratory Distress Syndrome [Internet]. JAMA. 2008. p. 637. doi:10.1001/jama.299.6.637
10. Hodgson C, Goligher E C, Young M E, Keating J L, Holland A E, Romero L, et al. Recruitment manoeuvres for adults with acute respiratory distress syndrome receiving mechanical ventilation. Cochrane Database Syst Rev. 2016; 11: CD006667.
11. Walkey A J, Del Sorbo L, Hodgson C L, Adhikari N K J, Wunsch H, Meade M O, et al. Higher PEEP versus Lower PEEP Strategies for Patients with Acute Respiratory Distress Syndrome. A Systematic Review and Meta-Analysis [Internet]. Annals of the American Thoracic Society. 2017. pp. S297-S303. doi:10.1513/annalsats.201704-338ot
12. Investigators WGFTARFARDST (art), Writing Group for the Alveolar Recruitment for Acute Respiratory Distress Syndrome Trial (ART) Investigators, Cavalcanti A B, Suzumura É A, Laranjeira L N, de Moraes Paisani D, et al. Effect of Lung Recruitment and Titrated Positive End-Expiratory Pressure (PEEP) vs Low PEEP on Mortality in Patients With Acute Respiratory Distress Syndrome [Internet]. JAMA. 2017. p. 1335. doi:10.1001/jama.2017.14171
13. Antcliffe D B, Burnham K L, Al-Beidh F, Santhakumaran S, Brett S J, Hinds C J, et al. Transcriptomic Signatures in Sepsis and a Differential Response to Steroids. From the VANISH Randomized Trial [Internet]. American Journal of Respiratory and Critical Care Medicine. 2019. pp. 980-986. doi:10.1164/rccm.201807-1419oc
14. Calfee C S, Delucchi K L, Sinha P, Matthay M A, Hackett J, Shankar-Hari M, et al. Acute respiratory distress syndrome subphenotypes and differential response to simvastatin: secondary analysis of a randomised controlled trial. Lancet Respir Med. 2018; 6: 691-698.
15. Rhee C, Dantes R, Epstein L, Murphy D J, Seymour C W, Iwashyna T J, et al. Incidence and Trends of Sepsis in U S Hospitals Using Clinical vs Claims Data, 2009-2014. JAMA. 2017; 318: 1241-1249.
16. Paoli C J, Reynolds M A, Sinha M, Gitlin M, Crouser E. Epidemiology and Costs of Sepsis in the United States—An Analysis Based on Timing of Diagnosis and Severity Level. Crit Care Med. 2018; 46: 1889-1897.
17. Rhee C, Jones T M, Hamad Y, Pande A, Varon J, O'Brien C, et al. Prevalence, Underlying Causes, and Preventability of Sepsis-Associated Mortality in US Acute Care Hospitals. JAMA Netw Open. 2019; 2: e187571.
18. Levy M M, Evans L E, Rhodes A. The Surviving Sepsis Campaign Bundle: 2018 update [Internet]. Intensive Care Medicine. 2018. pp. 925-928. doi:10.1007/s00134-018-5085-0
19. Jones S L, Ashton C M, Kiehne L B, Nicolas J C, Rose A L, Shirkey B A, et al. Outcomes and Resource Use of Sepsis-associated Stays by Presence on Admission, Severity, and Hospital Type. Med Care. 2016; 54: 303-310.
20. Lamontagne F, Rochwerg B, Lytvyn L, Guyatt G H, Møller M H, Annane D, et al. Corticosteroid therapy for sepsis: a clinical practice guideline. BMJ. 2018; 362: k3284.
21. Hahn E O, Houser H B, Rammelkamp C H Jr, Denny F W, Wannamaker L W. Effect of cortisone on acute streptococcal infections and poststreptococcal complications. J Clin Invest. 1951; 30: 274-281.
22. Schumer W. Steroids in the Treatment of Clinical Septic Shock [Internet]. Annals of Surgery. 1976. pp. 333-341. doi:10.1097/00000658-197609000-00011
23. Sprung C L, Caralis P V, Marcial E H, Pierce M, Celbard M A, Long W N, et al. The Effects of High-Dose Corticosteroids in Patients with Septic Shock, a Prospective, Controlled Study [Internet]. Survey of Anesthesiology. 1985. p. 238. doi:10.1097/00132586-198508000-00028
24. Bone R C, Fisher C J, Clemmer T P, Slotman G J, Metz C A, Balk R A, et al. A Controlled Clinical Trial of High-Dose Methylprednisolone in the Treatment of Severe Sepsis and Septic Shock [Internet]. New England Journal of Medicine. 1987. pp. 653-658. doi:10.1056/nejm198709103171101
25. Bollaert P E, Charpentier C, Levy B, Debouverie M, Audibert G, Larcan A. Reversal of late septic shock with supraphysiologic doses of hydrocortisone. Crit Care Med. 1998; 26: 645-650.
26. Annane, Annane, Bellissant, Sebille, Lesieur, Mathieu, et al. Impaired pressor sensitivity to noradrenaline in septic shock patients with and without impaired adrenal function reserve [Internet]. British Journal of Clinical Pharmacology. 2002. pp. 589-597. doi:10.1046/j.1365-2125.1998.00833.x
27. Rothwell P. Cortisol response to corticotropin and survival in septic shock [Internet]. The Lancet. 1991. pp. 582-583. doi:10.1016/0140-6736(91)91641-7
28. Moran J L, Chapman M J, O'Fathartaigh M S, Peisach A R, Pannall P R, Leppard P. Hypocortisolaemia and adrenocortical responsiveness at onset of septic shock [Internet]. Intensive Care Medicine. 1994. pp. 489-495. doi:10.1007/bf01711901
29. Annane D. A 3-Level Prognostic Classification in Septic Shock Based on Cortisol Levels and Cortisol Response to Corticotropin [Internet]. JAMA. 2000. p. 1038. doi:10.1001/jama.283.8.1038
30. Annane D. Effect of Treatment With Low Doses of Hydrocortisone and Fludrocortisone on Mortality in Patients With Septic Shock [Internet]. JAMA. 2002. p. 862. doi:10.1001/jama.288.7.862
31. Gordon A C, Mason A J, Thirunavukkarasu N, Perkins G D, Cecconi M, Cepkova M, et al. Effect of Early Vasopressin vs Norepinephrine on Kidney Failure in Patients With Septic Shock: The VANISH Randomized Clinical Trial. JAMA. 2016; 316: 509-518.
32. Dellinger R P, The Surviving Sepsis Campaign Guidelines Committee including The Pediatric Subgroup*, Levy M M, Rhodes A, Annane D, Gerlach H, et al. Surviving Sepsis Campaign: International Guidelines for Management of Severe Sepsis and Septic Shock, 2012 [Internet]. Intensive Care Medicine. 2013. pp. 165-228. doi:10.1007/s00134-012-2769-8
33. Rhodes A, Evans L E, Alhazzani W, Levy M M, Antonelli M, Ferrer R, et al. Surviving Sepsis Campaign: International Guidelines for Management of Sepsis and Septic Shock: 2016. Intensive Care Med. 2017; 43: 304-377.
34. Venkatesh B, Finfer S, Cohen J, Rajbhandari D, Arabi Y, Bellomo R, et al. Adjunctive Glucocorticoid Therapy in Patients with Septic Shock. N Engl J Med. 2018; 378: 797-808.
35. Annane D, Renault A, Brun-Buisson C, Megarbane B, Quenot J-P, Siami S, et al. Hydrocortisone plus Fludrocortisone for Adults with Septic Shock [Internet]. New England Journal of Medicine. 2018. pp. 809-818. doi:10.1056/nejmoa1705716
36. Identification numbers for sepsis-related clinical trials currently registered on clinicaltrials.gov as of Jul. 1, 2019: NCT03509350, NCT03401398, NCT02473263, NCT03872011, NCT03592693, NCT02602210, NCT03710187, NCT03333278, NCT03380507, NCT03389555, NCT03422159, NCT03649633, NCT03913468, NCT03649009, NCT01817153, NCT03540628, NCT03821714, NCT03258619, NCT03336814, NCT03748537. In: ClinicalTrials.gov [Internet]. [cited Jul. 1, 2019]. Available: www.clinicaltrials.gov
37. Stress Hydrocortisone In Pediatric Septic Shock—Full Text View—ClinicalTrials.gov [Internet]. [cited 16 Jul. 2019]. Available: https://clinicaltrials.gov/ct2/show/NCT03401398
38. Wong H R, Atkinson S J, Cvijanovich N Z, Anas N, Allen G L, Thomas N J, et al. Combining Prognostic and Predictive Enrichment Strategies to Identify Children With Septic Shock Responsive to Corticosteroids. Crit Care Med. 2016; 44: e1000-3.
39. Hager D N, Hooper M H, Bernard G R, Busse L W, Ely E W, Fowler A A, et al. The Vitamin C, Thiamine and Steroids in Sepsis (VICTAS) Protocol: a prospective, multi-center, double-blind, adaptive sample size, randomized, placebo-controlled, clinical trial. Trials. 2019; 20: 197.
40. Marshall J C, Vincent J-L, Fink M P, Cook D J, Rubenfeld G, Foster D, et al. Measures, markers, and mediators: Toward a staging system for clinical sepsis. A Report of the Fifth Toronto Sepsis Roundtable, Toronto, Ontario, Canada, Oct. 25-26, 2000 [Internet]. Critical Care Medicine. 2003. pp. 1560-1567. doi:10.1097/01.ccm.0000065186.67848.3a
41. Pachot A, Lepape A, Vey S, Bienvenu J, Mougin B, Monneret G. Systemic transcriptional analysis in survivor and non-survivor septic shock patients: a preliminary study. Immunol Lett. 2006; 106: 63-71.
42. Wong H R, Cvijanovich N, Lin R, Allen G L, Thomas N J, Willson D F, et al. Identification of pediatric septic shock subclasses based on genome-wide expression profiling. BMC Med. 2009; 7: 34.
43. Maslove D M, Tang B M, McLean A S. Identification of sepsis subtypes in critically ill adults using gene expression profiling. Crit Care. 2012; 16: R183.
44. Davenport E E, Burnham K L, Radhakrishnan J, Humburg P, Hutton P, Mills T C, et al. Genomic landscape of the individual host response and outcomes in sepsis: a prospective cohort study. Lancet Respir Med. 2016; 4: 259-271.
45. Scicluna B P, van Vught L A, Zwinderman A H, Wiewel M A, Davenport E E, Burnham K L, et al. Classification of patients with sepsis according to blood genomic endotype: a prospective cohort study. Lancet Respir Med. 2017; 5: 816-826.
46. Sweeney T E, Azad T D, Donato M, Haynes W A, Perumal T M, Henao R, et al. Unsupervised Analysis of Transcriptomics in Bacterial Sepsis Across Multiple Datasets Reveals Three Robust Clusters. Crit Care Med. 2018; 46: 915-925.
47. Sweeney T E, Wong H R, Khatri P. Robust classification of bacterial and viral infections via integrated host gene expression diagnostics. Sci Transl Med. 2016; 8: 346ra91.
48. Tusher V G, Tibshirani R, Chu G. Significance analysis of microarrays applied to the ionizing radiation response. Proc Natl Acad Sci USA. 2001; 98: 5116-5121.
49. Sweeney T E, Chen A C, Gevaert O. Combined Mapping of Multiple clUsteriNg ALgorithms (COMMUNAL): A Robust Method for Selection of Cluster Number, K. Sci Rep. 2015; 5: 16971.
50. Plassais, Jonathan, et al. “Transcriptome modulation by hydrocortisone in severe burn shock: ancillary analysis of a prospective randomized trial.” Critical Care, vol. 21, no. 158, 16 Jun. 2017, doi:https://www.ncbi.nlm.nih.gov/pmc/articles/PMC5473974/.
51. Timothy, Sweeney E., et al. “A comprehensive time-course-based multicohort analysis of sepsis and sterile inflammation reveals a robust diagnostic gene set.” Science Translational Medicine, vol. 7, no. 287, 13 May 2015, doi:https://stm.sciencemag.org/content/7/287/287ra71.
52. Balk, Robert A. “Systemic inflammatory response syndrome (SIRS) Where did it come from and is it still relevant today?” Virulence, vol. 5, no. 1, 13 Nov. 2013, pp. 20-26, doi:https://www.ncbi.nlm.nih.gov/pmc/articles/PMC3916374/.
53. Benjamini, Y., and Hochberg, Y. (1995). Controlling the false discovery rate: a practical and powerful approach to multiple testing. Journal of the Royal Statistical Society Series B, 57, 289-300. http://www.jstor.org/stable/2346101.

What is claimed is:

1. A method for determining a patient subtype, the method comprising:
obtaining or having obtained quantitative data for at least one biomarker set selected from the group consisting of the biomarker sets of group 1, group 2, group 3, group 4, or group 5,
wherein group 1 comprises biomarker 1, biomarker 2, and biomarker 3,
wherein biomarker 1 is one of EVL, BTN3A2, HLA-DPA1, IDH3A, ACBD3, EXOSC10, SNRK, or MMP8,
wherein biomarker 2 is one of SERPINB1 or GSPT1, and
wherein biomarker 3 is one of MPP1, HMBS, TAL1, C9orf78, POLR2L, SLC27A3, BTN3A2, DDX50, FCHSD2, GSTK1, UBE2E1, TNFRSF1A, PRPF3, or TOMM70A,
wherein group 2 comprises biomarker 4, biomarker 5, and biomarker 6,
wherein biomarker 4 is one of ZNF831, MME, CD3G, or STOM,
wherein biomarker 5 is one of ECSIT, LAT, or NCOA4, and
wherein biomarker 6 is one of SLC1A5, IGF2BP2, or ANXA3,
wherein group 3 comprises biomarker 7, biomarker 8, and biomarker 9,
wherein biomarker 7 is one of C14orf159 or PUM2,
wherein biomarker 8 is one of EPB42 or RPS6KA5, and
wherein biomarker 9 is one of EPB42 or GBP2; and
wherein group 4 comprises biomarker 10, biomarker 11, and biomarker 12,
wherein biomarker 10 is one of MSH2, DCTD, or MMP8,
wherein biomarker 11 is one of HK3, UCP2, or NUP88, and
wherein biomarker 12 is one of GABARAPL2 or CASP4; and
wherein group 5 comprises biomarker 13, biomarker 14, and biomarker 15,
wherein biomarker 13 is one of STOM, MME, BNT3A2, HLA-DPA1, ZNF831, or CD3G,
wherein biomarker 14 is one of EPB42, GSPT1, LAT, HK3, or SERPINB1, and
wherein biomarker 15 is one of SLC1A5, IGF2BP2, ANXA3, GBP2, TNFRSF1, BTN3A2, OR TNFRSF1A; and
determining a classification of a subject based on the quantitative data using a patient subtype classifier.

2. The method of claim 1, wherein the at least one biomarker set is group 5, and wherein biomarker 13 is one of STOM, MME, BNT3A2, or HLA-DPA1.

3. The method of claim 1 or 2, wherein the at least one biomarker set is group 5, and wherein biomarker 14 is one of EPB42, GSPT1, LAT, HK3, or SERPINB1.

4. The method of any one of claims 1-3, wherein the at least one biomarker set is group 5, and wherein biomarker 15 is one of SLC1A5, IGF2BP2, or ANXA3.

5. A method for determining a therapy recommendation for a patient, the method comprising:
obtaining or having obtained quantitative data for two or more biomarkers selected from the group consisting of EVL, BTN3A2, HLA-DPA1, IDH3A, ACBD3, EXOSC10, SNRK, MMP8, SERPINB1, GSPT1, MPP1, HMBS, TAL1, C9orf78, POLR2L, SLC27A3, DDX50, FCHSD2, GSTK1, UBE2E1, TNFRSF1A, PRPF3, TOMM70A, ZNF831, MME, CD3G, STOM, ECSIT, LAT, NCOA4, SLC1A5, IGF2BP2, ANXA3, C14orf159, PUM2, EPB42, RPS6KA5, GBP2, MSH2, DCTD, HK3, UCP2, NUP88, GABARAPL2, and CASP4; and
determining a classification of a subject based on the quantitative data using a patient subtype classifier.

6. A method for determining a therapy recommendation for a patient, the method comprising:
obtaining or having obtained quantitative data for at least one biomarker set selected from the group consisting of the biomarker sets of group 1, group 2, group 3, group 4, or group 5,
wherein group 1 comprises two or more biomarkers selected from a group consisting of EVL, BTN3A2, HLA-DPA1, IDH3A, ACBD3, EXOSC10, SNRK, MMP8, SERPINB1 GSPT1, MPP1, HMBS, TALL C9orf78, POLR2L, SLC27A3, BTN3A2, DDX50, FCHSD2, GSTK1, UBE2E1, TNFRSF1A, PRPF3, and TOMM70A,
wherein group 2 comprises two or more biomarkers selected from a group consisting of ZNF831, MME, CD3G, STOM, ECSIT, LAT, NCOA4, SLC1A5, IGF2BP2, and ANXA3,
wherein group 3 comprises two or more biomarkers selected from a group consisting of C14orf159, PUM2, EPB42, RPS6KA5, EPB42, and GBP2; and
wherein group 4 comprises two or more biomarkers selected from a group consisting of MSH2, DCTD, MMP8, HK3, UCP2, NUP88, GABARAPL2, and CASP4; and
wherein group 5 comprises two or more biomarkers selected from a group consisting of STOM, MME, BNT3A2, HLA-DPA1, ZNF831, CD3G, EPB42, GSPT1, LAT, HK3, SERPINB1, SLC1A5, IGF2BP2, ANXA3, GBP2, TNFRSF1, BTN3A2, and TNFRSF1A; and
determining a classification of a subject based on the quantitative data using a patient subtype classifier.

7. The method of any one of claims 1-6, further comprising identifying a therapy recommendation for the subject based at least in part on the classification.

8. A method for determining a therapy recommendation for a patient, the method comprising:
obtaining a classification of a subject exhibiting a dysregulated host response, the classification having been determined by:
obtaining or having obtained quantitative data for at least one biomarker set selected from the group consisting of the biomarker sets of group 1, group 2, group 3, group 4, or group 5,
wherein group 1 comprises biomarker 1, biomarker 2, and biomarker 3,
wherein biomarker 1 is one of EVL, BTN3A2, HLA-DPA1, IDH3A, ACBD3, EXOSC10, SNRK, or MMP8,
wherein biomarker 2 is one of SERPINB1 or GSPT1, and
wherein biomarker 3 is one of MPP1, HMBS, TAL1, C9orf78, POLR2L, SLC27A3, BTN3A2, DDX50, FCHSD2, GSTK1, UBE2E1, TNFRSF1A, PRPF3, or TOMM70A,
wherein group 2 comprises biomarker 4, biomarker 5, and biomarker 6,
wherein biomarker 4 is one of ZNF831, MME, CD3G, or STOM,
wherein biomarker 5 is one of ECSIT, LAT, or NCOA4, and
wherein biomarker 6 is one of SLC1A5, IGF2BP2, or ANXA3,
wherein group 3 comprises biomarker 7, biomarker 8, and biomarker 9,
wherein biomarker 7 is one of C14orf159 or PUM2,
wherein biomarker 8 is one of EPB42 or RPS6KA5, and
wherein biomarker 9 is one of EPB42 or GBP2; and
wherein group 4 comprises biomarker 10, biomarker 11, and biomarker 12,
wherein biomarker 10 is one of MSH2, DCTD, or MMP8,
wherein biomarker 11 is one of HK3, UCP2, or NUP88, and
wherein biomarker 12 is one of GABARAPL2 or CASP4; and
wherein group 5 comprises biomarker 13, biomarker 14, and biomarker 15,
wherein biomarker 13 is one of STOM, MME, BNT3A2, HLA-DPA1, ZNF831, or CD3G,
wherein biomarker 14 is one of EPB42, GSPT1, LAT, HK3, or SERPINB1, and
wherein biomarker 15 is one of SLC1A5, IGF2BP2, ANXA3, GBP2, TNFRSF1, BTN3A2, OR TNFRSF1A; and
determining the classification based on the quantitative data using a patient subtype classifier; and
identifying a therapy recommendation for the subject based at least in part on the classification.

9. The method of claim 8, wherein the dysregulated host response of the subject comprises one of sepsis and dysregulated host response not caused by infection.

10. The method of any one of claims 1-9, wherein the classification of the subject comprises one of subtype A or subtype B.

11. The method of any one of claims 1-9, wherein the classification of the subject comprises one of subtype A, subtype B, or subtype C.

12. The method of claim 10 or 11, wherein responsive to the classification of the subject comprising subtype A, the therapy recommendation identified for the subject comprises at least no immunosuppressive therapy.

13. The method of claim 10 or 11, wherein responsive to the classification of the subject comprising subtype A, the therapy recommendation identified for the subject further comprises at least no corticosteroid therapy.

14. The method of claim 13, wherein the therapy recommendation identified for the subject further comprises at least one of no hydrocortisone.

15. The method of claim 10 or 11, wherein responsive to the classification of the subject comprising subtype B, the therapy recommendation identified for the subject comprises at least one of no therapy recommendation, immune stimulation therapy, suppression of immune regulation therapy, blocking of immune suppression therapy, blocking of complement activity therapy, and anti-inflammatory therapy.

16. The method of claim 10 or 11, wherein responsive to the classification of the subject comprising subtype B, the therapy recommendation identified for the subject further comprises at least one of a checkpoint inhibitor, a blocker of complement components, a blocker of complement component receptors, and a blocker of a pro-inflammatory cytokine.

17. The method of claim 16, wherein the therapy recommendation identified for the subject further comprises at least one of GM-CSF, anti-PD-1, anti-PD-L1, anti-CLTA-4, anti-CEACAM-1, anti-TIM-3, anti-BTLA, IL-7, INF-gamma, IFN-beta 1a regulator, IL-22 agonist, IFN-alpha regulator, IFN-lambda regulator, IFN-alpha 2b stimulant, anti-C5a, anti-C3a, anti-C5aR, anti-C3aR, anti-TNF-alpha, and anti-IL-6, Anti-HMGB1, ST2 antibody, IL-33 antibody.

18. The method of claim 11, wherein responsive to the classification of the subject comprising subtype C, the therapy recommendation identified for the subject comprises at least one of no therapy recommendation, immune stimulation therapy, suppression of immune regulation therapy, blocking of immune suppression therapy, modulators of coagulation therapy, and modulators of vascular permeability therapy.

19. The method of claim 11, wherein responsive to the classification of the subject comprising subtype C, the therapy recommendation identified for the subject further comprises at least one of a checkpoint inhibitor and an anticoagulant.

20. The method of claim 19, wherein the therapy recommendation identified for the subject further comprises at least one of GM-CSF, anti-PD-1, anti-PD-L1, anti-CLTA-4, anti-CEACAM-1, anti-TIM-3, anti-BTLA, IL-7, INF-gamma, IFN-beta 1a regulator, IL-22 agonist, IFN-alpha regulator, IFN-lambda regulator, IFN-alpha 2b stimulant, activated protein C, antithrombin, and thrombomodulin.

21. The method of any one of claims 7-20, further comprising administering or having administered therapy to the subject based on the therapy recommendation.

22. The method of any one of claims 1-21, wherein obtaining or having obtained quantitative data comprises:
obtaining a sample from a subject exhibiting dysregulated host response, wherein the sample comprises a plurality of biomarkers; and
determining the quantitative data from the obtained sample.

23. The method of claim 22, wherein the obtained sample comprises a blood sample from the subject.

24. The method of any one of claim 1 or 7-23, wherein the subject exhibiting dysregulated host response does not exhibit shock, and wherein the at least one biomarker set is one of group 1, group 3, or group 4.

25. The method of any one of claim 1 or 7-23, wherein the subject exhibiting dysregulated host response is further exhibiting shock, and wherein the at least one biomarker set is one of group 1, group 2, group 4, group 5, group 6, group 7, or group 8.

26. The method of any one of claim 1 or 7-23, wherein the subject exhibiting dysregulated host response is an adult subject, and wherein the at least one biomarker set is one of group 1, group 2, group 3, group 5, group 6, group 7, or group 8.

27. The method of any one of claim 1 or 7-23, wherein the subject exhibiting dysregulated host response is a pediatric subject, and wherein the at least one biomarker set is one of group 1, group 4, group 5, group 6, group 7, or group 8.

28. The method of any one of claims 1-27, wherein the quantitative data is determined by one of RT-qPCR (quantitative reverse transcription polymerase chain reaction), qPCR (quantitative polymerase chain reaction), PCR (polymerase chain reaction), RT-PCR (reverse transcription polymerase chain reaction), SDA (strand displacement amplification), RPA (recombinase polymerase amplification), MDA (multiple displacement amplification), HDA (helicase dependent amplification), LAMP (loop-mediated isothermal amplification), RCA (rolling circle amplification), NASBA (nucleic acid-sequence-based amplification), and any other isothermal or thermocycled amplification reaction.

29. The method of any one of claims 1-28, wherein the quantitative data is determined by:
contacting a sample with a reagent;
generating a plurality of complexes between the reagent and the plurality of biomarkers in the sample; and
detecting the plurality of complexes to obtain a dataset associated with the sample, wherein the dataset comprises the quantitative data.

30. The method of any one of claims 1-29, wherein the classification of the subject is determined by:
determining, for at least one candidate classification of the subject, a classification-specific score for the subject;
determining, by the patient subtype classifier, based on the classification-specific score, the classification of the subject.

31. The method of claim 30, wherein determining the classification-specific score comprises:
determining a first subscore of the quantitative data for the subject for one or more biomarkers of the candidate classification, wherein the quantitative data for the subject for the one or more biomarkers of the candidate classification are increased relative to the quantitative data for the one or more biomarkers for one or more control subjects;
determining a second subscore of the quantitative expression for the subject for one or more additional biomarkers of the candidate classification, wherein the quantitative data for the subject for the one or more additional biomarkers of the candidate classification are decreased relative to the quantitative data for the one or more additional biomarkers for the one or more control subjects; and
determining a difference between the first subscore and the second subscore, the first and second geometric subscore optionally subject to scaling, and the difference comprising the classification-specific score for the subject; and

32. The method of claim 31, wherein one or both of the first subscore and the second subscore are geometric means.

33. The method of any one of claims 1-32, wherein the patient subtype classifier is a machine-learned model.

34. The method of claim 33, wherein the machine-learned model is a support vector machine (SVM).

35. The method of claim 34, where the support vector machine receives, as input, one or more classification-specific scores and outputs the classification of the subject.

36. The method of claim 30 or 31, wherein the patient subtype classifier determines the classification of the subject by:
comparing the classification-specific scores to one or more threshold values; and
determining the classification of the subject based on the comparisons.

37. The method of claim 36, wherein at least one of the one or more threshold values is a fixed value.

38. The method of claim 36, wherein at least one of the one or more threshold values is determined using training samples, the at least one threshold value representing a value on a ROC curve nearest to maximum sensitivity or maximum specificity.

39. The method of any one of claims 1-38, further comprising, prior to determining a classification of the subject using a patient subtype classifier, normalizing the quantitative data based on quantitative data for one or more housekeeping genes.

40. The method of any one of claims 30-39, wherein the candidate classifications of the subject comprise subtype A, subtype B, and subtype C.

41. The method of any one of claim 1 or 7-40, wherein the at least one biomarker set is group 1, and wherein the patient subtype classifier has an average accuracy of at least 82.93%.

42. The method of any one of claim 1 or 7-40, wherein the at least one biomarker set is group 2, and wherein the patient subtype classifier has an average accuracy of at least 89.6%.

43. The method of any one of claim 1 or 7-40, wherein the at least one biomarker set is group 3, and wherein the patient subtype classifier has an average accuracy of at least 86.3%.

44. The method of any one of claim 1 or 7-40, wherein the at least one biomarker set is group 4, and wherein the patient subtype classifier has an average accuracy of at least 98.3%.

45. The method of claim 7 or 8, wherein:
the therapy recommendation identified for the subject further comprises corticosteroid therapy, no corticosteroid therapy, or no therapy recommendation.

46. The method of claim 45, wherein the therapy recommendation comprises a no corticosteroid therapy, wherein the no corticosteroid therapy is identified by determining that a statistical significance of a reduction in mortality of subjects exhibiting dysregulated host response not provided corticosteroid therapy is greater than or equal to a threshold statistical significance.

47. The method of claim 45, wherein the therapy recommendation comprises a no corticosteroid therapy, wherein the no corticosteroid therapy is identified by determining that the classification of the subject comprises a subtype likely to be adversely responsive to corticosteroid therapy.

48. The method of claim 47, wherein the subtype is subtype A or subtype C.

49. The method of claim 45, wherein the therapy recommendation comprises a corticosteroid therapy, wherein the corticosteroid therapy is identified by determining that a statistical significance of a reduction in mortality of subjects exhibiting dysregulated host response and provided corticosteroid therapy is greater than or equal to a threshold statistical significance.

50. The method of claim 45, wherein the therapy recommendation comprises a corticosteroid therapy, wherein the corticosteroid therapy is identified by determining that the classification of the subject comprises a subtype likely to be favorably responsive to corticosteroid therapy.

51. The method of claim 50, wherein the subtype is subtype B.

52. The method of claim 45, wherein the therapy recommendation identified for the subject comprises a no therapy recommendation, wherein the no therapy recommendation is identified at least by:
determining that a statistical significance of a reduction in mortality of subjects exhibiting dysregulated host response and not provided corticosteroid therapy is less than a threshold statistical significance; and
determining that a statistical significance of a reduction in mortality of subjects exhibiting dysregulated host response and provided corticosteroid therapy is less than a threshold statistical significance.

53. The method of any one of claims 46-52, wherein a statistical significance comprises a p-value, and wherein the threshold statistical significance comprises at least 0.1.

54. The method of claim 45, wherein the therapy recommendation identified for the subject comprises a no corticosteroid therapy, wherein the dysregulated host response comprises dysregulated host response not caused by infection, and wherein the at least one biomarker set is group 1 or group 4.

55. The method of claim 54, wherein the no corticosteroid therapy is identified by determining that the classification of the subject comprises a subtype likely to be adversely responsive to corticosteroid therapy.

56. The method of claim 55, wherein the subtype is subtype A or subtype C.

57. The method of claim 45, wherein the therapy recommendation identified for the subject further comprises no therapy recommendation, wherein the no therapy recommendation is identified by determining that the classification of the subject comprises subtype B.

58. The method of claim 45, wherein the therapy recommendation identified for the subject comprises a no corticosteroid therapy, wherein the dysregulated host response comprises sepsis, wherein the at least one biomarker set is one of group 2, group 3, or group 4.

59. The method of claim 58, wherein the no corticosteroid therapy is identified by determining that the classification of the subject comprises a subtype likely to be adversely responsive to corticosteroid therapy.

60. The method of claim 59, wherein the subtype is subtype A

61. The method of claim 45, wherein the therapy recommendation identified for the subject further comprises a no therapy recommendation, wherein the no therapy recommendation is identified by determining that the classification of the subject comprises a subtype likely to be non-responsive to corticosteroid therapy.

62. The method of claim 61, wherein the subtype is subtype B or subtype C.

63. The method of claim 45, wherein the therapy recommendation identified for the subject further comprises a no corticosteroid therapy, wherein the dysregulated host response comprises dysregulated host response not caused by infection, and wherein the at least one biomarker set is group 2.

64. The method of claim 63, wherein the no corticosteroid therapy is identified by determining that the classification of the subject comprises a subtype like to be adversely responsive to corticosteroid therapy.

65. The method of claim 64, wherein the subtype is subtype C.

66. The method of claim 45, wherein the therapy recommendation identified for the subject further comprises a no therapy recommendation, wherein the no therapy recommendation is identified by determining that the classification of the subject comprises a subtype like to be non-responsive to corticosteroid therapy.

67. The method of claim 66, wherein the subtype is subtype A or subtype B.

68. The method of claim 45, wherein the therapy recommendation identified for the subject comprises a no corticosteroid therapy, wherein the dysregulated host response comprises dysregulated host response not caused by infection, and wherein the at least one biomarker set is group 3.

69. The method of claim 68, wherein the no corticosteroid therapy is identified by determining that the classification of the subject comprises a subtype like to be non-responsive to corticosteroid therapy.

70. The method of claim 69, wherein the subtype is subtype A or subtype C.

71. The method of claim 45, wherein the therapy recommendation identified for the subject further comprises corticosteroid therapy, wherein the corticosteroid therapy is identified by determining that the classification of the subject comprises a subtype likely to be responsive to corticosteroid therapy.

72. The method of claim 71, wherein the subtype is subtype B.

73. A method for identifying a candidate therapeutic, the method comprising:
accessing a differentially expressed gene database comprising gene level fold changes between patients of different subtypes;
determining at least a threshold number of genes are differentially expressed in patients of a first subtype in comparison to patients of a second subtype, wherein each of the differentially expressed genes is involved in a common biological pathway; and
determining a candidate therapeutic likely to be effective for patients of the first subtype, wherein the candidate therapeutic is effective in modulating expression of at least one of the genes that are differentially expressed in patients of the first subtype.

74. The method of claim 73, wherein the differentially expressed gene database is generated by:
obtaining labeled patient data, wherein labels of the labeled patient data identify patients that are classified into one of two or more subtypes;
generating the differentially expressed gene database for at least one or more genes by at least determining gene-level fold changes between patient data with a label indicating a first subtype and patient data with a label indicating a second subtype.

75. The method of claim 73 or 74, wherein the labels of the labeled patient data are generated by applying a clustering analysis or by applying a patient subtype classifier.

76. The method of any one of claims 73-75, wherein at least the threshold number of genes is at least three genes, at least four genes, at least five genes, at least six genes, at least seven genes, at least eight genes, at least nine genes, or at least ten genes.

77. The method of any one of claims 73-76, wherein determining a candidate therapeutic for patients of the first subtype further comprises:
analyzing one or both of:
therapeutic pharmacology data comprising data for the candidate therapeutic; and
host response pathobiology comprising data for patients of the first subtype.

78. A non-transitory computer readable medium for determining a patient subtype, the non-transitory computer readable medium comprising instructions that, when executed by a processor, cause the processor to:
obtain quantitative data for at least one biomarker set selected from the group consisting of the biomarker sets of group 1, group 2, group 3, group 4, or group 5,
wherein group 1 comprises biomarker 1, biomarker 2, and biomarker 3,
wherein biomarker 1 is one of EVL, BTN3A2, HLA-DPA1, IDH3A, ACBD3, EXOSC10, SNRK, or MMP8,
wherein biomarker 2 is one of SERPINB1 or GSPT1, and
wherein biomarker 3 is one of MPP1, HMBS, TAL1, C9orf78, POLR2L, SLC27A3, BTN3A2, DDX50, FCHSD2, GSTK1, UBE2E1, TNFRSF1A, PRPF3, or TOMM70A,
wherein group 2 comprises biomarker 4, biomarker 5, and biomarker 6,
wherein biomarker 4 is one of ZNF831, MME, CD3G, or STOM,
wherein biomarker 5 is one of ECSIT, LAT, or NCOA4, and
wherein biomarker 6 is one of SLC1A5, IGF2BP2, or ANXA3,
wherein group 3 comprises biomarker 7, biomarker 8, and biomarker 9,
wherein biomarker 7 is one of C14orf159 or PUM2,
wherein biomarker 8 is one of EPB42 or RPS6KA5, and
wherein biomarker 9 is one of EPB42 or GBP2; and
wherein group 4 comprises biomarker 10, biomarker 11, and biomarker 12,
wherein biomarker 10 is one of MSH2, DCTD, or MMP8,
wherein biomarker 11 is one of HK3, UCP2, or NUP88, and
wherein biomarker 12 is one of GABARAPL2 or CASP4; and
wherein group 5 comprises biomarker 13, biomarker 14, and biomarker 15,
wherein biomarker 13 is one of STOM, MME, BNT3A2, HLA-DPA1, ZNF831, or CD3G,
wherein biomarker 14 is one of EPB42, GSPT1, LAT, HK3, or SERPINB1, and
wherein biomarker 15 is one of SLC1A5, IGF2BP2, ANXA3, GBP2, TNFRSF1, BTN3A2, OR TNFRSF1A; and
determine a classification of a subject based on the quantitative data using a patient subtype classifier.

79. The non-transitory computer readable medium of claim 78, wherein the at least one biomarker set is group 5, and wherein biomarker 13 is one of STOM, MME, BNT3A2, or HLA-DPA1.

80. The non-transitory computer readable medium of claim 78 or 79, wherein the at least one biomarker set is group 5, and wherein biomarker 14 is one of EPB42, GSPT1, LAT, HK3, or SERPINB1.

81. The non-transitory computer readable medium of any one of claims 78-80, wherein the at least one biomarker set is group 5, and wherein biomarker 15 is one of SLC1A5, IGF2BP2, or ANXA3.

82. A non-transitory computer readable medium for determining a therapy recommendation for a patient, the non-transitory computer readable medium comprising instructions that, when executed by a processor, cause the processor to:
obtain quantitative data for two or more biomarkers selected from the group consisting of EVL, BTN3A2, HLA-DPA1, IDH3A, ACBD3, EXOSC10, SNRK, MMP8, SERPINB1, GSPT1, MPP1, HMBS, TAL1, C9orf78, POLR2L, SLC27A3, DDX50, FCHSD2, GSTK1, UBE2E1, TNFRSF1A, PRPF3, TOMM70A, ZNF831, MME, CD3G, STOM, ECSIT, LAT, NCOA4, SLC1A5, IGF2BP2, ANXA3, C14orf159, PUM2, EPB42, RPS6KA5, GBP2, MSH2, DCTD, HK3, UCP2, NUP88, GABARAPL2, and CASP4; and
determine a classification of a subject based on the quantitative data using a patient subtype classifier.

83. A non-transitory computer readable medium for determining a therapy recommendation for a patient, the non-transitory computer readable medium comprising instructions that, when executed by a processor, cause the processor to:
obtain quantitative data for at least one biomarker set selected from the group consisting of the biomarker sets of group 1, group 2, group 3, group 4, or group 5,
wherein group 1 comprises two or more biomarkers selected from a group consisting of EVL, BTN3A2, HLA-DPA1, IDH3A, ACBD3, EXOSC10, SNRK, MMP8, SERPINB1 GSPT1, MPP1, HMBS, TALL C9orf78, POLR2L, SLC27A3, BTN3A2, DDX50, FCHSD2, GSTK1, UBE2E1, TNFRSF1A, PRPF3, and TOMM70A,
wherein group 2 comprises two or more biomarkers selected from a group consisting of ZNF831, MME, CD3G, STOM, ECSIT, LAT, NCOA4, SLC1A5, IGF2BP2, and ANXA3,
wherein group 3 comprises two or more biomarkers selected from a group consisting of C14orf159, PUM2, EPB42, RPS6KA5, EPB42, and GBP2; and
wherein group 4 comprises two or more biomarkers selected from a group consisting of MSH2, DCTD, MMP8, HK3, UCP2, NUP88, GABARAPL2, and CASP4; and
wherein group 5 comprises two or more biomarkers selected from a group consisting of STOM, MME, BNT3A2, HLA-DPA1, ZNF831, CD3G, EPB42, GSPT1, LAT, HK3, SERPINB1, SLC1A5, IGF2BP2, ANXA3, GBP2, TNFRSF1, BTN3A2, and TNFRSF1A; and
determine a classification of a subject based on the quantitative data using a patient subtype classifier.

84. The non-transitory computer readable medium of any one of claims 78-83, further comprising instructions that, when executed by the processor, cause the processor to identify a therapy recommendation for the subject based at least in part on the classification.

85. A non-transitory computer readable medium for determining a therapy recommendation for a subject, the non-transitory computer readable medium comprising instructions that, when executed by a processor, cause the processor to:
obtain a classification of the subject exhibiting a dysregulated host response, the classification having been determined by:
obtaining or having obtained quantitative data for at least one biomarker set selected from the group consisting of the biomarker sets of group 1, group 2, group 3, group 4, or group 5,
wherein group 1 comprises biomarker 1, biomarker 2, and biomarker 3,
wherein biomarker 1 is one of EVL, BTN3A2, HLA-DPA1, IDH3A, ACBD3, EXOSC10, SNRK, or MMP8,
wherein biomarker 2 is one of SERPINB1 or GSPT1, and
wherein biomarker 3 is one of MPP1, HMBS, TAL1, C9orf78, POLR2L, SLC27A3, BTN3A2, DDX50, FCHSD2, GSTK1, UBE2E1, TNFRSF1A, PRPF3, or TOMM70A,
wherein group 2 comprises biomarker 4, biomarker 5, and biomarker 6,
wherein biomarker 4 is one of ZNF831, MME, CD3G, or STOM,
wherein biomarker 5 is one of ECSIT, LAT, or NCOA4, and
wherein biomarker 6 is one of SLC1A5, IGF2BP2, or ANXA3,
wherein group 3 comprises biomarker 7, biomarker 8, and biomarker 9,
wherein biomarker 7 is one of C14orf159 or PUM2,
wherein biomarker 8 is one of EPB42 or RPS6KA5, and
wherein biomarker 9 is one of EPB42 or GBP2; and
wherein group 4 comprises biomarker 10, biomarker 11, and biomarker 12,
wherein biomarker 10 is one of MSH2, DCTD, or MMP8,
wherein biomarker 11 is one of HK3, UCP2, or NUP88, and
wherein biomarker 12 is one of GABARAPL2 or CASP4; and
wherein group 5 comprises biomarker 13, biomarker 14, and biomarker 15,
wherein biomarker 13 is one of STOM, MME, BNT3A2, HLA-DPA1, ZNF831, or CD3G,
wherein biomarker 14 is one of EPB42, GSPT1, LAT, HK3, or SERPINB1, and
wherein biomarker 15 is one of SLC1A5, IGF2BP2, ANXA3, GBP2, TNFRSF1, BTN3A2, OR TNFRSF1A; and
determining the classification based on the quantitative data using a patient subtype classifier; and
identify a therapy recommendation for the subject based at least in part on the classification.

86. The non-transitory computer readable medium of claim 85, wherein the dysregulated host response of the subject comprises one of sepsis and dysregulated host response not caused by infection.

87. The non-transitory computer readable medium of any one of claims 78-86, wherein the classification of the subject comprises one of subtype A or subtype B.

88. The non-transitory computer readable medium of any one of claims 78-86, wherein the classification of the subject comprises one of subtype A, subtype B, or subtype C.

89. The non-transitory computer readable medium of claim 87 or 88, wherein responsive to the classification of the subject comprising subtype A, the therapy recommendation identified for the subject comprises at least no immunosuppressive therapy.

90. The non-transitory computer readable medium of claim 87 or 88, wherein responsive to the classification of the subject comprising subtype A, the therapy recommendation identified for the subject further comprises at least no corticosteroid therapy.

91. The non-transitory computer readable medium of claim 90, wherein the therapy recommendation identified for the subject further comprises at least one of no hydrocortisone.

92. The non-transitory computer readable medium of claim 87 or 88, wherein responsive to the classification of the subject comprising subtype B, the therapy recommendation identified for the subject comprises at least one of no therapy recommendation, immune stimulation therapy, suppression of immune regulation therapy, blocking of immune suppression therapy, blocking of complement activity therapy, and anti-inflammatory therapy.

93. The non-transitory computer readable medium of claim 87 or 88, wherein responsive to the classification of the subject comprising subtype B, the therapy recommendation identified for the subject further comprises at least one of a checkpoint inhibitor, a blocker of complement components, a blocker of complement component receptors, and a blocker of a pro-inflammatory cytokine.

94. The non-transitory computer readable medium of claim 93, wherein the therapy recommendation identified for the subject further comprises at least one of GM-CSF, anti-PD-1, anti-PD-L1, anti-CLTA-4, anti-CEACAM-1, anti-TIM-3, anti-BTLA, IL-7, INF-gamma, IFN-beta 1a regulator, IL-22 agonist, IFN-alpha regulator, IFN-lambda regulator, IFN-alpha 2b stimulant, anti-C5a, anti-C3a, anti-C5aR, anti-C3aR, anti-TNF-alpha, and anti-IL-6, Anti-HMGB1, ST2 antibody, IL-33 antibody.

95. The non-transitory computer readable medium of claim 88, wherein responsive to the classification of the subject comprising subtype C, the therapy recommendation identified for the subject comprises at least one of no therapy recommendation, immune stimulation therapy, suppression of immune regulation therapy, blocking of immune suppression therapy, modulators of coagulation therapy, and modulators of vascular permeability therapy.

96. The non-transitory computer readable medium of claim 88, wherein responsive to the classification of the subject comprising subtype C, the therapy recommendation identified for the subject further comprises at least one of a checkpoint inhibitor and an anticoagulant.

97. The non-transitory computer readable medium of claim 96, wherein the therapy recommendation identified for the subject further comprises at least one of GM-CSF, anti-PD-1, anti-PD-L1, anti-CLTA-4, anti-CEACAM-1, anti-TIM-3, anti-BTLA, IL-7, INF-gamma, IFN-beta 1a regulator, IL-22 agonist, IFN-alpha regulator, IFN-lambda regulator, IFN-alpha 2b stimulant, activated protein C, antithrombin, and thrombomodulin.

98. The non-transitory computer readable medium of any one of claims 78-97, wherein the instructions that cause the processor to obtain quantitative data further comprises instructions that, when executed by the processor, cause the processor to:
obtain a sample from a subject exhibiting dysregulated host response, wherein the sample comprises a plurality of biomarkers; and
determine the quantitative data from the obtained sample.

99. The non-transitory computer readable medium of claim 98, wherein the obtained sample comprises a blood sample from the subject.

100. The non-transitory computer readable medium of any one of claim 78 or 84-99, wherein the subject exhibiting dysregulated host response does not exhibit shock, and wherein the at least one biomarker set is one of group 1, group 3, or group 4.

101. The non-transitory computer readable medium of any one of claim 78 or 84-99, wherein the subject exhibiting dysregulated host response is further exhibiting shock, and wherein the at least one biomarker set is one of group 1, group 2, group 4, group 5, group 6, group 7, or group 8.

102. The non-transitory computer readable medium of any one of claim 78 or 84-99, wherein the subject exhibiting dysregulated host response is an adult subject, and wherein the at least one biomarker set is one of group 1, group 2, group 3, group 5, group 6, group 7, or group 8.

103. The non-transitory computer readable medium of any one of claim 78 or 84-99, wherein the subject exhibiting dysregulated host response is a pediatric subject, and wherein the at least one biomarker set is one of group 1, group 4, group 5, group 6, group 7, or group 8.

104. The non-transitory computer readable medium of any one of claims 78-103, wherein the quantitative data is determined by one of RT-qPCR (quantitative reverse transcription polymerase chain reaction), qPCR (quantitative polymerase chain reaction), PCR (polymerase chain reaction), RT-PCR (reverse transcription polymerase chain reaction), SDA (strand displacement amplification), RPA (recombinase polymerase amplification), MDA (multiple displacement amplification), HDA (helicase dependent amplification), LAMP (loop-mediated isothermal amplification), RCA (rolling circle amplification), NASBA (nucleic acid-sequence-based amplification), and any other isothermal or thermocycled amplification reaction.

105. The non-transitory computer readable medium of any one of claims 78-104, wherein the quantitative data is determined by:
contacting a sample with a reagent;
generating a plurality of complexes between the reagent and the plurality of biomarkers in the sample; and
detecting the plurality of complexes to obtain a dataset associated with the sample, wherein the dataset comprises the quantitative data.

106. The non-transitory computer readable medium of any one of claims 78-105, wherein the classification of the subject is determined by:
determining, for at least one candidate classification of the subject, a classification-specific score for the subject;
determining, by the patient subtype classifier, based on the classification-specific score, the classification of the subject.

107. The non-transitory computer readable medium of claim 106, wherein the instructions that cause the processor to determine the classification-specific score further comprises instructions that, when executed by the processor, cause the processor to:
determine a first subscore of the quantitative data for the subject for one or more biomarkers of the candidate classification, wherein the quantitative data for the subject for the one or more biomarkers of the candidate classification are increased relative to the quantitative data for the one or more biomarkers for one or more control subjects;
determine a second subscore of the quantitative expression for the subject for one or more additional biomarkers of the candidate classification, wherein the quantitative data for the subject for the one or more additional biomarkers of the candidate classification are decreased relative to the quantitative data for the one or more additional biomarkers for the one or more control subjects; and
determine a difference between the first subscore and the second subscore, the first and second geometric subscore optionally subject to scaling, and the difference comprising the classification-specific score for the subject; and

108. The non-transitory computer readable medium of claim 107, wherein one or both of the first subscore and the second subscore are geometric means.

109. The non-transitory computer readable medium of any one of claims 78-108, wherein the patient subtype classifier is a machine-learned model.

110. The non-transitory computer readable medium of claim 109, wherein the machine-learned model is a support vector machine (SVM).

111. The non-transitory computer readable medium of claim 110, where the support vector machine receives, as input, one or more classification-specific scores and outputs the classification of the subject.

112. The non-transitory computer readable medium of claim 110 or 111, wherein the patient subtype classifier determines the classification of the subject by:
comparing the classification-specific scores to one or more threshold values; and
determining the classification of the subject based on the comparisons.

113. The non-transitory computer readable medium of claim 112, wherein at least one of the one or more threshold values is a fixed value.

114. The non-transitory computer readable medium of claim 112, wherein at least one of the one or more threshold values is determined using training samples, the at least one threshold value representing a value on a ROC curve nearest to maximum sensitivity or maximum specificity.

115. The non-transitory computer readable medium of any one of claims 78-114, further comprising, prior to determining a classification of the subject using a patient subtype classifier, normalizing the quantitative data based on quantitative data for one or more housekeeping genes.

116. The non-transitory computer readable medium of any one of claims 106-115, wherein the candidate classifications of the subject comprise subtype A, subtype B, and subtype C.

117. The non-transitory computer readable medium of any one of claim 78 or 85-116, wherein the at least one biomarker set is group 1, and wherein the patient subtype classifier has an average accuracy of at least 82.93%.

118. The non-transitory computer readable medium of any one of claim 78 or 85-116, and wherein the patient subtype classifier has an average accuracy of at least 89.6%.

119. The non-transitory computer readable medium of any one of claim 78 or 85-116, and wherein the patient subtype classifier has an average accuracy of at least 86.3%.

120. The non-transitory computer readable medium of any one of claim 78 or 85-116, wherein the at least one biomarker set is group 4, and wherein the patient subtype classifier has an average accuracy of at least 98.3%.

121. The non-transitory computer readable medium of claim 84 or 85, wherein:
the therapy recommendation identified for the subject further comprises corticosteroid therapy, no corticosteroid therapy, or no therapy recommendation.

122. The non-transitory computer readable medium of claim 121, wherein the therapy recommendation comprises a no corticosteroid therapy, wherein the no corticosteroid therapy is identified by determining that a statistical significance of a reduction in mortality of subjects exhibiting dysregulated host response not provided corticosteroid therapy is greater than or equal to a threshold statistical significance.

123. The non-transitory computer readable medium of claim 121, wherein the therapy recommendation comprises a no corticosteroid therapy, wherein the no corticosteroid therapy is identified by determining that the classification of the subject comprises a subtype likely to be adversely responsive to corticosteroid therapy.

124. The non-transitory computer readable medium of claim 123, wherein the subtype is subtype A or subtype C.

125. The non-transitory computer readable medium of claim 121, wherein the therapy recommendation comprises a corticosteroid therapy, wherein the corticosteroid therapy is identified by determining that a statistical significance of a reduction in mortality of subjects exhibiting dysregulated host response and provided corticosteroid therapy is greater than or equal to a threshold statistical significance.

126. The non-transitory computer readable medium of claim 121, wherein the therapy recommendation comprises a corticosteroid therapy, wherein the corticosteroid therapy is identified by determining that the classification of the subject comprises a subtype likely to be favorably responsive to corticosteroid therapy.

127. The non-transitory computer readable medium of claim 126, wherein the subtype is subtype B.

128. The non-transitory computer readable medium of claim 121, wherein the therapy recommendation identified for the subject comprises a no therapy recommendation, wherein the no therapy recommendation is identified at least by:
determining that a statistical significance of a reduction in mortality of subjects exhibiting dysregulated host response and not provided corticosteroid therapy is less than a threshold statistical significance; and
determining that a statistical significance of a reduction in mortality of subjects exhibiting dysregulated host response and provided corticosteroid therapy is less than a threshold statistical significance.

129. The non-transitory computer readable medium of claim 122-128, wherein a statistical significance comprises a p-value, and wherein the threshold statistical significance comprises at least 0.1.

130. The non-transitory computer readable medium of claim 121, wherein the therapy recommendation identified for the subject comprises a no corticosteroid therapy, wherein the dysregulated host response comprises dysregulated host response not caused by infection, and wherein the at least one biomarker set is group 1 or group 4.

131. The non-transitory computer readable medium of claim 130, wherein the no corticosteroid therapy is identified by determining that the classification of the subject comprises a subtype likely to be adversely responsive to corticosteroid therapy.

132. The non-transitory computer readable medium of claim 131, wherein the subtype is subtype A or subtype C.

133. The non-transitory computer readable medium of claim 121, wherein the therapy recommendation identified for the subject further comprises no therapy recommendation, wherein the no therapy recommendation is identified by determining that the classification of the subject comprises subtype B.

134. The non-transitory computer readable medium of claim 121, wherein the therapy recommendation identified for the subject comprises a no corticosteroid therapy, wherein the dysregulated host response comprises sepsis, wherein the at least one biomarker set is one of group 2, group 3, or group 4.

135. The non-transitory computer readable medium of claim 134, wherein the no corticosteroid therapy is identified by determining that the classification of the subject comprises a subtype likely to be adversely responsive to corticosteroid therapy.

136. The non-transitory computer readable medium of claim 135, wherein the subtype is subtype A.

137. The non-transitory computer readable medium of claim 121, wherein the therapy recommendation identified for the subject further comprises a no therapy recommendation, wherein the no therapy recommendation is identified by determining that the classification of the subject comprises a subtype likely to be non-responsive to corticosteroid therapy.

138. The non-transitory computer readable medium of claim 137, wherein the subtype is subtype B or subtype C.

139. The non-transitory computer readable medium of claim 121, wherein the therapy recommendation identified for the subject further comprises a no corticosteroid therapy, wherein the dysregulated host response comprises dysregulated host response not caused by infection, and wherein the at least one biomarker set is group 2.

140. The non-transitory computer readable medium of claim 139, wherein the no corticosteroid therapy is identified by determining that the classification of the subject comprises a subtype like to be adversely responsive to corticosteroid therapy.

141. The non-transitory computer readable medium of claim 140, wherein the subtype is subtype C.

142. The non-transitory computer readable medium of claim 121, wherein the therapy recommendation identified for the subject further comprises a no therapy recommendation, wherein the no therapy recommendation is identified by determining that the classification of the subject comprises a subtype like to be non-responsive to corticosteroid therapy.

143. The non-transitory computer readable medium of claim 142, wherein the subtype is subtype A or subtype B.

144. The non-transitory computer readable medium of claim 121, wherein the therapy recommendation identified for the subject comprises a no corticosteroid therapy, wherein the dysregulated host response comprises dysregulated host response not caused by infection, and wherein the at least one biomarker set is group 3.

145. The non-transitory computer readable medium of claim 144, wherein the no corticosteroid therapy is identified by determining that the classification of the subject comprises a subtype like to be non-responsive to corticosteroid therapy.

146. The non-transitory computer readable medium of claim 145, wherein the subtype is subtype A or subtype C.

147. The non-transitory computer readable medium of claim 121, wherein the therapy recommendation identified for the subject further comprises corticosteroid therapy, wherein the corticosteroid therapy is identified by determining that the classification of the subject comprises a subtype likely to be responsive to corticosteroid therapy.

148. The non-transitory computer readable medium of claim 147, wherein the subtype is subtype B.

149. A non-transitory computer readable medium for identifying a candidate therapeutic, the non-transitory computer readable medium comprising instructions that, when executed by a processor, cause the processor to:
access a differentially expressed gene database comprising gene level fold changes between patients of different subtypes;
determine at least a threshold number of genes are differentially expressed in patients of a first subtype in comparison to patients of a second subtype, wherein each of the differentially expressed genes is involved in a common biological pathway; and
determine a candidate therapeutic likely to be effective for patients of the first subtype, wherein the candidate therapeutic is effective in modulating expression of at least one of the genes that are differentially expressed in patients of the first subtype.

150. The non-transitory computer readable medium of claim 149, wherein the differentially expressed gene database is generated by:
obtaining labeled patient data, wherein labels of the labeled patient data identify patients that are classified into one of two or more subtypes;
generating the differentially expressed gene database for at least one or more genes by at least determining gene-level fold changes between patient data with a label indicating a first subtype and patient data with a label indicating a second subtype.

151. The non-transitory computer readable medium of claim 149 or 150, wherein the labels of the labeled patient data are generated by applying a clustering analysis or by applying a patient subtype classifier.

152. The non-transitory computer readable medium of any one of claims 149-151, wherein at least the threshold number of genes is at least three genes, at least four genes, at least five genes, at least six genes, at least seven genes, at least eight genes, at least nine genes, or at least ten genes.

153. The non-transitory computer readable medium of any one of claims 149-152, wherein determining a candidate therapeutic for patients of the first subtype further comprises:
analyzing one or both of:
therapeutic pharmacology data comprising data for the candidate therapeutic; and
host response pathobiology comprising data for patients of the first subtype.

154. A system for determining a patient subtype, the system comprising:
a set of reagents used for determining quantitative data for at least one biomarker set from a test sample from a subject, the at least one biomarker set selected from the group consisting of the biomarker sets of group 1, group 2, group 3, group 4, or group 5,
wherein group 1 comprises biomarker 1, biomarker 2, and biomarker 3,
wherein biomarker 1 is one of EVL, BTN3A2, HLA-DPA1, IDH3A, ACBD3, EXOSC10, SNRK, or MMP8,
wherein biomarker 2 is one of SERPINB1 or GSPT1, and
wherein biomarker 3 is one of MPP1, HMBS, TALL C9orf78, POLR2L, SLC27A3, BTN3A2, DDX50, FCHSD2, GSTK1, UBE2E1, TNFRSF1A, PRPF3, or TOMM70A,
wherein group 2 comprises biomarker 4, biomarker 5, and biomarker 6,
wherein biomarker 4 is one of ZNF831, MME, CD3G, or STOM,
wherein biomarker 5 is one of ECSIT, LAT, or NCOA4, and
wherein biomarker 6 is one of SLC1A5, IGF2BP2, or ANXA3,
wherein group 3 comprises biomarker 7, biomarker 8, and biomarker 9,
wherein biomarker 7 is one of C14orf159 or PUM2,
wherein biomarker 8 is one of EPB42 or RPS6KA5, and
wherein biomarker 9 is one of EPB42 or GBP2; and
wherein group 4 comprises biomarker 10, biomarker 11, and biomarker 12,
wherein biomarker 10 is one of MSH2, DCTD, or MMP8,
wherein biomarker 11 is one of HK3, UCP2, or NUP88, and
wherein biomarker 12 is one of GABARAPL2 or CASP4; and
wherein group 5 comprises biomarker 13, biomarker 14, and biomarker 15,
wherein biomarker 13 is one of STOM, MME, BNT3A2, HLA-DPA1, ZNF831, or CD3G,
wherein biomarker 14 is one of EPB42, GSPT1, LAT, HK3, or SERPINB1, and
wherein biomarker 15 is one of SLC1A5, IGF2BP2, ANXA3, GBP2, TNFRSF1, BTN3A2, OR TNFRSF1A; and
an apparatus configured to receive a mixture of one or more reagents in the set and the test sample and to measure the quantitative data for the at least one biomarker set from the test sample; and
a computer system communicatively coupled to the apparatus to obtain the quantitative data for the at least one biomarker set and to determine a classification of the subject based on the quantitative data using a patient subtype classifier.

155. The system of claim 154, wherein the at least one biomarker set is group 5, and wherein biomarker 13 is one of STOM, MME, BNT3A2, or HLA-DPA1.

156. The system of claim 154 or 155, wherein the at least one biomarker set is group 5, and wherein biomarker 14 is one of EPB42, GSPT1, LAT, HK3, or SERPINB1.

157. The system of any one of claims 154-156, wherein the at least one biomarker set is group 5, and wherein biomarker 15 is one of SLC1A5, IGF2BP2, or ANXA3.

158. A system for determining a patient subtype, the system comprising:
a set of reagents used for determining quantitative data for two or more biomarkers selected from the group consisting of EVL, BTN3A2, HLA-DPA1, IDH3A, ACBD3, EXOSC10, SNRK, MMP8, SERPINB1, GSPT1, MPP1, HMBS, TAL1, C9orf78, POLR2L, SLC27A3, DDX50, FCHSD2, GSTK1, UBE2E1, TNFRSF1A, PRPF3, TOMM70A, ZNF831, MME, CD3G, STOM, ECSIT, LAT, NCOA4, SLC1A5, IGF2BP2, ANXA3, C14orf159, PUM2, EPB42, RPS6KA5, GBP2, MSH2, DCTD, HK3, UCP2, NUP88, GABARAPL2, and CASP4; and
an apparatus configured to receive a mixture of one or more reagents in the set and the test sample and to measure the quantitative data for the at least one biomarker set from the test sample; and
a computer system communicatively coupled to the apparatus to obtain the quantitative data for the at least one biomarker set and to determine a classification of the subject based on the quantitative data using a patient subtype classifier.

159. A system for determining a patient subtype, the system comprising:
a set of reagents used for determining quantitative data for at least one biomarker set selected from the group consisting of the biomarker sets of group 1, group 2, group 3, group 4, or group 5,
wherein group 1 comprises two or more biomarkers selected from a group consisting of EVL, BTN3A2, HLA-DPA1, IDH3A, ACBD3, EXOSC10, SNRK, MMP8, SERPINB1 GSPT1, MPP1, HMBS, TAL1, C9orf78, POLR2L, SLC27A3, BTN3A2, DDX50, FCHSD2, GSTK1, UBE2E1, TNFRSF1A, PRPF3, and TOMM70A,
wherein group 2 comprises two or more biomarkers selected from a group consisting of ZNF831, MME, CD3G, STOM, ECSIT, LAT, NCOA4, SLC1A5, IGF2BP2, and ANXA3,
wherein group 3 comprises two or more biomarkers selected from a group consisting of C14orf159, PUM2, EPB42, RPS6KA5, EPB42, and GBP2; and
wherein group 4 comprises two or more biomarkers selected from a group consisting of MSH2, DCTD, MMP8, HK3, UCP2, NUP88, GABARAPL2, and CASP4; and
wherein group 5 comprises two or more biomarkers selected from a group consisting of STOM, MME, BNT3A2, HLA-DPA1, ZNF831, CD3G, EPB42, GSPT1, LAT, HK3, SERPINB1, SLC1A5, IGF2BP2, ANXA3, GBP2, TNFRSF1, BTN3A2, and TNFRSF1A; and
an apparatus configured to receive a mixture of one or more reagents in the set and the test sample and to measure the quantitative data for the at least one biomarker set from the test sample; and
a computer system communicatively coupled to the apparatus to obtain the quantitative data for the at least one biomarker set and to determine a classification of the subject based on the quantitative data using a patient subtype classifier.

160. The system of any one of claims 154-159, wherein the computer system is configured to identify a therapy recommendation for the subject based at least in part on the classification.

161. A system for determining a therapy recommendation for a subject, the system comprising:
a computer system configured to:
obtain a classification of the subject exhibiting a dysregulated host response, the classification having been determined by:
obtaining or having obtained quantitative data for at least one biomarker set obtained from the subject, the at least one biomarker set selected from the group consisting of the biomarker sets of group 1, group 2, group 3, group 4, or group 5,
wherein group 1 comprises biomarker 1, biomarker 2, and biomarker 3,
wherein biomarker 1 is one of EVL, BTN3A2, HLA-DPA1, IDH3A, ACBD3, EXOSC10, SNRK, or MMP8,
wherein biomarker 2 is one of SERPINB1 or GSPT1, and
wherein biomarker 3 is one of MPP1, HMBS, TAL1, C9orf78, POLR2L, SLC27A3, BTN3A2, DDX50, FCHSD2, GSTK1, UBE2E1, TNFRSF1A, PRPF3, or TOMM70A,
wherein group 2 comprises biomarker 4, biomarker 5, and biomarker 6,
wherein biomarker 4 is one of ZNF831, MME, CD3G, or STOM,
wherein biomarker 5 is one of ECSIT, LAT, or NCOA4, and
wherein biomarker 6 is one of SLC1A5, IGF2BP2, or ANXA3,
wherein group 3 comprises biomarker 7, biomarker 8, and biomarker 9,
wherein biomarker 7 is one of C14orf159 or PUM2,
wherein biomarker 8 is one of EPB42 or RPS6KA5, and
wherein biomarker 9 is one of EPB42 or GBP2; and
wherein group 4 comprises biomarker 10, biomarker 11, and biomarker 12,
wherein biomarker 10 is one of MSH2, DCTD, or MMP8,
wherein biomarker 11 is one of HK3, UCP2, or NUP88, and
wherein biomarker 12 is one of GABARAPL2 or CASP4; and
wherein group 5 comprises biomarker 13, biomarker 14, and biomarker 15,
wherein biomarker 13 is one of STOM, MME, BNT3A2, HLA-DPA1, ZNF831, or CD3G,
wherein biomarker 14 is one of EPB42, GSPT1, LAT, HK3, or SERPINB1, and
wherein biomarker 15 is one of SLC1A5, IGF2BP2, ANXA3, GBP2, TNFRSF1, BTN3A2, OR TNFRSF1A; and
determine the classification based on the quantitative data using a patient subtype classifier; and
identify a therapy recommendation for the subject based at least in part on the classification.

162. The system of claim 161, wherein the dysregulated host response of the subject comprises one of sepsis and dysregulated host response not caused by infection.

163. The system of any one of claims 154-162, wherein the classification of the subject comprises one of subtype A or subtype B.

164. The system of any one of claims 154-162, wherein the classification of the subject comprises one of subtype A, subtype B, or subtype C.

165. The system of claim 163 or 164, wherein responsive to the classification of the subject comprising subtype A, the therapy recommendation identified for the subject comprises at least no immunosuppressive therapy.

166. The system of claim 163 or 164, wherein responsive to the classification of the subject comprising subtype A, the therapy recommendation identified for the subject further comprises at least no corticosteroid therapy.

167. The system of claim 166, wherein the therapy recommendation identified for the subject further comprises at least one of no hydrocortisone.

168. The system of claim 163 or 164, wherein responsive to the classification of the subject comprising subtype B, the therapy recommendation identified for the subject comprises at least one of no therapy recommendation, immune stimulation therapy, suppression of immune regulation therapy, blocking of immune suppression therapy, blocking of complement activity therapy, and anti-inflammatory therapy.

169. The system of claim 163 or 164, wherein responsive to the classification of the subject comprising subtype B, the therapy recommendation identified for the subject further comprises at least one of a checkpoint inhibitor, a blocker of complement components, a blocker of complement component receptors, and a blocker of a pro-inflammatory cytokine.

170. The system of claim 163, wherein the therapy recommendation identified for the subject further comprises at least one of GM-CSF, anti-PD-1, anti-PD-L1, anti-CLTA-4, anti-CEACAM-1, anti-TIM-3, anti-BTLA, IL-7, INF-gamma, IFN-beta 1a regulator, IL-22 agonist, IFN-alpha regulator, IFN-lambda regulator, IFN-alpha 2b stimulant, anti-C5a, anti-C3a, anti-C5aR, anti-C3aR, anti-TNF-alpha, and anti-IL-6, Anti-HMGB1, ST2 antibody, IL-33 antibody.

171. The system of claim 164, wherein responsive to the classification of the subject comprising subtype C, the therapy recommendation identified for the subject comprises at least one of no therapy recommendation, immune stimulation therapy, suppression of immune regulation therapy, blocking of immune suppression therapy, modulators of coagulation therapy, and modulators of vascular permeability therapy.

172. The system of claim 164, wherein responsive to the classification of the subject comprising subtype C, the therapy recommendation identified for the subject further comprises at least one of a checkpoint inhibitor and an anticoagulant.

173. The system of claim 172, wherein the therapy recommendation identified for the subject further comprises at least one of GM-CSF, anti-PD-1, anti-PD-L1, anti-CLTA-4, anti-CEACAM-1, anti-TIM-3, anti-BTLA, IL-7, INF-gamma, IFN-beta 1a regulator, IL-22 agonist, IFN-alpha regulator, IFN-lambda regulator, IFN-alpha 2b stimulant, activated protein C, antithrombin, and thrombomodulin.

174. The system of any one of claims 154-163, wherein the sample comprises a blood sample from the subject.

175. The system of any one of claim 154 or 161-174, wherein the subject exhibiting dysregulated host response does not exhibit shock, and wherein the at least one biomarker set is one of group 1, group 3, or group 4.

176. The system of any one of claim 154 or 161-174, wherein the subject exhibiting dysregulated host response is further exhibiting shock, and wherein the at least one biomarker set is one of group 1, group 2, group 4, group 5, group 6, group 7, or group 8.

177. The system of any one of claim 154 or 161-174, wherein the subject exhibiting dysregulated host response is an adult subject, and wherein the at least one biomarker set is one of group 1, group 2, group 3, group 5, group 6, group 7, or group 8.

178. The system of any one of claim 154 or 161-174, wherein the subject exhibiting dysregulated host response is a pediatric subject, and wherein the at least one biomarker set is one of group 1, group 4, group 5, group 6, group 7, or group 8.

179. The system of any one of claims 154-178, wherein the quantitative data is determined by one of RT-qPCR (quantitative reverse transcription polymerase chain reaction), qPCR (quantitative polymerase chain reaction), PCR (polymerase chain reaction), RT-PCR (reverse transcription polymerase chain reaction), SDA (strand displacement amplification), RPA (recombinase polymerase amplification), MDA (multiple displacement amplification), HDA (helicase dependent amplification), LAMP (loop-mediated isothermal amplification), RCA (rolling circle amplification), NASBA (nucleic acid-sequence-based amplification), and any other isothermal or thermocycled amplification reaction.

180. The system of any one of claims 154-179, wherein the classification of the subject is determined by:
determining, for at least one candidate classification of the subject, a classification-specific score for the subject;
determining, by the patient subtype classifier, based on the classification-specific score, the classification of the subject.

181. The system of claim 180, wherein determine the classification-specific score further comprises:
determine a first subscore of the quantitative data for the subject for one or more biomarkers of the candidate classification, wherein the quantitative data for the subject for the one or more biomarkers of the candidate classification are increased relative to the quantitative data for the one or more biomarkers for one or more control subjects;
determine a second subscore of the quantitative expression for the subject for one or more additional biomarkers of the candidate classification, wherein the quantitative data for the subject for the one or more additional biomarkers of the candidate classification are decreased relative to the quantitative data for the one or more additional biomarkers for the one or more control subjects; and
determine a difference between the first subscore and the second subscore, the first and second geometric subscore optionally subject to scaling, and the difference comprising the classification-specific score for the subject; and

182. The system of claim 181, wherein one or both of the first subscore and the second subscore are geometric means.

183. The system of any one of claims 154-182, wherein the patient subtype classifier is a machine-learned model.

184. The system of claim 183, wherein the machine-learned model is a support vector machine (SVM).

185. The system of claim 184, where the support vector machine receives, as input, one or more classification-specific scores and outputs the classification of the subject.

186. The system of claim 180 or 181, wherein the patient subtype classifier determines the classification of the subject by:
comparing the classification-specific scores to one or more threshold values; and
determining the classification of the subject based on the comparisons.

187. The system of claim 186, wherein at least one of the one or more threshold values is a fixed value.

188. The system of claim 186, wherein at least one of the one or more threshold values is determined using training samples, the at least one threshold value representing a value on a ROC curve nearest to maximum sensitivity or maximum specificity.

189. The system of any one of claims 154-188, further comprising, prior to determining a classification of the subject using a patient subtype classifier, normalizing the quantitative data based on quantitative data for one or more housekeeping genes.

190. The system of any one of claims 180-189, wherein the candidate classifications of the subject comprise subtype A, subtype B, and subtype C.

191. The system of any one of claim 154 or 161-190, wherein the at least one biomarker set is group 1, and wherein the patient subtype classifier has an average accuracy of at least 82.93%.

192. The system of any one of claim 154 or 161-190, and wherein the patient subtype classifier has an average accuracy of at least 89.6%.

193. The system of any one of claim 154 or 161-190, and wherein the patient subtype classifier has an average accuracy of at least 86.3%.

194. The system of any one of claim 154 or 161-190, wherein the at least one biomarker set is group 4, and wherein the patient subtype classifier has an average accuracy of at least 98.3%.

195. The system of claim 158 or 161, wherein the therapy recommendation identified for the subject further comprises corticosteroid therapy, no corticosteroid therapy, or no therapy recommendation.

196. The system of claim 195, wherein the therapy recommendation comprises a no corticosteroid therapy, wherein the no corticosteroid therapy is identified by determining that a statistical significance of a reduction in mortality of subjects exhibiting dysregulated host response not provided corticosteroid therapy is greater than or equal to a threshold statistical significance.

197. The system of claim 195, wherein the therapy recommendation comprises a no corticosteroid therapy, wherein the no corticosteroid therapy is identified by determining that the classification of the subject comprises a subtype likely to be adversely responsive to corticosteroid therapy.

198. The system of claim 197, wherein the subtype is subtype A or subtype C.

199. The system of claim 195, wherein the therapy recommendation comprises a corticosteroid therapy, wherein the corticosteroid therapy is identified by determining that a statistical significance of a reduction in mortality of subjects exhibiting dysregulated host response and provided corticosteroid therapy is greater than or equal to a threshold statistical significance.

200. The system of claim 195, wherein the therapy recommendation comprises a corticosteroid therapy, wherein the corticosteroid therapy is identified by determining that the classification of the subject comprises a subtype likely to be favorably responsive to corticosteroid therapy.

201. The system of claim 200, wherein the subtype is subtype B.

202. The system of claim 195, wherein the therapy recommendation identified for the subject comprises a no therapy recommendation, wherein the no therapy recommendation is identified at least by:
determining that a statistical significance of a reduction in mortality of subjects exhibiting dysregulated host response and not provided corticosteroid therapy is less than a threshold statistical significance; and
determining that a statistical significance of a reduction in mortality of subjects exhibiting dysregulated host response and provided corticosteroid therapy is less than a threshold statistical significance.

203. The system of any one of claims 196-202, wherein a statistical significance comprises a p-value, and wherein the threshold statistical significance comprises at least 0.1.

204. The system of claim 195, wherein the therapy recommendation identified for the subject comprises a no corticosteroid therapy, wherein the dysregulated host response comprises dysregulated host response not caused by infection, and wherein the at least one biomarker set is group 1 or group 4.

205. The system of claim 204, wherein the no corticosteroid therapy is identified by determining that the classification of the subject comprises a subtype likely to be adversely responsive to corticosteroid therapy.

206. The system of claim 205, wherein the subtype is subtype A or subtype C.

207. The system of claim 195, wherein the therapy recommendation identified for the subject further comprises no therapy recommendation, wherein the no therapy recommendation is identified by determining that the classification of the subject comprises subtype B.

208. The system of claim 195, wherein the therapy recommendation identified for the subject comprises a no corticosteroid therapy, wherein the dysregulated host response comprises sepsis, wherein the at least one biomarker set is one of group 2, group 3, or group 4.

209. The system of claim 208, wherein the no corticosteroid therapy is identified by determining that the classification of the subject comprises a subtype likely to be adversely responsive to corticosteroid therapy.

210. The system of claim 209, wherein the subtype is subtype A.

211. The system of claim 195, wherein the therapy recommendation identified for the subject further comprises a no therapy recommendation, wherein the no therapy recommendation is identified by determining that the classification of the subject comprises a subtype likely to be non-responsive to corticosteroid therapy.

212. The system of claim 211, wherein the subtype is subtype B or subtype C.

213. The system of claim 195, wherein the therapy recommendation identified for the subject further comprises a no corticosteroid therapy, wherein the dysregulated host response comprises dysregulated host response not caused by infection, and wherein the at least one biomarker set is group 2.

214. The system of claim 213, wherein the no corticosteroid therapy is identified by determining that the classification of the subject comprises a subtype like to be adversely responsive to corticosteroid therapy.

215. The system of claim 214, wherein the subtype is subtype C.

216. The system of claim 195, wherein the therapy recommendation identified for the subject further comprises a no therapy recommendation, wherein the no therapy recommendation is identified by determining that the classification of the subject comprises a subtype like to be non-responsive to corticosteroid therapy.

217. The system of claim 216, wherein the subtype is subtype A or subtype B.

218. The system of claim 195, wherein the therapy recommendation identified for the subject comprises a no corticosteroid therapy, wherein the dysregulated host response comprises dysregulated host response not caused by infection, and wherein the at least one biomarker set is group 3.

219. The system of claim 218, wherein the no corticosteroid therapy is identified by determining that the classification of the subject comprises a subtype like to be non-responsive to corticosteroid therapy.

220. The system of claim 219, wherein the subtype is subtype A or subtype C.

221. The system of claim 195, wherein the therapy recommendation identified for the subject further comprises corticosteroid therapy, wherein the corticosteroid therapy is identified by determining that the classification of the subject comprises a subtype likely to be responsive to corticosteroid therapy.

222. The system of claim 221, wherein the subtype is subtype B.

223. A system for identifying a candidate therapeutic, the system comprising:
a storage device storing a differentially expressed gene database comprising gene level fold changes between patients of different subtypes;
a computational device configured to:
access one or more gene level fold changes corresponding to differentially expressed genes in the differentially expressed gene database;
determine at least a threshold number of genes are differentially expressed in patients of a first subtype in comparison to patients of a second subtype, wherein each of the differentially expressed genes is involved in a common biological pathway; and
determine a candidate therapeutic likely to be effective for patients of the first subtype, wherein the candidate therapeutic is effective in modulating expression of at least one of the genes that are differentially expressed in patients of the first subtype.

224. The system of claim 223, wherein the differentially expressed gene database is generated by:
obtaining labeled patient data, wherein labels of the labeled patient data identify patients that are classified into one of two or more subtypes;
generating the differentially expressed gene database for at least one or more genes by at least determining gene-level fold changes between patient data with a label indicating a first subtype and patient data with a label indicating a second subtype.

225. The system of claim 223 or 224, wherein the labels of the labeled patient data are generated by applying a clustering analysis or by applying a patient subtype classifier.

226. The system of any one of claims 223-225, wherein at least the threshold number of genes is at least three genes, at least four genes, at least five genes, at least six genes, at least seven genes, at least eight genes, at least nine genes, or at least ten genes.

227. The system of any one of claims 223-226, wherein determining a candidate therapeutic for patients of the first subtype further comprises:
analyzing one or both of:
therapeutic pharmacology data comprising data for the candidate therapeutic; and
host response pathobiology comprising data for patients of the first subtype.

228. A kit for determining a patient subtype, the kit comprising:
a set of reagents for determining quantitative data for at least one biomarker set from a test sample from a subject, the at least one biomarker set selected from the group consisting of the biomarker sets of group 1, group 2, group 3, group 4, or group 5,
wherein group 1 comprises biomarker 1, biomarker 2, and biomarker 3,
wherein biomarker 1 is one of EVL, BTN3A2, HLA-DPA1, IDH3A, ACBD3, EXOSC10, SNRK, or MMP8,
wherein biomarker 2 is one of SERPINB1 or GSPT1, and
wherein biomarker 3 is one of MPP1, HMBS, TAL1, C9orf78, POLR2L, SLC27A3, BTN3A2, DDX50, FCHSD2, GSTK1, UBE2E1, TNFRSF1A, PRPF3, or TOMM70A,
wherein group 2 comprises biomarker 4, biomarker 5, and biomarker 6,
wherein biomarker 4 is one of ZNF831, MME, CD3G, or STOM,
wherein biomarker 5 is one of ECSIT, LAT, or NCOA4, and
wherein biomarker 6 is one of SLC1A5, IGF2BP2, or ANXA3,
wherein group 3 comprises biomarker 7, biomarker 8, and biomarker 9,
wherein biomarker 7 is one of C14orf159 or PUM2,
wherein biomarker 8 is one of EPB42 or RPS6KA5, and
wherein biomarker 9 is one of EPB42 or GBP2; and
wherein group 4 comprises biomarker 10, biomarker 11, and biomarker 12,
wherein biomarker 10 is one of MSH2, DCTD, or MMP8,
wherein biomarker 11 is one of HK3, UCP2, or NUP88, and
wherein biomarker 12 is one of GABARAPL2 or CASP4; and
wherein group 5 comprises biomarker 13, biomarker 14, and biomarker 15,
wherein biomarker 13 is one of STOM, MME, BNT3A2, HLA-DPA1, ZNF831, or CD3G,
wherein biomarker 14 is one of EPB42, GSPT1, LAT, HK3, or SERPINB1, and
wherein biomarker 15 is one of SLC1A5, IGF2BP2, ANXA3, GBP2, TNFRSF1, BTN3A2, OR TNFRSF1A; and
instructions for using the set of reagents to determine the quantitative data for the at least one biomarker set.

229. The kit of claim 228, wherein the at least one biomarker set is group 5, and wherein biomarker 13 is one of STOM, MME, BNT3A2, or HLA-DPA1.

230. The kit of claim 228 or 229, wherein the at least one biomarker set is group 5, and wherein biomarker 14 is one of EPB42, GSPT1, LAT, HK3, or SERPINB1.

231. The kit of any one of claims 228-230, wherein the at least one biomarker set is group 5, and wherein biomarker 15 is one of SLC1A5, IGF2BP2, or ANXA3.

232. A kit for determining a patient subtype, the kit comprising:
a set of reagents for determining quantitative data for two or more biomarkers selected from the group consisting of EVL, BTN3A2, HLA-DPA1, IDH3A, ACBD3, EXOSC10, SNRK, MMP8, SERPINB1, GSPT1, MPP1, HMBS, TAL1, C9orf78, POLR2L, SLC27A3, DDX50, FCHSD2, GSTK1, UBE2E1, TNFRSF1A, PRPF3, TOMM70A, ZNF831, MME, CD3G, STOM, ECSIT, LAT, NCOA4, SLC1A5, IGF2BP2, ANXA3, C14orf159, PUM2, EPB42, RPS6KA5, GBP2, MSH2, DCTD, HK3, UCP2, NUP88, GABARAPL2, and CASP4; and
instructions for using the set of reagents to determine the quantitative data for the at least one biomarker set.

233. A kit for determining a patient subtype, the kit comprising:
a set of reagents for determining quantitative data for at least one biomarker set selected from the group consisting of the biomarker sets of group 1, group 2, group 3, group 4, or group 5,
wherein group 1 comprises two or more biomarkers selected from a group consisting of EVL, BTN3A2, HLA-DPA1, IDH3A, ACBD3, EXOSC10, SNRK, MMP8, SERPINB1 GSPT1, MPP1, HMBS, TAL1, C9orf78, POLR2L, SLC27A3, BTN3A2, DDX50, FCHSD2, GSTK1, UBE2E1, TNFRSF1A, PRPF3, and TOMM70A,
wherein group 2 comprises two or more biomarkers selected from a group consisting of ZNF831, MME, CD3G, STOM, ECSIT, LAT, NCOA4, SLC1A5, IGF2BP2, and ANXA3,
wherein group 3 comprises two or more biomarkers selected from a group consisting of C14orf159, PUM2, EPB42, RPS6KA5, EPB42, and GBP2; and
wherein group 4 comprises two or more biomarkers selected from a group consisting of MSH2, DCTD, MMP8, HK3, UCP2, NUP88, GABARAPL2, and CASP4; and
wherein group 5 comprises two or more biomarkers selected from a group consisting of STOM, MME, BNT3A2, HLA-DPA1, ZNF831, CD3G, EPB42, GSPT1, LAT, HK3, SERPINB1, SLC1A5, IGF2BP2, ANXA3, GBP2, TNFRSF1, BTN3A2, and TNFRSF1A; and
instructions for using the set of reagents to determine the quantitative data for the at least one biomarker set.

234. The kit of any one of claims 228-233, wherein the instructions comprise instructions for determining the quantitative data by performing one of RT-qPCR (quantitative reverse transcription polymerase chain reaction), qPCR (quantitative polymerase chain reaction), PCR (polymerase chain reaction), RT-PCR (reverse transcription polymerase chain reaction), SDA (strand displacement amplification), RPA (recombinase polymerase amplification), MDA (multiple displacement amplification), HDA (helicase dependent amplification), LAMP (loop-mediated isothermal amplification), RCA (rolling circle amplification), NASBA (nucleic acid-sequence-based amplification), and any other isothermal or thermocycled amplification reaction.

235. The kit of any one of claims 228-234, wherein the set of reagents comprises at least three primer sets for amplifying at least three biomarkers,
wherein the at least three primer sets comprise pairs of single-stranded DNA primers for amplifying the at least three biomarkers, and
wherein at least one of the at least three biomarkers is selected from the group consisting of the biomarkers EVL, BTN3A2, HLA-DPA1, IDH3A, ACBD3, EXOSC10, SNRK, MMP8, ZNF831, MME, CD3G, STOM, C14orf159, PUM2, MSH2, DCTD, BNT3A2, or HLA-DPA1,
at least one biomarker of the at least three biomarkers is selected from the group consisting of the biomarkers SERPINB1, GSPT1, ECSIT, LAT, NCOA4, EPB42, RPS6KA5, HK3, UCP2, or NUP88, and
at least one biomarker of the at least three biomarkers is selected from the group consisting of the biomarkers MPP1, HMBS, TAL1, C9orf78, POLR2L, SLC27A3, BTN3A2, DDX50, FCHSD2, GSTK1, UBE2E1, PRPF3, TOMM70A, EPB42, GABARAPL2, CASP4, SLC1A5, IGF2BP2, ANXA3, GBP2, TNFRSF1, OR TNFRSF1A.

236. The kit of claim 235, wherein the at least one of the at least three primer sets is selected from the group consisting of:
a forward primer comprising at least 15 contiguous nucleotides of SEQ ID NO. 7 and a reverse primer comprising at least 15 contiguous nucleotides of SEQ ID NO. 8,
a forward primer comprising at least 15 contiguous nucleotides of SEQ ID NO. 9 and a reverse primer comprising at least 15 contiguous nucleotides of SEQ ID NO. 10,
a forward primer comprising at least 15 contiguous nucleotides of SEQ ID NO. 11 and a reverse primer comprising at least 15 contiguous nucleotides of SEQ ID NO. 12, and
a forward primer comprising at least 15 contiguous nucleotides of SEQ ID NO. 13 and a reverse primer comprising at least 15 contiguous nucleotides of SEQ ID NO. 14,
wherein at least one of the at least three primer sets is selected from the group consisting of:
a forward primer comprising at least 15 contiguous nucleotides of SEQ ID NO. 15 and a reverse primer comprising at least 15 contiguous nucleotides of SEQ ID NO. 16,
a forward primer comprising at least 15 contiguous nucleotides of SEQ ID NO. 17 and a reverse primer comprising at least 15 contiguous nucleotides of SEQ ID NO. 18, and
a forward primer comprising at least 15 contiguous nucleotides of SEQ ID NO. 19 and a reverse primer comprising at least 15 contiguous nucleotides of SEQ ID NO. 20, and
wherein at least one of the at least three primer sets is selected from the group consisting of:
a forward primer comprising at least 15 contiguous nucleotides of SEQ ID NO. 1 and a reverse primer comprising at least 15 contiguous nucleotides of SEQ ID NO. 2;
a forward primer comprising at least 15 contiguous nucleotides of SEQ ID NO. 3 and a reverse primer comprising at least 15 contiguous nucleotides of SEQ ID NO. 4, and
a forward primer comprising at least 15 contiguous nucleotides of SEQ ID NO. 5 and a reverse primer comprising at least 15 contiguous nucleotides of SEQ ID NO. 6.

237. The kit of claim 235, wherein at least one of the at least three primer sets is selected from the group consisting of:
a forward primer comprising SEQ ID NO. 7 and a reverse primer comprising SEQ ID NO. 8,
a forward primer comprising SEQ ID NO. 9 and a reverse primer comprising SEQ ID NO. 10,
a forward primer comprising SEQ ID NO. 11 and a reverse primer comprising SEQ ID NO. 12, and
a forward primer comprising SEQ ID NO. 13 and a reverse primer comprising SEQ ID NO. 14,
wherein at least one of the at least three primer sets is selected from the group consisting of:
a forward primer comprising SEQ ID NO. 15 and a reverse primer comprising SEQ ID NO. 16,
a forward primer comprising SEQ ID NO. 17 and a reverse primer comprising SEQ ID NO. 18, and
a forward primer comprising SEQ ID NO. 19 and a reverse primer comprising SEQ ID NO. 20, and
wherein at least one of the at least three primer sets is selected from the group consisting of:
a forward primer comprising SEQ ID NO. 1 and a reverse primer comprising SEQ ID NO. 2;
a forward primer comprising SEQ ID NO. 3 and a reverse primer comprising SEQ ID NO. 4, and
a forward primer comprising SEQ ID NO. 5 and a reverse primer comprising SEQ ID NO. 6.

238. The kit of claim 235, wherein the at least one of the at least three primer sets is selected from the group consisting of:
a forward primer comprising at least 15 contiguous nucleotides of SEQ ID NO. 21 and a reverse primer comprising at least 15 contiguous nucleotides of SEQ ID NO. 22, and
a forward primer comprising at least 15 contiguous nucleotides of SEQ ID NO. 23 and a reverse primer comprising at least 15 contiguous nucleotides of SEQ ID NO. 24,
wherein at least one of the at least three primer sets is selected from the group consisting of:
a forward primer comprising at least 15 contiguous nucleotides of SEQ ID NO. 25 and a reverse primer comprising at least 15 contiguous nucleotides of SEQ ID NO. 26, and
a forward primer comprising at least 15 contiguous nucleotides of SEQ ID NO. 29 and a reverse primer comprising at least 15 contiguous nucleotides of SEQ ID NO. 30, and
wherein at least one of the at least three primer sets is selected from the group consisting of:
a forward primer comprising at least 15 contiguous nucleotides of SEQ ID NO. 25 and a reverse primer comprising at least 15 contiguous nucleotides of SEQ ID NO. 26, and
a forward primer comprising at least 15 contiguous nucleotides of SEQ ID NO. 27 and a reverse primer comprising at least 15 contiguous nucleotides of SEQ ID NO. 28.

239. The kit of claim 235, wherein at least one of the at least three primer sets is selected from the group consisting of:
a forward primer comprising SEQ ID NO. 21 and a reverse primer comprising SEQ ID NO. 22, and
a forward primer comprising SEQ ID NO. 23 and a reverse primer comprising SEQ ID NO. 24,
wherein at least one of the at least three primer sets is selected from the group consisting of:
a forward primer comprising SEQ ID NO. 25 and a reverse primer comprising SEQ ID NO. 26, and
a forward primer comprising SEQ ID NO. 29 and a reverse primer comprising SEQ ID NO. 30, and
wherein at least one of the at least three primer sets is selected from the group consisting of:
a forward primer comprising SEQ ID NO. 25 and a reverse primer comprising SEQ ID NO. 26, and
a forward primer comprising SEQ ID NO. 27 and a reverse primer comprising SEQ ID NO. 28.

240. The kit of any one of claims 228-234, wherein the set of reagents comprises at least three primer sets for amplifying at least three biomarkers,
wherein each primer set of the at least three primer sets comprises a forward outer primer, a backward outer primer, a forward inner primer, a backward inner primer, a forward loop primer, and a backward loop primer for amplifying one of the at least three biomarkers, and
wherein at least one of the at least three biomarkers is selected from the group consisting of EVL, BTN3A2, HLA-DPA1, IDH3A, ACBD3, EXOSC10, SNRK, MMP8, ZNF831, MME, CD3G, STOM, C14orf159, PUM2, MSH2, DCTD, BNT3A2, or HLA-DPA1,
at least one biomarker of the at least three biomarkers is selected from the group consisting of SERPINB1, GSPT1, ECSIT, LAT, NCOA4, EPB42, RPS6KA5, HK3, UCP2, or NUP88, and
at least one biomarker of the at least three biomarkers is selected from the group consisting of MPP1, HMBS, TALL C9orf78, POLR2L, SLC27A3, BTN3A2, DDX50, FCHSD2, GSTK1, UBE2E1, PRPF3, TOMM70A, EPB42, GABARAPL2, CASP4, SLC1A5, IGF2BP2, ANXA3, GBP2, TNFRSF1, OR TNFRSF1A.

241. The kit of claim 240, wherein at least one of the at least three primer sets is selected from the group consisting of:
a forward outer primer, a backward outer primer, a forward inner primer, a backward inner primer, a forward loop primer, and a backward loop primer, each of which is configured to enable amplification of at least one biomarker selected from the group consisting of: EVL, BTN3A2, HLA-DPA1, IDH3A, ACBD3, EXOSC10, SNRK, MMP8, ZNF831, MME, CD3G, STOM, C14orf159, PUM2, MSH2, DCTD, BNT3A2, or HLA-DPA1,
a forward outer primer, a backward outer primer, a forward inner primer, a backward inner primer, a forward loop primer, and a backward loop primer, each of which is configured to enable amplification of at least one biomarker selected from the group consisting of: SERPINB1, GSPT1, ECSIT, LAT, NCOA4, EPB42, RPS6KA5, HK3, UCP2, or NUP88, and
a forward outer primer, a backward outer primer, a forward inner primer, a backward inner primer, a forward loop primer, and a backward loop primer, each of which is configured to enable amplification of at least one biomarker selected from the group consisting of: MPP1, HMBS, TAL1, C9orf78, POLR2L, SLC27A3, BTN3A2, DDX50, FCHSD2, GSTK1, UBE2E1, PRPF3, TOMM70A, EPB42, GABARAPL2, CASP4, SLC1A5, IGF2BP2, ANXA3, GBP2, TNFRSF1, OR TNFRSF1A.


SEQUENCE LISTING (42 sequences)

SEQ ID NO: 1 - DNA, 19 bp, Artificial Sequence
  Description of Artificial Sequence Synthetic primer
atcaccatcctggtcacgg

SEQ ID NO: 2 - DNA, 20 bp, Artificial Sequence
  Description of Artificial Sequence Synthetic primer
ccacagccaggatcaaggag

SEQ ID NO: 3 - DNA, 20 bp, Artificial Sequence
  Description of Artificial Sequence Synthetic primer
aagaccgtgaacgaactgca

SEQ ID NO: 4 - DNA, 20 bp, Artificial Sequence
  Description of Artificial Sequence Synthetic primer
tttccctgatcttgcgctgt

SEQ ID NO: 5 - DNA, 20 bp, Artificial Sequence
  Description of Artificial Sequence Synthetic primer
cgagccttgaagggtattgg

SEQ ID NO: 6 - DNA, 22 bp, Artificial Sequence
  Description of Artificial Sequence Synthetic primer
tgttcgaatgtccaaaaggtca

SEQ ID NO: 7 - DNA, 20 bp, Artificial Sequence
  Description of Artificial Sequence Synthetic primer
acctgggtgcgaagaagaag

SEQ ID NO: 8 - DNA, 20 bp, Artificial Sequence
  Description of Artificial Sequence Synthetic primer
ggtgattctgaggtggcaca

SEQ ID NO: 9 - DNA, 20 bp, Artificial Sequence
  Description of Artificial Sequence Synthetic primer
aactttgcacaggtgtggtg

SEQ ID NO: 10 - DNA, 20 bp, Artificial Sequence
  Description of Artificial Sequence Synthetic primer
gcagagttctgcaaagtccc

SEQ ID NO: 11 - DNA, 20 bp, Artificial Sequence
  Description of Artificial Sequence Synthetic primer
gcccctcaaggatcgagaag

SEQ ID NO: 12 - DNA, 20 bp, Artificial Sequence
  Description of Artificial Sequence Synthetic primer
aggaggagaacacctggact

SEQ ID NO: 13 - DNA, 20 bp, Artificial Sequence
  Description of Artificial Sequence Synthetic primer
aaaggtggagcgtgtggaaa

SEQ ID NO: 14 - DNA, 20 bp, Artificial Sequence
  Description of Artificial Sequence Synthetic primer
aagggctgcaggagattcag

SEQ ID NO: 15 - DNA, 20 bp, Artificial Sequence
  Description of Artificial Sequence Synthetic primer
ccggaggagtggaacctcta

SEQ ID NO: 16 - DNA, 20 bp, Artificial Sequence
  Description of Artificial Sequence Synthetic primer
catgcacatggcgaagacag

SEQ ID NO: 17 - DNA, 20 bp, Artificial Sequence
  Description of Artificial Sequence Synthetic primer
tgtgtcccaggaactgcatc

SEQ ID NO: 18 - DNA, 20 bp, Artificial Sequence
  Description of Artificial Sequence Synthetic primer
cagctcctgcagattctcgt

SEQ ID NO: 19 - DNA, 20 bp, Artificial Sequence
  Description of Artificial Sequence Synthetic primer
gggcaacctcagccagttat

SEQ ID NO: 20 - DNA, 20 bp, Artificial Sequence
  Description of Artificial Sequence Synthetic primer
caaactgcagggaggccata

SEQ ID NO: 21 - DNA, 20 bp, Artificial Sequence
  Description of Artificial Sequence Synthetic primer
ccctcccgtcggtcattaag

SEQ ID NO: 22 - DNA, 20 bp, Artificial Sequence
  Description of Artificial Sequence Synthetic primer
ttctggatcatctcggcgtg

SEQ ID NO: 23 - DNA, 21 bp, Artificial Sequence
  Description of Artificial Sequence Synthetic primer
tgcacaagattcgacctcaca

SEQ ID NO: 24 - DNA, 20 bp, Artificial Sequence
  Description of Artificial Sequence Synthetic primer
ggtggtcctccaataggtcc

SEQ ID NO: 25 - DNA, 21 bp, Artificial Sequence
  Description of Artificial Sequence Synthetic primer
tgccatcaagatgccagagaa

SEQ ID NO: 26 - DNA, 20 bp, Artificial Sequence
  Description of Artificial Sequence Synthetic primer
ctctctgtgaatgagccccc

SEQ ID NO: 27 - DNA, 19 bp, Artificial Sequence
  Description of Artificial Sequence Synthetic primer
cagggcccagttaatggca

SEQ ID NO: 28 - DNA, 22 bp, Artificial Sequence
  Description of Artificial Sequence Synthetic primer
ggctccaatgatttgcttctca

SEQ ID NO: 29 - DNA, 20 bp, Artificial Sequence
  Description of Artificial Sequence Synthetic primer
agcaaccttccacgccttta

SEQ ID NO: 30 - DNA, 20 bp, Artificial Sequence
  Description of Artificial Sequence Synthetic primer
actctcactggaactgctgc

SEQ ID NO: 31 - DNA, 20 bp, Artificial Sequence
  Description of Artificial Sequence Synthetic primer
gacttccctcagatgggtcg

SEQ ID NO: 32 - DNA, 21 bp, Artificial Sequence
  Description of Artificial Sequence Synthetic primer
tcacagtattgtgcagggtca

SEQ ID NO: 33 - DNA, 20 bp, Artificial Sequence
  Description of Artificial Sequence Synthetic primer
aagaccgtgaacgaactgca

SEQ ID NO: 34 - DNA, 20 bp, Artificial Sequence
  Description of Artificial Sequence Synthetic primer
tttccctgatcttgcgctgt

SEQ ID NO: 35 - DNA, 20 bp, Artificial Sequence
  Description of Artificial Sequence Synthetic primer
gaacgctctacaagctgcac

SEQ ID NO: 36 - DNA, 20 bp, Artificial Sequence
  Description of Artificial Sequence Synthetic primer
ctctgactgcaggaacgtga

SEQ ID NO: 37 - DNA, 20 bp, Artificial Sequence
  Description of Artificial Sequence Synthetic primer
tcctgctgccggatgacatt

SEQ ID NO: 38 - DNA, 22 bp, Artificial Sequence
  Description of Artificial Sequence Synthetic primer
gtccactcatgcaacttttcca

SEQ ID NO: 39 - DNA, 22 bp, Artificial Sequence
  Description of Artificial Sequence Synthetic primer
gctgacttattggtatcggacg

SEQ ID NO: 40 - DNA, 23 bp, Artificial Sequence
  Description of Artificial Sequence Synthetic primer
cagagcgggaaataagcctaaga

SEQ ID NO: 41 - DNA, 20 bp, Artificial Sequence
  Description of Artificial Sequence Synthetic primer
ccaggggaccctgtgaaata

SEQ ID NO: 42 - DNA, 21 bp, Artificial Sequence
  Description of Artificial Sequence Synthetic primer
agagcttgaagggtcagcaat